SEQUENCE LISTING (2 sequences)

SEQ ID NO: 1 - RNA, 23 nt, Synthetic construct
  source 1..23: mol_type = other RNA; note = Cy3-siRNA sequence / sense strand
  modified_base 22..23: mod_base = OTHER; note = thymine
ttctccgaacgtgtgtcacgttt

SEQ ID NO: 2 - RNA, 21 nt, Synthetic construct
  source 1..21: mol_type = other RNA; note = Cy3-siRNA sequence / antisense strand
  modified_base 20..21: mod_base = OTHER; note = thymine
acgtgacacgttcggagaatt

(19) United States
(12) Patent Application Publication
(10) Pub. No.: US 2025/0090472 A1
(43) Pub. Date: Mar. 20, 2025

(54) LIPID NANOPARTICLE COMPOSITIONS AND USES THEREOF

(71) Applicants: BEIJING JITAI PHARMACEUTICAL TECHNOLOGY CO., LTD., Beijing (CN); HANGZHOU JITAI PHARMACEUTICAL TECHNOLOGY CO., LTD., Hangzhou, Zhejiang (CN)

(72) Inventors: Zhuorui SUN, Beijing (CN); Jiayan LANG, Beijing (CN); Liu YANG, Beijing (CN); Xue WANG, Beijing (CN); Jiani LEI, Beijing (CN); Andong LIU, Beijing (CN); Wenshou WANG, Beijing (CN); Caida LAI, Beijing (CN)

(21) Appl. No.: 18/688,308

(22) PCT Filed: Jan. 9, 2024
(86) PCT No.: PCT/CN2024/071295

§ 371 Date: Feb. 29, 2024

(30) Foreign Application Priority Data
Jan. 10, 2023 (CN) 202310032396.7
Nov. 3, 2023 (WO) PCT/CN2023/129705
Dec. 28, 2023 (CN) 202311831236.5

(51) Int. Cl. A61K 9/51; A61K 31/7088

(52) U.S. Cl. CPC A61K 9/5123; A61K 31/7088

(57) ABSTRACT

The present application relates to lipid nanoparticles having a diameter above certain value, as well as the preparation and uses of such compositions. Lipid nanoparticles presented in the application generally have a diameter of at least 160 nm. Such lipid nanoparticles are useful in the delivery of therapeutic agents, such as nucleic acids, in vivo to none-hepatic organs (e.g., lung) for the treatment or prevention of certain diseases or disorders.

### CROSS-REFERENCE TO RELATED APPLICATIONS

This application claims priority to Chinese Patent Application No. 202310032396.7 filed on Jan. 10, 2023, International Patent Application No. PCT/CN2023/129705 filed on Nov. 3, 2023, and Chinese Patent Application No. 202311831236.5 filed on Dec. 28, 2023, the entirely of each of which is incorporated herein by reference.

### SEQUENCE LISTING

This application is submitted concurrently with a computer readable Sequence Listing in XML file format, the entire content of which is incorporated by reference herein in its entirety. The Sequence Listing XML file submitted is entitled “14783-008-228_SEQLISTING.xml”, was created on Dec. 26, 2023, and is 4,168 bytes in size.

### 1. FIELD

The present application relates to the field of pharmaceutical therapies, particularly lipid nanoparticles. This application also relates to the preparation of said lipid nanoparticles, and the use of said lipid nanoparticles for delivery of biologically active molecules, such as nucleic acids (e.g. mRNA, miRNA, siRNA, saRNA, ASO, DNA) and polypeptides (e.g. antibodies).

### 2. BACKGROUND

In recent years, with the clinic approval of COVID-19 mRNA vaccines, nucleic acid-based drugs, such as messenger ribonucleic acid (mRNA) based drugs, have been rapidly developed. Lipid Nanoparticles (LNPs) are one of the mostly widely used delivery platforms for nucleic acid delivery in vivo. LNPs typically comprise four lipid components, that is ionizable lipid, phospholipid, cholesterol, and polyethylene glycosylated lipid (PEG lipid). These four lipids form stable lipid nanoparticles capable of encapsulating nucleic acids. LNPs can prevent nucleic acids from degradation in vivo, such as by nucleases in the body, and deliver them into cells of interest. Since classic LNPs tend to accumulate in the liver, most LNPs-based delivery systems deliver the nucleic acids to the liver. LNPs targeting non-liver organs, such as the lung, may achieve local enrichment in designated organs, thereby improving therapeutic effect and reducing off-target side-effects. The present application describes such LNPs that deliver nucleic acids to non-liver organs, particularly the lung.

### 3. SUMMARY

In one aspect, provided herein is a lipid nanoparticle for use in delivering or expressing a therapeutic agent in the lung of a subject.

In certain embodiment, provided herein is a lipid nanoparticle for use in delivering or expressing a therapeutic agent in the lung of a subject, wherein the lipid nanoparticle is administered intravenously, intraarterially, or intraperitoneally to the subject, wherein the lipid nanoparticle has a positive surface charge, and wherein the lipid nanoparticle has a diameter of from about 160 nm to about 900 nm. In certain embodiment, the lipid nanoparticle comprises a permanently cationic lipid and an ionizable lipid.

In certain embodiment, provided herein is a lipid nanoparticle for use in delivering or expressing a therapeutic agent in the lung of a subject, wherein the lipid nanoparticle comprises a permanently cationic lipid and an ionizable lipid, and wherein the lipid nanoparticle has a diameter of from about 160 nm to about 900 nm.

In certain embodiment, the lipid nanoparticle has a diameter of from 180 nm to about 900 nm, from about 300 nm to about 900 nm, from about 180 nm to about 600 nm, from about 180 nm to about 400 nm, from about 180 nm to about 350 nm, or from about 180 nm to about 300 nm. In certain embodiment, the lipid nanoparticle has a diameter of from about 180 nm to about 300 nm.

In certain embodiment, the lipid nanoparticle has a greater than neutral zeta potential at physiologic pH. In certain embodiment, the lipid nanoparticle has a zeta potential of from about 0 mV to about 25 mV, from about 0 mV to about 20 mV, or from about 2 mV to about 15 mV.

In certain embodiment, the amount of the permanently cationic lipid is from about 15 mol % to about 90 mol %, from about 20 mol % to about 80 mol %, from about 30 mol % to about 70 mol %, from about 40 mol % to about 60 mol %, or from about 45 mol % to about 55 mol % of the total lipid present in the lipid nanoparticle.

In certain embodiment, the permanently cationic lipid has a pKa of greater than about 10, or greater than about 13.

In certain embodiment, the permanently cationic lipid comprises a quaternary ammonium group.

In certain embodiment, the permanently cationic lipid is a compound of formula (I):

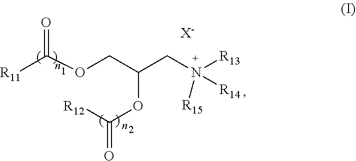

or a stereoisomer, a mixture of stereoisomers, or a pharmaceutically acceptable salt thereof, wherein R₁₁ and R₁₂ are each independently C₆₋₃₀ alkyl, C₆₋₃₀ alkenyl, or C₆₋₃₀ alkynyl, and wherein the alkyl, alkenyl and alkynyl are independently optionally substituted with one or more groups selected from hydroxyl, halogen, cyano, C₁₋₃₀ alkyl, C₁₋₃₀ haloalkyl, C₁₋₃₀ alkoxy, —S—C₁₋₃₀ alkyl, amino, —NH—C₁₋₃₀ alkyl, and —N(C₁₋₃₀ alkyl)₂;
   R₁₃, R₁₄, and R₁₅ are each independently C₁₋₆ alkyl, C₁₋₆ haloalkyl, C₂₋₆ alkenyl, C₂₋₆ alkynyl, or any two of R₃, R₁₄, and R₁₅ together with the nitrogen atom they are attached to form a 4 to 8-membered ring, and wherein the alkyl, haloalkyl, alkenyl, alkynyl, and ring are optionally substituted;
   X⁻ is an anion; and
   n₁ and n₂ are each independently 0 or 1.

In certain embodiment, R₁₁ and R₁₂ are each independently C₁₅₋₂₀ alkyl, C₁₅₋₂₀ alkenyl, or C₁₅₋₂₀ alkynyl, and wherein the alkyl, alkenyl and alkynyl are independently optionally substituted with one or more groups selected from hydroxyl, halogen, cyano, C₁₋₂₀ alkyl, C₁₋₂₀ haloalkyl, C₁₋₂₀ alkoxy, —S—C₁₋₂₀ alkyl, amino, —NH—C₁₋₂₀ alkyl, and —N(C₁₋₂₀ alkyl)₂.

In certain embodiment, R₁₃, R₁₄, and R₁₅ are each independently C₁₋₆ alkyl optionally substituted with hydroxyl, halogen, cyano, C₁₋₆alkoxy, —S—C₁₋₆ alkyl, amino, —NH—C₁₋₆ alkyl, or —N(C₁₋₆ alkyl)₂.

In certain embodiment, the permanently cationic lipid is a compound of formula (II):

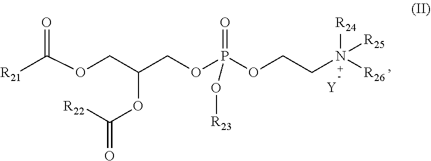

or a stereoisomer, a mixture of stereoisomers, or a pharmaceutically acceptable salt thereof, wherein R₂₁ and R₂₂ are each independently C₆₋₃₀ alkyl, C₆₋₃₀ alkenyl, or C₆₋₃₀ alkynyl, and wherein the alkyl, alkenyl and alkynyl are independently optionally substituted with one or more groups selected from hydroxyl, halogen, cyano, C₁₋₃₀ alkyl, C₁₋₃₀ haloalkyl, C₁₋₃₀ alkoxy, —S—C₁₋₃₀ alkyl, amino, —NH—C₁₋₃₀ alkyl, and —N(C₁₋₃₀ alkyl)₂;
   R₂₃ is C₁₋₆ alkyl, C₁₋₆ haloalkyl, C₂₋₆ alkenyl, or C₂₋₆ alkynyl, and wherein R₂₃ is optionally substituted with one or more groups selected from halogen, hydroxyl, C₁₋₆ alkyl, C₁₋₆ haloalkyl, C₁₋₆alkoxy, —OC(═O)R₂ₐ, —C(═O)OR₂ₐ, —C(═O)NHR₂ₐ, and —NHC(═O)R₂ₐ;
   R₂ₐ is hydrogen, C₁₋₆ alkyl, or C₁₋₆ haloalkyl;
   R₂₄, R₂₅, and R₂₆ are each independently C₁₋₆ alkyl, C₁₋₆ haloalkyl, C₂₋₆ alkenyl, C₂₋₆ alkynyl, or any two of R₂₄, R₃₅, and R₂₆ together with the nitrogen atom they are attached to form a 4 to 8-membered ring, and wherein the alkyl, haloalkyl, alkenyl, alkynyl, and ring are optionally substituted; and
   Y⁻ is an anion.

In certain embodiment, R₂₁ and R₂₂ are each independently C₁₀₋₂₅ alkyl, C₁₀₋₂₅ alkenyl, or C₁₀₋₂₅ alkynyl, and wherein the alkyl, alkenyl and alkynyl are independently optionally substituted with one or more groups selected from hydroxyl, halogen, cyano, C₁₋₂₅ alkyl, C₁₋₂₅ haloalkyl, C₁₋₂₅ alkoxy, —S—C₁₋₂₅ alkyl, amino, —NH—C₁₋₂₅ alkyl, and —N(C₁₋₂₅ alkyl)₂.

In certain embodiment, R₂₃ is C₁₋₆ alkyl or C₁₋₆ haloalkyl.

In certain embodiment, R₂₄, R₂₅, and R₂₆ are each independently C₁₋₆ alkyl optionally substituted with hydroxyl, halogen, cyano, C₁₋₆alkoxy, —S—C₁₋₆ alkyl, amino, —NH—C₁₋₆ alkyl, or —N(C₁₋₆ alkyl)₂, or any two of R₂₄, R₂₅, and R₂₆ together with the nitrogen atom they are attached to form a 5 to 6-membered ring.

In certain embodiment, the permanently cationic lipid is a pharmaceutically acceptable salt of:

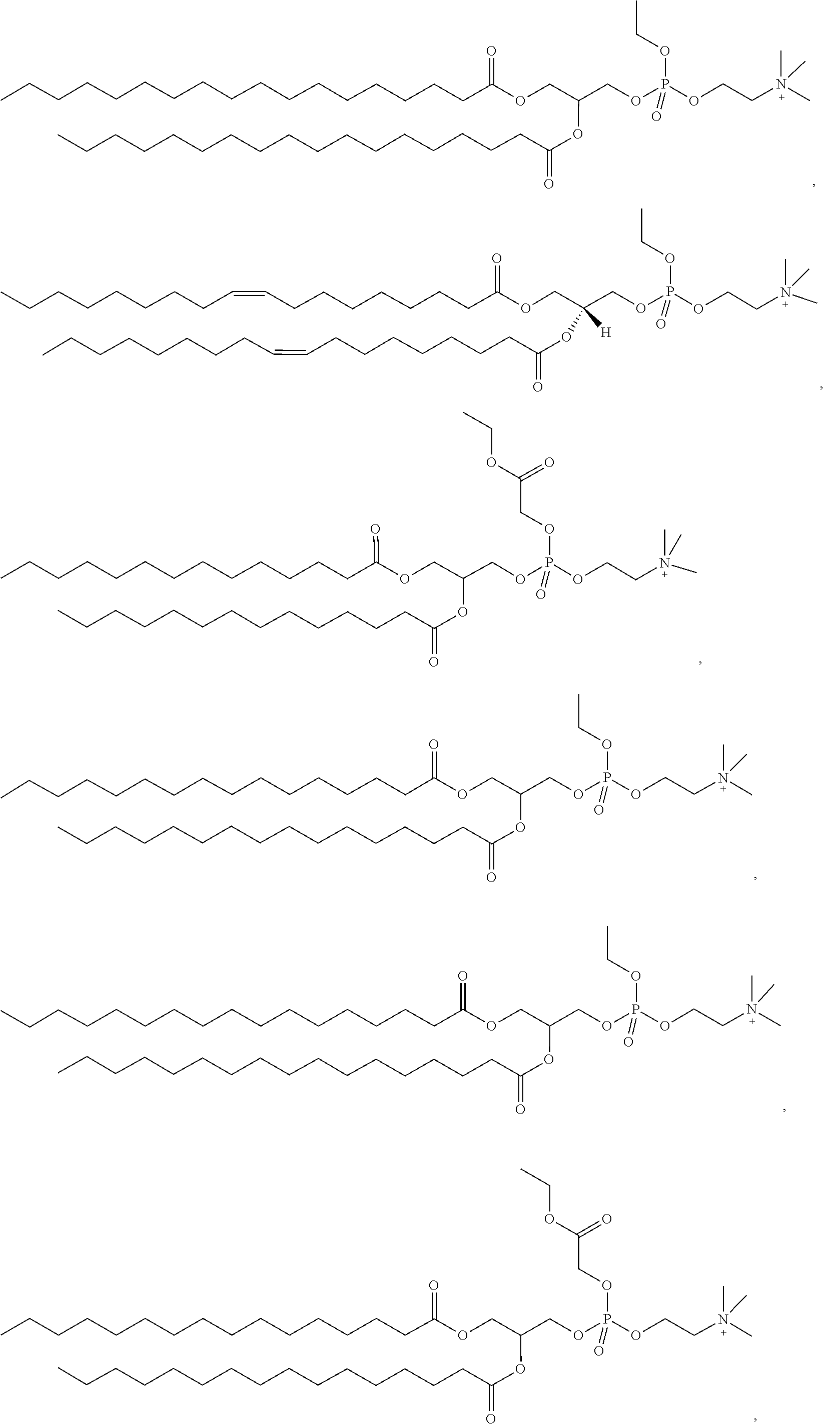

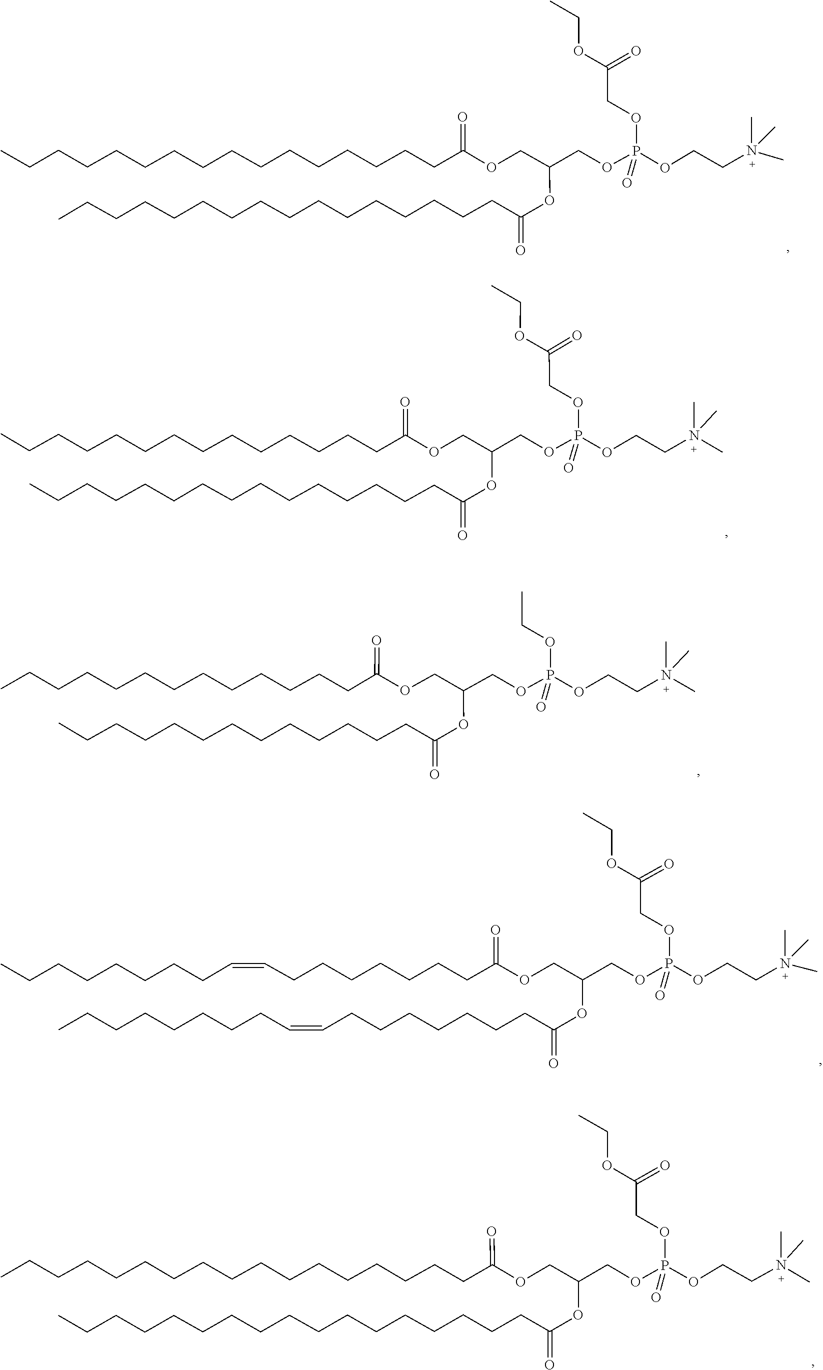

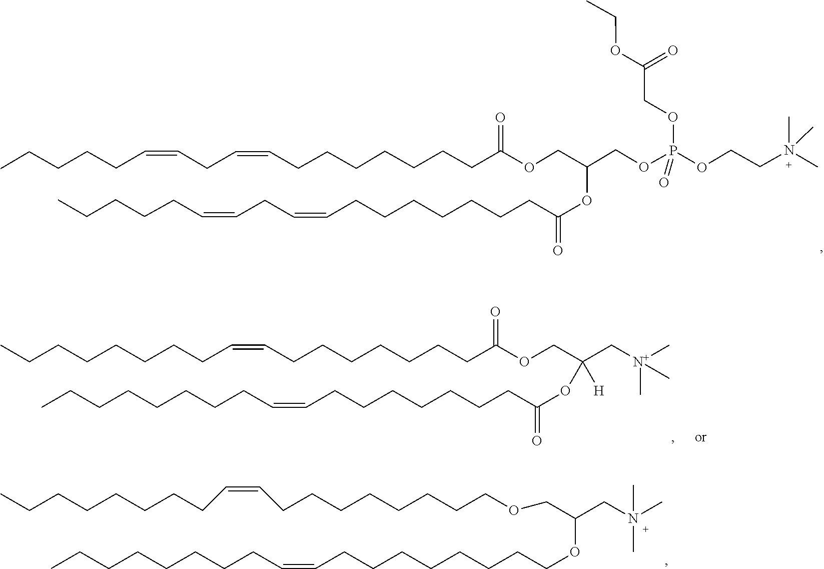

or a stereoisomer, or a mixture of stereoisomers thereof.

In certain embodiment, the permanently cationic lipid is DOTMA, DOTAP, MVL5, DOGS, DC-Chol, DDAB, EPC, or a mixture thereof. In certain embodiment, the amount of the ionizable lipid is from about 15 mol % to about 60 mol % of the total lipid present in the lipid nanoparticle. In certain embodiment, the amount of the ionizable lipid is from about 15 mol % to about 40 mol %, or from about 20 mol % to about 30 mol % of the total lipid present in the lipid nanoparticle.

In certain embodiment, the amount of the permanently cationic lipid is from about 15 mol % to about 90 mol % of the total lipid present in the lipid nanoparticle, and the amount of the ionizable lipid is from about 15 mol % to about 60 mol % of the total lipid present in the lipid nanoparticle. In certain embodiment, the amount of the permanently cationic lipid is from about 40 mol % to about 60 mol % of the total lipid present in the lipid nanoparticle, and the amount of the ionizable lipid is from about 15 mol % to about 40 mol % of the total lipid present in the lipid nanoparticle. In certain embodiment, the amount of the permanently cationic lipid is from about 45 mol % to about 55 mol % of the total lipid present in the lipid nanoparticle, and the amount of the ionizable lipid is from about 20 mol % to about 30 mol % of the total lipid present in the lipid nanoparticle.

In certain embodiment, the ionizable lipid has a pKa of from about 7 to about 13, from about 7 to about 11, or from about 7 to about 9.

In certain embodiment, the lipid nanoparticle further comprises a phospholipid. In certain embodiment, the phospholipid is DSPC, DMPC, DOPC, DPPC, POPC, DOPE, DMPE, POPOE, or DPPE, or a mixture thereof.

In certain embodiment, the lipid nanoparticle does not comprise a phospholipid or comprises a phospholipid in an amount less than about 15 mol %, less than about 10 mol %, less than about 8 mol %, less than about 5 mol %, less than about 3 mol %, or less than about 1 mol % of the total lipid present in the lipid nanoparticle.

In certain embodiment, the lipid nanoparticle further comprises a steroid. In certain embodiment, the steroid is cholesterol, campesterol, stigmasterol, sitosterol, brassicasterol, ergosterol, solanine, ursolic acid, alpha-tocopherol, beta-sitosterol, avenasterol, calciferol, or canola sterol. In certain embodiment, the amount of the steroid is from about 5 mol % to about 60 mol %, from about 10 mol % to about 50 mol %, from about 10 mol % to about 40 mol %, from about 20 mol % to about 30 mol %, or about 25 mol % of the total lipid present in the lipid nanoparticle.

In certain embodiment, the lipid nanoparticle further comprises a pegylated lipid. In certain embodiment, a pegylated moiety of the pegylated lipid has a molecule weight of from about 1000 Da to about 10,000 Da, from about 1000 Da to about 5000 Da, or from about 1000 Da to about 2000 Da. In certain embodiment, the pegylated lipid is ALC-0159, DMG-PEG2000, DMPE-PEG1000, DPPE-PEG1000, DSPE-PEG1000, DOPE-PEG1000, Ceramide-PEG2000, DMPE-PEG2000, DPPE-PEG2000, DSPE-PEG2000, DSPE-PEG2000-Mannose, Ceramide-PEG5000, DSPE-PEG5000, or DSPE-PEG2000 amine.

In certain embodiment, the amount of the pegylated lipid is from about 0.1 mol to about 5 mol %, from about 0.1 mol to about 3 mol %, from about 0.25 mol to about 2 mol %, from about 0.5 mol to about 1.5 mol %, or about 1 mol % of the total lipid present in the lipid nanoparticle.

In certain embodiment, the lipid nanoparticle comprises a permanently cationic lipid in an amount from about 15 mol % to about 90 mol % of the total lipid present in the lipid nanoparticle, an ionizable lipid in an amount from about 15 mol % to about 60 mol % of the total lipid present in the lipid nanoparticle, a steroid in an amount from about 5 mol % to about 60 mol % of the total lipid present in the lipid nanoparticle and a pegylated lipid in an amount from about 0.1 mol % to about 5 mol % of the total lipid present in the lipid nanoparticle. In certain embodiment, the lipid nanoparticle comprises a permanently cationic lipid in an amount from about 30 mol % to about 70 mol % of the total lipid present in the lipid nanoparticle, an ionizable lipid in an amount from about 15 mol % to about 40 mol % of the total lipid present in the lipid nanoparticle, a steroid in an amount from about 15 mol % to about 40 mol % of the total lipid present in the lipid nanoparticle, and a pegylated lipid in an amount from about 0.25 mol % to about 3 mol % of the total lipid present in the lipid nanoparticle. In certain embodiment, the lipid nanoparticle comprises a permanently cationic lipid in an amount from about 45 mol % to about 55 mol % of the total lipid present in the lipid nanoparticle, an ionizable lipid in an amount from about 20 mol % to about 30 mol % of the total lipid present in the lipid nanoparticle, a steroid in an amount from about 20 mol % to about 30 mol % of the total lipid present in the lipid nanoparticle, and a pegylated lipid in an amount from about 0.5 mol % to about 1.5 mol % of the total lipid present in the lipid nanoparticle.

In certain embodiment, the therapeutic agent is nucleic acid. In certain embodiment, the nucleic acid is antisense oligonucleotide (ASO), DNA, or RNA, optionally wherein the RNA is RNA interference (RNAi), small interfering RNA (siRNA), short hairpin RNA (shRNA), antisense RNA (aRNA), messenger RNA (mRNA), modified messenger RNA (mmRNA), long noncoding RNA (lncRNA), microRNA (miRNA), small activating RNA (saRNA), multicoding nucleic acid (MCNA), polymer-coded nucleic acid (PCNA), guide RNA (gRNA), CRISPR RNA (crRNA), or any other RNA in the ribozyme.

In certain embodiment, the ratio of total number of nitrogen atoms in the permanently cationic lipid and ionizable lipid and total number of phosphate atoms in the nucleic acid is from about 1:1 to about 20:1, about 1:1 to about 15:1, from about 3:1 to about 12:1, or from about 4:1 to about 9:1.

In certain embodiment, the lipid nanoparticle has an apparent pKa of greater than about 7, greater than about 8, greater than about 9, greater than about 10, from about 7 to about 10, or greater than about 10.

In certain embodiment, the amount of the therapeutic agent delivered or expressed in the lung of the subject is higher than the amount of the therapeutic agent delivered or expressed in the liver of the subject. In certain embodiment, the amount of the therapeutic agent delivered or expressed in the lung of the subject is at least 1 time, at least 5 times, at least 10 times, at least 20 times, at least 40 times, at least 60 times, or at least 100 times higher than the amount of the therapeutic agent delivered or expressed in the liver of the subject. In certain embodiment, the subject has a lung disease.

In another aspect, provided herein is a lipid nanoparticle comprising:
(i) a permanently cationic lipid in an amount from about 15 mol % to about 90 mol % of the total lipid present in the lipid nanoparticle; and
(ii) an ionizable lipid in an amount from about 15 mol % to about 60 mol % of the total lipid present in the lipid nanoparticle,
wherein the lipid nanoparticle has a diameter of from about 160 nm to about 900 nm; and wherein the permanently cationic lipid is a compound of formula (I) or (II);

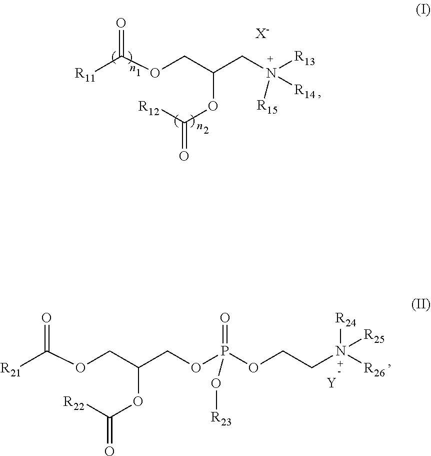

or a stereoisomer, a mixture of stereoisomers, or a pharmaceutically acceptable salt thereof, wherein R₁₁, R₁₂, R₂₁ and R₂₂ are each independently C₆₋₃₀ alkyl, C₆₋₃₀ alkenyl, or C₆₋₃₀ alkynyl, and wherein the alkyl, alkenyl and alkynyl are independently optionally substituted with one or more groups selected from hydroxyl, halogen, cyano, C₁₋₃₀alkyl, C₁₋₃₀haloalkyl, C₁₋₃₀alkoxy, —S—C₁₋₃₀ alkyl, amino, —NH—C₁₋₃₀ alkyl, and —N(C₁₋₃₀ alkyl)₂;
   R₁₃, R₁₄, and R₁₅ are each independently C₁₋₆ alkyl, C₁₋₆ haloalkyl, C₂₋₆ alkenyl, C₂₋₆ alkynyl, or any two of R₁₃, R₁₄, and R₁₅ together with the nitrogen atom they are attached to form a 4 to 8-membered ring, and wherein the alkyl, haloalkyl, alkenyl, alkynyl, and ring are optionally substituted;
   R₂₄, R₂₅, and R₂₆ are each independently C₁₋₆ alkyl, C₁₋₆ haloalkyl, C₂₋₆ alkenyl, C₂₋₆ alkynyl, or any two of R₂₄, R₃₅, and R₂₆ together with the nitrogen atom they are attached to form a 4 to 8-membered ring, and wherein the alkyl, haloalkyl, alkenyl, alkynyl, and ring are optionally substituted;
   R₂₃ is C₁₋₆ alkyl, C₁₋₆ haloalkyl, C₂₋₆ alkenyl, or C₂₋₆ alkynyl, and wherein R₂₃ is optionally substituted with one or more groups selected from halogen, hydroxyl, C₁₋₆ alkyl, C₁₋₆ haloalkyl, C₁₋₆alkoxy, —OC(═O)R₂ₐ, —C(═O)OR₂ₐ, —C(═O)NHR₂ₐ, and —NHC(═O)R₂ₐ;
   R₂ₐ is hydrogen, C₁₋₆ alkyl, or C₁₋₆ haloalkyl
   X⁻ and Y⁻ are each independently an anion; and
   n₁ and n₂ are each independently 0 or 1.

In another aspect, provided herein is a population of lipid nanoparticles comprising the lipid nanoparticle described herein, wherein the population of lipid nanoparticles have an average diameter of from about 160 nm to about 900 nm.

In certain embodiment, the average diameter of the population of lipid nanoparticles is determined by dynamic light scattering (DLS).

In yet another aspect, provided herein a pharmaceutical composition comprising the lipid nanoparticle described herein or the population of lipid nanoparticles described herein and a pharmaceutically acceptable carrier.

In yet another aspect, provided herein is a method of delivering or expressing a therapeutic agent in the lung of a subject or treating or preventing a lung disease in a subject.

In certain embodiment, provided herein is a lipid nanoparticle for use in delivering or expressing a therapeutic agent in the lung of a subject, wherein the lipid nanoparticle is administered intravenously, intraarterially, or intraperitoneally to the subject, wherein the lipid nanoparticle has a positive surface charge, and wherein the lipid nanoparticle has a diameter of from about 160 nm to about 900 nm. In certain embodiment, the lipid nanoparticle comprises a permanently cationic lipid and an ionizable lipid.

In certain embodiment, provided herein is a method of delivering or expressing a therapeutic agent in the lung of a subject or treating or preventing a lung disease in a subject, wherein the method comprises using a lipid nanoparticle comprising the therapeutic agent, wherein the lipid nanoparticle comprises a permanently cationic lipid and an ionizable lipid, and wherein the lipid nanoparticle has a diameter of from about 160 nm to about 900 nm.

In yet another aspect, provided herein is a method of treating or preventing a lung disease in a subject, comprising administering to the subject a therapeutically effective amount of the lipid nanoparticle described herein, the population of lipid nanoparticles described herein, or the pharmaceutical composition described herein.

In certain embodiment, the administration is intravenous administration, intraarterial administration, or intraperitoneal administration.

In yet another aspect, provided herein is a method of producing the lipid nanoparticle described herein or a population of lipid nanoparticles described herein comprising the steps of:
(i) dissolving in a first solution a mixture comprising a permanently cationic lipid and an ionizable lipid to form a lipid solution, wherein the lipid solution is formed in an organic solvent;
(ii) dissolving in a second solution a therapeutic agent to form a therapeutic agent solution; and
(iii) mixing the lipid solution and the therapeutic agent solution at a mixing speed of about 1 mL/min to about 18 mL/min, about 1 mL/min to about 10 mL/min, or about 2 mL/min to about 6 mL/min.

In certain embodiment, the organic solvent is ethanol. In certain embodiment, the second solution is a sodium acetate buffer having a pH of about 4.5. In certain embodiment, the lipid solution and the therapeutic agent solution are mixed at a volumetric ratio of from about 1:1 to about 1:10, about 1:1 to about 1:6, or about 1:1 to about 1:4.

### 4. BRIEF DESCRIPTION OF THE DRAWINGS

FIG. 1 depicts the expression level of luciferase in the liver and lung of mice after administration of LNPs comprising 1,2-dioleoyl-3-trimethylammonium-propane (chloride salt) (DOTAP) and Compound 46 with different particle sizes.

FIG. 2 depicts the ratio of expression level of luciferase in the lung and liver (lung/liver) of mice after administration of LNPs comprising DOTAP and Compound 46 with different particle sizes.

FIG. 3 depicts the expression level of luciferase in the liver and lung of mice after administration of LNPs comprising 1,2-di-O-octadecenyl-3-trimethylammonium propane (chloride salt) (DOTMA) and Compound 46 with different particle sizes.

FIG. 4 depicts the ratio of expression level of luciferase in the lung and liver (lung/liver) of mice after administration of LNPs comprising DOTMA and Compound 46 with different particle sizes.

FIG. 5 depicts the expression level of luciferase in the liver and lung of mice after administration of LNPs comprising DOTAP and SM102 with different particle sizes.

FIG. 6 depicts the expression level of luciferase in the liver and lung of mice after administration of LNPs comprising DOTAP and MC3 with different particle sizes.

FIG. 7 depicts the distribution level of Cy3 fluorescently labeled siRNA in the liver and lung of mice after administration of LNPs comprising 1,2-dioleoyl-3-trimethylammonium-propane (chloride salt) (DOTAP) and Compound 46 with different particle sizes.

### 5. DETAILED DESCRIPTION

Provided herein are lipid nanoparticle compositions, preparations, and uses therefore. In one aspect, provided herein is a lipid nanoparticle (LNP, such as a LNP described in Section 5.2) for use in delivering or expressing a therapeutic agent in the lung of a subject, wherein the lipid nanoparticle has a diameter of from about 160 nm to about 900 nm. In one embodiment, the LNP comprises a permanently cationic lipid (Section 5.2.1). In one embodiment, the LNP comprises an ionizable lipid (Section 5.2.2). In one embodiment, the LNP further comprises a phospholipid lipid (Section 5.2.3). In one embodiment, the LNP does not comprise a phospholipid lipid. In one embodiment, the LNP further comprises a steroid (Section 5.2.4) In one embodiment, the LNP further comprises a pegylated lipid (Section 5.2.5) In one embodiment, the LNP comprises a therapeutic agent (Section 5.2.6). In another aspect, provided herein is a population of lipid nanoparticles (Section 5.3). In one embodiment, provided herein is a LNP having a diameter of at least 160 nm. In one embodiment, the LNP described herein preferably delivers to a non-hepatic organ (e.g. lung) when administered to a subject. In one embodiment, the delivery efficiency to the non-hepatic organ increases with the increase of the size of the LNP. In one embodiment, provided herein are LNP compositions for use in treating a disease in a non-hepatic organ. In one embodiment, provided herein are LNP compositions for use in treating lung disease. In one embodiment, provided herein are LNP compositions produced from a method described in Section 5.5. In another aspect, provided here are pharmaceutical compositions comprising the LNP compositions (Section 5.4). In one aspect, provided herein is a LNP of certain size (Section 5.6). Also provided herein is a process of making the LNP compositions described herein (see Section 5.7). Also provided herein is a method of treating diseases using the LNP compositions described herein (Section 5.8) that comprises a therapeutic agent (see Section 5.2.6). In one embodiment, provided herein is a method of treating lung diseases.

One aspect of the present application relates to the discovery that the ability of LNPs to target non-hepatic organs, particularly lungs, is affected by the size of said LNPs. More specifically, it was unexpectedly found that LNPs having a diameter above certain value showed improved lung-targeting properties. Another aspect of the present application relates to the discovery that the phospholipid component, which is an essential component in classic LNPs, can be readily removed while still achieving efficient delivery of nucleic acids to non-hepatic organs.

### 5.1 Definitions

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

It should be noted that if there is a discrepancy between a depicted structure and a name for that structure, the depicted structure is to be accorded more weight.

When a range of values is listed, it is intended to encompass each value and sub-range within the range. For example, “C₁₋₆ alkyl” is intended to include C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅ and C₅₋₆ alkyl.

“C₁₋₂₈ alkyl” refers to a radical of a linear or branched, saturated hydrocarbon group having 1 to 28 carbon atoms. In some embodiments, C₄₋₂₈ alkyl, C₄₋₂₄ alkyl, C₄₋₂₀ alkyl, C₈₋₁₀ alkyl, C₂₋₈ alkyl, C₇₋₉ alkyl, C₄₋₆ alkyl, C₁₋₂₀ alkyl, C₁₋₁₄ alkyl, C₂₋₁₄ alkyl, C₁₋₁₃ alkyl, C₁₋₁₂ alkyl, C₁₋₁₀ alkyl, C₁₋₈ alkyl, C₁₋₇ alkyl, C₂₋₇ alkyl, C₁₋₆ alkyl, C₁₋₅ alkyl, C₃ alkyl, C₁₋₄ alkyl, C₂₋₄ alkyl, C₁₋₃ alkyl, C₂₋₃ alkyl, C₁₋₂ alkyl and Me are alternative. Examples of C₁₋₆ alkyl include methyl (C₁), ethyl (C₂), n-propyl (C₃), iso-propyl (C₃), n-butyl (C₄), tert-butyl (C₄), sec-butyl (C₄), iso-butyl (C₄), n-pentyl (C₅), 3-pentyl (C₅), pentyl (C₅), neopentyl (C₅), 3-methyl-2-butyl (C₅), tert-pentyl (C₅) and n-hexyl (C₆). The term “C₁₋₆ alkyl” also includes heteroalkyl, wherein one or more (e.g., 1, 2, 3 or 4) carbon atoms are substituted with heteroatoms (e.g., oxygen, sulfur, nitrogen, boron, silicon, phosphorus). Alkyl groups can be optionally substituted with one or more substituents, for example, with 1 to 5 substituents, 1 to 3 substituents or 1 substituent. Conventional abbreviations of alkyl include Me (—CH₃), Et (—CH₂CH₃), iPr (—CH(CH₃)₂), nPr (—CH₂CH₂CH₃), n-Bu (—CH₂CH₂CH₂CH₃) or i-Bu (—CH₂CH(CH₃)₂).

“C₂₋₂₀ alkenyl” refers to a radical of a linear or branched hydrocarbon group having 2 to 20 carbon atoms and at least one carbon-carbon double bond. “C₄₋₂₈ alkenyl” refers to a radical of a linear or branched hydrocarbon group having 4 to 28 carbon atoms and at least one carbon-carbon double bond. In some embodiments, C₄₋₂₀ alkenyl, C₂₋₁₃ alkenyl, C₂₋₁₀ alkenyl, C₂₋₆ alkenyl, and C₂₋₄ alkenyl is alternative. Examples of C₂₋₆ alkenyl include vinyl (C₂), 1-propenyl (C₃), 2-propenyl (C₃), 1-butenyl (C₄), 2-butenyl (C₄), butadienyl (C₄), pentenyl (C₅), pentadienyl (C₅), hexenyl (C₆), etc. The term “C₂₋₆ alkenyl” also includes heteroalkenyl, wherein one or more (e.g., 1, 2, 3 or 4) carbon atoms are replaced by heteroatoms (e.g., oxygen, sulfur, nitrogen, boron, silicon, phosphorus). The alkenyl groups can be optionally substituted with one or more substituents, for example, with 1 to 5 substituents, 1 to 3 substituents or 1 substituent.

“C₂₋₂₀ alkynyl” refers to a radical of a linear or branched hydrocarbon group having 2 to 20 carbon atoms, at least one carbon-carbon triple bond and optionally one or more carbon-carbon double bonds. “C₄₋₂₈ alkynyl” refers to a radical of a linear or branched hydrocarbon group having 4 to 28 carbon atoms, at least one carbon-carbon triple bond and optionally one or more carbon-carbon double bonds. In some embodiments, C₄₋₂₀ alkynyl, C₂₋₁₃ alkynyl, C₂₋₁₀ alkynyl, C₂₋₆ alkynyl, and C₂₋₄ alkynyl is alternative. Examples of C₂₋₆ alkynyl include, but are not limited to, ethynyl (C₂), 1-propynyl (C₃), 2-propynyl (C₃), 1-butynyl (C₄), 2-butynyl (C₄), pentynyl (C₅), hexynyl (C₆), etc. The term “C₂₋₆ alkynyl” also includes heteroalkynyl, wherein one or more (e.g., 1, 2, 3 or 4) carbon atoms are replaced by heteroatoms (e.g., oxygen, sulfur, nitrogen, boron, silicon, phosphorus). The alkynyl groups can be substituted with one or more substituents, for example, with 1 to 5 substituents, 1 to 3 substituents or 1 substituent.

“C₁₋₂₀ alkylene” refers to a divalent group formed by removing another hydrogen of the C₁₋₂₀ alkyl, and can be substituted or unsubstituted. In some embodiments, C₄₋₂₀ alkylene, C₈₋₁₀ alkylene, C₂₋₈ alkylene, C₇₋₉ alkylene, C₄₋₆ alkylene, C₁₋₂₀ alkylene, C₁₋₁₄ alkylene, C₂₋₁₄ alkylene, C₁₋₁₃ alkylene, C₁₋₁₂ alkylene, C₁₋₁₀ alkylene, C₁₋₈ alkylene, C₁₋₇ alkylene, C₂₋₇ alkylene, C₁₋₆ alkylene, C₁₋₅ alkylene, C₅ alkylene, C₁₋₄ alkylene, C₂₋₄ alkylene, C₁₋₃ alkylene, C₂₋₃ alkylene, C₁₋₂ alkylene, and methylene are alternative. The unsubstituted alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), etc. Examples of substituted alkylene groups, such as those substituted with one or more alkyl (methyl) groups, include, but are not limited to, substituted methylene (—CH(CH₃)—, —C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—), etc.

“C₂₋₁₃ alkenylene” refers to a C₂₋₁₃ alkenyl group wherein another hydrogen is removed to provide a divalent radical of alkenylene, and which may be substituted or unsubstituted. In some embodiments, C₂₋₁₀ alkenyl, C₂₋₆ alkenyl, and C₂₋₄ alkenylene is yet alternative. Exemplary unsubstituted alkenylene groups include, but are not limited to, ethylene (—CH═CH—) and propenylene (e.g., —CH═CHCH₂—, —CH₂—CH═CH—). Exemplary substituted alkenylene groups, e.g., substituted with one or more alkyl (methyl) groups, include but are not limited to, substituted ethylene (—C(CH₃)═CH—, —CH═C(CH₃)—), substituted propylene (e.g., —C(CH₃)═CHCH₂—, —CH═C(CH₃)CH₂—, —CH═CHCH(CH₃)—, —CH═CHC(CH₃)₂—, —CH(CH₃)—CH═CH—, —C(CH₃)₂—CH═CH—, —CH₂—C(CH₃)═CH—, —CH₂—CH═C(CH₃)—), and the like.

“C₂₋₁₃ alkynylene” refers to a C₂₋₁₃ alkynyl group wherein another hydrogen is removed to provide a divalent radical of alkynylene, and which may be substituted or unsubstituted. In some embodiments, C₂₋₁₀ alkynylene, C₂₋₆ alkynylene, and C₂₋₄ alkynylene is yet alternative. Exemplary alkynylene groups include, but are not limited to, ethynylene (—C≡C—), substituted or unsubstituted propynylene (—C≡CCH₂—), and the like.

“C₀₋₆ alkylene” refers to the chemical bond and the “C₁₋₆ alkylene” described above, “C₀₋₄ alkylene” refers to the chemical bond and the “C₁₋₄ alkylene” described above.

The term “variable A and variable B have a total length of x carbon atoms” means that the total number of carbon atoms of the main chain in the group represented by variable A and the number of carbon atoms of the main chain in the group represented by variable B is x.

“Halo” or “halogen” refers to fluorine (F), chlorine (Cl), bromine (Br), or iodine (I).

Thus, “C₁₋₁₀ haloalkyl” refers to the above “C₁₋₁₀ alkyl”, which is substituted by one or more halogen. In some embodiments, C₁₋₆ haloalkyl and C₁₋₄ haloalkyl is yet alternative, and still alternatively C₁₋₂ haloalkyl. Exemplary haloalkyl groups include, but are not limited to, —CF₃, —CH₂F, —CHF₂, —CHFCH₂F, —CH₂CHF₂, —CF₂CF₃, —CCl₃, —CH₂Cl, —CHCl₂, 2,2,2-trifluoro-1,1-dimethyl-ethyl, and the like. The haloalkyl can be substituted at any available point of attachment, for example, with 1 to 5 substituents, 1 to 3 substituents or 1 substituent.

“C₃₋₁₄ cycloalkyl” or “3- to 14-membered cycloalkyl” refers to a radical of a non-aromatic cyclic hydrocarbon group having from 3 to 14 ring carbon atoms and zero heteroatoms, optionally wherein 1, 2 or 3 double or triple bonds are contained. In some embodiments, 3- to 10-membered cycloalkyl, 5- to 10-membered cycloalkyl, 3- to 8-membered cycloalkyl, 3- to 7-membered cycloalkyl, 3- to 6-membered cycloalkyl yet alternative, and still alternatively 5- to 7-membered cycloalkyl, 4- to 6-membered cycloalkyl, 5- to 6-membered cycloalkyl, 5-membered cycloalkyl, and 6-membered cycloalkyl. The cycloalkyl also includes a ring system in which the cycloalkyl ring described above is fused with one or more aryl or heteroaryl groups, wherein the point of attachment is on the cycloalkyl ring, and in such case, the number of carbon atoms continues to represent the number of carbon atoms in the cycloalkyl system. The cycloalkyl further comprises the cycloalkyl described above, in which the substituents on any non-adjacent carbon atoms are connected to form a bridged ring, together forming a polycyclic alkane sharing two or more carbon atoms. The cycloalkyl further comprises the cycloalkyl described above, in which the substituents on the same carbon atom are connected to form a ring, together forming a polycyclic alkane sharing one carbon atom. Exemplary cycloalkyl groups include, but are not limited to, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), etc. The cycloalkyl can be substituted with one or more substituents, for example, with 1 to 5 substituents, 1 to 3 substituents or 1 substituent.

“C₃₋₁₀ cycloalkylene” refers to a divalent radical formed by removing another hydrogen of C₃₋₁₀ cycloalkyl group and may be substituted or unsubstituted. In some embodiments, C₃₋₆ cycloalkylene and C₃₋₄ cycloalkylene groups are particularly alternative, especially alternatively cyclopropylene.

“3- to 14-membered heterocyclyl” refers to a saturated or unsaturated radical of 3- to 14-membered non-aromatic ring system having ring carbon atoms and 1 to 5 ring heteroatoms, wherein each of the heteroatoms is independently selected from nitrogen, oxygen, sulfur, boron, phosphorus and silicon, optionally wherein 1, 2 or 3 double or triple bonds are contained. In the heterocyclyl containing one or more nitrogen atoms, the point of attachment can be a carbon or nitrogen atom as long as the valence permits. In some embodiments, 3- to 10-membered heterocyclyl is alternative, which is a radical of 3- to 10-membered non-aromatic ring system having ring carbon atoms and 1 to 5 ring heteroatoms; in some embodiments, 5- to 10-membered heterocyclyl is alternative, which is a radical of 5- to 10-membered non-aromatic ring system having ring carbon atoms and 1 to 5 ring heteroatoms; in some embodiments, 3- to 8-membered heterocyclyl is alternative, which is a radical of 3- to 8-membered non-aromatic ring system having ring carbon atoms and 1 to 4 ring heteroatoms; in some embodiments, 3- to 7-membered heterocyclyl is alternative, which is a radical of 3- to 7-membered non-aromatic ring system having ring carbon atoms and 1 to 4 ring heteroatoms; 5- to 7-membered heterocyclyl is alternative, which is a radical of 5- to 7-membered non-aromatic ring system having ring carbon atoms and 1 to 3 ring heteroatoms; 3- to 6-membered heterocyclyl is alternative, which is a radical of 3- to 6-membered non-aromatic ring system having ring carbon atoms and 1 to 3 ring heteroatoms; 4- to 6-membered heterocyclyl is alternative, which is a radical of 4- to 6-membered non-aromatic ring system having ring carbon atoms and 1 to 3 ring heteroatoms; 5- to 6-membered heterocyclyl is more alternative, which is a radical of 5- to 6-membered non-aromatic ring system having ring carbon atoms and 1 to 3 ring heteroatoms; 5-membered heterocyclyl is more alternative, which is a radical of 5-membered non-aromatic ring system having ring carbon atoms and 1 to 3 ring heteroatoms; 6-membered heterocyclyl is more alternative, which is a radical of 6-membered non-aromatic ring system having ring carbon atoms and 1 to 3 ring heteroatoms. The heterocyclyl also includes a ring system wherein the heterocyclyl described above is fused with one or more cycloalkyl groups, wherein the point of attachment is on the heterocyclyl ring, or the heterocyclyl described above is fused with one or more aryl or heteroaryl groups, wherein the point of attachment is on the heterocyclyl ring; and in such cases, the number of ring members continues to represent the number of ring members in the heterocyclyl ring system. The heterocyclyl further comprises the heterocyclyl described above, in which the substituents on any non-adjacent carbon or nitrogen atoms are connected to form a bridge ring, together forming a polycyclic xazolidine sharing two or more carbon or nitrogen atoms. The heterocyclyl further comprises the heterocyclyl described above, in which the substituents on the same carbon atom are connected to form a ring, together forming a polycyclic xazolidine sharing one carbon atom. Exemplary 3-membered heterocyclyl groups containing one heteroatom include, but are not limited to, aziridinyl, oxiranyl and thiorenyl. Exemplary 4-membered heterocyclyl groups containing one heteroatom include, but are not limited to, azetidinyl, oxetanyl and thietanyl. Exemplary 5-membered heterocyclyl groups containing one heteroatom include, but are not limited to, tetrahydrofuranyl, dihydrofuranyl, tetrahydrothiophenyl, dihydrothienyl, pyrrolidinyl, dihydropyrrolyl and pyrrolyl-2,5-dione. Exemplary 5-membered heterocyclyl groups containing two heteroatoms include, but are not limited to, pyrazolidyl, dioxolanyl, oxasulfuranyl, disulfuranyl, and xazolidine-2-one. Exemplary 5-membered heterocyclyl groups containing three heteroatoms include, but are not limited to, triazolinyl, oxadiazolinyl, and thiadiazolinyl. Exemplary 6-membered heterocyclyl groups containing one heteroatom include, but are not limited to, piperidyl, tetrahydropyranyl, dihydropyridyl and thianyl. Exemplary 6-membered heterocyclyl groups containing two heteroatoms include, but are not limited to, piperazinyl, morpholinyl, dithianyl and dioxanyl. Exemplary 6-membered heterocyclyl groups containing three heteroatoms include, but are not limited to, triazinanyl. Exemplary 7-membered heterocyclyl groups containing one heteroatom include, but are not limited to, azepanyl, oxepanyl and thiepanyl. Exemplary 5-membered heterocyclyl groups fused with a C₆ aryl (also referred as 5,6-bicyclic heterocyclyl herein) include, but are not limited to, indolinyl, isoindolinyl, dihydrobenzofuranyl, dihydrobenzothiophenyl, benzoxazolinonyl, etc. Exemplary 6-membered heterocyclyl groups fused with a C₆ aryl (also referred as 6,6-bicyclic heterocyclyl herein) include, but are not limited to, tetrahydroquinolinyl, tetrahydroisoquinolinyl, etc. The heterocyclyl further includes the heterocyclyl described above sharing one or two atoms with a cycloalkyl, heterocyclyl, aryl or heteroaryl to form a bridged or spiro ring, as long as the valence permits, where the shared atom may be carbon or nitrogen atoms. The heterocyclyl further includes the heterocyclyl described above, which optionally can be substituted with one or more substituents, e.g., with 1 to 5 substituents, 1 to 3 substituents or 1 substituent.

“C₆₋₁₀ aryl” refers to a radical of monocyclic or polycyclic (e.g., bicyclic) 4n+2 aromatic ring system having 6-10 ring carbon atoms and zero heteroatoms (e.g., having 6 or 10 shared π electrons in a cyclic array). In some embodiments, the aryl group has six ring carbon atoms (“C₆ aryl”; for example, phenyl). In some embodiments, the aryl group has ten ring carbon atoms (“C₁₀ aryl”; for example, naphthyl, e.g., 1-naphthyl and 2-naphthyl). The aryl group also includes a ring system in which the aryl ring described above is fused with one or more cycloalkyl or heterocyclyl groups, and the point of attachment is on the aryl ring, in which case the number of carbon atoms continues to represent the number of carbon atoms in the aryl ring system. The aryl can be substituted with one or more substituents, for example, with 1 to 5 substituents, 1 to 3 substituents or 1 substituent.

“5- to 14-membered heteroaryl” refers to a radical of 5- to 14-membered monocyclic or bicyclic 4n+2 aromatic ring system (e.g., having 6, 10 or 14 shared π electrons in a cyclic array) having ring carbon atoms and 1-4 ring heteroatoms, wherein each heteroatom is independently selected from nitrogen, oxygen and sulfur. In the heteroaryl group containing one or more nitrogen atoms, the point of attachment can be a carbon or nitrogen atom as long as the valence permits. Heteroaryl bicyclic systems may include one or more heteroatoms in one or two rings. Heteroaryl also includes ring systems wherein the heteroaryl ring described above is fused with one or more cycloalkyl or heterocyclyl groups, and the point of attachment is on the heteroaryl ring. In such case, the number the carbon atoms continues to represent the number of carbon atoms in the heteroaryl ring system. In some embodiments, 5- to 10-membered heteroaryl groups are alternative, which are radicals of 5- to 10-membered monocyclic or bicyclic 4n+2 aromatic ring systems having ring carbon atoms and 1-4 ring heteroatoms. In other embodiments, 5- to 6-membered heteroaryl groups are yet alternative, which are radicals of 5- to 6-membered monocyclic or bicyclic 4n+2 aromatic ring systems having ring carbon atoms and 1-4 ring heteroatoms. Exemplary 5-membered heteroaryl groups containing one heteroatom include, but are not limited to, pyrrolyl, furyl and thienyl. Exemplary 5-membered heteroaryl groups containing two heteroatoms include, but are not limited to, imidazolyl, pyrazolyl, oxazolyl, isoxazolyl, thiazolyl, and isothiazolyl. Exemplary 5-membered heteroaryl groups containing three heteroatoms include, but are not limited to, triazolyl, oxadiazolyl (such as, 1,2,4-oxadiazolyl), and thiadiazolyl. Exemplary 5-membered heteroaryl groups containing four heteroatoms include, but are not limited to, tetrazolyl. Exemplary 6-membered heteroaryl groups containing one heteroatom include, but are not limited to, pyridyl or pyridonyl. Exemplary 6-membered heteroaryl groups containing two heteroatoms include, but are not limited to, pyridazinyl, pyrimidinyl, and pyrazinyl. Exemplary 6-membered heteroaryl groups containing three or four heteroatoms include, but are not limited to, triazinyl and tetrazinyl, respectively. Exemplary 7-membered heteroaryl groups containing one heteroatom include, but are not limited to, azepinyl, oxepinyl, and thiepinyl. Exemplary 5,6-bicyclic heteroaryl groups include, but are not limited to, indolyl, isoindolyl, indazolyl, benzotriazolyl, benzothiophenyl, isobenzothiophenyl, benzofuranyl, benzoisofuranyl, benzimidazolyl, benzoxazolyl, benzoisoxazolyl, benzoxadiazolyl, benzothiazolyl, benzoisothiazolyl, benzothiadiazolyl, indolizinyl and purinyl. Exemplary 6,6-bicyclic heteroaryl groups include, but are not limited to, naphthyridinyl, pteridinyl, quinolyl, isoquinolyl, cinnolinyl, quinoxalinyl, phthalazinyl and quinazolinyl. The heteroaryl can be substituted with one or more substituents, for example, with 1 to 5 substituents, 1 to 3 substituents or 1 substituent.

“Hydroxyalkyl” refers to an alkyl group that is substituted with one or more hydroxyl groups.

“Alkoxy” refers to an oxyether form of a linear or branched-chain alkyl group, i.e., an —O— alkyl group. Similarly, “methoxy” refers to —O—CH₃.

“Optionally substituted with” means that it can be substituted with the specified substituents or unsubstituted.

The divalent groups formed by removing another hydrogen from the groups defined above such as alkyl, alkenyl, alkynyl, cycloalkyl, heterocyclyl, aryl and heteroaryl are collectively referred to as “-ylene”. Ring-forming groups such as cycloalkyl, heterocyclyl, aryl and heteroaryl are collectively referred to as “cyclic groups”.

Alkyl, alkenyl, alkynyl, cycloalkyl, heterocyclyl, aryl and heteroaryl as defined herein are optionally substituted groups.

Exemplary substituents on carbon atoms include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —Osi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{cc})₂, —B(OR^{cc})₂, —BR^{aa}(OR^{cc}), alkyl, haloalkyl, alkenyl, alkynyl, cycloalkyl, heterocyclyl, aryl and heteroaryl, wherein each of the alkyl, alkenyl, alkynyl, cycloalkyl, heterocyclyl, aryl and heteroaryl is independently substituted with 0, 1, 2, 3, 4 or 5 R^{dd} groups; or two geminal hydrogen on a carbon atom are replaced with ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb} or ═NOR^{cc} groups;

Each of the R^{aa} is independently selected from alkyl, haloalkyl, alkenyl, alkynyl, cycloalkyl, heterocyclyl, aryl and heteroaryl, or two of the R^{aa} groups are combined to form a heterocyclyl or heteroaryl ring, wherein each of the alkyl, alkenyl, alkynyl, cycloalkyl, heterocyclyl, aryl and heteroaryl is independently substituted with 0, 1, 2, 3, 4 or 5 R^{dd} groups;

Each of the R^{bb} is independently selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, alkyl, haloalkyl, alkenyl, alkynyl, cycloalkyl, heterocyclyl, aryl and heteroaryl, or two R^{bb} groups are combined to form a heterocyclyl or a heteroaryl ring, wherein each of the alkyl, alkenyl, alkynyl, cycloalkyl, heterocyclyl, aryl and heteroaryl is independently substituted with 0, 1, 2, 3, 4 or 5 R^{dd} groups;

Each of the R^{cc} is independently selected from hydrogen, alkyl, haloalkyl, alkenyl, alkynyl, cycloalkyl, heterocyclyl, aryl and heteroaryl, or two R^{cc} groups are combined to form a heterocyclyl or a heteroaryl ring, wherein each of the alkyl, alkenyl, alkynyl, cycloalkyl, heterocyclyl, aryl and heteroaryl is independently substituted with 0, 1, 2, 3, 4 or 5 R^{dd} groups;

Each of the R^{dd} is independently selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff)}OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —Osi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, alkyl, haloalkyl, alkenyl, alkynyl, cycloalkyl, heterocyclyl, aryl, heteroaryl, wherein each of the alkyl, alkenyl, alkynyl, cycloalkyl, heterocyclyl, aryl and heteroaryl is independently substituted with 0, 1, 2, 3, 4 or 5 R^{gg} groups, or two geminal R^{dd} substituents can be combined to form ═O or ═S;

Each of the R^{ee} is independently selected from alkyl, haloalkyl, alkenyl, alkynyl, cycloalkyl, aryl, heterocyclyl, and heteroaryl, wherein each of the alkyl, alkenyl, alkynyl, cycloalkyl, heterocyclyl, aryl and heteroaryl is independently substituted with 0, 1, 2, 3, 4 or 5 R^{gg} groups;

Each of the R^{ff} is independently selected from hydrogen, alkyl, haloalkyl, alkenyl, alkynyl, cycloalkyl, heterocyclyl, aryl and heteroaryl, or two R^{ff} groups are combined to form a heterocyclyl or a heteroaryl ring, wherein each of the alkyl, alkenyl, alkynyl, cycloalkyl, heterocyclyl, aryl and heteroaryl is independently substituted with 0, 1, 2, 3, 4 or 5 R^{gg} groups;

Each of the R^{gg} is independently selected from halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —Osi(C₁₋₆ alkyl)₃, —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ haloalkyl, C₂-C₆ alkenyl, C₂-C₆ alkynyl, C₃-C₇ cycloalkyl, C₆-C₁₀ aryl, C₃-C₇ heterocyclyl, C₅-C₁₀ heteroaryl; or two geminal R^{gg} substituents may combine to form ═O or ═S; wherein X⁻ is a counter-ion.

Exemplary substituents on nitrogen atoms include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, alkyl, haloalkyl, alkenyl, alkynyl, cycloalkyl, heterocyclyl, aryl and heteroaryl, or two R^{cc} groups attached to a nitrogen atom combine to form a heterocyclyl or a heteroaryl ring, wherein each of the alkyl, alkenyl, alkynyl, cycloalkyl, heterocyclyl, aryl and heteroaryl is independently substituted with 0, 1, 2, 3, 4 or 5 R^{dd} groups, and wherein R^{aa}, R^{bb}, R^{cc} and R^{dd} are as described herein.

“Nucleic acids” refers to single- or double-stranded deoxyribonucleic acid (DNA) or ribonucleic acid (RNA) molecules and their heterozygous molecules. Examples of nucleic acid molecules include, but are not limited to, messenger RNA (mRNA), microRNA (miRNA), small interfering RNA (siRNA), self-amplified RNA (saRNA), and antisense oligonucleotides (ASO), etc. Nucleic acids may be further chemically modified, and the chemical modifier selected from one of, or a combination of: pseudouridine, N1-methyl-pseudouridine, 5-methoxyuridine, and 5-methylcytosine. mRNA molecules contain protein coding regions and may further contain expression regulatory sequences. Typical expression regulatory sequences include, but are not limited to, 5′ cap, 5′ untranslated region (5′ UTR), 3′ untranslated region (3′ UTR), polyadenylate sequence (PolyA), miRNA binding sites.

As used herein, the term “pKa” refers to the negative logarithm (p) of the acid dissociation constant (Ka) of an acid, and is equal to the pH value at which equal concentrations of the acid and its conjugate base form are present in solution. The term “pKa” as used herein can be measured using water or dimethyl sulfoxide as a solvent. Observed values previously reported as pKa in case of using water as a solvent may be employed as pKa as used herein. In some embodiments, pKa can be determined by experiments, such as titration experiments using hydrochloric acid or sodium hydroxide. In some embodiments, pKa is determine by 2-(p-toluidino) naphthalene-6-sulfonic acid (TNS) fluorescent method.

The term “zeta potential” as used herein refers to the overall surface charge that a nanoparticle acquires in a particular medium (e.g. water), and is a measure of electrostatic attraction and repulsion. Zeta potential values are indicative of dispersion stability, aggregation, and diffusion behavior. Zeta potential may be calculated from electrokinetic data obtained from, e.g., laser Doppler velocimetry. In this technique, a voltage is applied across a pair of electrodes at either end of a cell containing a nanoparticle dispersion. Charged nanoparticles are attracted to the oppositely charged electrode, and their velocity is measured and expressed in unit field strength as their electrophoretic mobility. Zeta values may be predictive in determining penetration through various cellular membranes. The zeta potential of a lipid nanoparticle described herein can be measured by Zetasizer Pro (e.g., one from Malvern Instruments, Ltd). LNPs can be diluted to certain level of total mRNA (e.g., 1.0 ng/μL) in an pH buffer (e.g., PBS pH=7.4) and loaded into a Disposable Folded Capillary Cell (e.g., DTS1070). The sample can be equilibrated for certain period (e.g., 120 seconds), duplicate for several times with certain time period (e.g., 20 seconds) between measurements.

The term “ionizable” or “ionizable group” as used herein refers to a chemical group that is either ionized or capable of ionization. An ionizable group may present as a neutral group, a positively charged group (cationic group) or a negatively charged group (anionic group). As used herein, a “charged moiety” is a chemical moiety that carries a formal electronic charge, e.g., monovalent (+1, or −1), divalent (+2, or −2), trivalent (+3, or −3), etc. The term “ionizable lipid” as used herein refers to a lipid having at least one ionizable group. In one embodiment, the ionizable lipid is an ionizable cationic lipid. In one embodiment, an ionizable lipid has a pKa of the protonatable group in the range of about 4 to about 7. In certain embodiment, an ionizable lipid comprises a tertiary amino group. Exemplary ionizable lipids are described in Section 5.2.2, and include, but are not limited to, compounds of formula (IV′), (V′), (VI′), and (VII′).

The term “pharmaceutically acceptable salt” as used herein refers to those carboxylate and amino acid addition salts of the compounds of the present disclosure, which are suitable for the contact with patients' tissues within a reliable medical judgment, and do not produce inappropriate toxicity, irritation, allergy, etc. They are commensurate with a reasonable benefit/risk ratio, and are effective for their intended use. The term includes, if possible, the zwitterionic form of the compounds of the disclosure.

The pharmaceutically acceptable base addition salts are formed with metals or amines, such as alkali metal and alkaline earth metal hydroxides or organic amines. Examples of the metals used as cations include sodium, potassium, magnesium, calcium, etc. Examples of suitable amines are N,N′-dibenzylethylenediamine, chloroprocaine, choline, diethanolamine, ethylenediamine, N-methylglucamine and procaine.

The base addition salt of the acidic compound can be prepared by contacting the free acid form with a sufficient amount of the required base to form a salt in a conventional manner. The free acid can be regenerated by contacting the salt form with an acid in a conventional manner and then isolating the free acid. The free acid forms are somewhat different from their respective salt forms in their physical properties, such as solubility in polar solvents. But for the purposes of the present disclosure, the salts are still equivalent to their respective free acids.

The salts can be prepared from the inorganic acids, which include sulfates, pyrosulfates, bisulfates, sulfites, bisulfites, nitrates, phosphates, monohydrogen phosphates, dihydrogen phosphates, metaphosphates, pyrophosphates, chlorides, bromides and iodides. Examples of the acids include hydrochloric acid, nitric acid, sulfuric acid, hydrobromic acid, hydroiodic acid, phosphoric acid, etc. The representative salts include hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthalate, methanesulfonate, glucoheptanate, lactobionate, lauryl sulfonate, isethionate, etc. The salts can also be prepared from the organic acids, which include aliphatic monocarboxylic and dicarboxylic acids, phenyl-substituted alkanoic acids, hydroxyalkanoic acids, alkanedioic acid, aromatic acids, aliphatic and aromatic sulfonic acids, etc. The representative salts include acetate, propionate, octanoate, isobutyrate, oxalate, malonate, succinate, suberate, sebacate, fumarate, maleate, mandelate, benzoate, chlorobenzoate, methyl benzoate, dinitrobenzoate, naphthoate, besylate, tosylate, phenylacetate, citrate, lactate, maleate, tartrate, methanesulfonate, etc. The pharmaceutically acceptable salts can include cations based on alkali metals and alkaline earth metals, such as sodium, lithium, potassium, calcium, magnesium, etc., as well as non-toxic ammonium, quaternary ammonium, and amine cations including, but not limited to, ammonium, tetramethylammonium, tetraethylammonium, methylamine, dimethylamine, trimethylamine, triethylamine, ethylamine, etc. Salts of amino acids are also included, such as arginine salts, gluconates, galacturonates, etc. (for example, see Berge S. M. et al., “Pharmaceutical Salts,” J. Pharm. Sci., 1977; 66: 1-19 for reference).

“Subjects” to which administration is contemplated include, but are not limited to, humans (e.g., males or females of any age group, e.g., paediatric subjects (e.g., infants, children, adolescents) or adult subjects (e.g., young adults, middle-aged adults or older adults) and/or non-human animals, such as mammals, e.g., primates (e.g., cynomolgus monkeys, rhesus monkeys), cattle, pigs, horses, sheep, goats, rodents, cats and/or dogs. In some embodiments, the subject is a human. In some embodiments, the subject is a non-human animal. The terms “human”, “patient” and “subject” can be used interchangeably herein.

“Disease”, “disorder”, and “condition” can be used interchangeably herein.

Unless otherwise indicated, the term “treatment” or “treating” as used herein includes the effect on a subject who is suffering from a particular disease, disorder, or condition, which reduces or reverses the severity of the disease, disorder, or condition, or delays or slows the progression of the disease, disorder or condition (“therapeutic treatment”). The term also includes the effect that occurs before the subject begins to suffer from a specific disease, disorder or condition (“prophylactic treatment”).

Generally, the “effective amount” of an active pharmaceutical ingredient (API) refers to an amount sufficient to elicit a target biological response. As understood by those skilled in the art, the effective amount of the pharmaceutical composition of the disclosure can vary depending on the following factors, such as the desired biological endpoint, the pharmacokinetics of the pharmaceutical composition, the diseases being treated, the mode of administration, and the age, health status and symptoms of the subjects. The effective amount includes therapeutically effective amount and prophylactically effective amount.

Unless otherwise indicated, the “therapeutically effective amount” of the pharmaceutical composition as used herein is an amount sufficient to provide therapeutic benefits in the course of treating a disease, disorder or condition, or to delay or minimize one or more symptoms associated with the disease, disorder or condition. The therapeutically effective amount of a pharmaceutical composition refers to the amount of the therapeutic agent that, when used alone or in combination with other therapies, provides a therapeutic benefit in the treatment of a disease, disorder or condition. The term “therapeutically effective amount” can include an amount that improves the overall treatment, reduces or avoids the symptoms or causes of the disease or condition, or enhances the therapeutic effect of other therapeutic agents.

### 5.2 Lipid Nanoparticle

In one aspect, provided herein is a lipid nanoparticle (LNP). In one embodiment, the LNP comprises a permanently cationic lipid (Section 5.2.1). In one embodiment, the LNP comprises an ionizable lipid (Section 5.2.2). In one embodiment, the LNP comprises a permanently cationic lipid (Section 5.2.1) and an ionizable lipid (Section 5.2.2). Unless otherwise specified, the permanently cationic lipid is different from the ionizable lipid. In one embodiment, the LNP further comprises a phospholipid lipid (Section 5.2.3). In one embodiment, the LNP does not comprises phospholipid lipid. In one embodiment, the LNP further comprises a steroid (Section 5.2.4). In one embodiment, the LNP further comprises a polymer-conjugated lipid (Section 5.2.5). In one embodiment, the LNP further comprises a therapeutic agent (Section 5.2.6).

In one embodiment, provided herein is a lipid nanoparticle for use in delivering or expressing a therapeutic agent in the lung of a subject, wherein the lipid nanoparticle has a diameter of from about 160 nm to about 900 nm.

In one embodiment, provided herein is a lipid nanoparticle for use in delivering or expressing a therapeutic agent in the lung of a subject, wherein the lipid nanoparticle is administered intravenously, intraarterially, or intraperitoneally to the subject, wherein the lipid nanoparticle has a positive surface charge, and wherein the lipid nanoparticle has a diameter of from about 160 nm to about 900 nm. In one embodiment, the lipid nanoparticle comprises a permanently cationic lipid and an ionizable lipid.

In one embodiment, provided herein is a lipid nanoparticle for use in delivering or expressing a therapeutic agent in the lung of a subject, wherein the lipid nanoparticle comprises a permanently cationic lipid and an ionizable lipid, and wherein the lipid nanoparticle has a diameter of from about 160 nm to about 900 nm.

In one embodiment, the lipid nanoparticle has a diameter of from about 180 nm to about 900 nm. In one embodiment, the lipid nanoparticle has a diameter of from about 160 nm to about 600 nm. In one embodiment, the lipid nanoparticle has a diameter of from about 160 nm to about 400 nm. In one embodiment, the lipid nanoparticle has a diameter of from about 160 nm to about 350 nm. In one embodiment, the lipid nanoparticle has a diameter of from about 180 nm to about 300 nm. In one embodiment, the lipid nanoparticle has a diameter of from about 300 nm to about 400 nm.

In one embodiment, provided herein is a lipid nanoparticle comprising
(i) a permanently cationic lipid in an amount from about 15 mol % to about 90 mol % of the total lipid present in the lipid nanoparticle; and
(ii) an ionizable lipid in an amount from about 20 mol % to about 60 mol % of the total lipid present in the lipid nanoparticle, and
wherein the lipid nanoparticle has a diameter has a diameter of from about 160 nm to about 900 nm.

In one embodiment, provided herein is a lipid nanoparticle comprising a permanently cationic lipid and an ionizable lipid, wherein the lipid nanoparticle has a diameter of from about 300 nm to about 900 nm. In one embodiment, provided herein is a lipid nanoparticle comprising a permanently cationic lipid and an ionizable lipid, wherein the lipid nanoparticle has a diameter of from about 180 nm to about 300 nm.

In one embodiment, the lipid nanoparticle has an apparent acid dissociation constant (pKa) of greater than 7. In one embodiment, the lipid nanoparticle has an apparent pKa of greater than 8. In one embodiment, the lipid nanoparticle has an apparent pKa of greater than 9. In one embodiment, the lipid nanoparticle has an apparent pKa of from about 7 to about 10. In one embodiment, the lipid nanoparticle has an apparent pKa of about 7, about 7.5, about 8, about 8.5, about 9, about 9.5, or about 10.

In one embodiment, the lipid nanoparticle has a positive surface charge. In one embodiment, the lipid nanoparticle has a positive surface charge at physiological pH. In one embodiment, the surface charge is determined by measuring zeta potential of the nanoparticle. In one embodiment, the lipid nanoparticle has a greater than neutral zeta potential at physiologic pH. In one embodiment, the zeta potential is from about 0 mV to about 50 mV. In one embodiment, the zeta potential is from about 5 mV to about 50 mV. In one embodiment, the zeta potential is from about 0 mV to about 25 mV. In one embodiment, the zeta potential is from about 0 mV to about 20 mV, In one embodiment, the zeta potential is from about 2 mV to about 15 mV. In one embodiment, the zeta potential is about 1 mV, about 5 mV, about 10 mV, about 15 mV, about 20 mV, about 25 mV, about 30 mV, about 35 mV, about 40 mV, or about 50 mV.

In one embodiment, provided herein is a lipid nanoparticle comprising a permanently cationic lipid and an ionizable lipid, wherein the lipid nanoparticle has a diameter of from about 160 nm to about 900 nm, and wherein the lipid nanoparticle has an apparent pKa of greater than 7.

In one embodiment, provided herein is a lipid nanoparticle comprising a permanently cationic lipid and an ionizable lipid, wherein the lipid nanoparticle has a diameter of from about 160 nm to about 900 nm, and wherein the lipid nanoparticle has a zeta potential from about 0 mV to about 50 mV.

In one embodiment, the lipid nanoparticle comprises about 15 mol % to about 90 mol % of permanently cationic lipid, and about 15 mol % to about 60 mol % of ionizable lipid. In one embodiment, the lipid nanoparticle comprises about 40 mol % to about 60 mol % of permanently cationic lipid, and about 15 mol % to about 40 mol % of ionizable lipid. In one embodiment, the lipid nanoparticle comprises about 45 mol % to about 55 mol % of permanently cationic lipid, and about 20 mol % to about 30 mol % of ionizable lipid.

In one embodiment, the lipid nanoparticle comprises about 15 mol % to about 90 mol % of permanently cationic lipid, about 15 mol % to about 60 mol % of ionizable lipid, about 5 mol % to about 60 mol % of steroid, and about 0.1 mol % to about 5 mol % of polymer-conjugated lipid.

In one embodiment, the lipid nanoparticle comprises about 30 mol % to about 70 mol % of permanently cationic lipid, about 15 mol % to about 40 mol % of ionizable lipid, about 15 mol % to about 40 mol % of steroid, and about 0.25 mol % to about 3 mol % of polymer-conjugated lipid.

In one embodiment, the lipid nanoparticle comprises about 45 mol % to about 55 mol % of permanently cationic lipid, about 20 mol % to about 30 mol % of ionizable lipid, about 20 mol % to about 30 mol % of steroid, and about 0.5 mol % to about 1.5 mol % of polymer-conjugated lipid.

In one embodiment, the lipid nanoparticle has a molar ratio of lipids as shown in Table 5B. In one embodiment, the molar ratio of Permanently Cationic Lipid:Ionizable Lipid:Phospholipid:Cholesterol:PEG-lipid in the lipid nanoparticle is about 50:24:0:25:1. In one embodiment, the molar ratio is about 50:29:0:20:1. In one embodiment, the molar ratio is about 50:34:0:15:1. In one embodiment, the molar ratio is about 45:24:0:30:1. In one embodiment, the molar ratio is about 45:29:0:25:1. In one embodiment, the molar ratio is about 45:34:0:20:1. In one embodiment, the molar ratio is about 55:24:0:20:1. In one embodiment, the molar ratio is about 55:29:0:15:1. In one embodiment, the molar ratio is about 55:34:0:10:1. In one embodiment, the molar ratio is about 50:24:0:24:2. In one embodiment, the molar ratio is about 50:29:0:19:2. In one embodiment, the molar ratio is about 50:34:0:14:2. In one embodiment, the molar ratio is about 45:24:0:29:2. In one embodiment, the molar ratio is about 45:29:0:24:2. In one embodiment, the molar ratio is about 45:34:0:19:2. In one embodiment, the molar ratio is about 55:24:0:19:2. In one embodiment, the molar ratio is about 55:29:0:14:2. In one embodiment, the molar ratio is about 55:34:0:9:2. In one embodiment, the molar ratio is about 45:24:5:25:1. In one embodiment, the molar ratio is about 45:29:5:20:1. In one embodiment, the molar ratio is about 45:34:5:15:1. In one embodiment, the molar ratio is about 40:24:10:30:1. In one embodiment, the molar ratio is about 40:29:10:25:1. In one embodiment, the molar ratio is about 40:34:10:20:1. In one embodiment, the molar ratio is about 45:24:5:24:2. In one embodiment, the molar ratio is about 45:29:5:19:2. In one embodiment, the molar ratio is about 45:34:5:14:2. In one embodiment, the molar ratio is about 40:24:10:29:2. In one embodiment, the molar ratio is about 40:29:10:24:2. In one embodiment, the molar ratio is about 40:34:10:19:2.

In one embodiment, the lipid nanoparticle is cationic. In one embodiment, the lipid nanoparticle is cationic under physiological conditions. In one embodiment, the lipid nanoparticle is cationic at pH from about 7 to about 9. In one embodiment, the lipid nanoparticle is cationic at pH about 7.4. In one embodiment, the lipid nanoparticle maintained cationic during the process of being used in a method of delivering or expressing a therapeutic agent or during the process of being used in a method of treating or preventing a lung disease. In one embodiment, the cationic lipid nanoparticle does not comprise a permanently cationic lipid. In one embodiment, the cationic lipid nanoparticle comprises a cationic lipid component, for example, a cationic phospholipid, a cationic polymer-conjugated lipid, or a cationic cholesterol. In one embodiment, the lipid nanoparticle comprises a cationic phospholipid. In one embodiment, the lipid nanoparticle comprises a cationic polymer-conjugated lipid. In one embodiment, the lipid nanoparticle comprises a cationic cholesterol. In one embodiment, the lipid nanoparticle comprises a quaternary ammonium group and a moiety derived from an ionizable lipid (e.g. the ionizable lipid of Section 5.2.2). In one embodiment, the lipid nanoparticle comprises a quaternary ammonium group and a moiety derived from a phospholipid lipid (e.g. the phospholipid of Section 5.2.3). In one embodiment, the lipid nanoparticle comprises a quaternary ammonium group and a moiety derived from a polymer-conjugated lipid (e.g. the polymer-conjugated lipid of Section 5.2.5).

In one embodiment, the ionizable lipid component comprises an ionizable group and two hydrophobic chains, wherein each hydrophobic chain comprises a biodegradable group. In one embodiment, the ionizable lipid component comprises a tertiary amine group, two C₆-C₃₀ hydrocarbon chains, and wherein each hydrocarbon chain comprises a biodegradable group. In one embodiment, the C₆-C₃₀ hydrocarbon chain is a C₆-C₃₀ alkyl, C₆-C₁₈ alkyl, or C₈-C₁₆ alkyl. In one embodiment, the C₆-C₃₀ hydrocarbon chain is a C₆-C₃₀ alkenyl, C₆-C₁₈ alkenyl, or C₈-C₁₆ alkenyl. In one embodiment, the biodegradable group is an ether group, an ester group, an amide group, a thioester group, a carbonate group, a carbamate group, a carbamothioester group, a urea group, or a disulfide. In one embodiment, the biodegradable group is selected from the group consisting of —O—, —OC(O)—, —OC(O)—, —SC(O)—, —C(O)S—, —OC(S)—, —C(S)O—, —S—S—, —CH═N—, —CH═N—O—, —C(O)(NH)—, —C(S)(NH)—, —NHC(O)—, —NHC(O)NH—, and —OC(O)O—.

### 5.2.1. Permanently Cationic Lipid

In one embodiment, the lipid nanoparticle comprises a permanently cationic lipid. A permanently cationic lipid is permanently positively charged regardless of the pH of its biological environment. The permanently cationic lipid provided herein is not limited any specific chemical structures.

In one embodiment, the permanently cationic lipid has no measurable pKa value. In one embodiment, the permanently cationic lipid has a pKa of greater than 8. In one embodiment, the permanently cationic lipid has a pKa of greater than 10.

In one embodiment, the permanently cationic lipid comprises a quaternary ammonium group. In one embodiment, the permanently cationic lipid comprises two quaternary ammonium group. In one embodiment, the permanently cationic lipid comprises three quaternary ammonium groups. In one embodiment, the permanently cationic lipid comprises a quaternary ammonium group and a tertiary amine group. In one embodiment, the permanently cationic lipid comprises a quaternary ammonium group and a moiety derived from an ionizable lipid. In one embodiment, the permanently cationic lipid comprises a quaternary ammonium group and a moiety derived from a phospholipid lipid. In one embodiment, the permanently cationic lipid comprises a quaternary ammonium group and a moiety derived from a polymer-conjugated lipid.

In one embodiment, the permanently cationic lipid is a compound of formula (I):

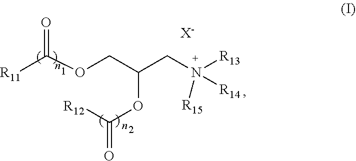

or a stereoisomer, a mixture of stereoisomers, or a pharmaceutically acceptable salt thereof, wherein R₁₁ and R₁₂ are each independently C₆₋₃₀ alkyl, C₆₋₃₀ alkenyl, or C₆₋₃₀ alkynyl, and wherein the alkyl, alkenyl and alkynyl are independently optionally substituted with one or more groups selected from hydroxyl, halogen, cyano, C₁₋₃₀alkyl, C₁₋₃₀haloalkyl, C₁₋₃₀alkoxy, —S—C₁₋₃₀alkyl, amino, —NH—C₁₋₃₀ alkyl, and —N(C₁₋₃₀ alkyl)₂;
   R₁₃, R₁₄, and R₁₅ are each independently C₁₋₆ alkyl, C₁₋₆ haloalkyl, C₂₋₆ alkenyl, C₂₋₆ alkynyl, or any two of R₁₃, R₁₄, and R₁₅ together with the nitrogen atom they are attached to form a 4 to 8-membered ring, and wherein the alkyl, haloalkyl, alkenyl, alkynyl, and ring are optionally substituted;
   X⁻ is an anion; and
   n₁ and n₂ are each independently 0 or 1.

In one embodiment, R₁₁ is C₆₋₃₀ alkyl. In one embodiment, R₁₁ is C₈₋₂₆ alkyl. In one embodiment, R₁₁ is C₁₀₋₂₄ alkyl. In one embodiment, R₁₁ is C₁₂₋₂₂ alkyl. In one embodiment, R₁₁ is C₁₄₋₂₀ alkyl. In one embodiment, R₁₁ is C₁₄ alkyl. In one embodiment, R₁₁ is C₁₅ alkyl. In one embodiment, R₁₁ is C₁₆ alkyl. In one embodiment, R₁₁ is C₁₇ alkyl. In one embodiment, R₁₁ is C₁₅ alkyl. In one embodiment, R₁₁ is C₁₉ alkyl. In one embodiment, R₁₁ is C₂₀ alkyl. In one embodiment, the alkyl in R₁₁ is unsubstituted. In one embodiment, the alkyl in R₁₁ is substituted. In one embodiment, the alkyl in R₁₁ is substituted with one or more hydroxyl, halogen, cyano, C₁₋₃₀ alkyl, C₁₋₃₀ haloalkyl, C₁₋₃₀ alkoxy, —S—C₁₋₃₀ alkyl, amino, —NH—C₁₋₃₀ alkyl, or —N(C₁₋₃₀ alkyl)₂. In one embodiment, the alkyl in R₁₁ is substituted with one or more hydroxyl, halogen, cyano, C₁₋₁₂ alkyl, C₁₋₁₂ haloalkyl, C₁₋₁₂ alkoxy, —S—C₁₋₁₂ alkyl, amino, —NH—C₁₋₁₂ alkyl, or —N(C₁₋₁₂ alkyl)₂. In one embodiment, the alkyl in R₁₁ is substituted with one or more hydroxyl, halogen, cyano, C₁₋₆ alkyl, C₁₋₆haloalkyl, C₁₋₆ alkoxy, —S—C₁₋₆ alkyl, amino, —NH—C₁₋₁₆ alkyl, or —N(C₁₋₆ alkyl)₂.

In one embodiment, R₁₁ is C₆₋₃₀ alkenyl. In one embodiment, R₁₁ is C₈₋₂₆ alkenyl. In one embodiment, R₁₁ is C₁₀₋₂₄ alkenyl. In one embodiment, R₁₁ is C₁₂₋₂₂ alkenyl. In one embodiment, R₁₁ is C₁₄₋₂₀ alkenyl. In one embodiment, R₁₁ is C₁₄ alkenyl. In one embodiment, R₁₁ is C₁₅ alkenyl. In one embodiment, R₁₁ is C₁₆ alkenyl. In one embodiment, R₁₁ is C₁₇ alkenyl. In one embodiment, R₁₁ is Cis alkenyl. In one embodiment, R₁₁ is C₁₉ alkenyl. In one embodiment, R₁₁ is C₂₀ alkenyl. In one embodiment, the alkenyl in R₁₁ has one C═C double bond. In one embodiment, the alkenyl in R₁₁ has two C═C double bond. In one embodiment, the alkenyl in R₁₁ has three C═C double bond. In one embodiment, the alkenyl in R₁₁ is unsubstituted. In one embodiment, the alkenyl in Ru is substituted. In one embodiment, the alkenyl in R₁₁ is substituted with one or more hydroxyl, halogen, cyano, C₁₋₃₀ alkyl, C₁₋₃₀ haloalkyl, C₁₋₃₀ alkoxy, —S—C₁₋₃₀ alkyl, amino, —NH—C₁₋₃₀ alkyl, or —N(C₁₋₃₀ alkyl)₂. In one embodiment, the alkenyl in R₁₁ is substituted with one or more hydroxyl, halogen, cyano, C₁₋₁₂ alkyl, C₁₋₁₂ haloalkyl, C₁₋₁₂ alkoxy, —S—C₁₋₁₂ alkyl, amino, —NH—C₁₋₁₂ alkyl, or —N(C₁₋₁₂ alkyl)₂. In one embodiment, the alkenyl in R₁₁ is substituted with one or more hydroxyl, halogen, cyano, C₁₋₆ alkyl, C₁₋₆haloalkyl, C₁₋₆ alkoxy, —S—C₁₋₆ alkyl, amino, —NH—C₁₋₁₆ alkyl, or —N(C₁₋₆ alkyl)₂.

In one embodiment, R₁₁ is C₆₋₃₀ alkynyl. In one embodiment, R₁₁ is C₈₋₂₆ alkynyl. In one embodiment, R₁₁ is C₁₀₋₂₄ alkynyl. In one embodiment, R₁₁ is C₁₂₋₂₂ alkynyl. In one embodiment, R₁₁ is C₁₄₋₂₀ alkynyl. In one embodiment, R₁₁ is C₁₄ alkynyl. In one embodiment, R₁₁ is C₁₅ alkynyl. In one embodiment, R₁₁ is C₁₆ alkynyl. In one embodiment, R₁₁ is C₁₇ alkynyl. In one embodiment, R₁₁ is C₁₈ alkynyl. In one embodiment, R₁₁ is C₁₉ alkynyl. In one embodiment, R₁₁ is C₂₀ alkynyl. In one embodiment, the alkenyl in R₁₁ has one carbon-carbon triple bond. In one embodiment, the alkenyl in R₁₁ has two carbon-carbon triple bond. In one embodiment, the alkynyl in R₁₁ is unsubstituted. In one embodiment, the alkynyl in R₁₁ is substituted. In one embodiment, the alkenyl in R₁₁ is substituted with one or more hydroxyl, halogen, cyano, C₁₋₃₀ alkyl, C₁₋₃₀ haloalkyl, C₁₋₃₀ alkoxy, —S—C₁₋₃₀ alkyl, amino, —NH—C₁₋₃₀ alkyl, or —N(C₁₋₃₀ alkyl)₂. In one embodiment, the alkenyl in R₁₁ is substituted with one or more hydroxyl, halogen, cyano, C₁₋₁₂ alkyl, C₁₋₁₂ haloalkyl, C₁₋₁₂ alkoxy, —S—C₁₋₁₂ alkyl, amino, —NH—C₁₋₁₂ alkyl, or —N(C₁₋₁₂ alkyl)₂. In one embodiment, the alkenyl in R₁₁ is substituted with one or more hydroxyl, halogen, cyano, C₁₋₆ alkyl, C₁₋₆haloalkyl, C₁₋₆alkoxy, —S—C₁₋₆ alkyl, amino, —NH—C₁₋₁₆ alkyl, or —N(C₁₋₆ alkyl)₂.

In one embodiment, R₁₂ is C₆₋₃₀ alkyl. In one embodiment, R₁₂ is C₈₋₂₆ alkyl. In one embodiment, R₁₂ is C₁₀₋₂₄ alkyl. In one embodiment, R₁₂ is C₁₂₋₂₂ alkyl. In one embodiment, R₁₂ is C₁₄₋₂₀ alkyl. In one embodiment, R₁₂ is C₁₄ alkyl. In one embodiment, R₁₂ is C₁₅ alkyl. In one embodiment, R₁₂ is C₁₆ alkyl. In one embodiment, R₁₂ is C₁₇ alkyl. In one embodiment, R₁₂ is C₁₈ alkyl. In one embodiment, R₁₂ is C₁₉ alkyl. In one embodiment, R₁₂ is C₂₀ alkyl. In one embodiment, the alkyl in R₁₂ is unsubstituted. In one embodiment, the alkyl in R₁₂ is substituted. In one embodiment, the alkyl in R₁₂ is substituted with one or more hydroxyl, halogen, cyano, C₁₋₃₀ alkyl, C₁₋₃₀ haloalkyl, C₁₋₃₀ alkoxy, —S—C₁₋₃₀ alkyl, amino, —NH—C₁₋₃₀ alkyl, or —N(C₁₋₃₀ alkyl)₂. In one embodiment, the alkyl in R₁₂ is substituted with one or more hydroxyl, halogen, cyano, C₁₋₁₂ alkyl, C₁₋₁₂ haloalkyl, C₁₋₁₂ alkoxy, —S—C₁₋₁₂ alkyl, amino, —NH—C₁₋₁₂ alkyl, or —N(C₁₋₁₂ alkyl)₂. In one embodiment, the alkyl in R₁₂ is substituted with one or more hydroxyl, halogen, cyano, C₁₋₆ alkyl, C₁₋₆haloalkyl, C₁₋₆ alkoxy, —S—C₁₋₆ alkyl, amino, —NH—C₁₋₁₆ alkyl, or —N(C₁₋₆ alkyl)₂.

In one embodiment, R₁₂ is C₆₋₃₀ alkenyl. In one embodiment, R₁₂ is C₈₋₂₆ alkenyl. In one embodiment, R₁₂ is C₁₀₋₂₄ alkenyl. In one embodiment, R₁₂ is C₁₂₋₂₂ alkenyl. In one embodiment, R₁₂ is C₁₄₋₂₀ alkenyl. In one embodiment, R₁₂ is C₁₄ alkenyl. In one embodiment, R₁₂ is C₁₅ alkenyl. In one embodiment, R₁₂ is C₁₆ alkenyl. In one embodiment, R₁₂ is C₁₇ alkenyl. In one embodiment, R₁₂ is C₁₈ alkenyl. In one embodiment, R₁₂ is C₁₉ alkenyl. In one embodiment, R₁₂ is C₂₀ alkenyl. In one embodiment, the alkenyl in R₁₂ has one C═C double bond. In one embodiment, the alkenyl in R₁₂ has two C═C double bond. In one embodiment, the alkenyl in R₁₂ has three C═C double bond. In one embodiment, the alkenyl in R₁₂ is unsubstituted. In one embodiment, the alkenyl in R₁₂ is substituted. In one embodiment, the alkenyl in R₁₂ is substituted with one or more hydroxyl, halogen, cyano, C₁₋₃₀ alkyl, C₁₋₃₀ haloalkyl, C₁₋₃₀ alkoxy, —S—C₁₋₃₀ alkyl, amino, —NH—C₁₋₃₀ alkyl, or —N(C₁₋₃₀ alkyl)₂. In one embodiment, the alkenyl in R₁₂ is substituted with one or more hydroxyl, halogen, cyano, C₁₋₁₂ alkyl, C₁₋₁₂ haloalkyl, C₁₋₁₂ alkoxy, —S—C₁₋₁₂ alkyl, amino, —NH—C₁₋₁₂ alkyl, or —N(C₁₋₁₂ alkyl)₂. In one embodiment, the alkenyl in R₁₂ is substituted with one or more hydroxyl, halogen, cyano, C₁₋₆ alkyl, C₁₋₆haloalkyl, C₁₋₆ alkoxy, —S—C₁₋₆ alkyl, amino, —NH—C₁₋₁₆ alkyl, or —N(C₁₋₆ alkyl)₂.

In one embodiment, R₁₂ is C₆₋₃₀ alkynyl. In one embodiment, R₁₂ is C₈₋₂₆ alkynyl. In one embodiment, R₁₂ is C₁₀₋₂₄ alkynyl. In one embodiment, R₁₂ is C₁₂₋₂₂ alkynyl. In one embodiment, R₁₂ is C₁₄₋₂₀ alkynyl. In one embodiment, R₁₂ is C₁₄ alkynyl. In one embodiment, R₁₂ is C₁₅ alkynyl. In one embodiment, R₁₂ is C₁₆ alkynyl. In one embodiment, R₁₂ is C₁₇ alkynyl. In one embodiment, R₁₂ is C₁₈ alkynyl. In one embodiment, R₁₂ is C₁₉ alkynyl. In one embodiment, R₁₂ is C₂₀ alkynyl. In one embodiment, the alkenyl in R₁₂ has one carbon-carbon triple bond. In one embodiment, the alkenyl in R₁₂ has two carbon-carbon triple bond. In one embodiment, the alkynyl in R₁₂ is unsubstituted. In one embodiment, the alkynyl in R₁₂ is substituted. In one embodiment, the alkenyl in R₁₂ is substituted with one or more hydroxyl, halogen, cyano, C₁₋₃₀ alkyl, C₁₋₃₀ haloalkyl, C₁₋₃₀ alkoxy, —S—C₁₋₃₀ alkyl, amino, —NH—C₁₋₃₀ alkyl, or —N(C₁₋₃₀ alkyl)₂. In one embodiment, the alkenyl in R₁₂ is substituted with one or more hydroxyl, halogen, cyano, C₁₋₁₂ alkyl, C₁₋₁₂ haloalkyl, C₁₋₁₂ alkoxy, —S—C₁₋₁₂ alkyl, amino, —NH—C₁₋₁₂ alkyl, or —N(C₁₋₁₂ alkyl)₂. In one embodiment, the alkenyl in R₁₂ is substituted with one or more hydroxyl, halogen, cyano, C₁₋₆ alkyl, C₁₋₆haloalkyl, C₁₋₆alkoxy, —S—C₁₋₆ alkyl, amino, —NH—C₁₋₁₆ alkyl, or —N(C₁₋₆ alkyl)₂.

In one embodiment, R₁₁ and R₁₂ are each independently C₁₅₋₂₀ alkyl, C₁₅₋₂₀ alkenyl, or C₁₅₋₂₀ alkynyl, and wherein the alkyl, alkenyl and alkynyl are independently optionally substituted with one or more groups selected from hydroxyl, halogen, cyano, C₁₋₂₀ alkyl, C₁₋₂₀ haloalkyl, C₁₋₂₀ alkoxy, —S—C₁₋₂₀ alkyl, amino, —NH—C₁₋₂₀ alkyl, and —N(C₁₋₂₀ alkyl)₂.

In one embodiment, R₁₁ is C₁₅₋₂₀ alkyl, and R₁₂ is C₁₅₋₂₀ alkyl. In one embodiment, R₁₁ is C₁₅₋₂₀ alkyl, and R₁₂ is C₁₅₋₂₀ alkenyl. In one embodiment, R₁₁ is C₁₅₋₂₀ alkenyl, and R₁₂ is C₁₅₋₂₀ alkenyl. In one embodiment, R₁₁ and R₁₂ are both unsubstituted.

In one embodiment, R₁₃ is C₁₋₆ alkyl. In one embodiment, R₁₃ is methyl. In one embodiment, R₁₃ is ethyl. In one embodiment, R₁₃ is C₃ alkyl. In one embodiment, R₁₃ is isopropyl. In one embodiment, R₁₃ is C₄ alkyl. In one embodiment, R₁₃ is C₅ alkyl. In one embodiment, R₁₃ is C₆ alkyl. In one embodiment, the alkyl in R₁₃ is unsubstituted. In one embodiment, the alkyl in R₁₃ is substituted. In one embodiment, the alkyl in R₁₃ is substituted with one or more hydroxyl, halogen, cyano, C₁₋₆ alkoxy, —S—C₁₋₆ alkyl, amino, —NH—C₁₋₆ alkyl, or —N(C₁₋₆ alkyl)₂.

In one embodiment, R₁₃ is C₁₋₆ haloalkyl. In one embodiment, R₁₃ is fluoromethyl. In one embodiment, R₁₃ is bromomethyl. In one embodiment, R₁₃ is difluoromethyl. In one embodiment, R₁₃ is trifluoromethyl. In one embodiment, R₁₃ is fluoroethyl. In one embodiment, R₁₃ is bromoethyl. In one embodiment, R₁₃ is difluoroethyl. In one embodiment, R₁₃ is trifluoroethyl. In one embodiment, R₁₃ is C₂ haloalkyl. In one embodiment, R₁₃ is C₃ haloalkyl. In one embodiment, R₁₃ is C₄ haloalkyl. In one embodiment, R₁₃ is C₅ haloalkyl. In one embodiment, R₁₃ is C₆ haloalkyl. In one embodiment, the haloalkyl in R₁₃ is unsubstituted. In one embodiment, the haloalkyl in R₁₃ is substituted. In one embodiment, the haloalkyl in R₁₃ is substituted with one or more hydroxyl, cyano, C₁₋₆ alkoxy, —S—C₁₋₆ alkyl, amino, —NH—C₁₋₆ alkyl, or —N(C₁₋₆ alkyl)₂.

In one embodiment, R₁₃ is C₂₋₆ alkenyl. In one embodiment, R₁₃ is ethenyl or vinyl. In one embodiment, R₁₃ is C₃ alkenyl. In one embodiment, R₁₃ is allyl. In one embodiment, R₁₃ is C₄ alkenyl. In one embodiment, R₁₃ is C₅ alkenyl. In one embodiment, R₁₃ is C₆ alkenyl. In one embodiment, the alkenyl in R₁₃ is unsubstituted. In one embodiment, the alkenyl in R₁₃ is substituted. In one embodiment, the alkenyl in R₁₃ is substituted with one or more hydroxyl, halogen, cyano, C₁₋₆ alkoxy, —S—C₁₋₆ alkyl, amino, —NH—C₁₋₆ alkyl, or —N(C₁₋₆ alkyl)₂.

In one embodiment, R₁₃ is C₂₋₆ alkynyl. In one embodiment, R₁₃ is ethyne. In one embodiment, R₁₃ is C₃ alkynyl. In one embodiment, R₁₃ is propyne. In one embodiment, R₁₃ is C₄ alkynyl. In one embodiment, R₁₃ is C₅ alkynyl. In one embodiment, R₁₃ is C₆ alkynyl. In one embodiment, the alkynyl in R₁₃ is unsubstituted. In one embodiment, the alkynyl in R₁₃ is substituted. In one embodiment, the alkynyl in R₁₃ is substituted with one or more hydroxyl, halogen, cyano, C₁₋₆ alkoxy, —S—C₁₋₆ alkyl, amino, —NH—C₁₋₆ alkyl, or —N(C₁₋₆ alkyl)₂.

In one embodiment, R₁₄ is C₁₋₆ alkyl. In one embodiment, R₁₄ is methyl. In one embodiment, R₁₄ is ethyl. In one embodiment, R₁₄ is C₃ alkyl. In one embodiment, R₁₄ is isopropyl. In one embodiment, R₁₄ is C₄ alkyl. In one embodiment, R₁₄ is C₅ alkyl. In one embodiment, R₁₄ is C₆ alkyl. In one embodiment, the alkyl in R₁₄ is unsubstituted. In one embodiment, the alkyl in R₁₄ is substituted. In one embodiment, the alkyl in R₁₄ is substituted with one or more hydroxyl, halogen, cyano, C₁₋₆ alkoxy, —S—C₁₋₆ alkyl, amino, —NH—C₁₋₆ alkyl, or —N(C₁₋₆ alkyl)₂.

In one embodiment, R₁₄ is C₁₋₆ haloalkyl. In one embodiment, R₁₄ is fluoromethyl. In one embodiment, R₁₄ is bromomethyl. In one embodiment, R₁₄ is difluoromethyl. In one embodiment, R₁₄ is trifluoromethyl. In one embodiment, R₁₄ is fluoroethyl. In one embodiment, R₁₄ is bromoethyl. In one embodiment, R₁₄ is difluoroethyl. In one embodiment, R₁₄ is trifluoroethyl. In one embodiment, R₁₄ is C₂ haloalkyl. In one embodiment, R₁₄ is C₃ haloalkyl. In one embodiment, R₁₄ is C₄ haloalkyl. In one embodiment, R₁₄ is C₅ haloalkyl. In one embodiment, R₁₄ is C₆ haloalkyl. In one embodiment, the haloalkyl in R₁₄ is unsubstituted. In one embodiment, the haloalkyl in R₁₄ is substituted. In one embodiment, the haloalkyl in R₁₄ is substituted with one or more hydroxyl, cyano, C₁₋₆ alkoxy, —S—C₁₋₆ alkyl, amino, —NH—C₁₋₆ alkyl, or —N(C₁₋₆ alkyl)₂.

In one embodiment, R₁₄ is C₂₋₆ alkenyl. In one embodiment, R₁₄ is ethenyl or vinyl. In one embodiment, R₁₄ is C₃ alkenyl. In one embodiment, R₁₄ is allyl. In one embodiment, R₁₄ is C₄ alkenyl. In one embodiment, R₁₄ is C₅ alkenyl. In one embodiment, R₁₄ is C₆ alkenyl. In one embodiment, the alkenyl in R₁₄ is unsubstituted. In one embodiment, the alkenyl in R₁₄ is substituted. In one embodiment, the alkenyl in R₁₄ is substituted with one or more hydroxyl, halogen, cyano, C₁₋₆ alkoxy, —S—C₁₋₆ alkyl, amino, —NH—C₁₋₆ alkyl, or —N(C₁₋₆ alkyl)₂.

In one embodiment, R₁₄ is C₂₋₆ alkynyl. In one embodiment, R₁₄ is ethyne. In one embodiment, R₁₄ is C₃ alkynyl. In one embodiment, R₁₄ is propyne. In one embodiment, R₁₄ is C₄ alkynyl. In one embodiment, R₁₄ is C₅ alkynyl. In one embodiment, R₁₄ is C₆ alkynyl. In one embodiment, the alkynyl in R₁₄ is unsubstituted. In one embodiment, the alkynyl in R₁₄ is substituted. In one embodiment, the alkynyl in R₁₄ is substituted with one or more hydroxyl, halogen, cyano, C₁₋₆ alkoxy, —S—C₁₋₆ alkyl, amino, —NH—C₁₋₆ alkyl, or —N(C₁₋₆ alkyl)₂.

In one embodiment, R₁₅ is C₁₋₆ alkyl. In one embodiment, R₁₅ is methyl. In one embodiment, R₁₅ is ethyl. In one embodiment, R₁₅ is C₃ alkyl. In one embodiment, R₁₅ is isopropyl. In one embodiment, R₁₅ is C₄ alkyl. In one embodiment, R₁₅ is C₅ alkyl. In one embodiment, R₁₅ is C₆ alkyl. In one embodiment, the alkyl in R₁₅ is unsubstituted. In one embodiment, the alkyl in R₁₅ is substituted. In one embodiment, the alkyl in R₁₅ is substituted with one or more hydroxyl, halogen, cyano, C₁₋₆ alkoxy, —S—C₁₋₆ alkyl, amino, —NH—C₁₋₆ alkyl, or —N(C₁₋₆ alkyl)₂.

In one embodiment, R₁₅ is C₁₋₆ haloalkyl. In one embodiment, R₁₅ is fluoromethyl. In one embodiment, R₁₅ is bromomethyl. In one embodiment, R₁₅ is difluoromethyl. In one embodiment, R₁₅ is trifluoromethyl. In one embodiment, R₁₅ is fluoroethyl. In one embodiment, R₁₅ is bromoethyl. In one embodiment, R₁₅ is difluoroethyl. In one embodiment, R₁₅ is trifluoroethyl. In one embodiment, R₁₅ is C₂ haloalkyl. In one embodiment, R₁₅ is C₃ haloalkyl. In one embodiment, R₁₅ is C₄ haloalkyl. In one embodiment, R₁₅ is C₅ haloalkyl. In one embodiment, R₁₅ is C₆ haloalkyl. In one embodiment, the haloalkyl in R₁₅ is unsubstituted. In one embodiment, the haloalkyl in R₁₅ is substituted. In one embodiment, the haloalkyl in R₁₅ is substituted with one or more hydroxyl, cyano, C₁₋₆ alkoxy, —S—C₁₋₆ alkyl, amino, —NH—C₁₋₆ alkyl, or —N(C₁₋₆ alkyl)₂.

In one embodiment, R₁₅ is C₂₋₆ alkenyl. In one embodiment, R₁₅ is ethenyl or vinyl. In one embodiment, R₁₅ is C₃ alkenyl. In one embodiment, R₁₅ is allyl. In one embodiment, R₁₅ is C₄ alkenyl. In one embodiment, R₁₅ is C₅ alkenyl. In one embodiment, R₁₅ is C₆ alkenyl. In one embodiment, the alkenyl in R₁₅ is unsubstituted. In one embodiment, the alkenyl in R₁₅ is substituted. In one embodiment, the alkenyl in R₁₅ is substituted with one or more hydroxyl, halogen, cyano, C₁₋₆ alkoxy, —S—C₁₋₆ alkyl, amino, —NH—C₁₋₆ alkyl, or —N(C₁₋₆ alkyl)₂.

In one embodiment, R₁₅ is C₂₋₆ alkynyl. In one embodiment, R₁₅ is ethyne. In one embodiment, R₁₅ is C₃ alkynyl. In one embodiment, R₁₅ is propyne. In one embodiment, R₁₅ is C₄ alkynyl. In one embodiment, R₁₅ is C₅ alkynyl. In one embodiment, R₁₅ is C₆ alkynyl. In one embodiment, the alkynyl in R₁₅ is unsubstituted. In one embodiment, the alkynyl in R₁₅ is substituted. In one embodiment, the alkynyl in R₁₅ is substituted with one or more hydroxyl, halogen, cyano, C₁₋₆ alkoxy, —S—C₁₋₆ alkyl, amino, —NH—C₁₋₆ alkyl, or —N(C₁₋₆ alkyl)₂.

In one embodiment, any two of R₁₃, R₁₄, and R₁₅ together with the nitrogen atom they are attached to form a 4 to 8-membered ring. In one embodiment, any two of R₁₃, R₁₄, and R₁₅ together with the nitrogen atom they are attached to form a C₄₋₈ cycloalkyl. In one embodiment, any two of R₁₃, R₁₄, and R₁₅ together with the nitrogen atom they are attached to form a 4- to 8-membered heterocyclyl.

In one embodiment, R₁₃, R₁₄, and R₁₅ are each independently C₁₋₆ alkyl optionally substituted with hydroxyl, halogen, cyano, C₁₋₆ alkoxy, —S—C₁₋₆ alkyl, amino, —NH—C₁₋₆ alkyl, or —N(C₁₋₆ alkyl)₂.

In one embodiment, R₁₃, R₁₄, and R₁₅ are all unsubstituted C₁₋₆ alkyl. In one embodiment, R₁₃, R₁₄, and R₁₅ are all methyl.

In one embodiment, n₁ is 0. In one embodiment, n₁ is 1. In one embodiment, n₂ is 0. In one embodiment, n₂ is 1. In one embodiment, n₁ is 0 and n₂ is 0. In one embodiment, n₁ is 0 and n₂ is 1. In one embodiment, n₁ is 1 and n₂ is 0. In one embodiment, n₁ is 1 and n₂ is 1.

In one embodiment, X is halide anion. In one embodiment, X is bromide. In one embodiment, X is chloride. In one embodiment, X is iodide. In one embodiment, X is hydroxide. In one embodiment, X is nitrate. In one embodiment, X is nitrite. In one embodiment, X is perchlorate. In one embodiment, X is thiocyanate.

In one embodiment, the permanently cationic lipid is a pharmaceutically acceptable salt of

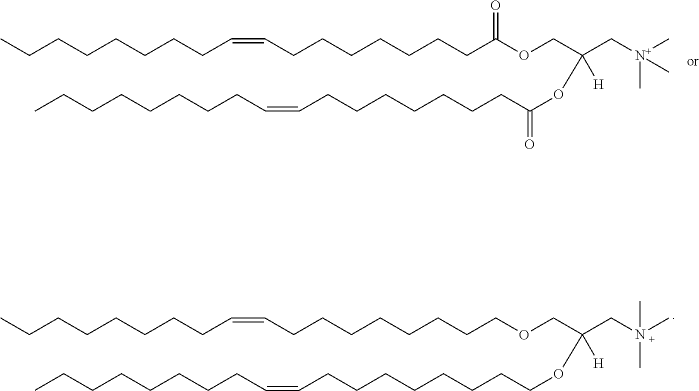

In one embodiment, the permanently cationic lipid is a compound of formula (II):

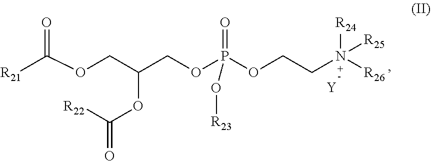

or a stereoisomer, a mixture of stereoisomers, or a pharmaceutically acceptable salt thereof, wherein R₂₁ and R₂ are each independently C₆₋₃₀ alkyl, C₆₋₃₀ alkenyl, or C₆₋₃₀ alkynyl, and wherein the alkyl, alkenyl and alkynyl are independently optionally substituted with one or more groups selected from hydroxyl, halogen, cyano, C₁₋₃₀ alkyl, C₁₋₃₀ haloalkyl, C₁₋₃₀ alkoxy, —S—C₁₋₃₀ alkyl, amino, —NH—C₁₋₃₀alkyl, and —N(C₁₋₃₀ alkyl)₂;
   R₂₃ is C₁₋₆ alkyl, C₁₋₆ haloalkyl, C₂₋₆ alkenyl, or C₂₋₆ alkynyl, and wherein R₂₃ is optionally substituted with one or more groups selected from halogen, hydroxyl, C₁₋₆ alkyl, C₁₋₆ haloalkyl, C₁₋₆alkoxy, —OC(═O)R₂ₐ, —C(═O)OR₂ₐ, —C(═O)NHR₂ₐ, and —NHC(═O)R₂ₐ;
   R₂ₐ is hydrogen, C₁₋₆ alkyl, or C₁₋₆ haloalkyl;
   R₂₄, R₂₅, and R₂₆ are each independently C₁₋₆ alkyl, C₁₋₆ haloalkyl, C₂₋₆ alkenyl, C₂₋₆ alkynyl, or any two of R₂₄, R₃₅, and R₂₆ together with the nitrogen atom they are attached to form a 4 to 8-membered ring, and wherein the alkyl, haloalkyl, alkenyl, alkynyl, and ring are optionally substituted; and
   Y⁻ is an anion.

In one embodiment, R₂₁ is C₆₋₃₀ alkenyl. In one embodiment, R₂₁ is C₈₋₂₆ alkenyl. In one embodiment, R₂₁ is C₁₀₋₂₄ alkenyl. In one embodiment, R₂₁ is C₁₂₋₂₂ alkenyl. In one embodiment, R₂₁ is C₁₄₋₂₀ alkenyl. In one embodiment, R₂₁ is C₁₄ alkenyl. In one embodiment, R₂₁ is C₁₅ alkenyl. In one embodiment, R₂₁ is C₁₆ alkenyl. In one embodiment, R₂₁ is C₁₇ alkenyl. In one embodiment, R₂₁ is C₁₈ alkenyl. In one embodiment, R₂₁ is C₁₉ alkenyl. In one embodiment, R₂₁ is C₂₀ alkenyl. In one embodiment, the alkenyl in R₂₁ has one C═C double bond. In one embodiment, the alkenyl in R₂₁ has two C═C double bond. In one embodiment, the alkenyl in R₂₁ has three C═C double bond. In one embodiment, the alkenyl in R₂₁ is unsubstituted. In one embodiment, the alkenyl in R₂₁ is substituted. In one embodiment, the alkenyl in R₂₁ is substituted with one or more hydroxyl, halogen, cyano, C₁₋₃₀ alkyl, C₁₋₃₀ haloalkyl, C₁₋₃₀ alkoxy, —S—C₁₋₃₀ alkyl, amino, —NH—C₁₋₃₀ alkyl, or —N(C₁₋₃₀ alkyl)₂. In one embodiment, the alkenyl in R₂₁ is substituted with one or more hydroxyl, halogen, cyano, C₁₋₁₂ alkyl, C₁₋₁₂ haloalkyl, C₁₋₁₂ alkoxy, —S—C₁₋₁₂ alkyl, amino, —NH—C₁₋₁₂ alkyl, or —N(C₁₋₁₂ alkyl)₂. In one embodiment, the alkenyl in R₂₁ is substituted with one or more hydroxyl, halogen, cyano, C₁₋₆ alkyl, C₁₋₆ haloalkyl, C₁₋₆ alkoxy, —S—C₁₋₆ alkyl, amino, —NH—C₁₋₁₆ alkyl, or —N(C₁₋₆ alkyl)₂.

In one embodiment, R₂₁ is C₆₋₃₀ alkynyl. In one embodiment, R₂₁ is C₈₋₂₆ alkynyl. In one embodiment, R₂₁ is C₁₀₋₂₄ alkynyl. In one embodiment, R₂₁ is C₁₂₋₂₂ alkynyl. In one embodiment, R₂₁ is C₁₄₋₂₀ alkynyl. In one embodiment, R₂₁ is C₁₄ alkynyl. In one embodiment, R₂₁ is C₁₅ alkynyl. In one embodiment, R₂₁ is C₁₆ alkynyl. In one embodiment, R₂₁ is C₁₇ alkynyl. In one embodiment, R₂₁ is C₁₈ alkynyl. In one embodiment, R₂₁ is C₁₉ alkynyl. In one embodiment, R₂₁ is C₂₀ alkynyl. In one embodiment, the alkenyl in R₂₁ has one carbon-carbon triple bond. In one embodiment, the alkenyl in R₂₁ has two carbon-carbon triple bond. In one embodiment, the alkynyl in R₂₁ is unsubstituted. In one embodiment, the alkynyl in R₂₁ is substituted. In one embodiment, the alkynyl in R₂₁ is substituted with one or more hydroxyl, halogen, cyano, C₁₋₃₀ alkyl, C₁₋₃₀ haloalkyl, C₁₋₃₀ alkoxy, —S—C₁₋₃₀ alkyl, amino, —NH—C₁₋₃₀ alkyl, or —N(C₁₋₃₀ alkyl)₂. In one embodiment, the alkynyl in R₂₁ is substituted with one or more hydroxyl, halogen, cyano, C₁₋₁₂ alkyl, C₁₋₁₂ haloalkyl, C₁₋₁₂ alkoxy, —S—C₁₋₁₂ alkyl, amino, —NH—C₁₋₁₂ alkyl, or —N(C₁₋₁₂ alkyl)₂. In one embodiment, the alkynyl in R₂₁ is substituted with one or more hydroxyl, halogen, cyano, C₁₋₆alkyl, C₁₋₆haloalkyl, C₁₋₆alkoxy, —S—C₁₋₆alkyl, amino, —NH—C₁₋₁₆alkyl, or —N(C₁₋₆ alkyl)₂.

In one embodiment, R₂₂ is C₆₋₃₀ alkenyl. In one embodiment, R₂₂ is C₈₋₂₆ alkenyl. In one embodiment, R₂₂ is C₁₀₋₂₄ alkenyl. In one embodiment, R₂₂ is C₁₂₋₂₂ alkenyl. In one embodiment, R₂₂ is C₁₄₋₂₀ alkenyl. In one embodiment, R₂₂ is C₁₄ alkenyl. In one embodiment, R₂₂ is C₁₅ alkenyl. In one embodiment, R₂₂ is C₁₆ alkenyl. In one embodiment, R₂₂ is C₁₇ alkenyl. In one embodiment, R₂₂ is C₁₈ alkenyl. In one embodiment, R₂₂ is C₁₉ alkenyl. In one embodiment, R₂₂ is C₂₀ alkenyl. In one embodiment, the alkenyl in R₂₂ has one C═C double bond. In one embodiment, the alkenyl in R₂₂ has two C═C double bond. In one embodiment, the alkenyl in R₂₂ has three C═C double bond. In one embodiment, the alkenyl in R₂₂ is unsubstituted. In one embodiment, the alkenyl in R₂₂ is substituted. In one embodiment, the alkenyl in R₂₂ is substituted with one or more hydroxyl, halogen, cyano, C₁₋₃₀ alkyl, C₁₋₃₀ haloalkyl, C₁₋₃₀ alkoxy, —S—C₁₋₃₀ alkyl, amino, —NH—C₁₋₃₀ alkyl, or —N(C₁₋₃₀ alkyl)₂. In one embodiment, the alkenyl in R₂₂ is substituted with one or more hydroxyl, halogen, cyano, C₁₋₁₂ alkyl, C₁₋₁₂ haloalkyl, C₁₋₁₂ alkoxy, —S—C₁₋₁₂ alkyl, amino, —NH—C₁₋₁₂ alkyl, or —N(C₁₋₁₂ alkyl)₂. In one embodiment, the alkenyl in R₂₂ is substituted with one or more hydroxyl, halogen, cyano, C₁₋₆ alkyl, C₁₋₆ haloalkyl, C₁₋₆ alkoxy, —S—C₁₋₆ alkyl, amino, —NH—C₁₋₁₆ alkyl, or —N(C₁₋₆ alkyl)₂.

In one embodiment, R₂₂ is C₆₋₃₀ alkynyl. In one embodiment, R₂₂ is C₈₋₂₆ alkynyl. In one embodiment, R₂₂ is C₁₀₋₂₄ alkynyl. In one embodiment, R₂₂ is C₁₂₋₂₂ alkynyl. In one embodiment, R₂₂ is C₁₄₋₂₀ alkynyl. In one embodiment, R₂₂ is C₁₄ alkynyl. In one embodiment, R₂₂ is C₁₅ alkynyl. In one embodiment, R₂₂ is C₁₆ alkynyl. In one embodiment, R₂₂ is C₁₇ alkynyl. In one embodiment, R₂₂ is C₁₈ alkynyl. In one embodiment, R₂₂ is C₁₉ alkynyl. In one embodiment, R₂₂ is C₂₀ alkynyl. In one embodiment, the alkenyl in R₂₂ has one carbon-carbon triple bond. In one embodiment, the alkenyl in R₂₂ has two carbon-carbon triple bond. In one embodiment, the alkynyl in R₂₂ is unsubstituted. In one embodiment, the alkynyl in R₂₂ is substituted. In one embodiment, the alkenyl in R₂₂ is substituted with one or more hydroxyl, halogen, cyano, C₁₋₃₀ alkyl, C₁₋₃₀ haloalkyl, C₁₋₃₀ alkoxy, —S—C₁₋₃₀ alkyl, amino, —NH—C₁₋₃₀ alkyl, or —N(C₁₋₃₀ alkyl)₂. In one embodiment, the alkenyl in R₂₂ is substituted with one or more hydroxyl, halogen, cyano, C₁₋₁₂ alkyl, C₁₋₁₂ haloalkyl, C₁₋₁₂ alkoxy, —S—C₁₋₁₂ alkyl, amino, —NH—C₁₋₁₂ alkyl, or —N(C₁₋₁₂ alkyl)₂. In one embodiment, the alkenyl in R₂₂ is substituted with one or more hydroxyl, halogen, cyano, C₁₋₆ alkyl, C₁₋₆ haloalkyl, C₁₋₆ alkoxy, —S—C₁₋₆ alkyl, amino, —NH—C₁₋₁₆ alkyl, or —N(C₁₋₆ alkyl)₂.

In one embodiment, R₂₁ and R₂₂ are each independently C₁₀₋₂₅ alkyl, C₁₀₋₂₅ alkenyl, or C₁₀₋₂₅ alkynyl, and wherein the alkyl, alkenyl and alkynyl are independently optionally substituted with one or more groups selected from hydroxyl, halogen, cyano, C₁₋₂₅ alkyl, C₁₋₂₅ haloalkyl, C₁₋₂₅ alkoxy, —S—C₁₋₂₅ alkyl, amino, —NH—C₁₋₂₅ alkyl, and —N(C₁₋₂₅ alkyl)₂.

In one embodiment, R₂₁ is C₁₅₋₂₀ alkyl, and R₂₂ is C₁₅₋₂₀ alkyl. In one embodiment, R₂₁ is C₁₅₋₂₀ alkyl, and R₂₂ is C₁₅₋₂₀ alkenyl. In one embodiment, R₂₁ is C₁₅₋₂₀ alkenyl, and R₂₂ is C₁₅₋₂₀ alkenyl. In one embodiment, R₂₁ and R₂₂ are both unsubstituted.

In one embodiment, R₂₃ is C₁₋₆ alkyl. In one embodiment, R₂₃ is methyl. In one embodiment, R₂₃ is ethyl. In one embodiment, R₂₃ is C₃ alkyl. In one embodiment, R₂₃ is isopropyl. In one embodiment, R₂₃ is C₄ alkyl. In one embodiment, R₂₃ is C₅ alkyl. In one embodiment, R₂₃ is C₆ alkyl. In one embodiment, the alkyl in R₂₃ is unsubstituted. In one embodiment, the alkyl in R₂₃ is substituted. In one embodiment, the alkyl in R₂₃ is substituted with one or more halogen, hydroxyl, C₁₋₆ alkoxy, —OC(═O)R₂ₐ, —C(═O)OR₂ₐ, —C(═O)NHR₂ₐ, or —NHC(═O)R₂ₐ.

In one embodiment, R₂₃ is C₁₋₆ alkyl substituted with —C(═O)OR₂ₐ. In one embodiment, R₂₃ is C₁₋₆ alkyl substituted with —OC(═O)R₂ₐ. In one embodiment, R₂₃ is C₁₋₆ alkyl substituted with —C(═O)NHR₂ₐ. In one embodiment, R₂₃ is C₁₋₆ alkyl substituted with —NHC(═O)R₂ₐ. In one embodiment, R₂₃ is —CH₂—C(═O)OR₂ₐ. In one embodiment, R₂₃ is —CH₂—OC(═O)R₂ₐ. In one embodiment, R₂₃ is —CH₂—C(═O)NHR₂ₐ. In one embodiment, R₂₃ is —CH₂—C(═O)OR₂ₐ. In one embodiment, R₂₃ is —CH₂—NHC(═O)R₂ₐ.

In one embodiment, R₂₃ is C₁₋₆ haloalkyl. In one embodiment, R₂₃ is fluoromethyl. In one embodiment, R₂₃ is bromomethyl. In one embodiment, R₂₃ is difluoromethyl. In one embodiment, R₂₃ is trifluoromethyl. In one embodiment, R₂₃ is fluoroethyl. In one embodiment, R₂₃ is bromoethyl. In one embodiment, R₂₃ is difluoroethyl. In one embodiment, R₂₃ is trifluoroethyl. In one embodiment, R₂₃ is C₂ haloalkyl. In one embodiment, R₂₃ is C₃ haloalkyl. In one embodiment, R₂₃ is C₄ haloalkyl. In one embodiment, R₂₃ is C₅ haloalkyl. In one embodiment, R₂₃ is C₆ haloalkyl. In one embodiment, the haloalkyl in R₂₃ is unsubstituted. In one embodiment, the haloalkyl in R₂₃ is substituted. In one embodiment, the haloalkyl in R₂₃ is substituted with one or more halogen, hydroxyl, C₁₋₆ alkoxy, —OC(═O)R₂ₐ, —C(═O)OR₂ₐ, —C(═O)NHR₂ₐ, or —NHC(═O)R₂ₐ.

In one embodiment, R₂₃ is C₂₋₆ alkenyl. In one embodiment, R₂₃ is ethenyl or vinyl. In one embodiment, R₂₃ is C₃ alkenyl. In one embodiment, R₂₃ is allyl. In one embodiment, R₂₃ is C₄ alkenyl. In one embodiment, R₂₃ is C₅ alkenyl. In one embodiment, R₂₃ is C₆ alkenyl. In one embodiment, the alkenyl in R₂₃ is unsubstituted. In one embodiment, the alkenyl in R₂₃ is substituted. In one embodiment, the alkenyl in R₂₃ is substituted with one or more halogen, hydroxyl, C₁₋₆ alkyl, C₁₋₆ haloalkyl, C₁₋₆ alkoxy, —OC(═O)R₂ₐ, —C(═O)OR₂ₐ, —C(═O)NHR₂ₐ, or —NHC(═O)R₂ₐ.

In one embodiment, R₂₃ is C₂₋₆ alkynyl. In one embodiment, R₂₃ is ethyne. In one embodiment, R₂₃ is C₃ alkynyl. In one embodiment, R₂₃ is propyne. In one embodiment, R₂₃ is C₄ alkynyl. In one embodiment, R₂₃ is C₅ alkynyl. In one embodiment, R₂₃ is C₆ alkynyl. In one embodiment, the alkynyl in R₂₃ is unsubstituted. In one embodiment, the alkynyl in R₂₃ is substituted. In one embodiment, the alkynyl in R₂₃ is substituted with one or more halogen, hydroxyl, C₁₋₆ alkyl, C₁₋₆ haloalkyl, C₁₋₆ alkoxy, —OC(═O)R₂ₐ, —C(═O)OR₂ₐ, —C(═O)NHR₂ₐ, or —NHC(═O)R₂ₐ.

In one embodiment, R₂ₐ is hydrogen. In one embodiment, R₂ₐ is C₁₋₆ alkyl. In one embodiment, R₂ₐ is C₁₋₆ haloalkyl. In one embodiment, R₂ₐ is methyl. In one embodiment, R₂ₐ is ethyl. In one embodiment, R₂ₐ is C₃ alkyl. In one embodiment, R₂ₐ is isopropyl. In one embodiment, R₂ₐ is C₄ alkyl. In one embodiment, R₂ₐ is C₅ alkyl. In one embodiment, R₂ₐ is C₆ alkyl. In one embodiment, R₂ₐ is fluoromethyl. In one embodiment, R₂ₐ is bromomethyl. In one embodiment, R₂ₐ is difluoromethyl. In one embodiment, R₂ₐ is trifluoromethyl. In one embodiment, R₂ₐ is C₂ haloalkyl. In one embodiment, R₂ₐ is C₃ haloalkyl. In one embodiment, R₂ₐ is C₄ haloalkyl. In one embodiment, R₂ₐ is C₅ haloalkyl. In one embodiment, R₂ₐ is C₆ haloalkyl.

In one embodiment, R₂₄ is C₁₋₆ alkyl. In one embodiment, R₂₄ is methyl. In one embodiment, R₂₄ is ethyl. In one embodiment, R₂₄ is C₃ alkyl. In one embodiment, R₂₄ is isopropyl. In one embodiment, R₂₄ is C₄ alkyl. In one embodiment, R₂₄ is C₅ alkyl. In one embodiment, R₂₄ is C₆ alkyl. In one embodiment, the alkyl in R₂₄ is unsubstituted. In one embodiment, the alkyl in R₂₄ is substituted. In one embodiment, the alkyl in R₂₄ is substituted with one or more hydroxyl, halogen, cyano, C₁₋₆ alkoxy, —S—C₁₋₆ alkyl, amino, —NH—C₁₋₆ alkyl, or —N(C₁₋₆ alkyl)₂.

In one embodiment, R₂₄ is C₁₋₆ haloalkyl. In one embodiment, R₂₄ is fluoromethyl. In one embodiment, R₂₄ is bromomethyl. In one embodiment, R₂₄ is difluoromethyl. In one embodiment, R₂₄ is trifluoromethyl. In one embodiment, R₂₄ is fluoroethyl. In one embodiment, R₂₄ is bromoethyl. In one embodiment, R₂₄ is difluoroethyl. In one embodiment, R₂₄ is trifluoroethyl. In one embodiment, R₂₄ is C₂ haloalkyl. In one embodiment, R₂₄ is C₃ haloalkyl. In one embodiment, R₂₄ is C₄ haloalkyl. In one embodiment, R₂₄ is C₅ haloalkyl. In one embodiment, R₂₄ is C₆ haloalkyl. In one embodiment, the haloalkyl in R₂₄ is unsubstituted. In one embodiment, the haloalkyl in R₂₄ is substituted. In one embodiment, the haloalkyl in R₂₄ is substituted with one or more hydroxyl, cyano, C₁₋₆ alkoxy, —S—C₁₋₆ alkyl, amino, —NH—C₁₋₆ alkyl, or —N(C₁₋₆ alkyl)₂.

In one embodiment, R₂₄ is C₂₋₆ alkenyl. In one embodiment, R₂₄ is ethenyl or vinyl. In one embodiment, R₂₄ is C₃ alkenyl. In one embodiment, R₂₄ is allyl. In one embodiment, R₂₄ is C₄ alkenyl. In one embodiment, R₂₄ is C₅ alkenyl. In one embodiment, R₂₄ is C₆ alkenyl. In one embodiment, the alkenyl in R₂₄ is unsubstituted. In one embodiment, the alkenyl in R₂₄ is substituted. In one embodiment, the alkenyl in R₂₄ is substituted with one or more hydroxyl, halogen, cyano, C₁₋₆ alkoxy, —S—C₁₋₆ alkyl, amino, —NH—C₁₋₆ alkyl, or —N(C₁₋₆ alkyl)₂.

In one embodiment, R₂₄ is C₂₋₆ alkynyl. In one embodiment, R₂₄ is ethyne. In one embodiment, R₂₄ is C₃ alkynyl. In one embodiment, R₂₄ is propyne. In one embodiment, R₂₄ is C₄ alkynyl. In one embodiment, R₂₄ is C₅ alkynyl. In one embodiment, R₂₄ is C₆ alkynyl. In one embodiment, the alkynyl in R₂₄ is unsubstituted. In one embodiment, the alkynyl in R₂₄ is substituted. In one embodiment, the alkynyl in R₂₄ is substituted with one or more hydroxyl, halogen, cyano, C₁₋₆ alkoxy, —S—C₁₋₆ alkyl, amino, —NH—C₁₋₆ alkyl, or —N(C₁₋₆ alkyl)₂.

In one embodiment, R₂₅ is C₁₋₆ alkyl. In one embodiment, R₂₅ is methyl. In one embodiment, R₂₅ is ethyl. In one embodiment, R₂₅ is C₃ alkyl. In one embodiment, R₂₅ is isopropyl. In one embodiment, R₂₅ is C₄ alkyl. In one embodiment, R₂₅ is C₅ alkyl. In one embodiment, R₂₅ is C₆ alkyl. In one embodiment, the alkyl in R₂₅ is unsubstituted. In one embodiment, the alkyl in R₂₅ is substituted. In one embodiment, the alkyl in R₂₅ is substituted with one or more hydroxyl, halogen, cyano, C₁₋₆ alkoxy, —S—C₁₋₆ alkyl, amino, —NH—C₁₋₆ alkyl, or —N(C₁₋₆ alkyl)₂.

In one embodiment, R₂₅ is C₁₋₆ haloalkyl. In one embodiment, R₂₅ is fluoromethyl. In one embodiment, R₂₅ is bromomethyl. In one embodiment, R₂₅ is difluoromethyl. In one embodiment, R₂₅ is trifluoromethyl. In one embodiment, R₂₅ is fluoroethyl. In one embodiment, R₂₅ is bromoethyl. In one embodiment, R₂₅ is difluoroethyl. In one embodiment, R₂₅ is trifluoroethyl. In one embodiment, R₂₅ is C₂ haloalkyl. In one embodiment, R₂₅ is C₃ haloalkyl. In one embodiment, R₂₅ is C₄ haloalkyl. In one embodiment, R₂₅ is C₅ haloalkyl. In one embodiment, R₂₅ is C₆ haloalkyl. In one embodiment, the haloalkyl in R₂₅ is unsubstituted. In one embodiment, the haloalkyl in R₂₅ is substituted. In one embodiment, the haloalkyl in R₂₅ is substituted with one or more hydroxyl, cyano, C₁₋₆ alkoxy, —S—C₁₋₆ alkyl, amino, —NH—C₁₋₆ alkyl, or —N(C₁₋₆ alkyl)₂.

In one embodiment, R₂₅ is C₂₋₆ alkenyl. In one embodiment, R₂₅ is ethenyl or vinyl. In one embodiment, R₂₅ is C₃ alkenyl. In one embodiment, R₂₅ is allyl. In one embodiment, R₂₅ is C₄ alkenyl. In one embodiment, R₂₅ is C₅ alkenyl. In one embodiment, R₂₅ is C₆ alkenyl. In one embodiment, the alkenyl in R₂₅ is unsubstituted. In one embodiment, the alkenyl in R₂₅ is substituted. In one embodiment, the alkenyl in R₂₅ is substituted with one or more hydroxyl, halogen, cyano, C₁₋₆ alkoxy, —S—C₁₋₆ alkyl, amino, —NH—C₁₋₆ alkyl, or —N(C₁₋₆ alkyl)₂.

In one embodiment, R₂₅ is C₂₋₆ alkynyl. In one embodiment, R₂₅ is ethyne. In one embodiment, R₂₅ is C₃ alkynyl. In one embodiment, R₂₅ is propyne. In one embodiment, R₂₅ is C₄ alkynyl. In one embodiment, R₂₅ is C₅ alkynyl. In one embodiment, R₂₅ is C₆ alkynyl. In one embodiment, the alkynyl in R₂₅ is unsubstituted. In one embodiment, the alkynyl in R₂₅ is substituted. In one embodiment, the alkynyl in R₂₅ is substituted with one or more hydroxyl, halogen, cyano, C₁₋₆ alkoxy, —S—C₁₋₆ alkyl, amino, —NH—C₁₋₆ alkyl, or —N(C₁₋₆ alkyl)₂.

In one embodiment, R₂₆ is C₁₋₆ alkyl. In one embodiment, R₂₆ is methyl. In one embodiment, R₂₆ is ethyl. In one embodiment, R₂₆ is C₃ alkyl. In one embodiment, R₂₆ is isopropyl. In one embodiment, R₂₆ is C₄ alkyl. In one embodiment, R₂₆ is C₅ alkyl. In one embodiment, R₂₆ is C₆ alkyl. In one embodiment, the alkyl in R₂₆ is unsubstituted. In one embodiment, the alkyl in R₂₆ is substituted. In one embodiment, the alkyl in R₂₆ is substituted with one or more hydroxyl, halogen, cyano, C₁₋₆ alkoxy, —S—C₁₋₆ alkyl, amino, —NH—C₁₋₆ alkyl, or —N(C₁₋₆ alkyl)₂.

In one embodiment, R₂₆ is C₁₋₆ haloalkyl. In one embodiment, R₂₆ is fluoromethyl. In one embodiment, R₂₆ is bromomethyl. In one embodiment, R₂₆ is difluoromethyl. In one embodiment, R₂₆ is trifluoromethyl. In one embodiment, R₂₆ is fluoroethyl. In one embodiment, R₂₆ is bromoethyl. In one embodiment, R₂₆ is difluoroethyl. In one embodiment, R₂₆ is trifluoroethyl. In one embodiment, R₂₆ is C₂ haloalkyl. In one embodiment, R₂₆ is C₃ haloalkyl. In one embodiment, R₂₆ is C₄ haloalkyl. In one embodiment, R₂₆ is C₅ haloalkyl. In one embodiment, R₂₆ is C₆ haloalkyl. In one embodiment, the haloalkyl in R₂₆ is unsubstituted. In one embodiment, the haloalkyl in R₂₆ is substituted. In one embodiment, the haloalkyl in R₂₆ is substituted with one or more hydroxyl, cyano, C₁₋₆ alkoxy, —S—C₁₋₆ alkyl, amino, —NH—C₁₋₆ alkyl, or —N(C₁₋₆ alkyl)₂.

In one embodiment, R₂₆ is C₂₋₆ alkenyl. In one embodiment, R₂₆ is ethenyl or vinyl. In one embodiment, R₂₆ is C₃ alkenyl. In one embodiment, R₂₆ is allyl. In one embodiment, R₂₆ is C₄ alkenyl. In one embodiment, R₂₆ is C₅ alkenyl. In one embodiment, R₂₆ is C₆ alkenyl. In one embodiment, the alkenyl in R₂₆ is unsubstituted. In one embodiment, the alkenyl in R₂₆ is substituted. In one embodiment, the alkenyl in R₂₆ is substituted with one or more hydroxyl, halogen, cyano, C₁₋₆ alkoxy, —S—C₁₋₆ alkyl, amino, —NH—C₁₋₆ alkyl, or —N(C₁₋₆ alkyl)₂.

In one embodiment, R₂₆ is C₂₋₆ alkynyl. In one embodiment, R₂₆ is ethyne. In one embodiment, R₂₆ is C₃ alkynyl. In one embodiment, R₂₆ is propyne. In one embodiment, R₂₆ is C₄ alkynyl. In one embodiment, R₂₆ is C₅ alkynyl. In one embodiment, R₂₆ is C₆ alkynyl. In one embodiment, the alkynyl in R₂₆ is unsubstituted. In one embodiment, the alkynyl in R₂₆ is substituted. In one embodiment, the alkynyl in R₂₆ is substituted with one or more hydroxyl, halogen, cyano, C₁₋₆ alkoxy, —S—C₁₋₆ alkyl, amino, —NH—C₁₋₆ alkyl, or —N(C₁₋₆ alkyl)₂.

In one embodiment, any two of R₂₄, R₂₅, and R₂₆ together with the nitrogen atom they are attached to form a 4 to 8-membered ring. In one embodiment, any two of R₂₄, R₂₅, and R₂₆ together with the nitrogen atom they are attached to form a 5 or 6-membered ring. In one embodiment, any two of R₂₄, R₂₅, and R₂₆ together with the nitrogen atom they are attached to form a C₄₋₈ cycloalkyl. In one embodiment, any two of R₂₄, R₂₅, and R₂₆ together with the nitrogen atom they are attached to form a 4- to 8-membered heterocyclyl.

In one embodiment, R₂₄, R₂₅, and R₂₆ are each independently C₁₋₆ alkyl optionally substituted with hydroxyl, halogen, cyano, C₁₋₆ alkoxy, —S—C₁₋₆ alkyl, amino, —NH—C₁₋₆ alkyl, or —N(C₁₋₆ alkyl)₂. In one embodiment, R₂₄, R₂₅, and R₂₆ are all unsubstituted C₁₋₆ alkyl. In one embodiment, R₂₄, R₂₅, and R₂₆ are all methyl.

In one embodiment, Y is halide anion. In one embodiment, Y is bromide. In one embodiment, Y is chloride. In one embodiment, Y is iodide. In one embodiment, Y is hydroxide. In one embodiment, Y is nitrate. In one embodiment, Y is nitrite. In one embodiment, Y is perchlorate. In one embodiment, Y is thiocyanate.

In one embodiment, the permanently cationic lipid is a pharmaceutically acceptable salt of:

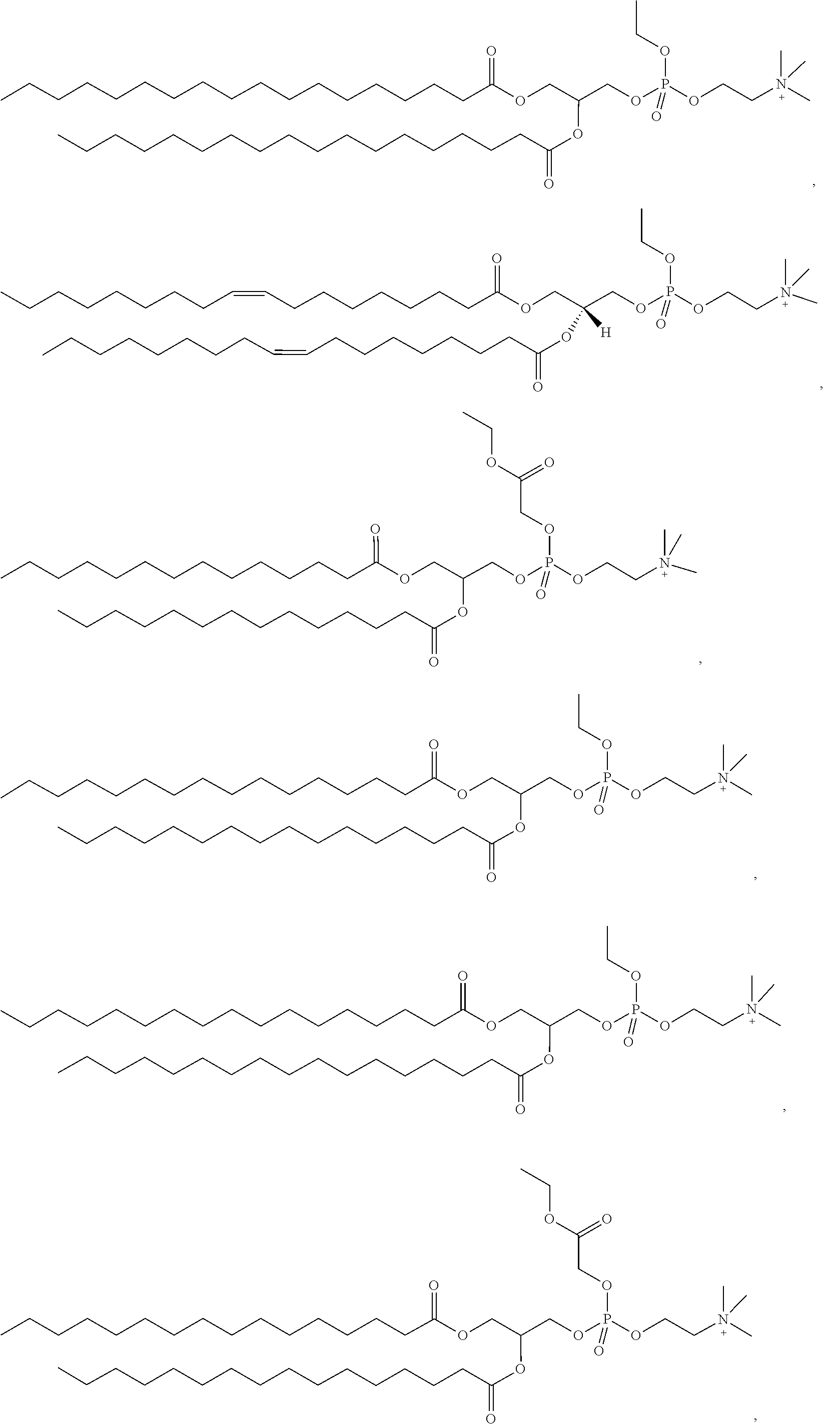

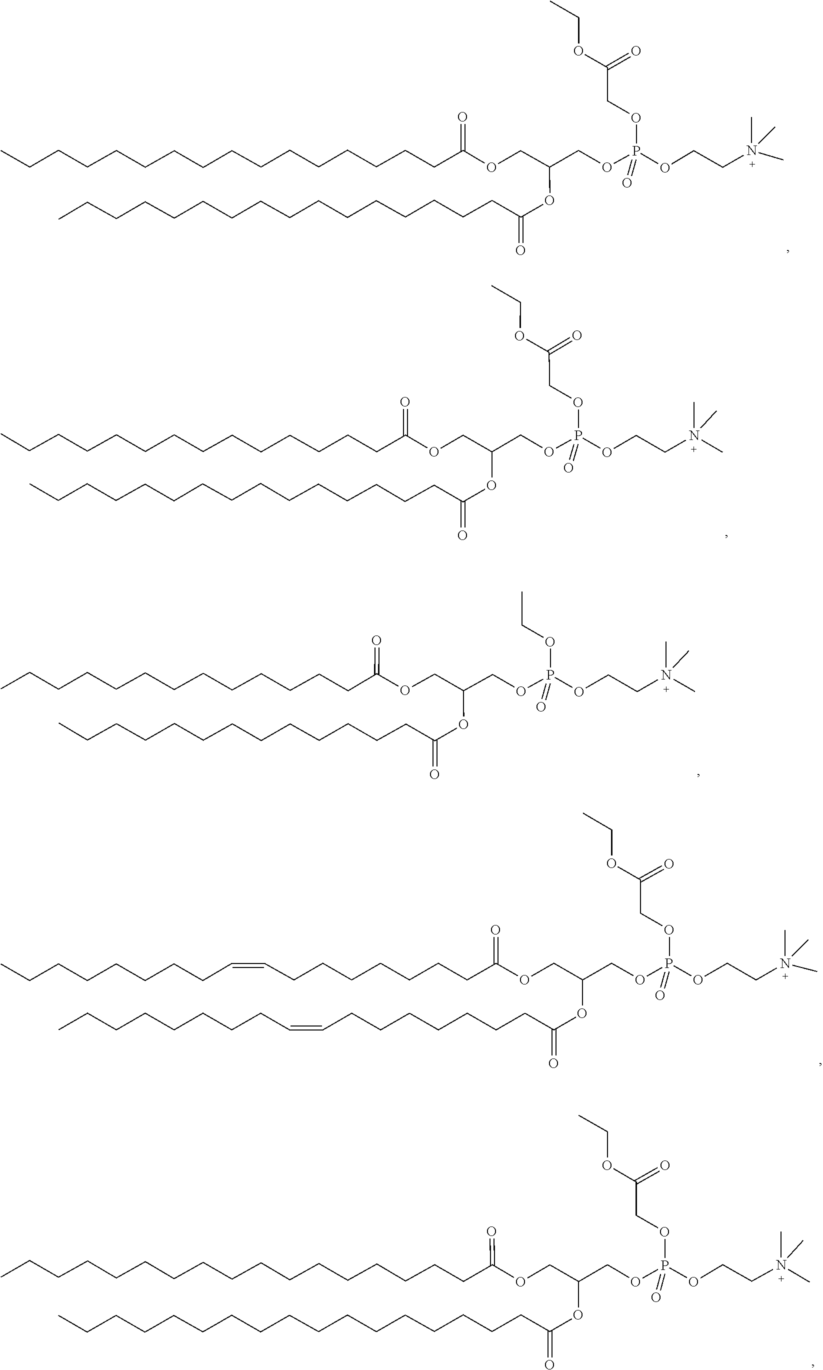

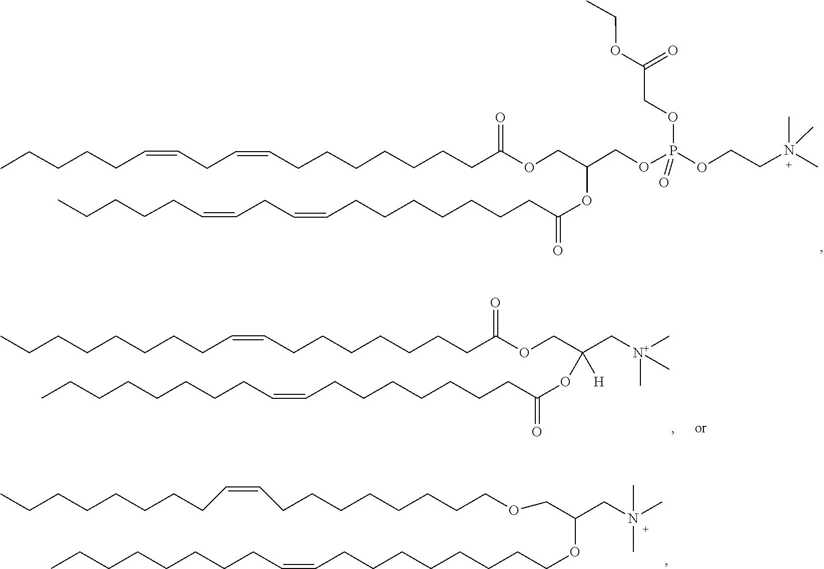

or a stereoisomer, or a mixture of stereoisomers thereof.

In one embodiment, the pharmaceutically acceptable salt is a bromide salt. In one embodiment, the pharmaceutically acceptable salt is a chloride salt. In one embodiment, the pharmaceutically acceptable salt is an iodide salt. In one embodiment, the pharmaceutically acceptable salt is a nitrate salt. In one embodiment, the pharmaceutically acceptable salt is a perchlorate salt. In one embodiment, the pharmaceutically acceptable salt is a thiocyanate.

In one embodiment, the permanently cationic lipid is N-[1-(2,3-dioleoyloxy)propyl]-N,N,N-trimethylammonium chloride (DOTMA). In one embodiment, the permanently cationic lipid is 1,2-Dioleoyl-3-trimethylammonium-propane (chloride salt) (DOTAP). In one embodiment, the permanently cationic lipid is N1-[2-((1S)-1-[(3-aminopropyl)amino]-4-[di(3-amino-propyl)amino]butylcarboxamido)ethyl]-3,4-di[oleyloxy]-benzamide (MVL5). In one embodiment, the permanently cationic lipid is Dioctadecylamidoglycylspermine hydrochloride (DOGS). In one embodiment, the permanently cationic lipid is 3ß-[N—(N′,N′-dimethylaminoethane)-carbamoyl]cholesterol hydrochloride (DC-Chol). In one embodiment, the permanently cationic lipid is Didodecyldimethylammonium Bromide (DDAB). In one embodiment, the permanently cationic lipid is 1,2-dimyristoyl-sn-glycero-3-ethylphosphocholine chloride (EPC).

In one embodiment, the amount of the permanently cationic lipid is from about 15 mol % to about 90 mol % of the total lipid present in the lipid nanoparticle. In one embodiment, the amount of the permanently cationic lipid is from about 20 mol % to about 80 mol %. In one embodiment, the amount of the permanently cationic lipid is from about 30 mol % to about 70 mol %. In one embodiment, the amount of the permanently cationic lipid is from about 40 mol % to about 60 mol %. In one embodiment, the amount of the permanently cationic lipid is from about 45 mol % to about 55 mol %. In one embodiment, the amount of the permanently cationic lipid is about 15 mol %, about 20 mol %, about 20 mol %, about 25 mol %, about 30 mol %, about 35 mol %, about 40 mol %, about 45 mol %, about 46 mol %, about 47 mol %, about 48 mol %, about 49 mol %, about 50 mol %, about 51 mol %, about 52 mol %, about 53 mol %, about 54 mol %, about 55 mol %, about 60 mol %, about 65 mol %, about 70 mol %, about 75 mol %, about 80 mol %, about 85 mol %, or about 90 mol % of the total lipid present in the lipid nanoparticle. In one embodiment, the amount of the permanently cationic lipid is about 50 mol % of the total lipid present in the lipid nanoparticle. In one embodiment, the amount of the permanently cationic lipid is 50 mol % of the total lipid present in the lipid nanoparticle.

In one embodiment, the amount of permanently cationic lipid is from about 40 mol % to about 55 mol % of the total lipid present in the lipid nanoparticle, and the amount of the ionizable lipid (see Section 5.2.2) is from about 10 mol % to about 30 mol % of the total lipid present in the lipid nanoparticle. In one embodiment, the amount of permanently cationic lipid is from about 45 mol % to about 55 mol %, and the amount of the ionizable lipid is from about 20 mol % to about 30 mol %.

In one embodiment, the molar ratio of the permanently cationic lipid and ionizable lipid in the lipid nanoparticle is from about 3:1 to about 1:3 (permanently cationic lipid:ionizable lipid). In one embodiment, the molar ratio is from about 2.5:1 to about 1:1. In one embodiment, the molar ratio is from about 2:1 to about 1:1. In one embodiment, the molar ratio is about 2.5:1, about 2.3:1, about 2:1, about 1.9:1, about 1.8:1, about 1.7:1, about 1.6:1, about 1.5:1, about 1.5:1, or about 1:1.

### 5.2.2. Ionizable Lipid

In one embodiment, the lipid nanoparticle comprises an ionizable lipid. In one embodiment, the ionizable lipid is not the permanently cationic lipid described in Section 5.2.1. In one embodiment, the lipid nanoparticle comprises an ionizable lipid and a permanently cationic lipid.

In one embodiment, the ionizable lipid has a pKa of from about 7 to about 13. In one embodiment, the ionizable lipid has a pKa of from about 7 to about 11. In one embodiment, the ionizable lipid has a pKa of from about 7 to about 13. In one embodiment, the ionizable lipid has a pKa of from about 7 to about 9. In one embodiment, the ionizable lipid has a pKa of from about 5 to about 7. In one embodiment, the ionizable lipid has a pKa of from about 6 to about 7. In one embodiment, the ionizable lipid has a pKa of about 4, about 5, about 6, about 7, about 8, about 9, about 10, or about 11. In one embodiment, the ionizable lipid has a pKa of 5.0, 5.1, 5.2, 5.3, 5.4, 5.5, 5.6, 5.7, 5.8, 5.9, 6.0, 6.1, 6.2, 6.3, 6.4, or 6.5. In one embodiment, the ionizable lipid becomes positively charged at physiological pH (i.e. pH 7.4).

In one embodiment, the ionizable lipid comprises one or more groups that is protonated at physiological pH but may deprotonated and has no charge at a pH above 8, above 9, or above 10. In one embodiment, the ionizable lipid comprises one or more tertiary amine groups. In one embodiment, the ionizable lipid comprises one, two, three, or four C₆-C₂₄ alkyl or alkenyl lipid groups. These lipid groups may be attached through a functional group (e.g. ester group or amide group) or may be further added through a Michael addition to a sulfur atom.

In one embodiment, the ionizable lipid is a compound of formula (IV′):

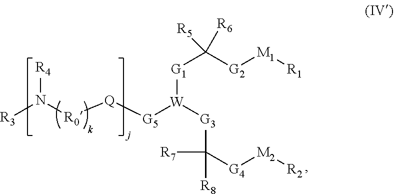

or a stereoisomer, a mixture of stereoisomers, or a pharmaceutically acceptable salt thereof, wherein
j is 0 or 1;
W is CH or N, provided that when W is N, j is 0;
k is an integral between 0 and 8;

Each instance of R₀′ is an independently optionally substituted methylene, or two substituents on a R₀′ together with the carbon they are attached to form a 3 to 8-membered cycloalkyl, and wherein the cycloalkyl is optionally substituted;
M₁ and M₂ are each independently —C(O)O—, —O—, —SC(O)O—, —OC(O)NRₐ—, —NRₐC(O)NRₐ—, —OC(O)S—, —OC(O)O—, —NRₐC(O)O—, —OC(O)—, —SC(O)—, —C(O)S—, —NRₐ—, —C(O)NRₐ—, —NRₐC(O)—, —NRₐC(O)S—, —SC(O)NRₐ—, —C(O)—, —OC(S)—, —C(S)O—, —OC(S)NRₐ—, —NRₐC(S)O—, —S—S—, or —S(O)₀₋₂—;
Q is a chemical bond, —C(O)O—, —O—, —SC(O)O—, —OC(O)NR_{b}—, —NR_{b}C(O)NR_{b}—, —OC(O)S—, —OC(O)O—, —NR_{b}C(O)O—, —OC(O)—, —SC(O)—, —C(O)S—, —NR_{b}—, —C(O)NR_{b}—, —NR_{b}C(O)—, —NR_{b}C(O)S—, —SC(O)NR_{b}—, —C(O)—, —OC(S)—, —C(S)O—, —OC(S)NR_{b}—, —NR_{b}C(S)O—, —S—S—, —S(O)₀₋₂—, phenylene, or pyridylidene, wherein the phenylene and pyridylidene are optionally substituted;
Rₐ and R_{b} are each independently H, C₁₋₂₀ alkyl, 3 to 14-membered cycloalkyl, or 3 to 14-membered heterocyclyl, wherein the alkyl, cycloalkyl and heterocyclyl are optionally substituted;
G₅ is a chemical bond or optionally substituted C₁₋₈ alkylene;
G₁, G₂, G₃ and G₄ are each independently a chemical bond, C₁₋₁₃ alkylene, C₂₋₁₃ alkenylene, or C₂₋₁₃ alkynylene, wherein the alkylene, alkenylene, and alkynylene are optionally substituted;
G₁ and G₂ have a total length of 3, 4, 5, 6, 7, 8, 9, 10, 11, 12 or 13 carbon atoms;
G₃ and G₄ have a total length of 3, 4, 5, 6, 7, 8, 9, 10, 11, 12 or 13 carbon atoms;
R₁ and R₂ are each independently C₄₋₂₀ alkyl, C₄₋₂₀ alkenyl or C₄₋₂₀ alkynyl, wherein one or more methylene units in R₁ and R₂ are independently optionally replaced by —NH— or —N(C₁₋₂₀ alkyl), wherein the alkyl, alkenyl and alkynyl are optionally substituted;
R₃ and R₄ are each independently H, C₁₋₁₀ alkyl, C₁₋₁₀ haloalkyl, C₂₋₁₀ alkenyl, C₂₋₁₀ alkynyl, 3- to 14-membered cycloalkyl, 3- to 14-membered heterocyclyl, C₆₋₁₀ aryl, or 5 to 14-membered heteroaryl, or R₃ and R₄ together with the N atom to which they are attached to form 3 to 14-membered heterocyclyl, or R₄ together with the nitrogen atom to which it is attached to and one of the R₀′ form a 3 to 14-membered heterocyclyl or 5 to 14-membered heteroaryl; and wherein the alkyl, haloalkyl, alkenyl, alkynyl, cycloalkyl, heterocyclyl, aryl and heteroaryl are optionally substituted; and
R₅, R₆, R₇ and R₈ are each independently optionally substituted C₁₋ₛ alkyl.

In one embodiment, the ionizable lipid is a compound of formula (V′), (VI′) or (VII′):

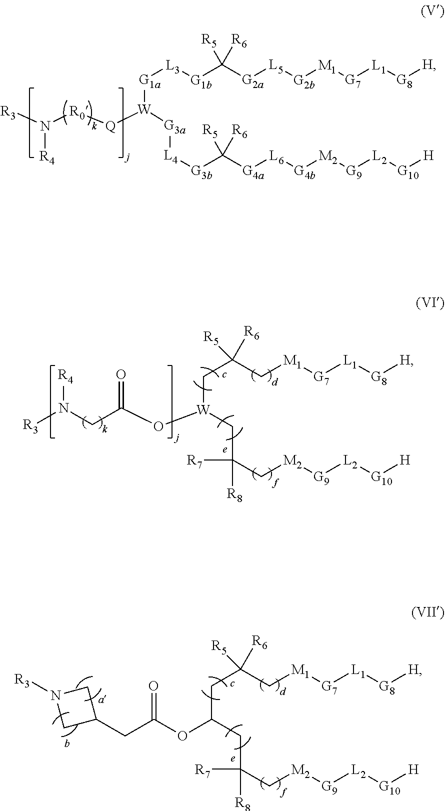

or a stereoisomer, a mixture of stereoisomers, or a pharmaceutically acceptable salt thereof, wherein
k is an integral between 0 and 6;
L₁ and L₂ are each independently —(CRR′)₂—, —CH═CH—, —C≡C— or —NR″—;
one of L₃ and L₅ is —(CR^{s}R^{s}′)₂—, —CH═CH—, or —C≡C—, and the other is a chemical bond;
one of L₄ and L₆ is —(CR^{s}R^{s}′)₂—, —CH═CH—, or —C≡C—, and the other is a chemical bond;
each of G₁ₐ, G_{1b}, G₂ₐ, G_{2b}, G₃ₐ, G_{3b}, G₄ₐ and G_{4b} is independently a chemical bond or optionally substituted C₁₋₇ alkylene;
G₁ₐ, G_{1b}, G₂ₐ and G_{2b} have a total length of 1, 2, 3, 4, 5, 6 or 7 carbon atoms;
G₃ₐ, G_{3b}, G₄ₐ and G_{4b} have a total length of 1, 2, 3, 4, 5, 6 or 7 carbon atoms;
each of R₅, R₆, R₇ and R₈ is independently optionally substituted C₁₋₆ alkyl;
each of G₇, G₈, G₉ and G₁₀ is independently a chemical bond or optionally substituted C₁₋₁₂ alkylene, provided that G₇ and G₈ have a total length of 4, 5, 6, 7, 8, 9, 10, 11 or 12 carbon atoms, and G₉ and G₁₀ have a total length of 4, 5, 6, 7, 8, 9, 10, 11 or 12 carbon atoms;
R^{s} and R^{s}′ are each independently H, C₁₋₁₀ alkyl, -L_{d}-OR_{d} or -L_{d}-NR_{d}R′_{d};
R′ is H, C₁₋₁₄ alkyl, -Lₐ-ORₐ or -Lₐ-NRₐR′ₐ;
R″ is H or C₁₋₁₄ alkyl;
L_{d} is a chemical bond or C₁₋₁₀ alkylene;
Lₐ is a chemical bond or optionally substituted C₁₋₁₄ alkylene;
Rₐ and R′ₐ are each independently H, C₁₋₁₄ alkyl, 3- to 10-membered cycloalkyl or 3- to 10-membered heterocyclyl, and wherein the alkyl, cycloalkyl and heterocyclyl are optionally substituted;
R_{d} and R′_{d} are each independently H or optionally substituted C₁₋₁₀ alkyl;
a′ and b are each independently an integral between 0 to 5, provided that at least one of a′ and b are not 0;
g is an integral between 0 and 5;
a′+g is an integral between 0 and 5; and
c, d, e, and f are each independently an integral between 0 and 7, provided that c+d is an integral between 2 and 9, and e+f is an integral between 2 and 9.

In one embodiment, R₁ is C₄₋₂₀ alkyl. In one embodiment, R₁ is C₆₋₁₈ alkyl. In one embodiment, R₁ is C₈₋₁₈ alkyl. In one embodiment, R₁ is C₈, C₉, C₁₀, C₁₁, C₁₂, C₁₃, C₁₄, C₁₅, C₁₆, C₁₇, C₁₈, C₁₉, or C₂₀ alkyl. In one embodiment, R₁ is C₄₋₂₀ alkenyl. In one embodiment, R₁ is C₆₋₁₈ alkenyl. In one embodiment, R₁ is C₈₋₁₈ alkenyl. In one embodiment, R₁ is C₈, C₉, C₁₀, C₁₁, C₁₂, C₁₃, C₁₄, C₁₅, C₁₆, C₁₇, C₁₈, C₁₉, or C₂₀ alkenyl. In one embodiment, R₁ is C₄₋₂₀ alkynyl. In one embodiment, R₁ is C₆₋₁₈ alkynyl. In one embodiment, R₁ is C₈₋₁₈ alkynyl. In one embodiment, R₁ is C₈, C₉, C₁₀, C₁₁, C₁₂, C₁₃, C₁₄, C₁₅, C₁₆, C₁₇, C₁₈, C₁₉, or C₂₀ alkynyl.

In one embodiment, one or more —CH₂— group in R₁ replaced by —NH—. In one embodiment, one or more —CH₂— group in R₁ is replaced by —N(C₁₋₂₀ alkyl)-. In one embodiment, one or more —CH₂— group in R₁ is replaced by —N(C₁₋₁₂ alkyl)-.

In one embodiment, R₁ is unsubstituted. In one embodiment, R₁ is substituted with -Lₐ-ORₐ. In one embodiment, R₁ is substituted with -Lₐ-SRₐ. In one embodiment, R₁ is substituted with -Lₐ-NRₐR′ₐ.

In one embodiment, R₂ is C₄₋₂₀ alkyl. In one embodiment, R₂ is C₆₋₁₈ alkyl. In one embodiment, R₂ is C₈₋₁₈ alkyl. In one embodiment, R₂ is C₈, C₉, C₁₀, C₁₁, C₁₂, C₁₃, C₁₄, C₁₅, C₁₆, C₁₇, C₁₈, C₁₉, or C₂₀ alkyl. In one embodiment, R₂ is C₄₋₂₀ alkenyl. In one embodiment, R₂ is C₆₋₁₈ alkenyl. In one embodiment, R₂ is C₈₋₁₈ alkenyl. In one embodiment, R₂ is C₈, C₉, C₁₀, C₁₁, C₁₂, C₁₃, C₁₄, C₁₅, C₁₆, C₁₇, C₁₈, C₁₉, or C₂₀ alkenyl. In one embodiment, R₂ is C₄₋₂₀ alkynyl. In one embodiment, R₂ is C₆₋₁₈ alkynyl. In one embodiment, R₂ is C₈₋₁₈ alkynyl. In one embodiment, R₂ is C₈, C₉, C₁₀, C₁₁, C₁₂, C₁₃, C₁₄, C₁₅, C₁₆, C₁₇, C₁₈, C₁₉, or C₂₀ alkynyl.

In one embodiment, one or more —CH₂— group in R₂ replaced by —NH—. In one embodiment, one or more —CH₂— group in R₂ is replaced by —N(C₁₋₂₀ alkyl)-. In one embodiment, one or more —CH₂— group in R₁ is replaced by —N(C₁₋₁₂ alkyl)-.

In one embodiment, R₂ is unsubstituted. In one embodiment, R₂ is substituted with -Lₐ-ORₐ. In one embodiment, R₂ is substituted with -Lₐ-SRₐ. In one embodiment, R₂ is substituted with -Lₐ-NRₐR′ₐ.

In one embodiment, Lₐ is absent. In one embodiment, Lₐ is C₁₋₁₄ alkylene. In one embodiment, Lₐ is C₁₋₆ alkylene. In one embodiment, Lₐ is methylene. In one embodiment, Lₐ is ethylene.

In one embodiment, Rₐ is C₁₋₁₄ alkyl. In one embodiment, Rₐ is C₃₋₁₀ cycloalkyl. In one embodiment, Rₐ is 3- to 10-membered heterocyclyl. In one embodiment, Rₐ is C₁₋₁₀ alkyl; In one embodiment, Rₐ is C₈₋₁₀ alkyl; In one embodiment, Rₐ is C₈₋₁₀ linear alkyl; In one embodiment, Rₐ is —(CH₂)₈CH₃; In one embodiment, Rₐ is optionally substituted with one or more of the following substituents: H, C₁₋₂₀ alkyl, -Lₑ-OR^{e}, -Lₑ-SRₑ and -Lₑ-NRₑR′ₑ.

In one embodiment, R′ₐ is C₁₋₁₄ alkyl. In one embodiment, R′ₐ is C₃₋₁₀ cycloalkyl. In one embodiment, R′ₐ is 3- to 10-membered heterocyclyl. In one embodiment, R′ₐ is C₈₋₁₀ alkyl; In one embodiment, R′ₐ is C₈₋₁₀ linear alkyl; In one embodiment, R′ₐ is —(CH₂)₈CH₃; In one embodiment, Rₐ is optionally substituted with one or more of the following substituents: H, C₁₋₂₀ alkyl, -Lₑ-ORₑ, -Lₑ-SRₑ and -Lₑ-NRₑR′ₑ.

In one embodiment, Rₐ and R′ₐ together with the nitrogen they are attached to form a 4 to 10-membered ring. In one embodiment, Rₐ and R′ₐ together with the nitrogen they are attached to form a 4 to 8-membered ring. In one embodiment, Rₐ and R′ₐ together with the nitrogen they are attached to form a 4 to 6-membered ring. In one embodiment, Rₐ and R′ₐ together with the nitrogen they are attached to form a 4 to 6-membered cycloalkyl. In one embodiment, Rₐ and R′ₐ together with the nitrogen they are attached to form a 4 to 6-membered heterocyclyl.

In one embodiment, R₃ is H. In one embodiment, R₃ is C₁₋₁₀ alkyl. In one embodiment, R₃ is C₁₋₁₀ haloalkyl. In one embodiment, R₃ is C₂₋₁₀ alkenyl. In one embodiment, R₃ is C₂₋₁₀ alkynyl. In one embodiment, R₃ is 3- to 14-membered cycloalkyl. In one embodiment, R₃ is 3- to 14-membered heterocyclyl. In one embodiment, R₃ is C₆₋₁₀ aryl. In one embodiment, R₃ is 5 to 14-membered heteroaryl. In one embodiment, R₃ is C₁₋₆ alkyl. In one embodiment, R₃ is C₁₋₆ haloalkyl. In one embodiment, R₃ is 3- to 10-membered cycloalkyl. In one embodiment, R₃ is 3- to 10-membered heterocyclyl. In one embodiment, R₃ is 3- to 7-membered cycloalkyl. In one embodiment, R₃ is 3- to 7-membered heterocyclyl. In one embodiment, R₃ is Me. In one embodiment, R₃ is —CH₂CH₃. In one embodiment, R₃ is —CH₂CH₂OH. In one embodiment, R₃ is —CH(CH₃)₂. In one embodiment, R₃ is unsubstituted. In one embodiment, R₃ is substituted with one or more R*, wherein each R* is independently halogen, cyano, C₁₋₁₀ alkyl, C₁₋₁₀ haloalkyl, -L_{b}-OR_{b}, -L_{b}-SR_{b} or -L_{b}-NR_{b}R′_{b}. In one embodiment, R₃ is optionally substituted with 1, 2, 3, 4 or 5 R*, wherein each R* is independently halogen, cyano, C₁₋₁₀ alkyl, C₁₋₁₀ haloalkyl, -L_{b}-OR_{b}, -L_{b}-SR_{b} or -L_{b}-NR_{b}R′_{b}.

In one embodiment, R₄ is H. In one embodiment, R₄ is C₁₋₁₀ alkyl. In one embodiment, R₄ is C₁₋₁₀ haloalkyl. In one embodiment, R₄ is C₂₋₁₀ alkenyl. In one embodiment, R₄ is C₂₋₁₀ alkynyl. In one embodiment, R₄ is 3- to 14-membered cycloalkyl. In one embodiment, R₄ is 3- to 14-membered heterocyclyl. In one embodiment, R₄ is C₆₋₁₀ aryl. In one embodiment, R₄ is 5 to 14-membered heteroaryl. In one embodiment, R₄ is C₁₋₆ alkyl. In one embodiment, R₄ is C₁₋₆ haloalkyl. In one embodiment, R₄ is 3- to 10-membered cycloalkyl. In one embodiment, R₄ is 3- to 10-membered heterocyclyl. In one embodiment, R₄ is 3- to 7-membered cycloalkyl. In one embodiment, R₄ is 3- to 7-membered heterocyclyl. In one embodiment, R₄ is Me. In one embodiment, R₃ is unsubstituted. In one embodiment, R₃ is substituted with one or more R*. In one embodiment, R₃ is optionally substituted with 1, 2, 3, 4 or 5 R*.

In one embodiment, R₃ and R₄ together with the N atom to which they are attached to form 3 to 14-membered heterocyclyl. In one embodiment, R₃, R₄ together with the N atom to which they are attached to form 3- to 10-membered heterocyclyl; In one embodiment, R₃, R₄ together with the N atom to which they are attached to form 3- to 7-membered heterocyclyl; In one embodiment, R₃, R₄ together with the N atom to which they are attached to form 5- to 7-membered heterocyclyl; In one embodiment, R₃, R₄ together with the N atom to which they are attached to form 4- to 6-membered heterocyclyl; In one embodiment, R₃, R₄ together with the N atom to which they are attached to form 5-membered heterocyclyl; In one embodiment, R₃, R₄ together with the N atom to which they are attached to form

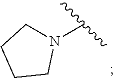 [SMILES: *N1CCCC1]

In one embodiment, R₃, R₄ together with the N atom to which they are attached to form

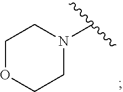 [SMILES: *N1CCOCC1]

In one embodiment, R₃, R₄ together with the N atom to which they are attached to form

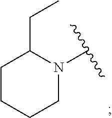 [SMILES: *N1CCCCC1CC]

In one embodiment, R₃, R₄ together with the N atom to which they are attached to form

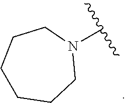 [SMILES: *N1CCCCCC1]

In one embodiment, the heterocyclyl formed by R₃ and R₄ together with the N atom to which they are attached is optionally substituted with one or more R*; In one embodiment, the heterocyclyl formed by R₃ and R₄ taken together with the N atom to which they are attached is optionally substituted with 1, 2, 3, 4 or 5 R*.

In one embodiment, R₄ together with the nitrogen atom to which it is attached to and one of the R₀′ form a 3 to 14-membered heterocyclyl. In one embodiment, R₄ together with the nitrogen atom to which it is attached to and one of the R₀′ form a 4 to 10-membered heterocyclyl. In one embodiment, R₄ together with the nitrogen atom to which it is attached to and one of the R₀′ form a 4 to 6-membered heterocyclyl. In one embodiment, R₄ together with the nitrogen atom to which it is attached to and one of the R₀′ form a 5 to 14-membered heteroaryl. In one embodiment, R₄ together with the nitrogen atom to which it is attached to and one of the R₀′ form a 5 to 10-membered heteroaryl. In one embodiment, R₄ together with the nitrogen atom to which it is attached to and one of the R₀′ form a 5 or 6-membered heteroaryl.

In one embodiment, R₅ is C₁₋₈ alkyl; In one embodiment, R₅ is C₁₋₆ alkyl; In one embodiment, R₅ is C₁₋₃ alkyl; In one embodiment, R₅ is Me; In one embodiment, R₅ is optionally substituted with one or more R*; In one embodiment, R₅ is optionally substituted with 1, 2, 3, 4 or 5 R*.

In one embodiment, R₆ is C₁₋₈ alkyl; In one embodiment, R₆ is C₁₋₆ alkyl; In one embodiment, R₆ is C₁₋₃ alkyl; In one embodiment, R₆ is Me; In one embodiment, R₆ is optionally substituted with one or more R*; In one embodiment, R₆ is optionally substituted with 1, 2, 3, 4 or 5 R*.

In one embodiment, R₇ is C₁₋₈ alkyl; In one embodiment, R₇ is C₁₋₆ alkyl; In one embodiment, R₇ is C₁₋₃ alkyl; In one embodiment, R₇ is Me; In one embodiment, R₇ is optionally substituted with one or more R*; In one embodiment, R₇ is optionally substituted with 1, 2, 3, 4 or 5 R*.

In one embodiment, R₈ is C₁₋₈ alkyl; In one embodiment, R₈ is C₁₋₆ alkyl; In one embodiment, R₈ is C₁₋₃ alkyl; In one embodiment, R₈ is Me; In one embodiment, R₈ is optionally substituted with one or more R*; In one embodiment, R₈ is optionally substituted with 1, 2, 3, 4 or 5 R*.

In one embodiment, R* is halogen, cyano, C₁₋₆ alkyl, C₁₋₆ haloalkyl, -L_{b}-OR_{b} or -L_{b}-NR_{b}R′_{b}; In one embodiment, R* is C₁₋₆ alkyl, C₁₋₆ haloalkyl or —OR_{b}; In one embodiment, R* is independently H, halogen, C₁₋₆ alkyl or C₁₋₆ haloalkyl; In one embodiment, R* is C₁₋₆ alkyl or C₁₋₆ haloalkyl; In one embodiment, R* is Me. In one embodiment, R* is OH. In one embodiment, R* is halogen; In one embodiment, R* is cyano; In one embodiment, R* is C₁₋₁₀ alkyl; In one embodiment, R* is C₁₋₁₀ haloalkyl; In one embodiment, R* is -L_{b}-OR_{b}; In one embodiment, R* is -L_{b}-SR_{b}; In one embodiment, R* is -L_{b}-NR_{b}R′_{b}; In one embodiment, R* is C₁₋₆ alkyl; In one embodiment, R* is C₁₋₆ haloalkyl; In one embodiment, R* is —OR_{b}.

In one embodiment, L_{b} is absent. In one embodiment, Lₐ is C₁₋₁₀ alkylene. In one embodiment, L_{b} is C₁₋₆ alkylene. In one embodiment, L_{b} is methylene. In one embodiment, L_{b} is ethylene.

In one embodiment, R_{b} is C₁₋₁₀ alkyl, 3- to 14-membered cycloalkyl, or 3- to 14-membered heterocyclyl, wherein R_{b} is optionally substituted with one or more of C₁₋₁₀ alkyl, -L_{f}-OR_{f}, -L_{f}-SR_{f} or -L_{f}-NR_{f}R′_{f}, wherein R_{f} and R′_{f} are each independently H or C₁₋₁₀ alkyl. In one embodiment, R_{b} is C₁₋₆ alkyl. In one embodiment, R_{b} is C₃₋₈ cycloalkyl. In one embodiment, R_{b} is 3- to 8-membered heterocyclyl.

In one embodiment, j is 0. In one embodiment, j is 1.

In one embodiment, W is CH. In one embodiment, W is N.

In one embodiment, k is 0. In one embodiment, k is 1. In one embodiment, k is 2. In one embodiment, k is 3. In one embodiment, k is 4. In one embodiment, k is 5. In one embodiment, k is 6. In one embodiment, k is 7. In one embodiment, k is 8.

In one embodiment, two substituents on R₀′ together with the carbon they are attached to form a 3 to 8-membered cycloalkyl. In one embodiment, two substituents on R₀′ together with the carbon they are attached to form a 3 to 6-membered cycloalkyl. In one embodiment, two substituents on R₀′ together with the carbon they are attached to form a 5 or 6-membered cycloalkyl.

In one embodiment, M₁ is —C(O)O—; In one embodiment, M₁ is —O—; In one embodiment, M₁ is —SC(O)O—; In one embodiment, M₁ is —OC(O)NRₐ—; In one embodiment, M₁ is —NRₐC(O)NRₐ—; In one embodiment, M₁ is —OC(O)S—; In one embodiment, M₁ is —OC(O)O—; In one embodiment, M₁ is —NRₐC(O)O—; In one embodiment, M₁ is —OC(O)—; In one embodiment, M₁ is —SC(O)—; In one embodiment, M₁ is —C(O)S—; In one embodiment, M₁ is —NRₐ—; In one embodiment, M₁ is —C(O)NRₐ—; In one embodiment, M₁ is —NRₐC(O)—; In one embodiment, M₁ is —NRₐC(O)S—; In one embodiment, M₁ is —SC(O)NRₐ—; In one embodiment, M₁ is —C(O)—; In one embodiment, M₁ is —OC(S)—; In one embodiment, M₁ is —C(S)O—; In one embodiment, M₁ is —OC(S)NRₐ—; In one embodiment, M₁ is —NRₐC(S)O—; In one embodiment, M₁ is —S—S—; In one embodiment, M₁ is —S(O)₀₋₂—.

In one embodiment, M₂ is —C(O)O—; In one embodiment, M₂ is —O—; In one embodiment, M₂ is —SC(O)O—; In one embodiment, M₂ is —OC(O)NRₐ—; In one embodiment, M₂ is —NRₐC(O)NRₐ—; In one embodiment, M₂ is —OC(O)S—; In one embodiment, M₂ is —OC(O)O—; In one embodiment, M₂ is —NRₐC(O)O—; In one embodiment, M₂ is —OC(O)—; In one embodiment, M₂ is —SC(O)—; In one embodiment, M₂ is —C(O)S—; In one embodiment, M₂ is —NRₐ—; In one embodiment, M₂ is —C(O)NRₐ—; In one embodiment, M₂ is —NRₐC(O)—; In one embodiment, M₂ is —NRₐC(O)S—; In one embodiment, M₂ is —SC(O)NRₐ—; In one embodiment, M₂ is —C(O)—; In one embodiment, M₂ is —OC(S)—; In one embodiment, M₂ is —C(S)O—; In one embodiment, M₂ is —OC(S)NRₐ—; In one embodiment, M₂ is —NRₐC(S)O—; In one embodiment, M₂ is —S—S—; In one embodiment, M₂ is —S(O)₀₋₂—.

In one embodiment, M₁ and M₂ are each independently selected from —C(O)O—, —SC(O)O—, —OC(O)NRₐ—, —NRₐC(O)NRₐ—, —OC(O)S—, —OC(O)O—, —NRₐC(O)O—, —C(O)S—, —C(O)NRₐ—, —NRₐC(O)S—, —SC(O)NRₐ—, —C(S)O—, —OC(S)NRₐ— and —NRₐC(S)O—; In one embodiment, M₁ and M₂ are independently —C(O)O—, —C(O)S—, —C(O)NRₐ—, or —C(S)O—; In one embodiment, M₁ and M₂ are independently —C(O)O—, —C(O)S— or —C(O)NRₐ—.

In one embodiment, Q is a chemical bond; in another embodiment, Q is —C(O)O—; in another embodiment, Q is —O—; in another embodiment, Q is —SC(O)O—; in another embodiment, Q is —OC(O)NR_{b}—; in another embodiment, Q is —NR_{b}C(O)NR_{b}—; in another embodiment, Q is —OC(O)S—; in another embodiment, Q is —OC(O)O—; in another embodiment, Q is —NR_{b}C(O)O—; in another embodiment, Q is —OC(O)—; in another embodiment, Q is —SC(O)—; in another embodiment, Q is —C(O)S—; in another embodiment, Q is —NR_{b}—; in another embodiment, Q is —C(O)NR_{b}—; in another embodiment, Q is —NR_{b}C(O)—; in another embodiment, Q is —NR_{b}C(O)S—; in another embodiment, Q is —SC(O)NR_{b}—; in another embodiment, Q is —C(O)—; in another embodiment, Q is —OC(S)—; in another embodiment, Q is —C(S)O—; in another embodiment, Q is —OC(S)NR_{b}—; in another embodiment, Q is —NR_{b}C(S)O—; in another embodiment, Q is —S—S—; in another embodiment, Q is —S(O)₀₋₂—; in another embodiment, Q is phenylene; in another embodiment, Q is pyridylidene; in another embodiment, the phenylene or pyridylidene is optionally substituted with one or more R*.

In one embodiment, Q is selected from a chemical bond, —C(O)O—, —O—, —SC(O)O—, —OC(O)NR_{b}—, —NR_{b}C(O)NR_{b}—, —OC(O)S—, —OC(O)O—, —NR_{b}C(O)O—, —OC(O)—, —SC(O)—, —C(O)S—, —NR_{b}—, —C(O)NR_{b}—, —NR_{b}C(O)—, —NR_{b}C(O)S—, —SC(O)NR_{b}—, —C(O)—, —OC(S)—, —C(S)O—, —OC(S)NR_{b}—, —NR_{b}C(S)O—, —S—S—, and —S(O)₀₋₂—; In one embodiment, Q is selected from —C(O)O—, —O—, —SC(O)O—, —OC(O)NH—, —NHC(O)NH—, —OC(O)S—, —OC(O)O—, —NHC(O)O—, —OC(O)—, —SC(O)—, —C(O)S—, —NH—, —C(O)NH—, —NHC(O)—, —NHC(O)S—, —SC(O)NH—, —C(O)—, —OC(S)—, —C(S)O—, —OC(S)NH— and —NHC(S)O—; In one embodiment, Q is selected from —C(O)O—, —O—, —SC(O)O—, —OC(O)NH—, —NHC(O)NH—, —OC(O)S—, —OC(O)O— and —NHC(O)O—; In one embodiment, Q is —C(O)O—.

In one embodiment, Rₐ is H; in another embodiment, Rₐ is C₁₋₂₀ alkyl; in another embodiment, Rₐ is 3- to 14-membered cycloalkyl; in another embodiment, Rₐ is 3- to 14-membered heterocyclyl; in another embodiment, Rₐ is C₁₋₁₄ alkyl; in another embodiment, Rₐ is C₁₋₁₀ alkyl; in another embodiment, Rₐ is C₈₋₁₀ alkyl; in another embodiment, Rₐ is C₁₀ linear alkyl; in another embodiment, Rₐ is —(CH₂)₈CH₃; in another embodiment, Rₐ is optionally substituted with one or more of the following substituents: H, C₁₋₂₀ alkyl, -Lₑ-ORₑ, -Lₑ-SRₑ and -Lₑ-NRₑR′ₑ, wherein Lₑ is absent or is C₁₋₂₀ alkylene, wherein Rₑ and R′ₑ are independently H or C₁₋₂₀ alkyl;

In one embodiment, R′ₐ is H; in another embodiment, R′ₐ is C₁₋₂₀ alkyl; in another embodiment, R′ₐ is 3- to 14-membered cycloalkyl; in another embodiment, R′ₐ is 3- to 14-membered heterocyclyl; in another embodiment, R′ₐ is C₁₋₁₄ alkyl; in another embodiment, R′ₐ is C₁₋₁₀ alkyl; in another embodiment, R′ₐ is C₈₋₁₀ alkyl; in another embodiment, R′ₐ is C₁₋₁₀ linear alkyl; in another embodiment, R′ₐ is —(CH₂)₈CH₃; in another embodiment, R′ₐ is optionally substituted with one or more of the following substituents: H, C₁₋₂₀ alkyl, -Lₑ-ORₑ, -Lₑ-SRₑ and -Lₑ-NRₑR′ₑ, wherein Lₑ is absent or is C₁₋₂₀ alkylene, wherein Rₑ and R′ₑ are independently H or C₁₋₂₀ alkyl.

In one embodiment, Le is absent. In one embodiment, Le is C₁₋₂₀ alkylene. In one embodiment, Le is C₁₋₁₆ alkylene. In one embodiment, Le is C₁₋₁₂ alkylene. In one embodiment, Le is C₁₋₆ alkylene. In one embodiment, Le is methylene. In one embodiment, Le is ethylene.

In one embodiment, Rₑ is hydrogen. In one embodiment, Rₑ is C₁₋₂₀ alkyl. In one embodiment, Rₑ is C₁₋₁₆ alkyl. In one embodiment, Rₑ is C₁₋₁₂ alkyl. In one embodiment, Rₑ is C₁₋₆ alkyl. In one embodiment, R′ₑ is hydrogen. In one embodiment, R′ₑ is C₁₋₂₀ alkyl. In one embodiment, R′ₑ is C₁₋₁₆ alkyl. In one embodiment, R′ₑ is C₁₋₁₂ alkyl. In one embodiment, R′ₑ is C₁₋₆ alkyl.

In one embodiment, G₁ is a chemical bond; in another embodiment, G₁ is C₁₋₁₃ alkylene; in another embodiment, G₁ is C₂₋₁₃ alkenylene; in another embodiment, G₁ is C₂₋₆ alkenylene; in another embodiment, G₁ is C₂₋₁₃ alkynylene; in another embodiment, G₁ is C₂₋₆ alkynylene; in another embodiment, G₁ is optionally substituted with one or more R^{s}, wherein R^{s} is independently H, C₁₋₁₄ alkyl, -L_{d}-OR_{d}, -L_{d}-SR_{d} or -L_{d}-NR_{d}R′_{d}, and wherein L_{d} is absent or C₁₋₁₄ alkylene; and wherein R_{d} and R′_{d} are independently H or C₁₋₁₄ alkyl.

In one embodiment, G₂ is a chemical bond; in another embodiment, G₂ is C₂₋₁₃ alkylene; in another embodiment, G₂ is C₂₋₆ alkenylene; in another embodiment, G₂ is C₂₋₁₃ alkenylene; in another embodiment, G₂ is C₂₋₆ alkenylene; in another embodiment, G₂ is C₂₋₁₃ alkynylene; in another embodiment, G₂ is optionally substituted with one or more R^{s}, wherein R^{s} is independently H, C₁₋₁₄ alkyl, -L_{d}-OR_{d}, -L_{d}-SR_{d} or -L_{d}-NR_{d}R′_{d}, and wherein Ld is absent or C₁₋₁₄ alkylene; and wherein R_{d} and R′_{d} are independently H or C₁₋₁₄ alkyl.

In one embodiment, G₁ and G₂ have a total length of 3 carbon atoms; in another embodiment, G₁ and G₂ have a total length of 4 carbon atoms; in another embodiment, G₁ and G₂ have a total length of 5 carbon atoms; in another embodiment, G₁ and G₂ have a total length of 6 carbon atoms; in another embodiment, G₁ and G₂ have a total length of 7 carbon atoms; in another embodiment, G₁ and G₂ have a total length of 8 carbon atoms; in another embodiment, G₁ and G₂ have a total length of 9 carbon atoms; in another embodiment, G₁ and G₂ have a total length of 10 carbon atoms; in another embodiment, G₁ and G₂ have a total length of 11 carbon atoms; in another embodiment, G₁ and G₂ have a total length of 12 carbon atoms; in another embodiment, G₁ and G₂ have a total length of 13 carbon atoms.

In one embodiment, G₃ is a chemical bond; in another embodiment, G₃ is C₁₋₁₃ alkylene; in another embodiment, G₃ is C₂₋₁₃ alkenylene; in another embodiment, G₃ is C₂₋₆ alkenylene; in another embodiment, G₃ is C₂₋₁₃ alkynylene; in another embodiment, G₃ is C₂₋₆ alkynylene; in another embodiment, G₃ is optionally substituted with one or more R^{s}, wherein R^{s} is independently H, C₁₋₁₄ alkyl, -L_{d}-OR_{d}, -L_{d}-SR_{d} or -L_{d}-NR_{d}R′_{d}, and wherein Ld is absent or C₁₋₁₄ alkylene; and wherein R_{d} and R′_{d} are independently H or C₁₋₁₄ alkyl.

In one embodiment, G₄ is a chemical bond; in another embodiment, G₄ is C₂₋₁₃ alkylene; in another embodiment, G₄ is C₂₋₆ alkenylene; in another embodiment, G₄ is C₂₋₁₃ alkenylene; in another embodiment, G₄ is C₂₋₆ alkenylene; in another embodiment, G₄ is C₂₋₁₃ alkynylene; in another embodiment, G₄ is optionally substituted with one or more R^{s}, wherein R^{s} is independently H, C₁₋₁₄ alkyl, -L_{d}-OR_{d}, -L_{d}-SR_{d} or -L_{d}-NR_{d}R′_{d}, and wherein Ld is absent or C₁₋₁₄ alkylene; and wherein R_{d} and R′_{d} are independently H or C₁₋₁₄ alkyl.

In one embodiment, G₃ and G₄ have a total length of 3 carbon atoms; in another embodiment, G₃ and G₄ have a total length of 4 carbon atoms; in another embodiment, G₃ and G₄ have a total length of 5 carbon atoms; in another embodiment, G₃ and G₄ have a total length of 6 carbon atoms; in another embodiment, G₃ and G₄ have a total length of 7 carbon atoms; in another embodiment, G₃ and G₄ have a total length of 8 carbon atoms; in another embodiment, G₃ and G₄ have a total length of 9 carbon atoms; in another embodiment, G₃ and G₄ have a total length of 10 carbon atoms; in another embodiment, G₃ and G₄ have a total length of 11 carbon atoms; in another embodiment, G₃ and G₄ have a total length of 12 carbon atoms; in another embodiment, G₃ and G₄ have a total length of 13 carbon atoms.

In one embodiment, R^{s} is H; in another embodiment, R^{s} is C₁₋₁₄ alkyl; in another embodiment, R^{s} is -L_{d}-OR_{d}; in another embodiment, R^{s} is -L_{d}-SR_{d}; in another embodiment, R^{s} is -L_{d}-NR_{d}R′_{d}; in another embodiment, R^{s} is C₁₋₁₀ alkyl; in another embodiment, R^{s} is C₁₋₆ alkyl. In one embodiment, R^{s} is H, C₁₋₁₀ alkyl, -L_{d}-OR_{d} or -L_{d}-NR_{d}R′_{d}; in another more specific embodiment, R^{s} is H or C₁₋₆ alkyl.

In one embodiment, G₅ is a chemical bond; in another embodiment, G₅ is C₁₋₈ alkylene; in another embodiment, G₅ is C₁₋₆ alkylene; in another embodiment, G₅ is C₁₋₃ alkylene; in another embodiment, G₅ is optionally substituted with one or more R**, wherein each R** is independently C₁_s alkyl, -L_{c}-OR_{c}, -L_{c}-SR_{c} or -L_{c}-NR_{c}R′_{c}, wherein R_{c} and R′_{c} are independently H or C₁₋₈ alkyl, and wherein Lc is absent or C₁₋₆ alkylene;

In one embodiment, R** is C₁₋₈ alkyl; in another embodiment, R** is -L_{c}-OR_{c}; in another embodiment, R** is -L_{c}-SR_{c}; in another embodiment, R** is -L_{c}-NR_{c}R′_{c}; in another embodiment, R** is C₁₋₆ alkyl.

In one embodiment, L₁ is —(CRR′)₂—. In one embodiment, L₁ is —CH═CH—. In one embodiment, L₁ is —C≡C—. In one embodiment, L₁ is —NR″—;

In one embodiment, L₂ is —(CRR′)₂—. In one embodiment, L₂ is —CH═CH—. In one embodiment, L₂ is —C≡C—. In one embodiment, L₂ is —NR″—;

In one embodiment, L₃ is —(CR^{s}R^{s}′)₂—, and L₅ is a chemical bond. In one embodiment, L₃ is —CH═CH—, and L₅ is a chemical bond. In one embodiment, L₃ is —C≡C—, and L₅ is a chemical bond. In one embodiment, L₅ is —(CR^{s}R^{s}′)₂—, and L₃ is a chemical bond. In one embodiment, L₅ is —CH═CH—, and L₃ is a chemical bond. In one embodiment, L₅ is —C≡C—, and L₃ is a chemical bond.

In one embodiment, L₄ is —(CR^{s}R^{s}′)₂—, and L₆ is a chemical bond. In one embodiment, L₄ is —CH═CH—, and L₆ is a chemical bond. In one embodiment, L₄ is —C≡C—, and L₆ is a chemical bond. In one embodiment, L₆ is —(CR^{s}R^{s}′)₂—, and L₄ is a chemical bond. In one embodiment, L₆ is —CH═CH—, and L₄ is a chemical bond. In one embodiment, L₆ is —C≡C—, and L₄ is a chemical bond.

In one embodiment, G₁ₐ is a chemical bond. In one embodiment, G₁ₐ is methylene. In one embodiment, G₁ₐ is ethylene. In one embodiment, G₁ₐ is C₃ alkylene. In one embodiment, G₁ₐ is C₄ alkylene. In one embodiment, G₁ₐ is C₅ alkylene. In one embodiment, G₁ₐ is C₆ alkylene. In one embodiment, G₁ₐ is C₇ alkylene. In one embodiment, G₁ₐ is unsubstituted. In one embodiment, G₁ₐ is substituted. In one embodiment, G₁ₐ is substituted with one or more R^{s}, wherein each R^{s} is independently H, C₁₋₁₄ alkyl, -L_{d}-OR_{d}, -L_{d}-SR_{d} or -L_{d}-NR_{d}R′_{d}, and wherein L_{d} is absent or C₁₋₁₄ alkylene; and wherein R_{d} and R′_{d} are independently H or C₁₋₁₄ alkyl.

In one embodiment, G_{1b} is a chemical bond. In one embodiment, G_{1b} is methylene. In one embodiment, G_{1b} is ethylene. In one embodiment, G_{1b} is C₃ alkylene. In one embodiment, G_{1b} is C₄ alkylene. In one embodiment, G_{1b} is C₅ alkylene. In one embodiment, G_{1b} is C₆ alkylene. In one embodiment, G_{1b} is C₇ alkylene. In one embodiment, G_{1b} is unsubstituted. In one embodiment, G_{1b} is substituted. In one embodiment, G_{1b} is substituted with one or more R^{s}, wherein each R^{s} is independently H, C₁₋₁₄ alkyl, -L_{d}-OR_{d}, -L_{d}-SR_{d} or -L_{d}-NR_{d}R′_{d}, and wherein L_{d} is absent or C₁₋₁₄ alkylene; and wherein R_{d} and R′_{d} are independently H or C₁₋₁₄ alkyl.

In one embodiment, G₂ₐ is a chemical bond. In one embodiment, G₂ₐ is methylene. In one embodiment, G₂ₐ is ethylene. In one embodiment, G₂ₐ is C₃ alkylene. In one embodiment, G₂ₐ is C₄ alkylene. In one embodiment, G₂ₐ is C₅ alkylene. In one embodiment, G₂ₐ is C₆ alkylene. In one embodiment, G₂ₐ is C₇ alkylene. In one embodiment, G₂ₐ is unsubstituted. In one embodiment, G₂ₐ is substituted. In one embodiment, G₂ₐ is substituted with one or more R^{s}, wherein each R^{s} is independently H, C₁₋₁₄ alkyl, -L_{d}-OR_{d}, -L_{d}-SR_{d} or -L_{d}-NR_{d}R′_{d}, and wherein L_{d} is absent or C₁₋₁₄ alkylene; and wherein R_{d} and R′_{d} are independently H or C₁₋₁₄ alkyl.

In one embodiment, G_{2b} is a chemical bond. In one embodiment, G_{2b} is methylene. In one embodiment, G_{2b} is ethylene. In one embodiment, G_{2b} is C₃ alkylene. In one embodiment, G_{2b} is C₄ alkylene. In one embodiment, G_{2b} is C₅ alkylene. In one embodiment, G_{2b} is C₆ alkylene. In one embodiment, G_{2b} is C₇ alkylene. In one embodiment, G_{2b} is unsubstituted. In one embodiment, G_{2b} is substituted. In one embodiment, G_{2b} is substituted with one or more R^{s}, wherein each R^{s} is independently H, C₁₋₁₄ alkyl, -L_{d}-OR_{d}, -L_{d}-SR_{d} or -L_{d}-NR_{d}R′_{d}, and wherein L_{d} is absent or C₁₋₁₄ alkylene; and wherein R_{d} and R′_{d} are independently H or C₁₋₁₄ alkyl.

In one embodiment, G₃ₐ is a chemical bond. In one embodiment, G₃ₐ is methylene. In one embodiment, G₃ₐ is ethylene. In one embodiment, G₃ₐ is C₃ alkylene. In one embodiment, G₃ₐ is C₄ alkylene. In one embodiment, G₃ₐ is C₅ alkylene. In one embodiment, G₃ₐ is C₆ alkylene. In one embodiment, G₃ₐ is C₇ alkylene. In one embodiment, G₃ₐ is unsubstituted. In one embodiment, G₃ₐ is substituted. In one embodiment, G₃ₐ is substituted with one or more R^{s}, wherein each R^{s} is independently H, C₁₋₁₄ alkyl, -L_{d}-OR_{d}, -L_{d}-SR_{d} or -L_{d}-NR_{d}R′_{d}, and wherein L_{d} is absent or C₁₋₁₄ alkylene; and wherein R_{d} and R′_{d} are independently H or C₁₋₁₄ alkyl.

In one embodiment, G_{3b} is a chemical bond. In one embodiment, G_{3b} is methylene. In one embodiment, G_{3b} is ethylene. In one embodiment, G_{3b} is C₃ alkylene. In one embodiment, G_{3b} is C₄ alkylene. In one embodiment, G_{3b} is C₅ alkylene. In one embodiment, G_{3b} is C₆ alkylene. In one embodiment, G_{3b} is C₇ alkylene. In one embodiment, G_{3b} is unsubstituted. In one embodiment, G_{3b} is substituted. In one embodiment, G_{3b} is substituted with one or more R^{s}, wherein each R^{s} is independently H, C₁₋₁₄ alkyl, -L_{d}-OR_{d}, -L_{d}-SR_{d} or -L_{d}-NR_{d}R′_{d}, and wherein L_{d} is absent or C₁₋₁₄ alkylene; and wherein R_{d} and R′_{d} are independently H or C₁₋₁₄ alkyl.

In one embodiment, G₄ₐ is a chemical bond. In one embodiment, G₄ₐ is methylene. In one embodiment, G₄ₐ is ethylene. In one embodiment, G₄ₐ is C₃ alkylene. In one embodiment, G₄ₐ is C₄ alkylene. In one embodiment, G₄ₐ is C₅ alkylene. In one embodiment, G₄ₐ is C₆ alkylene. In one embodiment, G₄ₐ is C₇ alkylene. In one embodiment, G₄ₐ is unsubstituted. In one embodiment, G₄ₐ is substituted. In one embodiment, G₄ₐ is substituted with one or more R^{s}, wherein each R^{s} is independently H, C₁₋₁₄ alkyl, -L_{d}-OR_{d}, -L_{d}-SR_{d} or -L_{d}-NR_{d}R′_{d}, and wherein L_{d} is absent or C₁₋₁₄ alkylene; and wherein R_{d} and R′_{d} are independently H or C₁₋₁₄ alkyl.

In one embodiment, G_{4b} is a chemical bond. In one embodiment, G_{4b} is methylene. In one embodiment, G_{4b} is ethylene. In one embodiment, G_{4b} is C₃ alkylene. In one embodiment, G_{4b} is C₄ alkylene. In one embodiment, G_{4b} is C₅ alkylene. In one embodiment, G_{4b} is C₆ alkylene. In one embodiment, G_{4b} is C₇ alkylene. In one embodiment, G_{4b} is unsubstituted. In one embodiment, G_{4b} is substituted. In one embodiment, G_{4b} is substituted with one or more R^{s}, wherein each R^{s} is independently H, C₁₋₁₄ alkyl, -L_{d}-OR_{d}, -L_{d}-SR_{d} or -L_{d}-NR_{d}R′_{d}, and wherein L_{d} is absent or C₁₋₁₄ alkylene; and wherein R_{d} and R′_{d} are independently H or C₁₋₁₄ alkyl.

In one embodiment, G₁ₐ, G_{1b}, G₂ₐ and G_{2b} have a total length of 1, 2, 3, 4, 5, 6 or 7 carbon atoms; in another more specific embodiment, G₁ₐ, G_{1b}, G₂ₐ and G_{2b} have a total length of 1, 2, 3, 4, 5 or 6 carbon atoms. In one embodiment, G₁ₐ, G_{1b}, G₂ₐ and G_{2b} have a total length of 1 carbon. In one embodiment, G₁ₐ, G_{1b}, G₂ₐ and G_{2b} have a total length of 2 carbons. In one embodiment, G₁ₐ, G_{1b}, G₂ₐ and G_{2b} have a total length of 3 carbons. In one embodiment, G₁ₐ, G_{1b}, G₂ₐ and G_{2b} have a total length of 4 carbons. In one embodiment, G₁ₐ, G_{1b}, G₂ₐ and G_{2b} have a total length of 5 carbons. In one embodiment, G₁ₐ, G_{1b}, G₂ₐ and G_{2b} have a total length of 6 carbons.

In one embodiment, G₃ₐ, G_{3b}, G₄ₐ and G_{4b} have a total length of 1, 2, 3, 4, 5, 6 or 7 carbons. In one embodiment, G₃ₐ, G_{3b}, G₄ₐ and G_{4b} have a total length of 1 carbon. In one embodiment, G₃ₐ, G_{3b}, G₄ₐ and G_{4b} have a total length of 2 carbons. In one embodiment, G₃ₐ, G_{3b}, G₄ₐ and G_{4b} have a total length of 3 carbons. In one embodiment, G₃ₐ, G_{3b}, G₄ₐ and G_{4b} have a total length of 4 carbons. In one embodiment, G₃ₐ, G_{3b}, G₄ₐ and G_{4b} have a total length of 5 carbons. In one embodiment, G₃ₐ, G_{3b}, G₄ₐ and G_{4b} have a total length of 6 carbons. In one embodiment, G₃ₐ, G_{3b}, G₄ₐ and G_{4b} have a total length of 7 carbons.

In one embodiment, G₇ is a chemical bond; in another embodiment, G₇ is C₁₋₁₂ alkylene; in another embodiment, G₇ is C₁₋₆ alkylene; in another embodiment, G₇ is C₁₋₅ alkylene; in another embodiment, G₇ is C₁₋₅ linear alkylene; in another embodiment, G₇ is —CH₂—; in another embodiment, G₇ is —(CH₂)₂—; in another embodiment, G₇ is —(CH₂)₄—; in another embodiment, G₇ is —(CH₂)₅—; in another embodiment, G₇ is optionally substituted with 1, 2, 3, 4, 5 or 6 R, wherein R is C₁₋₁₀ alkyl. In another embodiment, 1, 2 or 3 methylene in G₇ are optionally and independently substituted with 1 R; in another embodiment, 1 or 2 methylene in G₇ are optionally and independently substituted with 1 R; in another embodiment, the methylene of G₇ that is connected to M₁ is not substituted with R.

In one embodiment, G₈ is a chemical bond; in another embodiment, G₈ is C₁₋₁₂ alkylene; in another embodiment, G₈ is C₁₋₁₀ alkylene; in another embodiment, G₈ is C₁₋₈ alkylene; in another embodiment, G₈ is C₁₋₈ linear alkylene; in another embodiment, G₈ is —(CH₂)₂—; in another embodiment, G₈ is —(CH₂)₄—; in another embodiment, G₈ is —(CH₂)₆—; in another embodiment, G₈ is —(CH₂)₇—; in another embodiment, G₈ is —(CH₂)₈—; in another embodiment, G₈ is optionally substituted with 1, 2, 3, 4, 5 or 6 R, wherein R is C₁₋₁₀ alkyl; in another embodiment, 1, 2 or 3 methylene in G₈ are optionally and independently substituted with 1 R; in another embodiment, 1 or 2 alkylene in G₈ are optionally and independently substituted with 1 R.

In one embodiment, G₇ and G₈ have a total length of 4 carbon atoms; in another embodiment, G₇ and G₈ have a total length of 5 carbon atoms; in another embodiment, G₇ and G₈ have a total length of 6 carbon atoms; in another embodiment, G₇ and G₈ have a total length of 7 carbon atoms; in another embodiment, G₇ and G₈ have a total length of 8 carbon atoms; in another embodiment, G₇ and G₈ have a total length of 9 carbon atoms; in another embodiment, G₇ and G₈ have a total length of 10 carbon atoms; in another embodiment, G₇ and G₈ have a total length of 11 carbon atoms; in another embodiment, G₇ and G₈ have a total length of 12 carbon atoms.

In one embodiment, G₇ and G₈ have a total length of 6, 7, 8, 9 or 10 carbon atoms. In one embodiment, G₇ and G₈ have a total length of 6, 7 or 8 carbon atoms.

In one embodiment, G₉ is a chemical bond; in another embodiment, G₉ is C₁₋₁₂ alkylene; in another embodiment, G₉ is C₁₋₆ alkylene; in another embodiment, G₉ is C₁₋₅ alkylene; in another embodiment, G₉ is C₁₋₅ linear alkylene; in another embodiment, G₉ is —CH₂—; in another embodiment, G₉ is —(CH₂)₂—; in another embodiment, G₉ is —(CH₂)₄—; in another embodiment, G₉ is —(CH₂)₅—; in another embodiment, G₉ is optionally substituted with 1, 2, 3, 4, 5 or 6 R, wherein R is C₁₋₁₀ alkyl; in another embodiment, 1, 2 or 3 methylene in G₉ are optionally and independently substituted with 1 R; in another embodiment, 1 or 2 methylene in G₉ are optionally and independently substituted with 1 R; in another embodiment, the methylene of G₉ that is collected to M₂ is not substituted with R.

In one embodiment, G₁₀ is a chemical bond; in another embodiment, G₁₀ is C₁₋₁₂ alkylene; in another embodiment, G₁₀ is C₁₋₁₀ alkylene; in another embodiment, G₁₀ is C₁₋₈ alkylene; in another embodiment, G₁₀ is C₁₋₈ linear alkylene; in another embodiment, G₁₀ is —(CH₂)₂—; in another embodiment, G₁₀ is —(CH₂)₄—; in another embodiment, G₁₀ is —(CH₂)₆—; in another embodiment, G₁₀ is —(CH₂)₇—; in another embodiment, G₁₀ is —(CH₂)₈—; in another embodiment, G₁₀ is optionally substituted with 1, 2, 3, 4, 5 or 6 R, wherein R is C₁₋₁₀ alkyl; in another embodiment, 1, 2 or 3 methylene in G₁₀ are optionally and independently substituted with 1 R; in another embodiment, 1 or 2 methylene in G₁₀ are optionally and independently substituted with 1 R.

In one embodiment, G₉ and G₁₀ have a total length of 4 carbon atoms; in another embodiment, G₉ and G₁₀ have a total length of 5 carbon atoms; in another embodiment, G₉ and G₁₀ have a total length of 6 carbon atoms; in another embodiment, G₉ and G₁₀ have a total length of 7 carbon atoms; in another embodiment, G₉ and G₁₀ have a total length of 8 carbon atoms; in another embodiment, G₉ and G₁₀ have a total length of 9 carbon atoms; in another embodiment, G₉ and G₁₀ have a total length of 10 carbon atoms; in another embodiment, G₉ and G₁₀ have a total length of 11 carbon atoms; in another embodiment, G₉ and G₁₀ have a total length of 12 carbon atoms.

In one embodiment, G₉ and G₁₀ have a total length of 6, 7, 8, 9 or 10 carbon atoms. In one embodiment, G₉ and G₁₀ have a total length of 6, 7 or 8 carbon atoms.

In one embodiment, R^{s} is H. In one embodiment, R^{s} is C₁₋₁₀ alkyl. In one embodiment, R^{s} is C₁₋₆ alkyl. In one embodiment, R^{s} is methyl. In one embodiment, R^{s} is ethyl. In one embodiment, R^{s} is C₃ alkyl. In one embodiment, R^{s} is C₄ alkyl. In one embodiment, R^{s} is C₆ alkyl. In one embodiment, R^{s} is C₆ alkyl. In one embodiment, R^{s} is -L_{d}-OR_{d}. In one embodiment, R^{s} is -L_{d}-NR_{d}R′_{d}. In one embodiment, R^{s} is —OR_{d}. In one embodiment, R^{s} is —NR_{d}R′_{d}. In one embodiment, R^{s} is —CH₂—OR_{d}. In one embodiment, R^{s} is —CH₂—NR_{d}R′_{d}.

In one embodiment, R^{s′} is H. In one embodiment, R^{s′} is C₁₋₁₀ alkyl. In one embodiment, R^{s′} is C₁₋₆ alkyl. In one embodiment, R^{s′} is methyl. In one embodiment, R^{s′} is ethyl. In one embodiment, R^{s′} is C₃ alkyl. In one embodiment, R^{s′} is C₄ alkyl. In one embodiment, R^{s′} is C₅ alkyl. In one embodiment, R^{s′} is C₆ alkyl. In one embodiment, R^{s′} is -L_{d}-OR_{d}. In one embodiment, R^{s′} is -L_{d}-NR_{d}R′_{d}. In one embodiment, R^{s′} is —OR_{d}. In one embodiment, R^{s′} is —NR_{d}R′_{d}. In one embodiment, R^{s′} is —CH₂—OR_{d}. In one embodiment, R^{s′} is —CH₂—NR_{d}R′_{d}.

In one embodiment, R′ is H. In one embodiment, R′ is C₁₋₁₄ alkyl. In one embodiment, R is C₁₋₈ alkyl. In one embodiment, R′ is C₁₋₆ alkyl. In one embodiment, R′ is -Lₐ-ORₐ. In one embodiment, R′ is -Lₐ-NRₐR′ₐ. In one embodiment, R′ is —ORₐ. In one embodiment, R′ is —NRₐR′ₐ. In one embodiment, R′ is —CH₂—ORₐ. In one embodiment, R′ is —CH₂—NRₐR′ₐ.

In one embodiment, R″ is H. In one embodiment, R″ is C₁₋₁₄ alkyl. In one embodiment, R″ is C₁₋₈ alkyl. In one embodiment, R″ is C₁₋₆ alkyl. In one embodiment, R″ is methyl. In one embodiment, R″ is ethyl. In one embodiment, R″ is C₃ alkyl. In one embodiment, R″ is C₄ alkyl. In one embodiment, R″ is C₅ alkyl. In one embodiment, R″ is C₆ alkyl.

In one embodiment, L_{d} is a chemical bond. In one embodiment, L_{d} is C₁₋₁₀ alkylene. In one embodiment, L_{d} is C₁₋₆ alkylene. In one embodiment, L_{d} is methylene. In one embodiment, L_{d} is ethylene. In one embodiment, L_{d} is C₃ alkylene. In one embodiment, L_{d} is C₄ alkylene. In one embodiment, L_{d} is C₅ alkylene. In one embodiment, L_{d} is C₆ alkylene.

In one embodiment, R^{d} is H. In one embodiment, R^{d} is C₁₋₁₀ alkyl. In one embodiment, R^{d} is C₁₋₈ alkyl. In one embodiment, R^{d} is C₁₋₆ alkyl. In one embodiment, R^{d} is methyl. In one embodiment, R^{d} is ethyl. In one embodiment, R^{d} is C₃ alkyl. In one embodiment, R^{d} is C₄ alkyl. In one embodiment, R^{d} is C₅ alkyl. In one embodiment, R^{d} is C₆ alkyl. In one embodiment, R^{d} is C₇ alkyl. In one embodiment, R^{d} is C₈ alkyl. In one embodiment, R^{d} is C₉ alkyl. In one embodiment, R^{d} is C₁₀ alkyl.

In one embodiment, R′^{d} is H. In one embodiment, R′^{d} is C₁₋₁₀ alkyl. In one embodiment, R′^{d} is C₁₋₈ alkyl. In one embodiment, R′^{d} is C₁₋₆ alkyl. In one embodiment, R′^{d} is methyl. In one embodiment, R′^{d} is ethyl. In one embodiment, R′^{d} is C₃ alkyl. In one embodiment, R′^{d} is C₄ alkyl. In one embodiment, R′^{d} is C₅ alkyl. In one embodiment, R′^{d} is C₆ alkyl. In one embodiment, R′^{d} is C₇ alkyl. In one embodiment, R′^{d} is C₈ alkyl. In one embodiment, R′^{d} is C₉ alkyl. In one embodiment, R′^{d} is C₁₀ alkyl.

In one embodiment, a′ is 0. In one embodiment, a′ is 1. In one embodiment, a′ is 2. In one embodiment, a′ is 3. In one embodiment, a′ is 4. In one embodiment, a′ is 5.

In one embodiment, b is 0. In one embodiment, b is 1. In one embodiment, b is 2. In one embodiment, b is 3. In one embodiment, b is 4. In one embodiment, b is 5.

In one embodiment, g is 0. In one embodiment, g is 1. In one embodiment, g is 2. In one embodiment, g is 3. In one embodiment, g is 4. In one embodiment, g is 5.

In one embodiment, a′ is 2 and b is 2. In one embodiment, a′ is 0 and b is 2. In one embodiment, a′ is 2 and b is 0. In one embodiment, a′ is 1 and b is 2. In one embodiment, a′ is 2 and b is 1.

In one embodiment, a′+g equals 2. In one embodiment, a′+g equals 3. In one embodiment, a′+g equals 0. In one embodiment, a′+g equals 1. In one embodiment, a′+g equals 4. In one embodiment, a′+g equals 5.

In one embodiment, c is 0. In one embodiment, c is 1. In one embodiment, c is 2. In one embodiment, c is 3. In one embodiment, c is 4. In one embodiment, c is 5. In one embodiment, c is 6. In one embodiment, c is 7.

In one embodiment, d is 0. In one embodiment, d is 1. In one embodiment, d is 2. In one embodiment, d is 3. In one embodiment, d is 4. In one embodiment, d is 5. In one embodiment, d is 6. In one embodiment, d is 7.

In one embodiment, e is 0. In one embodiment, e is 1. In one embodiment, e is 2. In one embodiment, e is 3. In one embodiment, e is 4. In one embodiment, e is 5. In one embodiment, e is 6. In one embodiment, e is 7.

In one embodiment, f is 0. In one embodiment, f is 1. In one embodiment, f is 2. In one embodiment, f is 3. In one embodiment, f is 4. In one embodiment, f is 5. In one embodiment, f is 6. In one embodiment, f is 7.

In one embodiment, c+d equals 2. In one embodiment, c+d equals 3. In one embodiment, c+d equals 0. In one embodiment, c+d equals 1. In one embodiment, c+d equals 4. In one embodiment, c+d equals 5. In one embodiment, c+d equals 6. In one embodiment, c+d equals 7. In one embodiment, c+d equals 8. In one embodiment, c+d equals 9.

In one embodiment, e+f equals 2. In one embodiment, e+f equals 3. In one embodiment, e+f equals 0. In one embodiment, e+f equals 1. In one embodiment, e+f equals 4. In one embodiment, e+f equals 5. In one embodiment, e+f equals 6. In one embodiment, e+f equals 7. In one embodiment, e+f equals 8. In one embodiment, e+f equals 9.

In one embodiment,

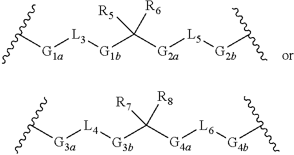

have a total length of 4, 5, 6, 7, 8 or 9 carbon atoms.

In one embodiment,

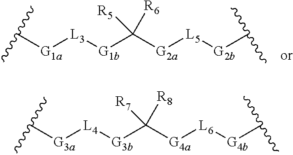

is independently selected from: —(CH₂)₃—C(CH₃)₂—, —(CH₂)₄—C(CH₃)₂—, —(CH₂)₅—C(CH₃)₂—, —(CH₂)₆—C(CH₃)₂—, —(CH₂)₇—C(CH₃)₂—, —(CH₂)₈—C(CH₃)₂—, —(CH₂)₃—CH═CH—C(CH₃)₂—, —(CH₂)₃—C≡C—C(CH₃)₂—, —(CH₂)₄—C(CH₃)₂—CH₂—, —(CH₂)₃—C(CH₃)₂—(CH₂)₂—, —(CH₂)₂—C(CH₃)₂—(CH₂)₃—, —(CH₂)₂—CH═CH—C(CH₃)₂—CH₂—, —(CH₂)₂—C(CH₃)₂—C≡C—CH₂—, —(CH₂)₂—C(CH₃)₂—CH═CH—CH₂—, —(CH₂)₂—C≡C—C(CH₃)₂—CH₂— and —(CH₂)₃—C(CH₃)₂—C≡C—; In in one embodiment

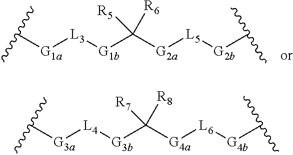

is independently —(CH₂)₄—C(CH₃)₂—, —(CH₂)₅—C(CH₃)₂— or —(CH₂)₆—C(CH₃)₂—; In in one embodiment,

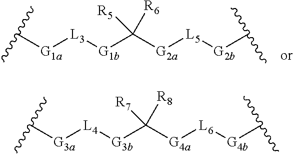

is —(CH₂)₅—C(CH₃)₂—.

In one embodiment, -G₇-L₁-G₈-H or -G₉-L₂-G₁₀-H is independently selected from: —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, —CH₂—C≡C—(CH₂)₅CH₃, —CH₂—C≡C—(CH₂)₆CH₃, —(CH₂)₂—C≡C—(CH₂)₅CH₃, —(CH₂)₄—C≡C—(CH₂)₃CH₃, —CH₂—CH═CH—(CH₂)₅CH₃, —CH₂—CH═CH—(CH₂)₆CH₃, —(CH₂)₂—CH═CH—(CH₂)₅CH₃, —(CH₂)₄—CH═CH—(CH₂)₃CH₃, —(CH₂)₅—CH═CH—CH₂CH₃,

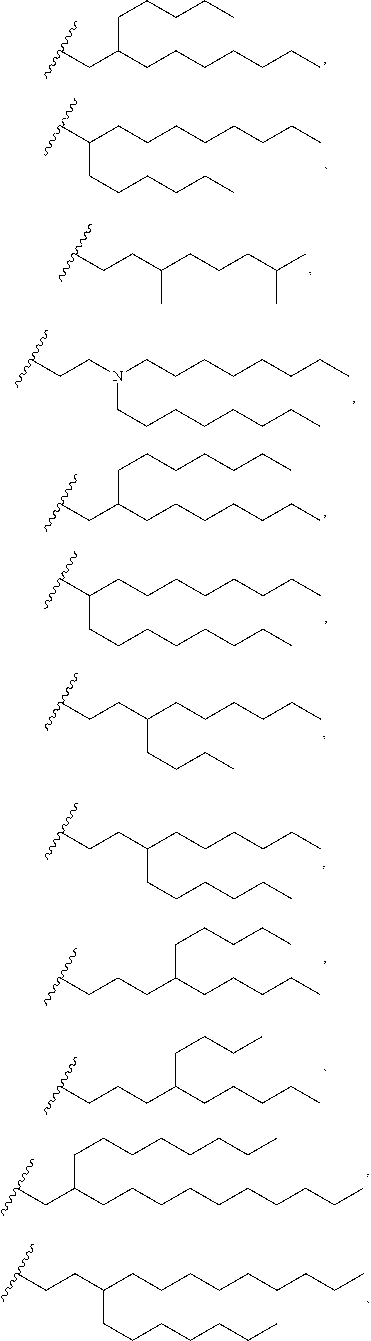

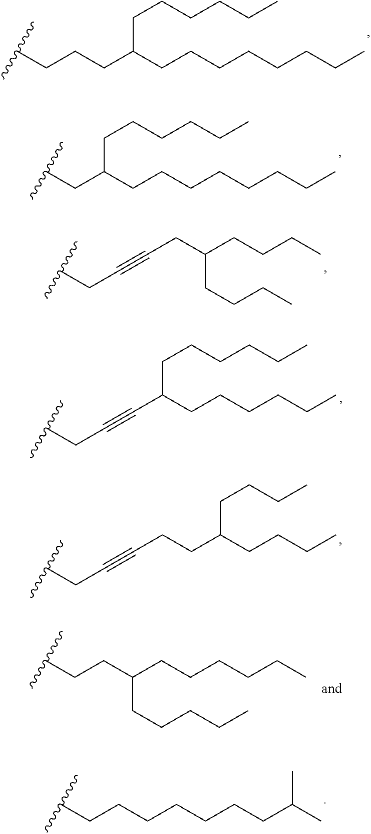

In one embodiment, -G₇-L₁-G₈-H or -G₉-L₂-G₁₀-H is independently selected from the following groups: —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, —CH₂—C═C—(CH₂)₅CH₃, —CH₂—C═C—(CH₂)₆CH₃, —(CH₂)₂—C═C—(CH₂)₅CH₃, —(CH₂)₄—C≡C—(CH₂)₃CH₃, —CH₂—CH═CH—(CH₂)₅CH₃, —CH₂—CH═CH—(CH₂)₆CH₃, —(CH₂)₂—CH═CH—(CH₂)₅CH₃, —(CH₂)₄—CH═CH—(CH₂)₃CH₃, —(CH₂)₅—CH═CH—CH₂CH₃,

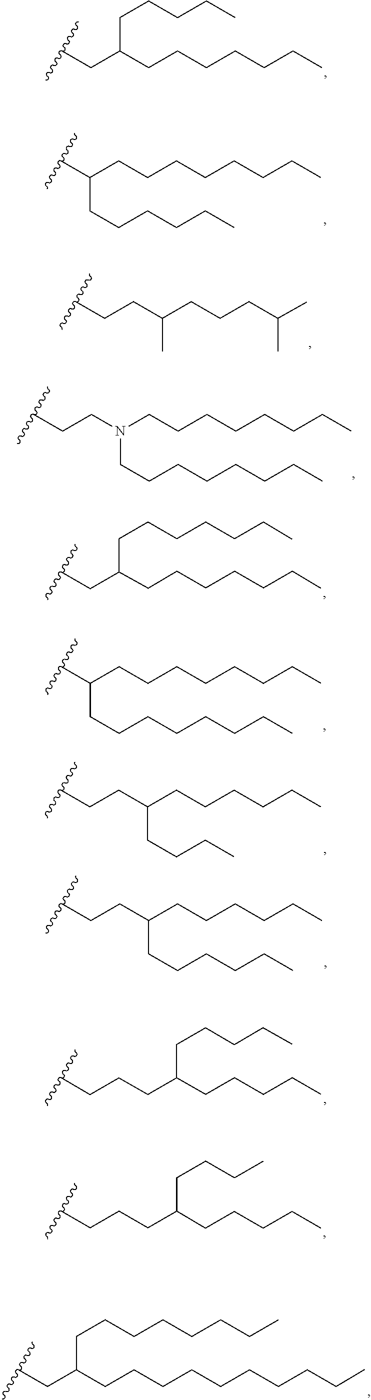

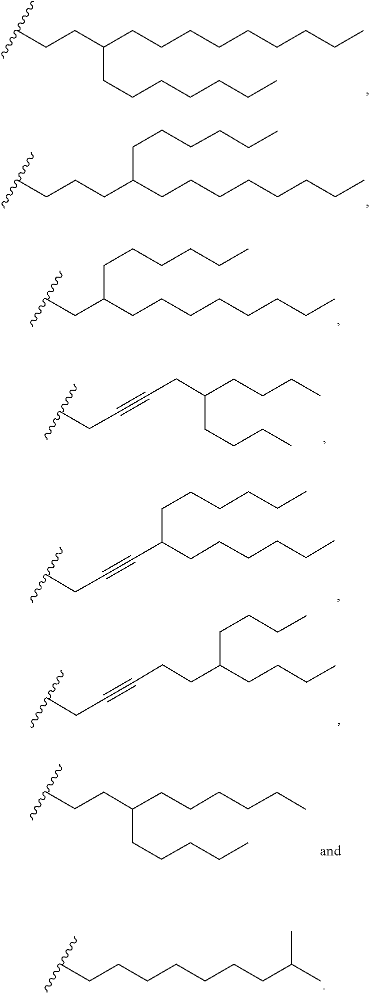

In one embodiment, the ionizable lipid is:

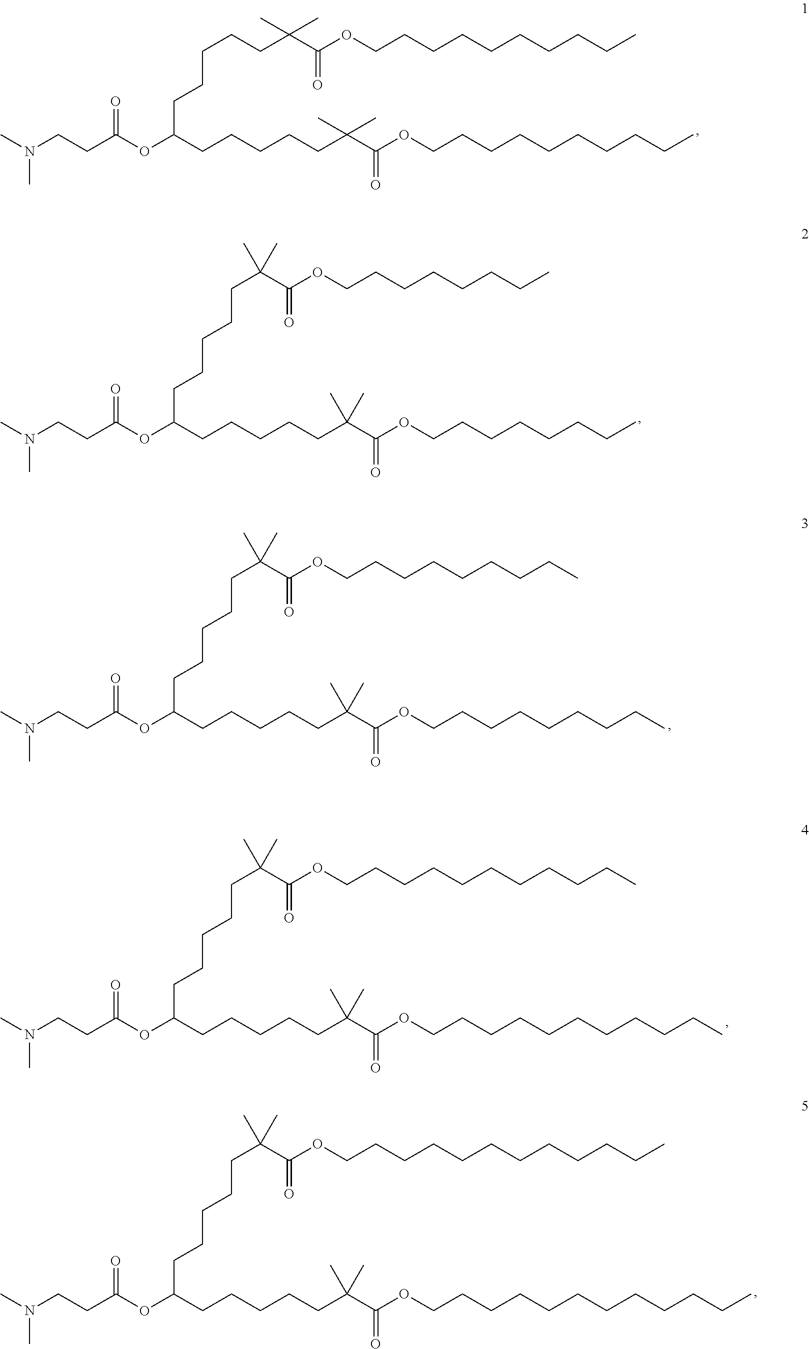

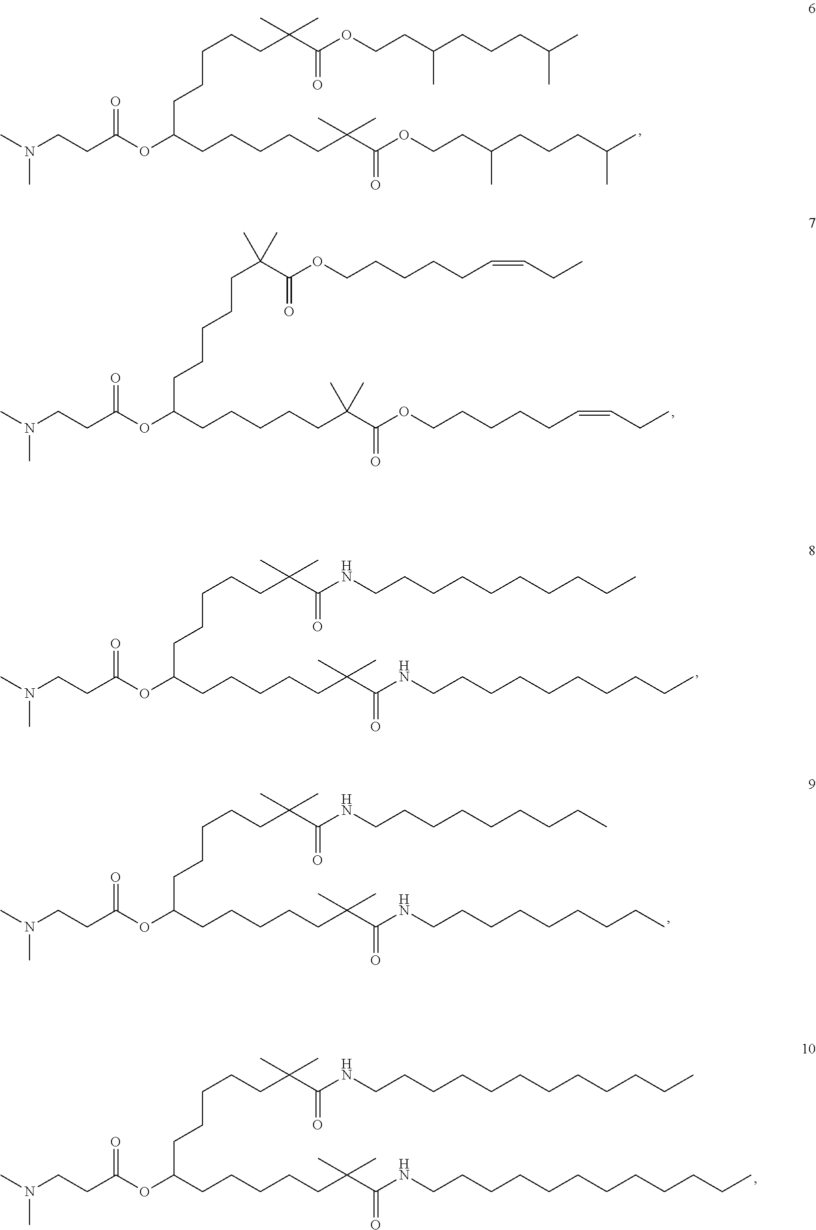

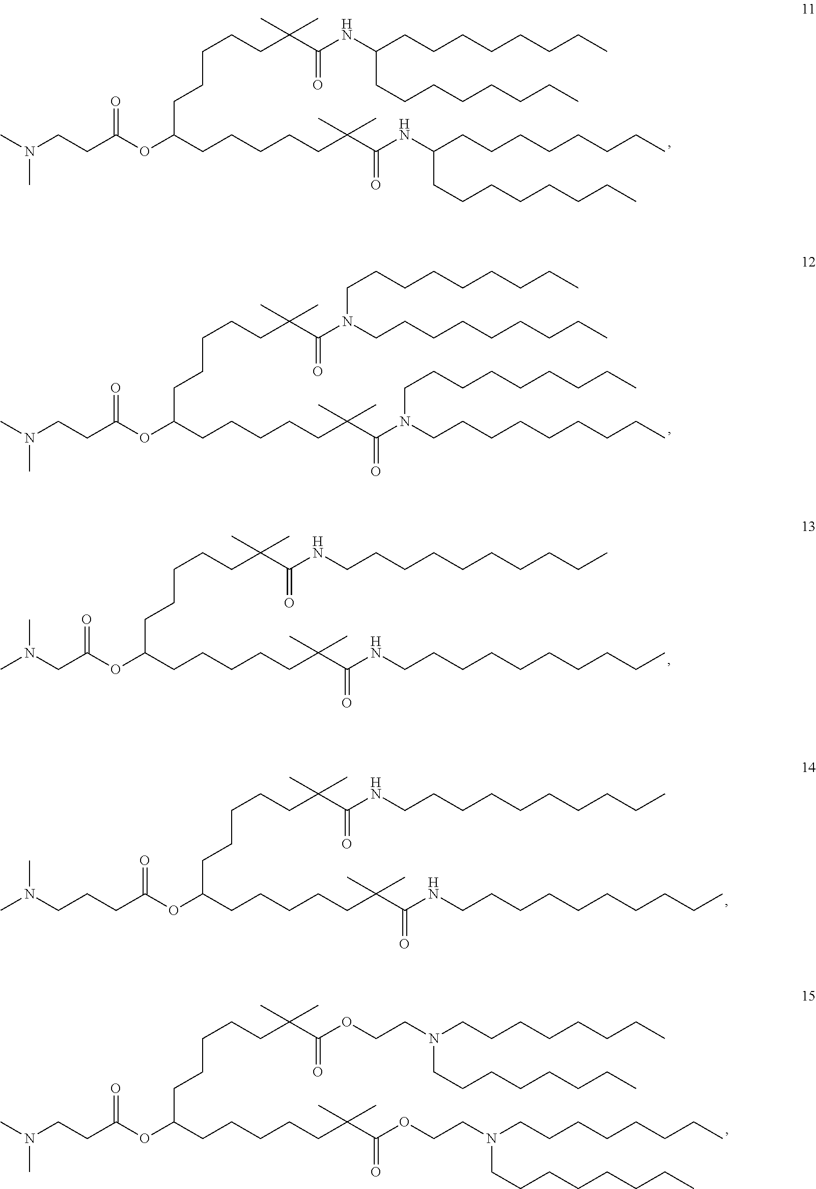

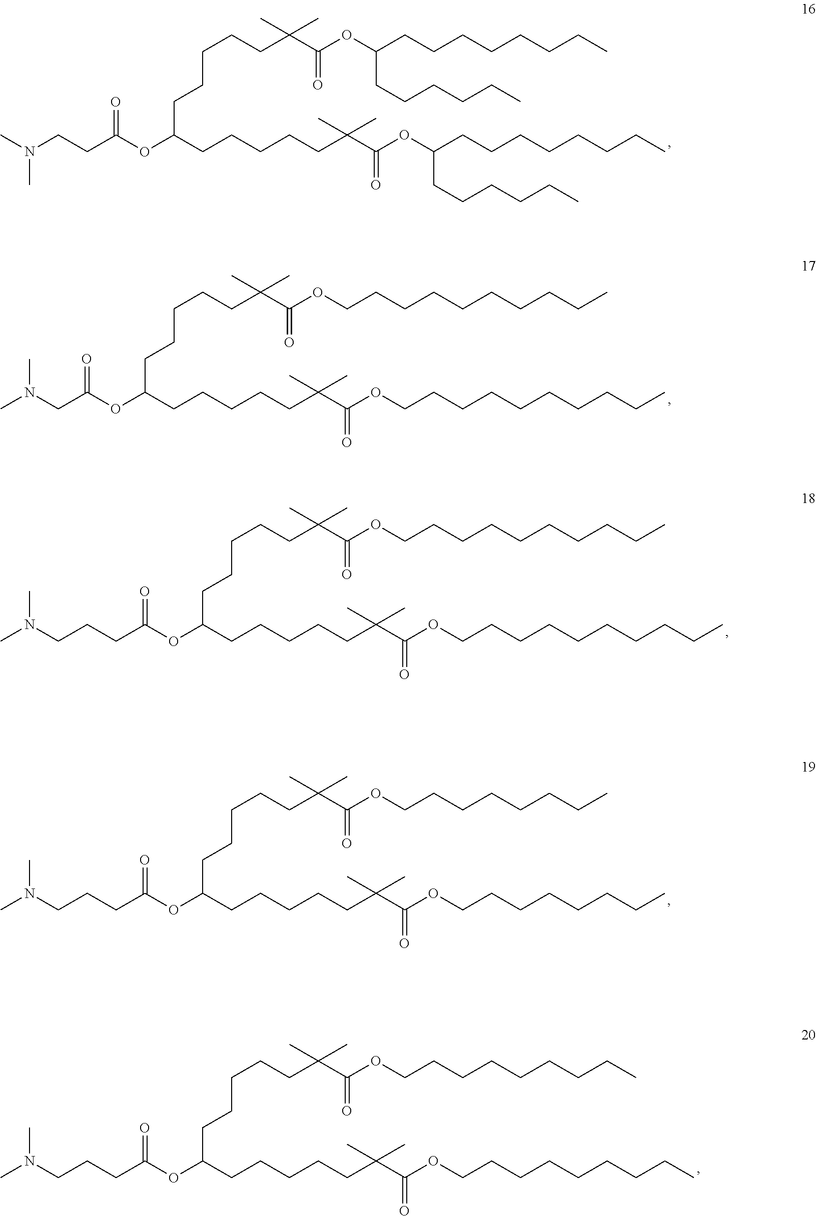

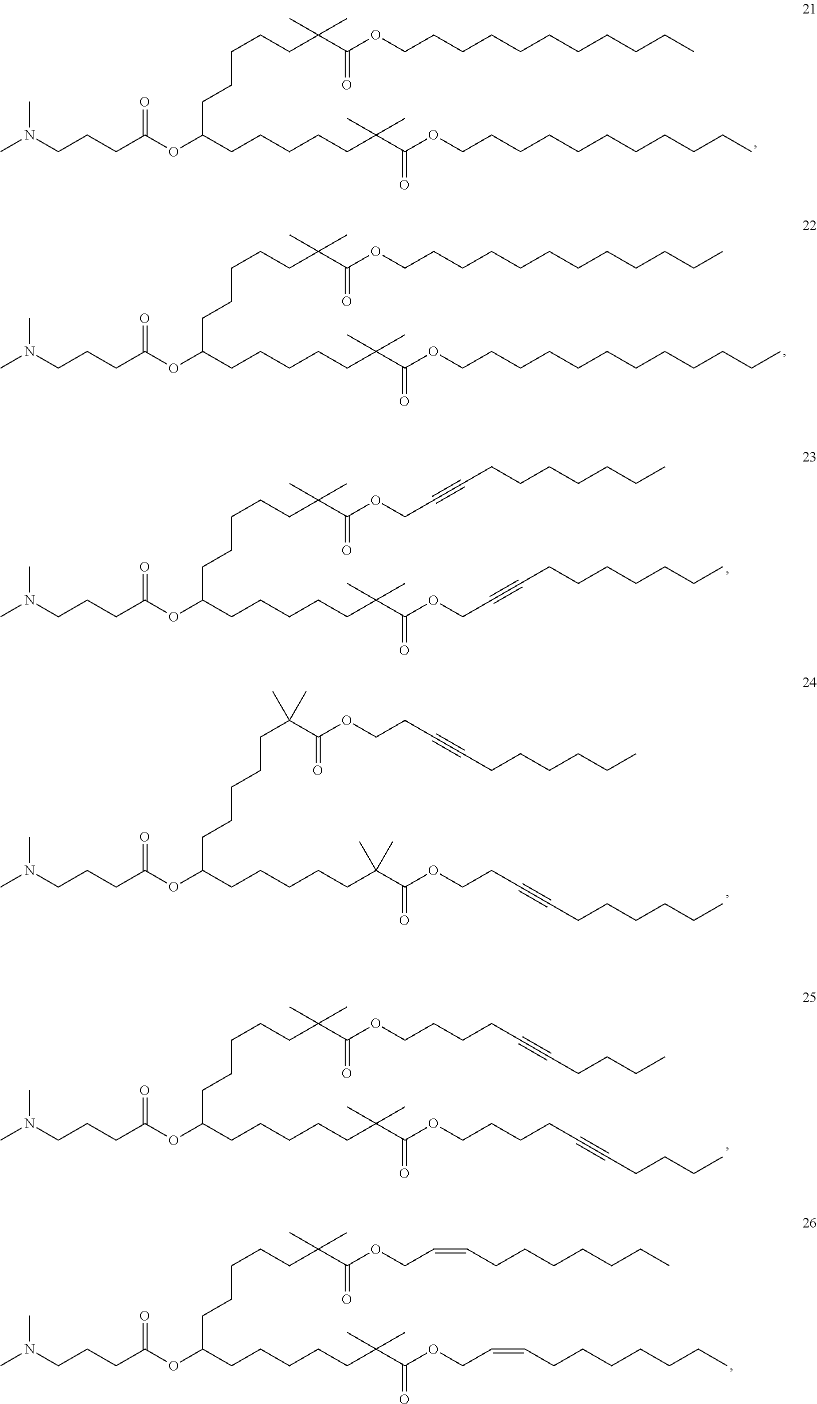

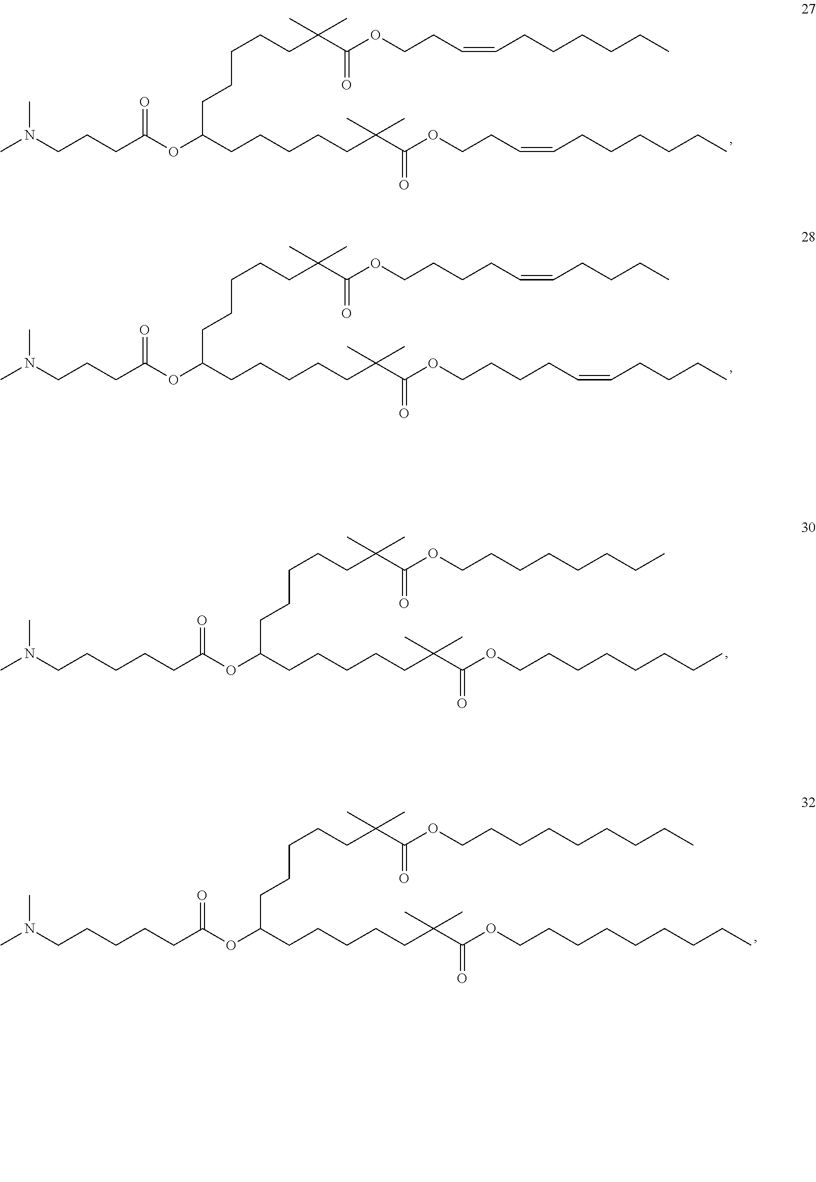

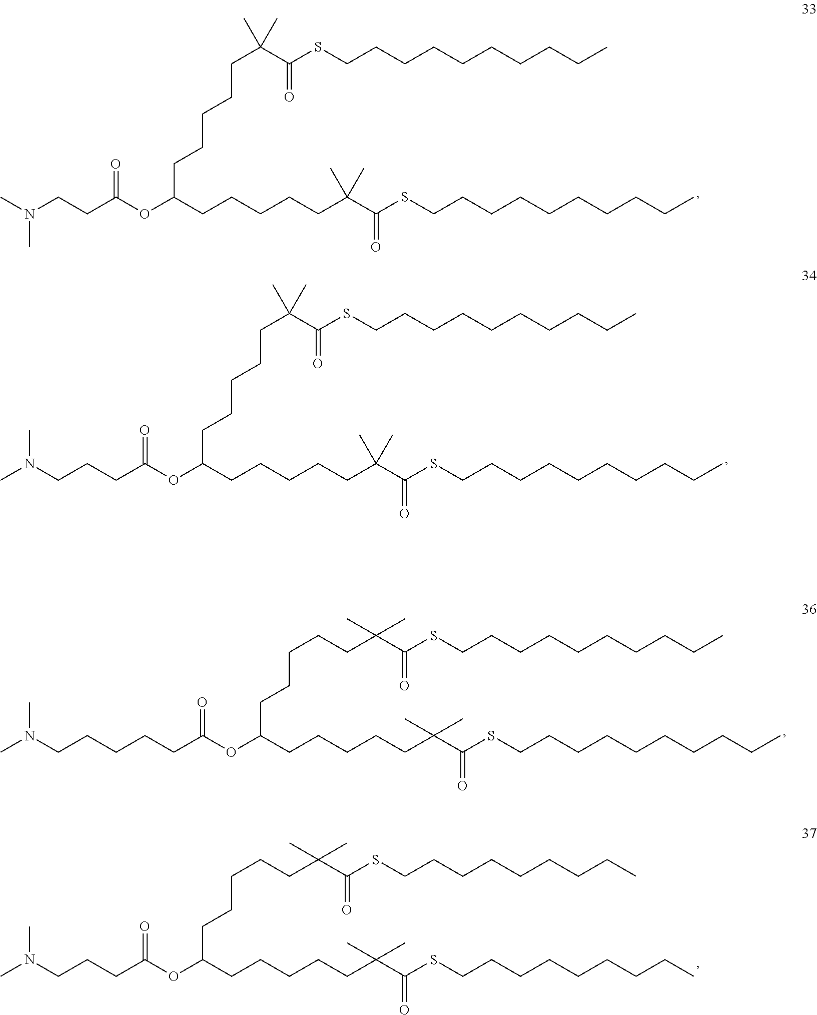

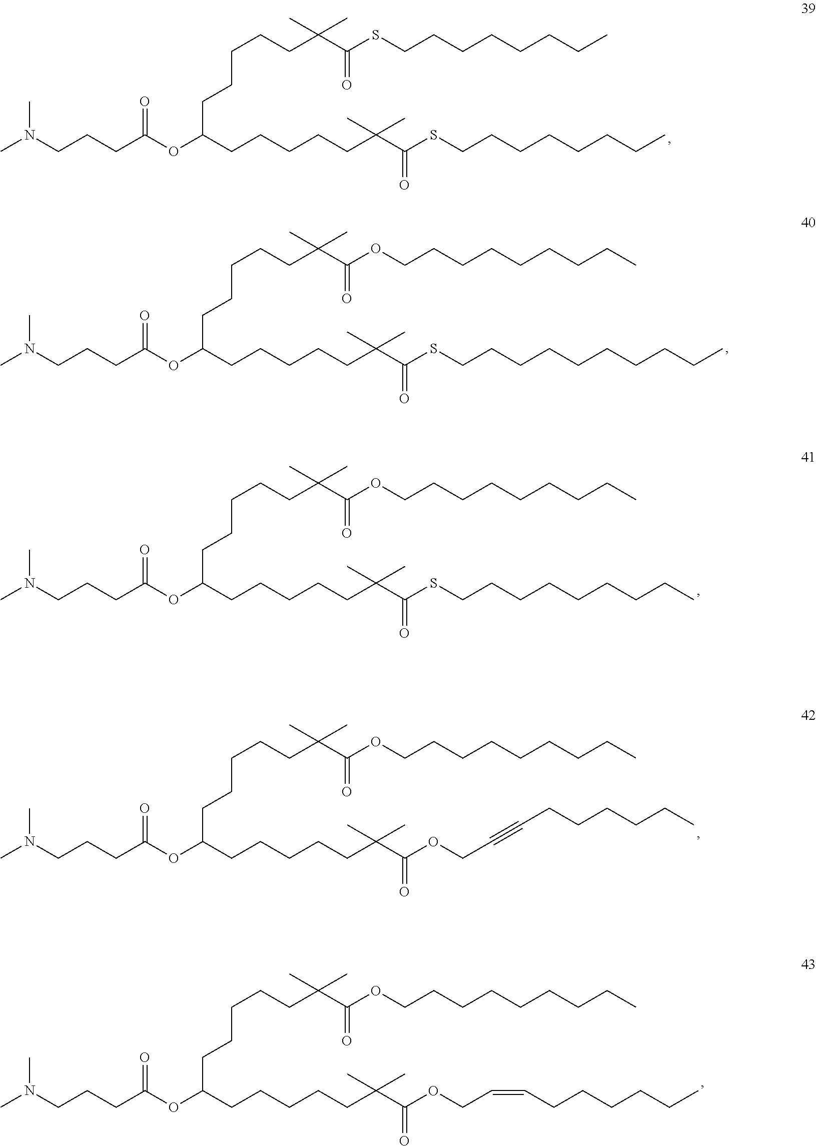

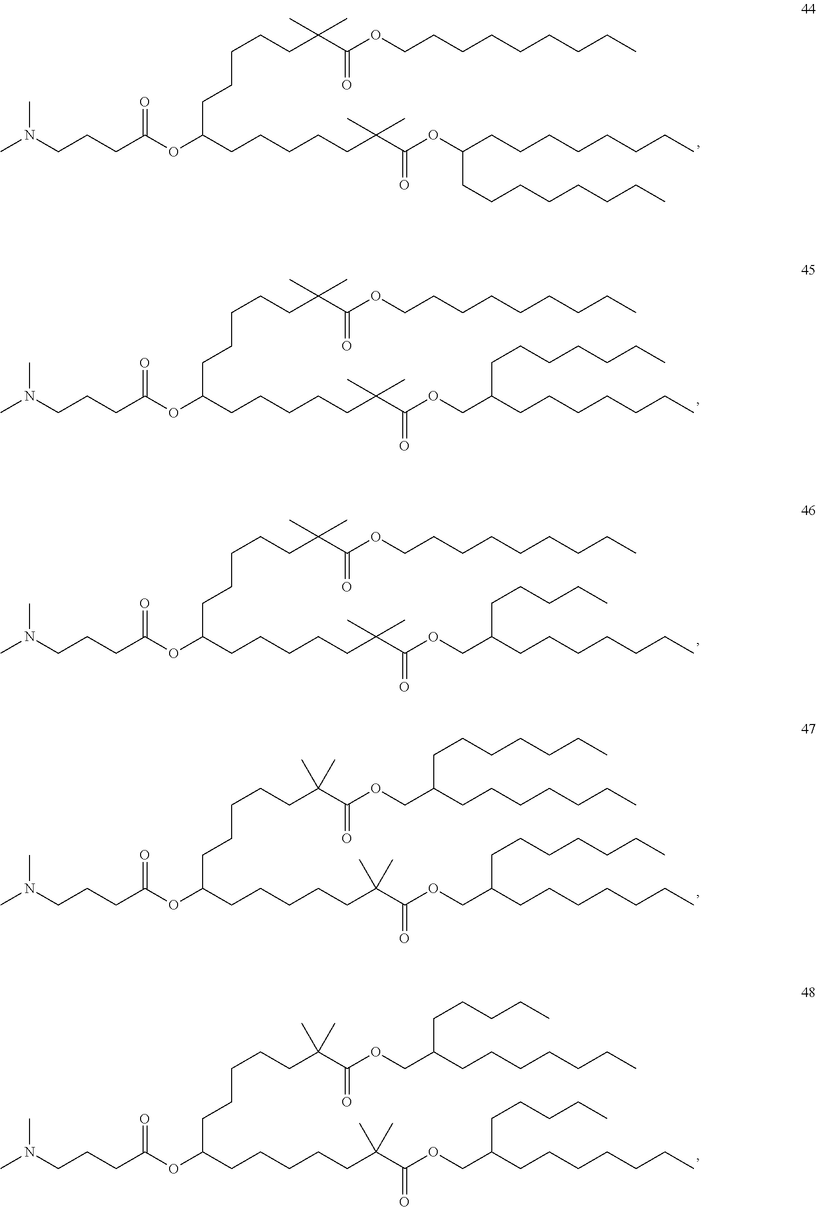

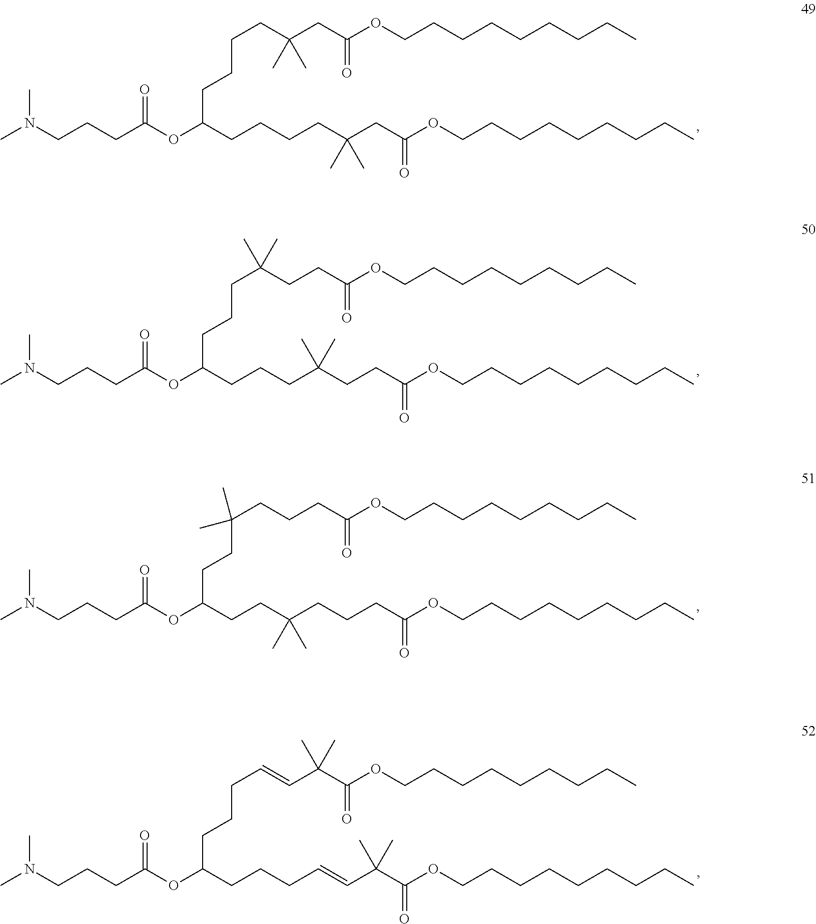

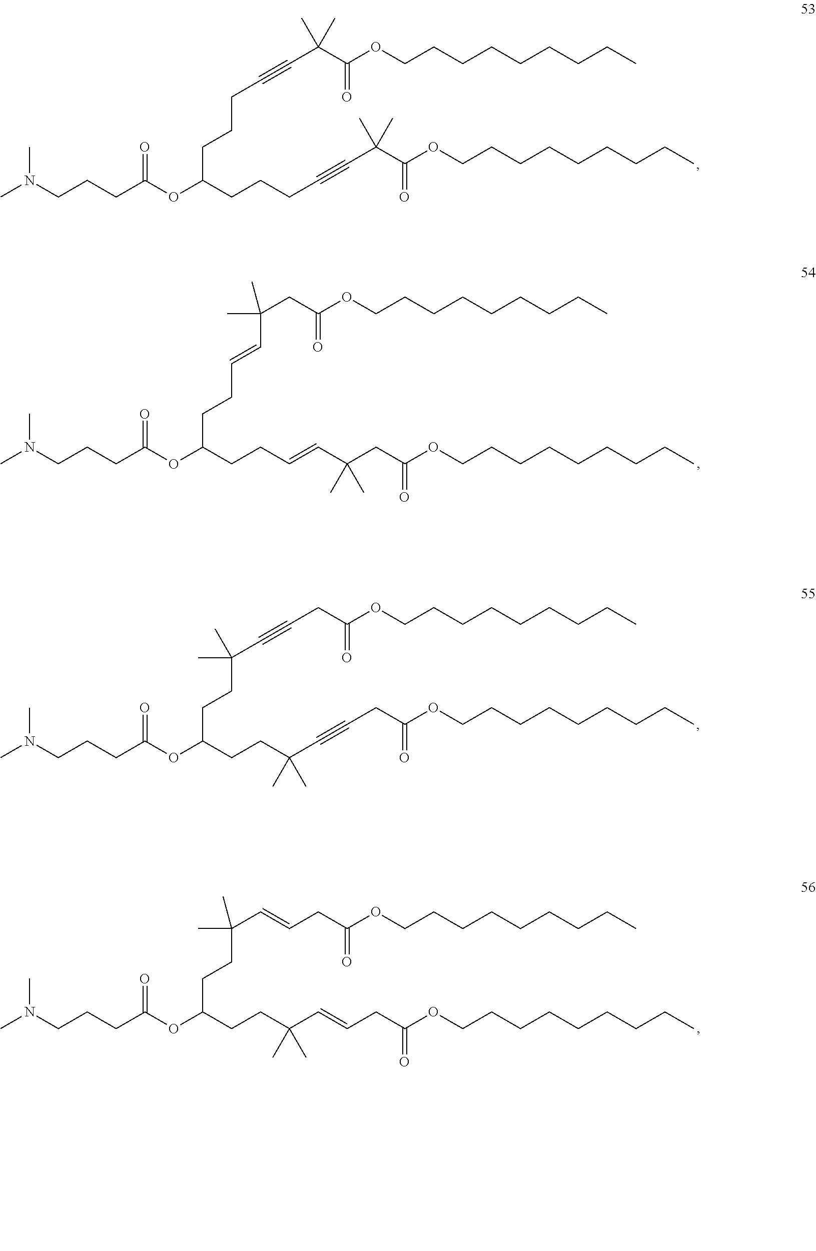

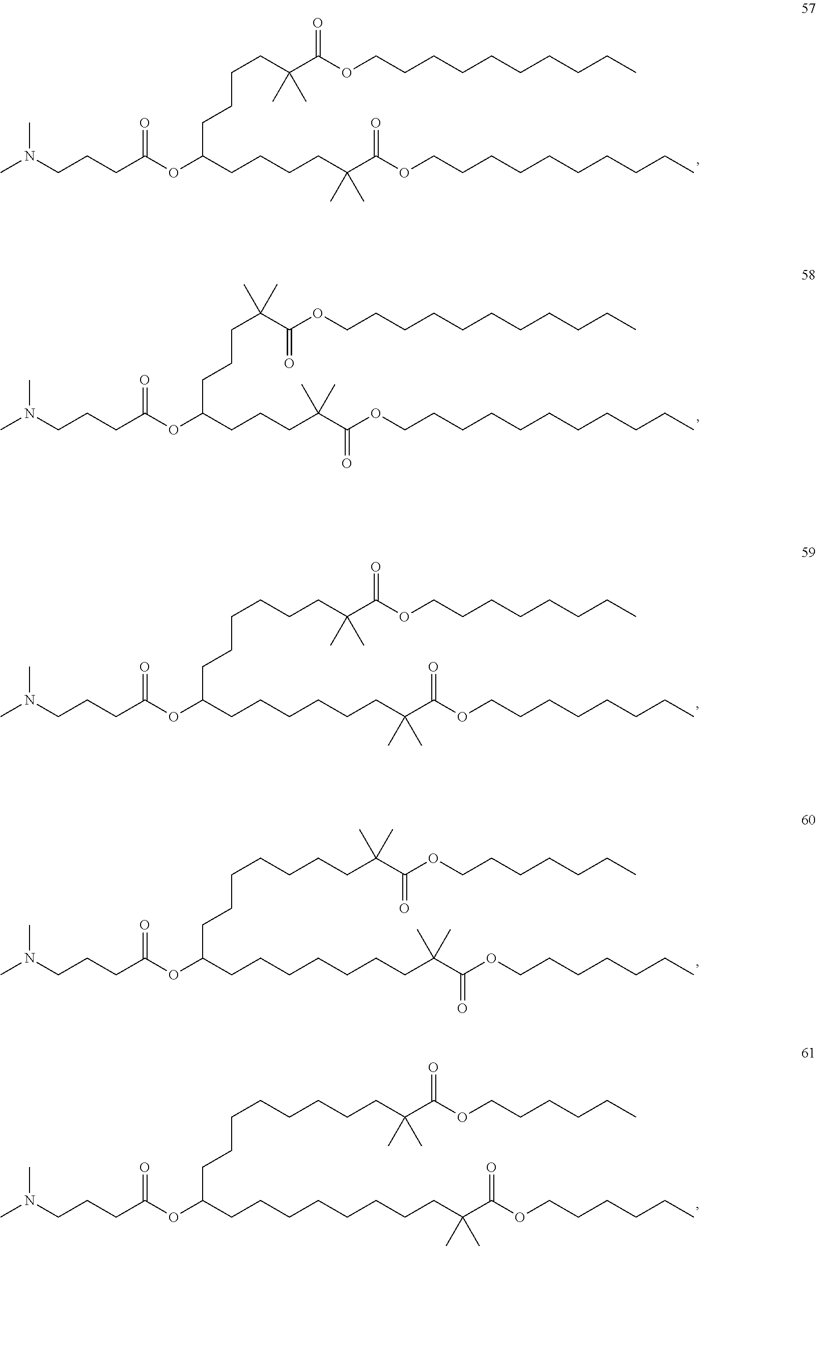

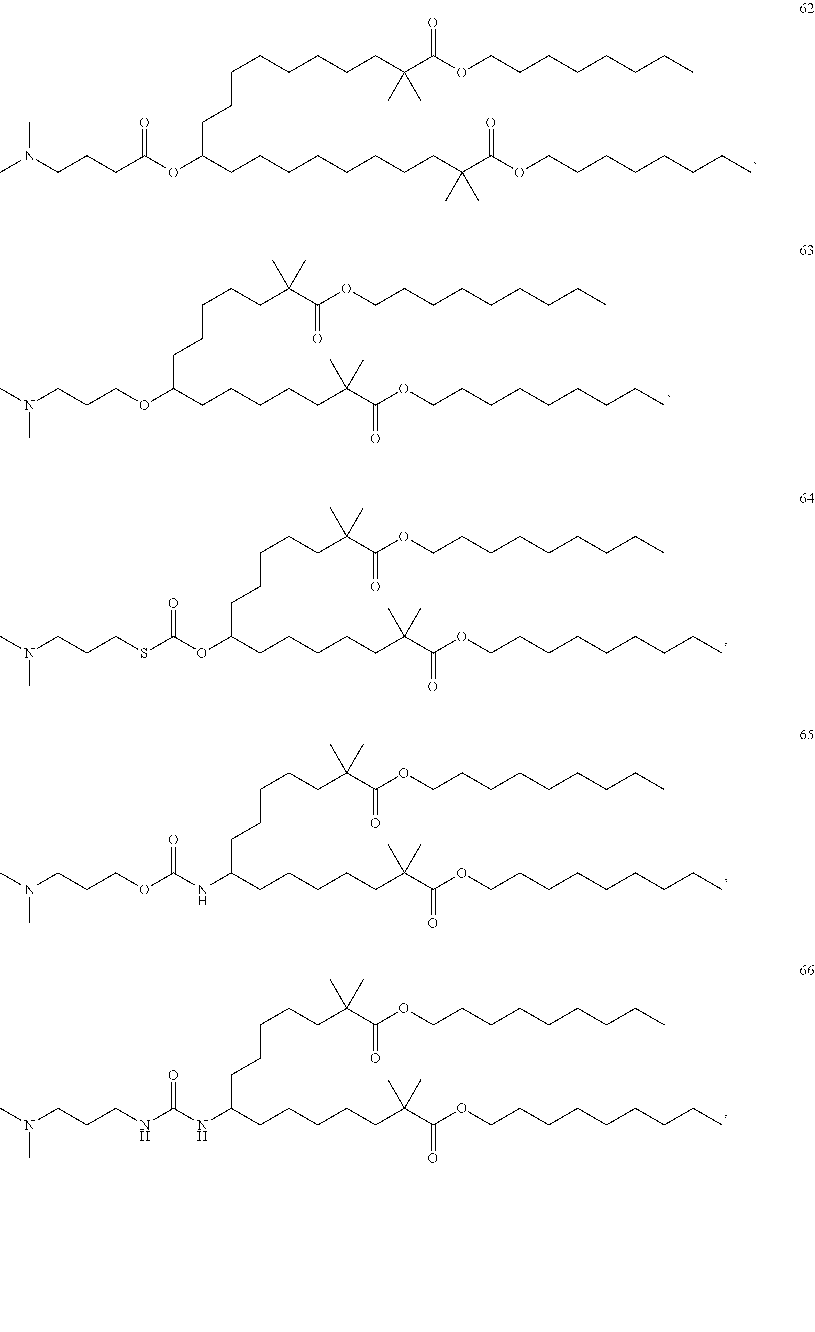

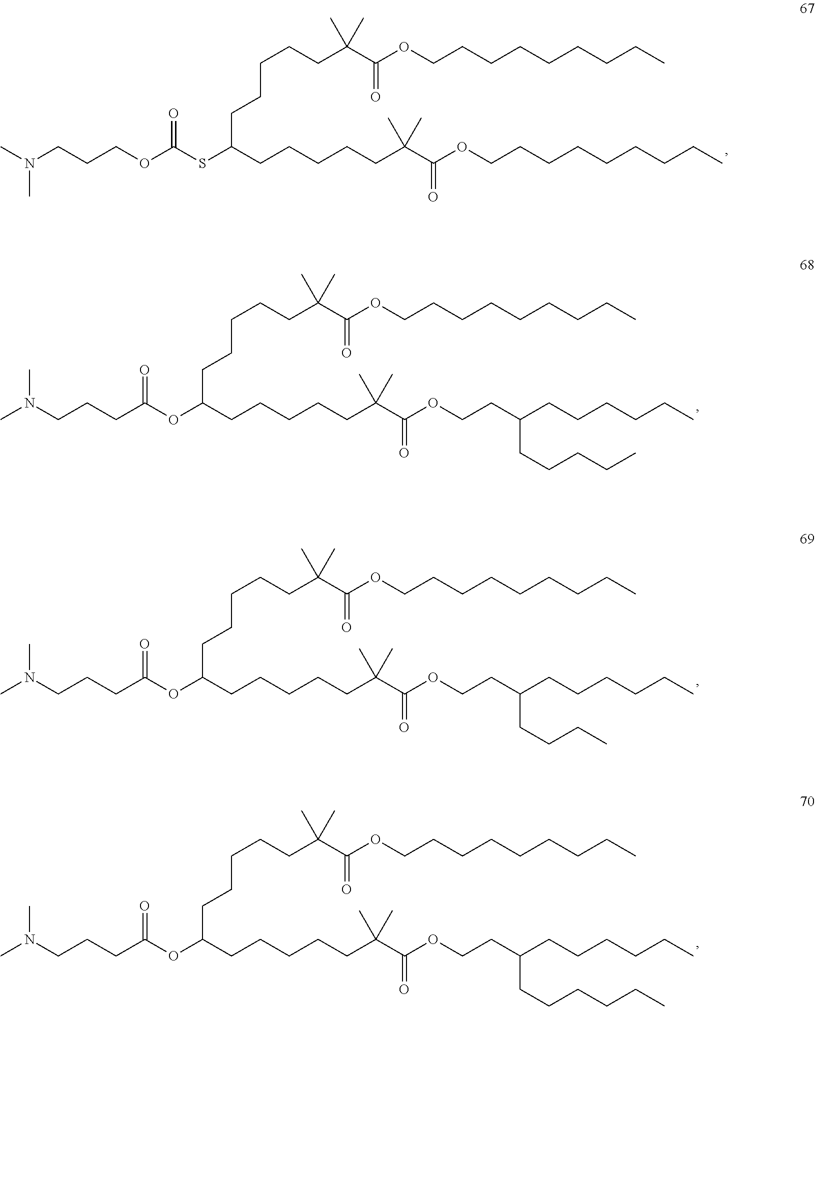

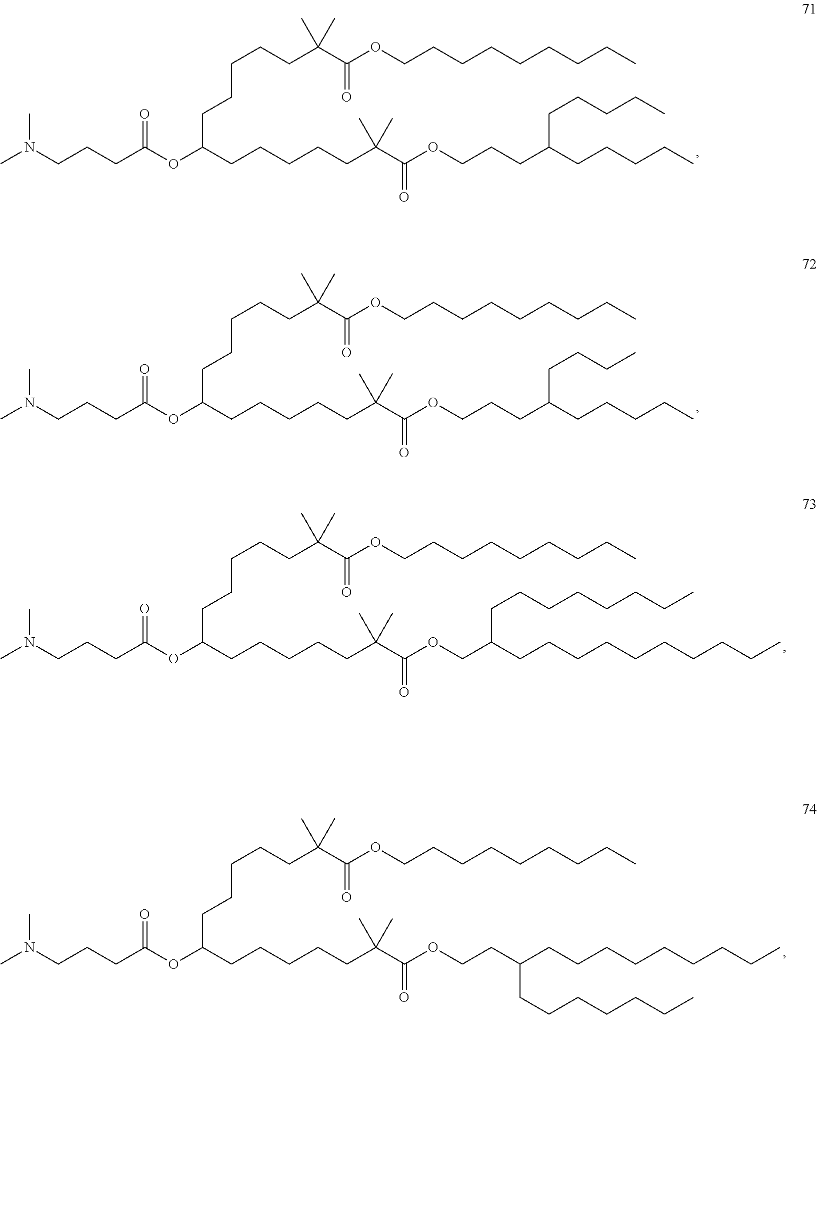

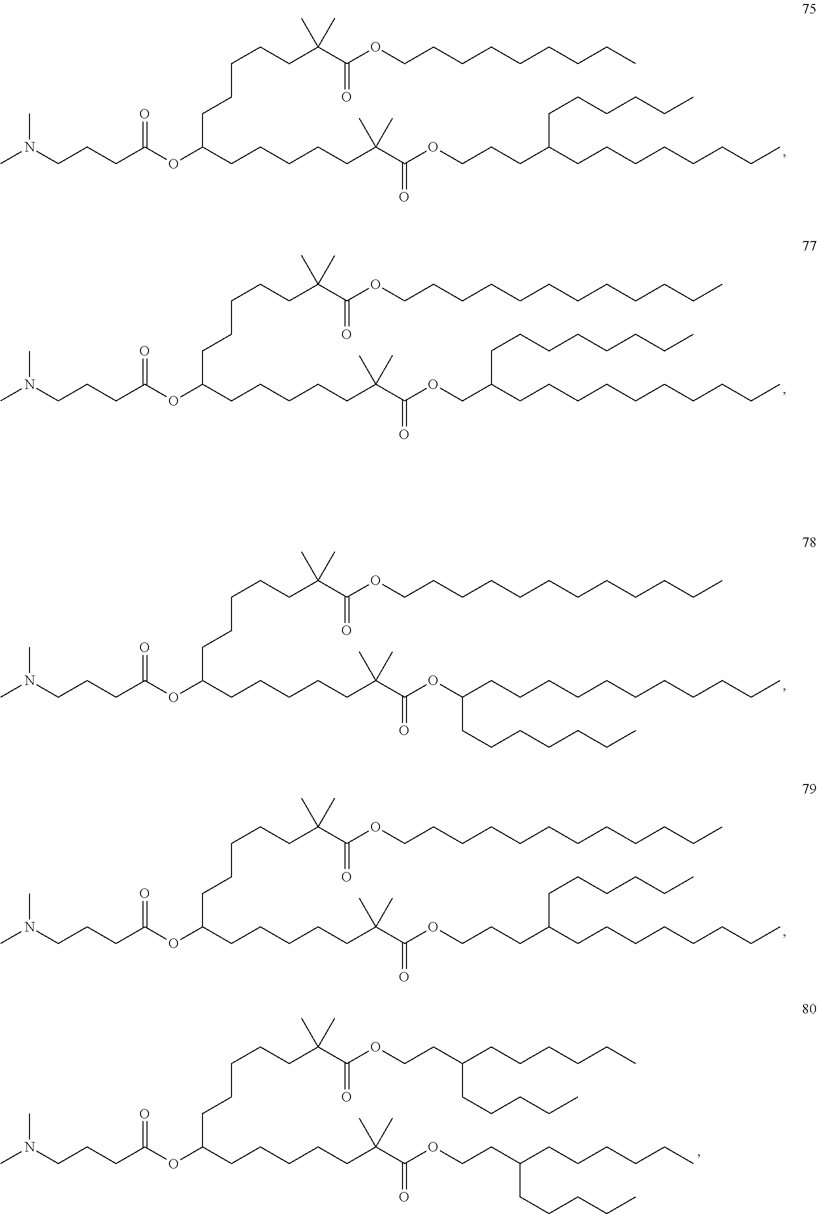

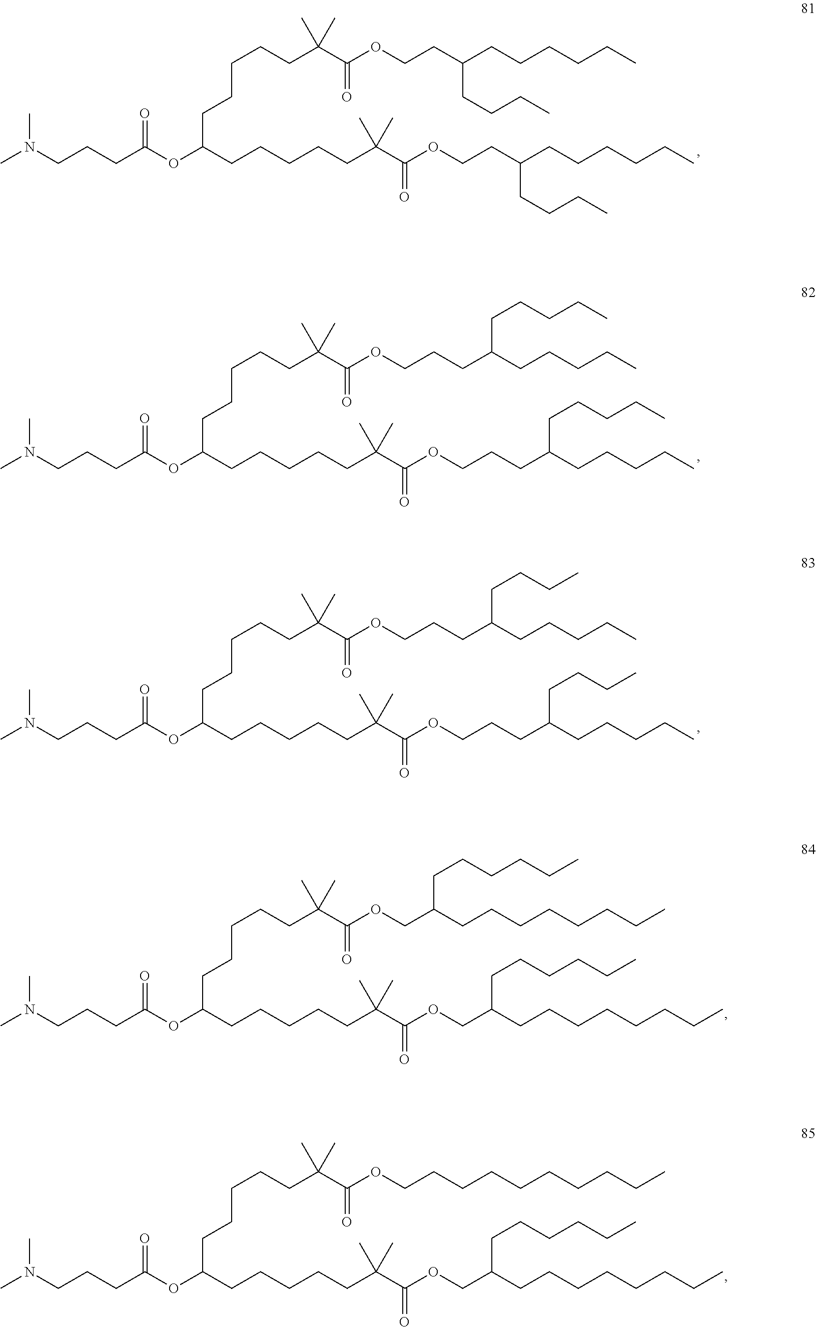

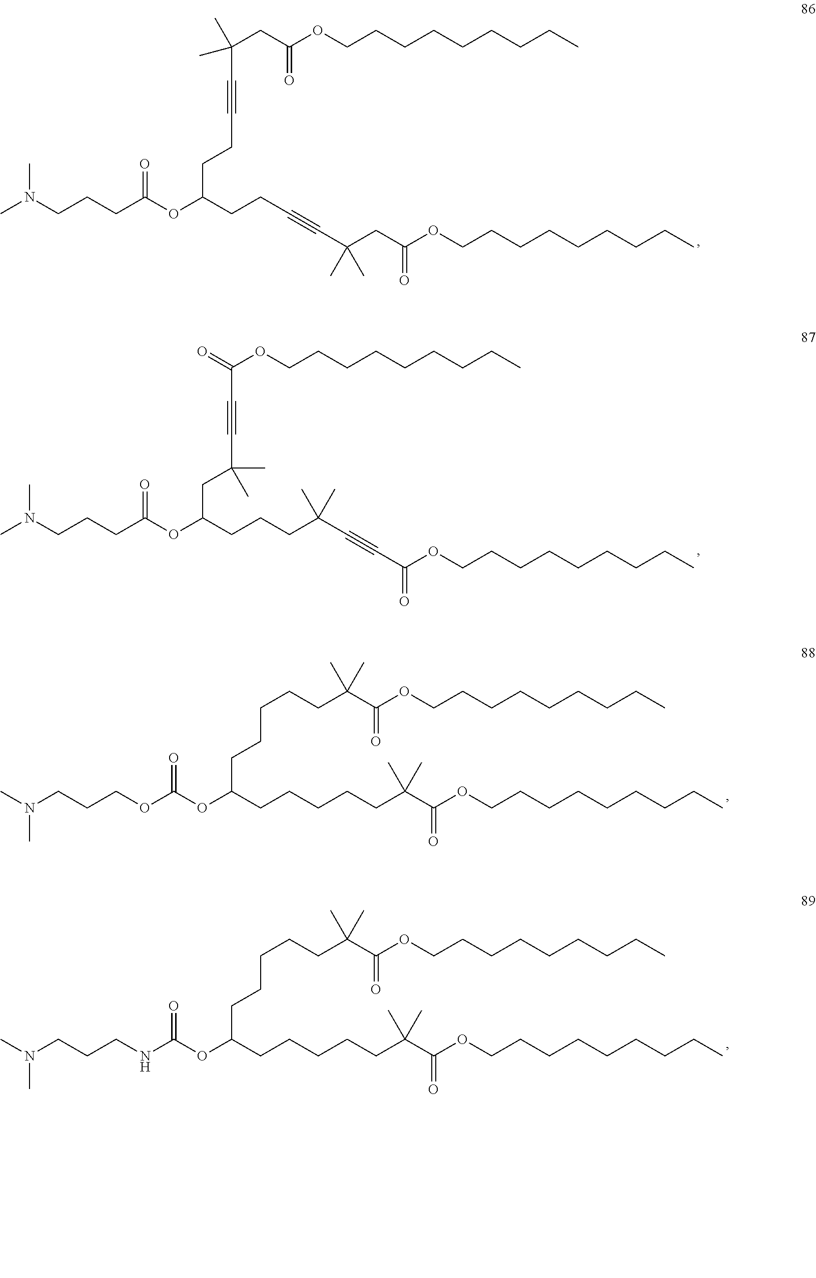

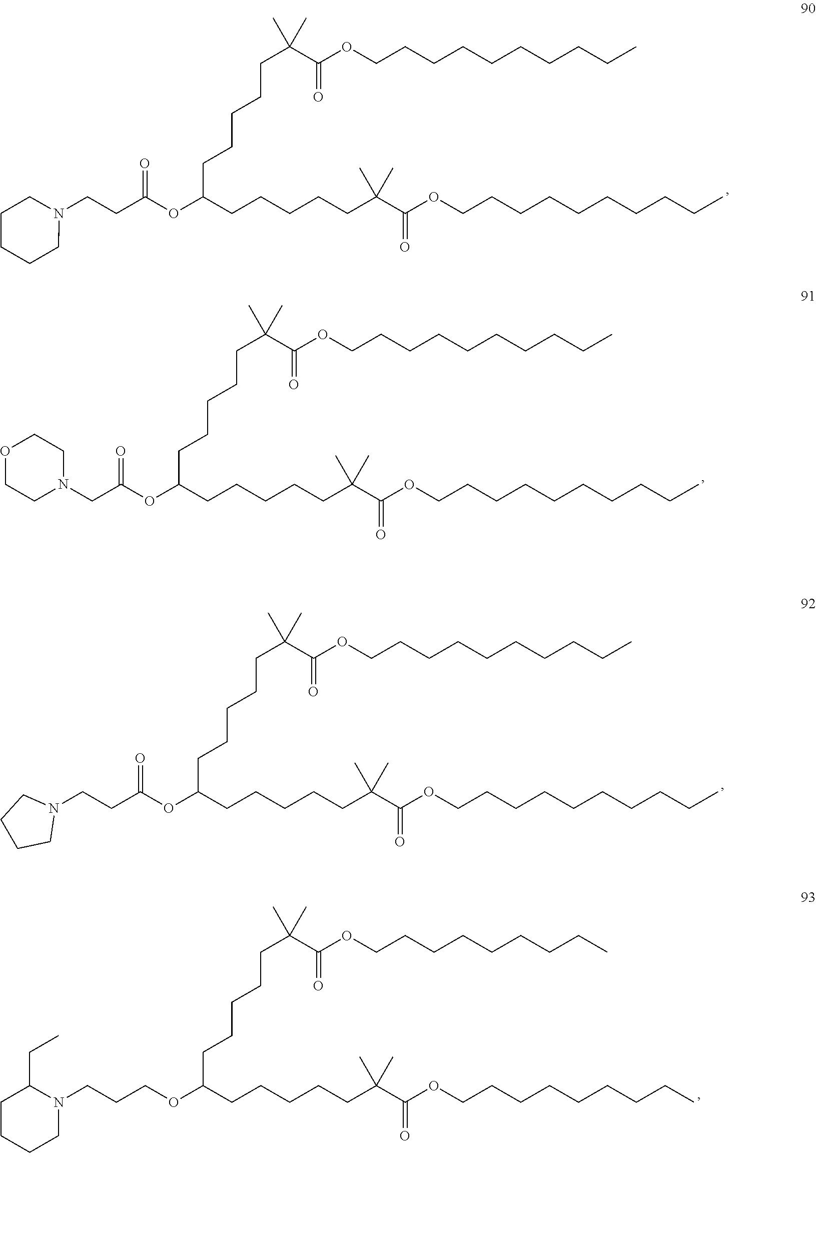 [SMILES: CCCCCCCCCCOC(=O)C(C)(C)CCCCCC(CCCCCC(C)(C)C(=O)OCCCCCCCCCC)OC(=O)CCN1CCCCC1 ; CCCCCCCCCCOC(=O)C(C)(C)CCCCCC(CCCCCC(C)(C)C(=O)OCCCCCCCCCC)OC(=O)CN1CCOCC1 ; CCCCCCCCCCOC(=O)C(C)(C)CCCCCC(CCCCCC(C)(C)C(=O)OCCCCCCCCCC)OC(=O)CCN1CCCC1 ; CCCCCCCCCOC(=O)C(C)(C)CCCCCC(CCCCCC(C)(C)C(=O)OCCCCCCCCC)OCCCN1CCCCC1CC]

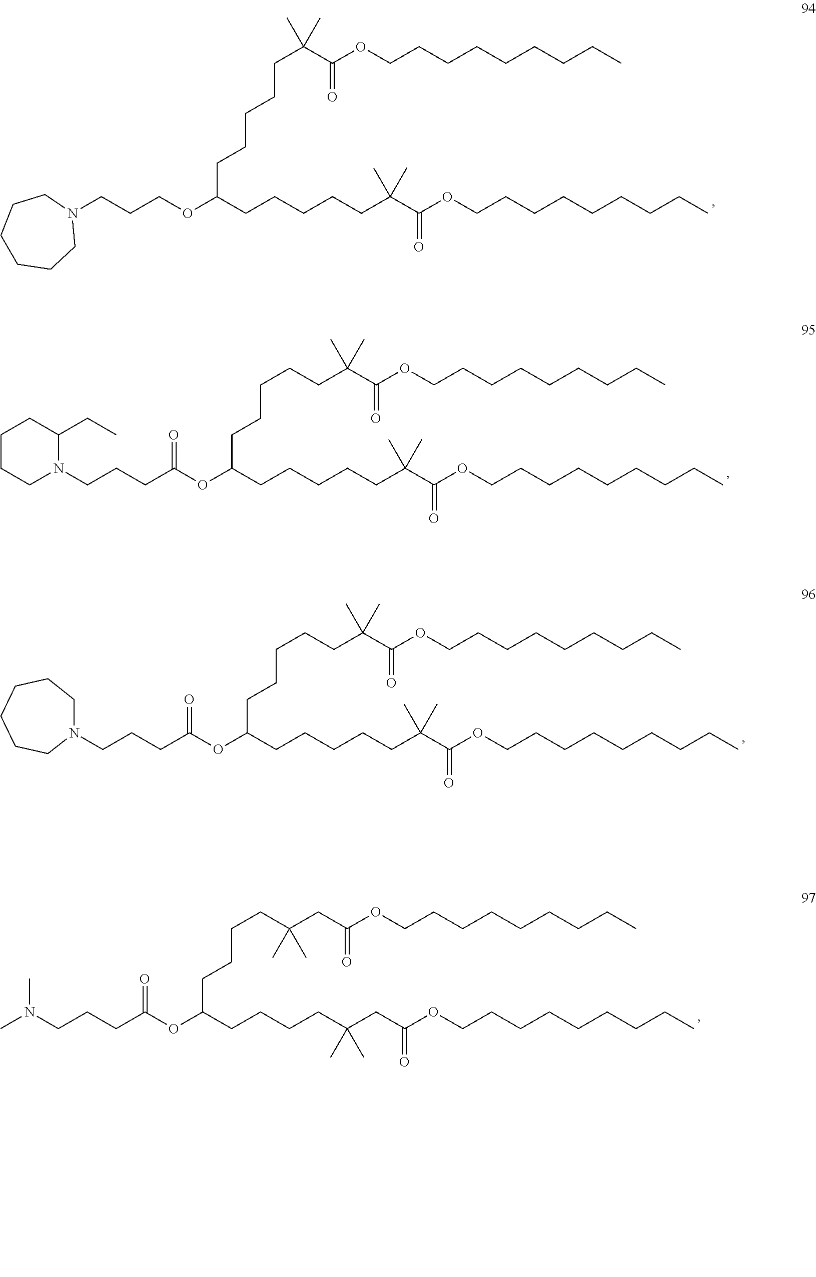

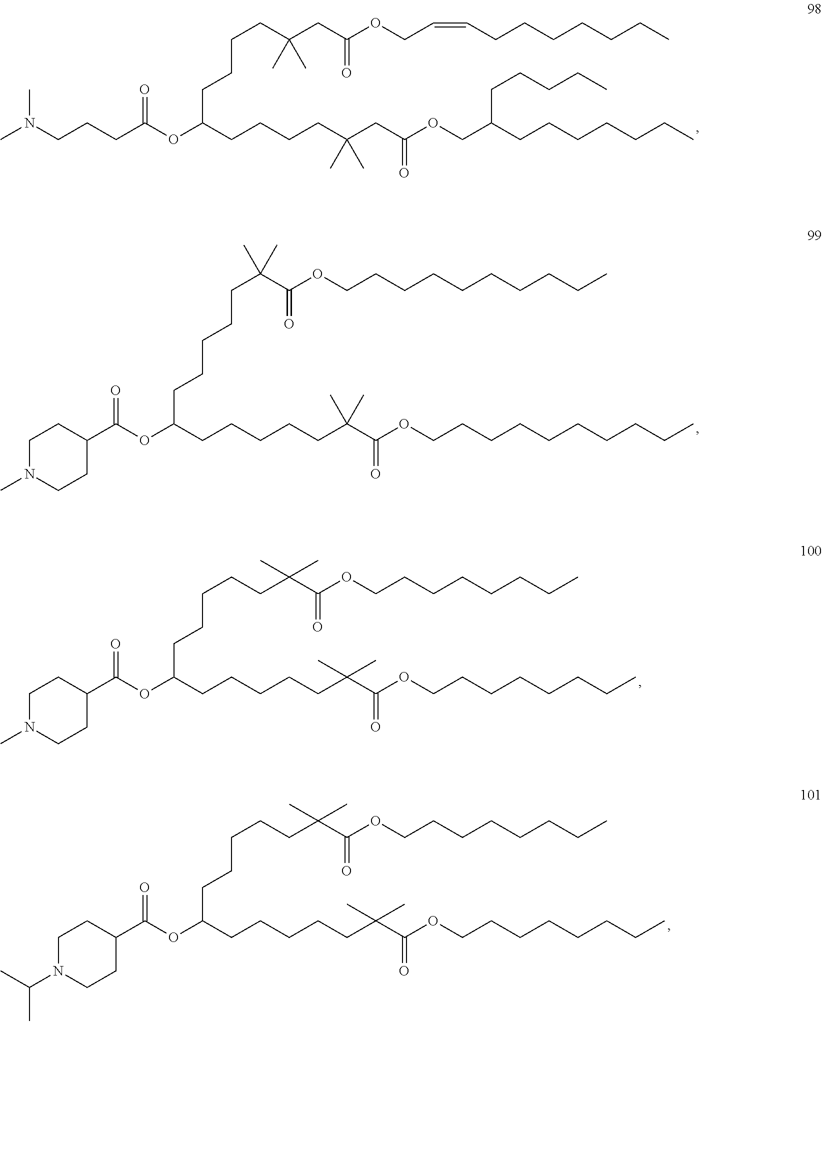

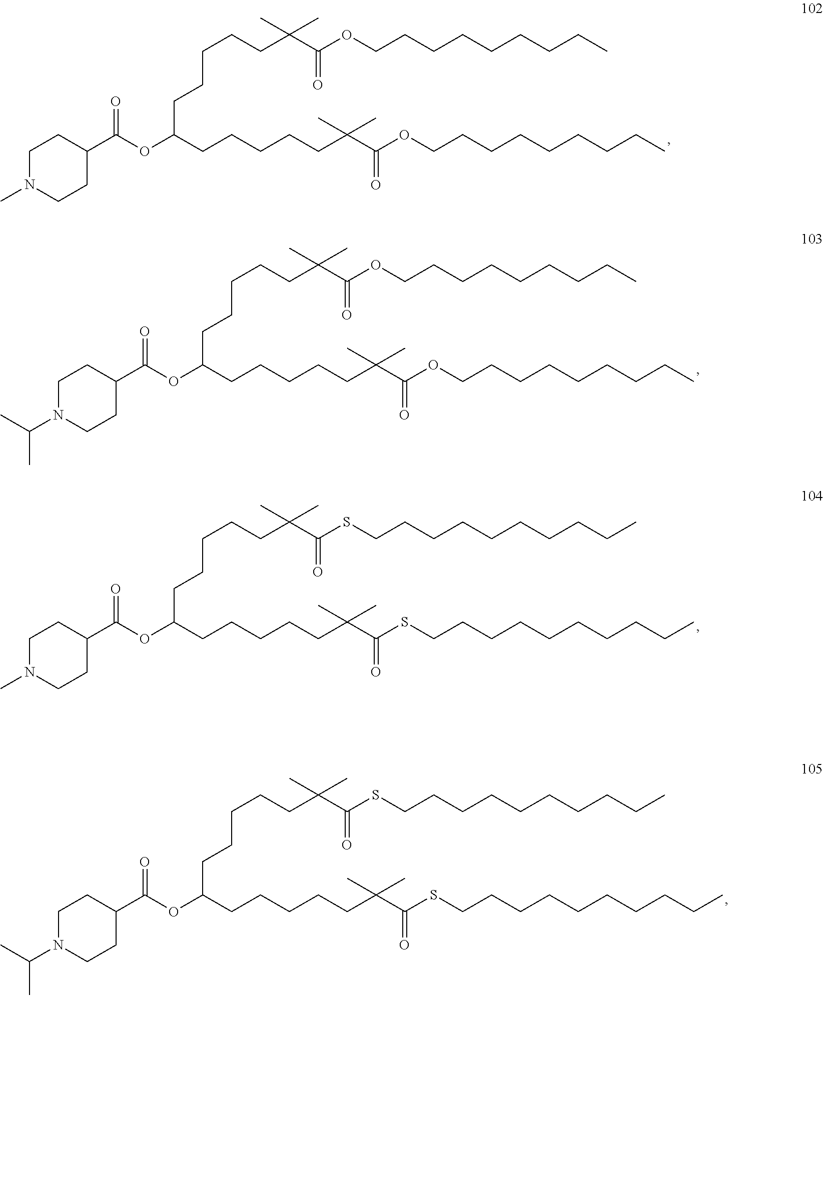

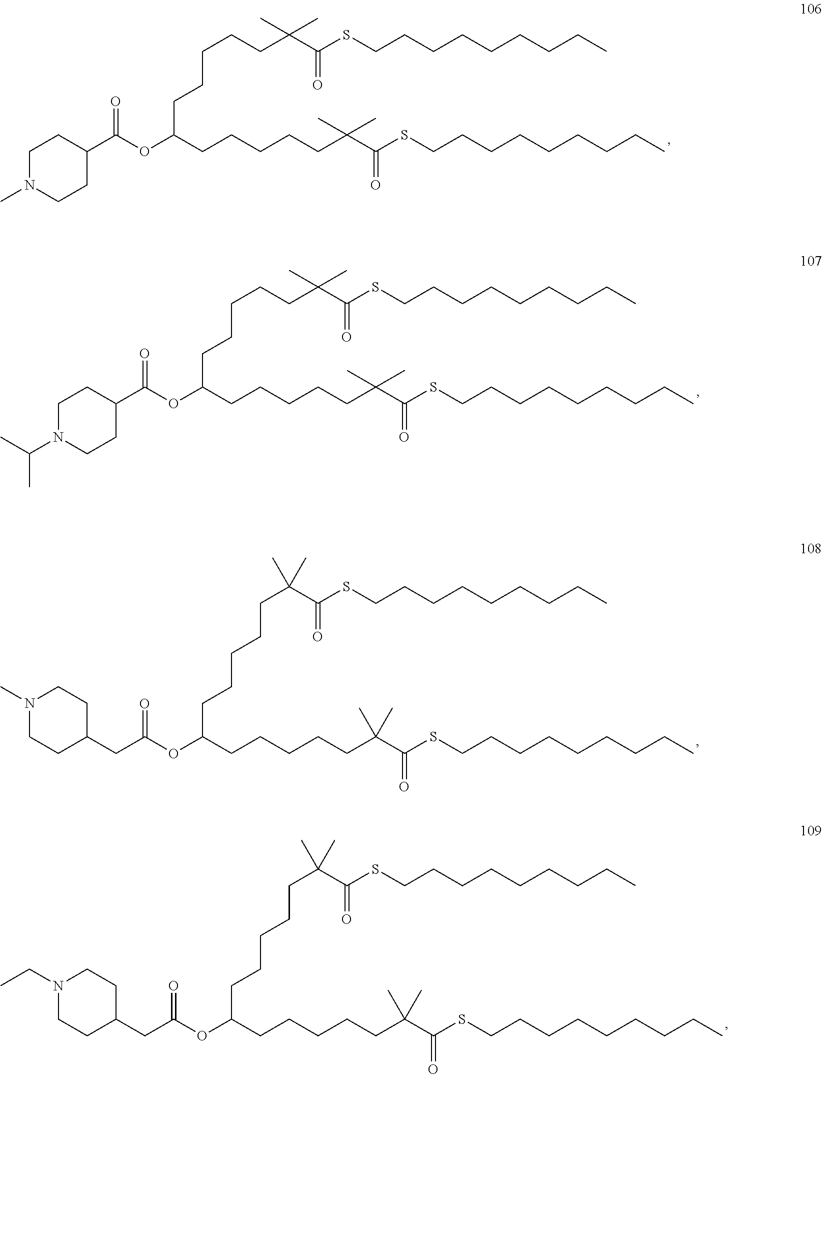

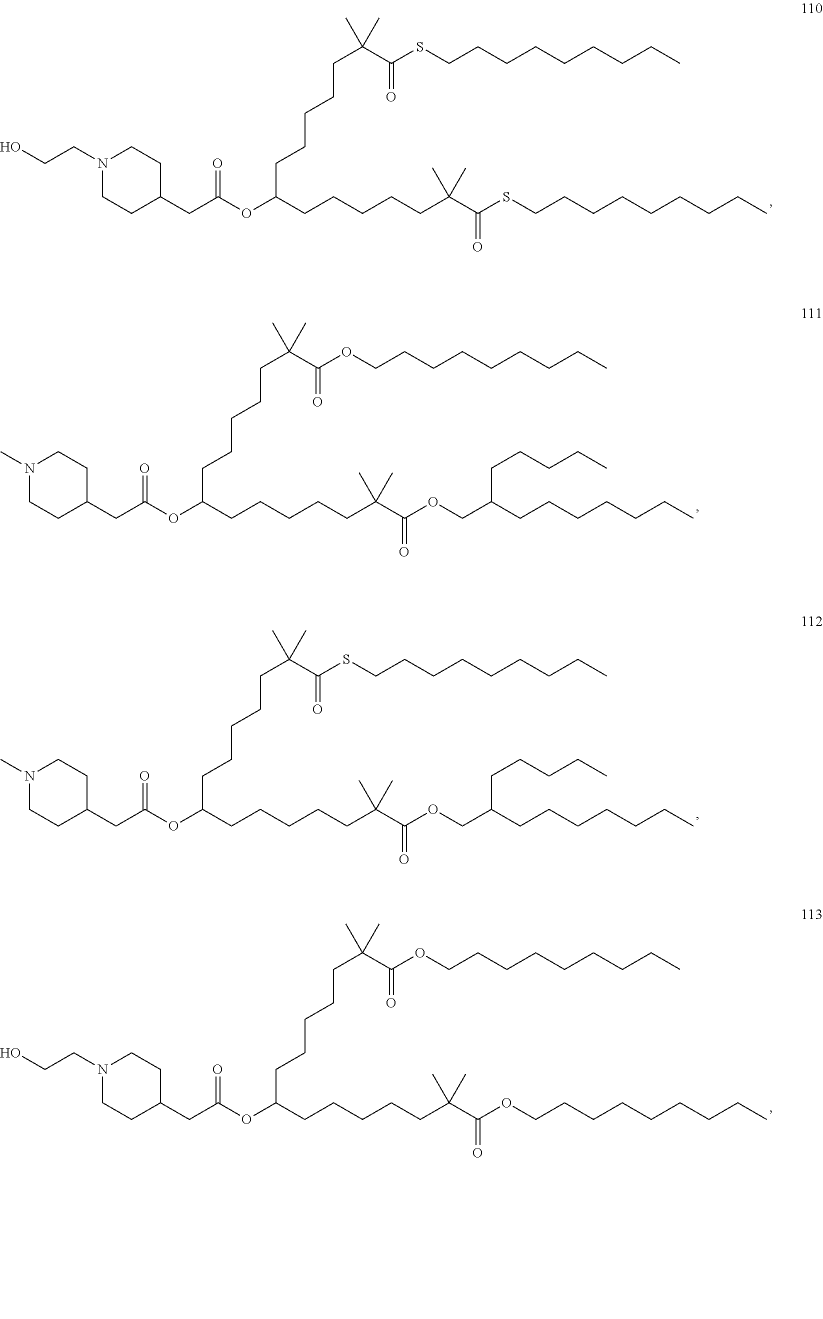

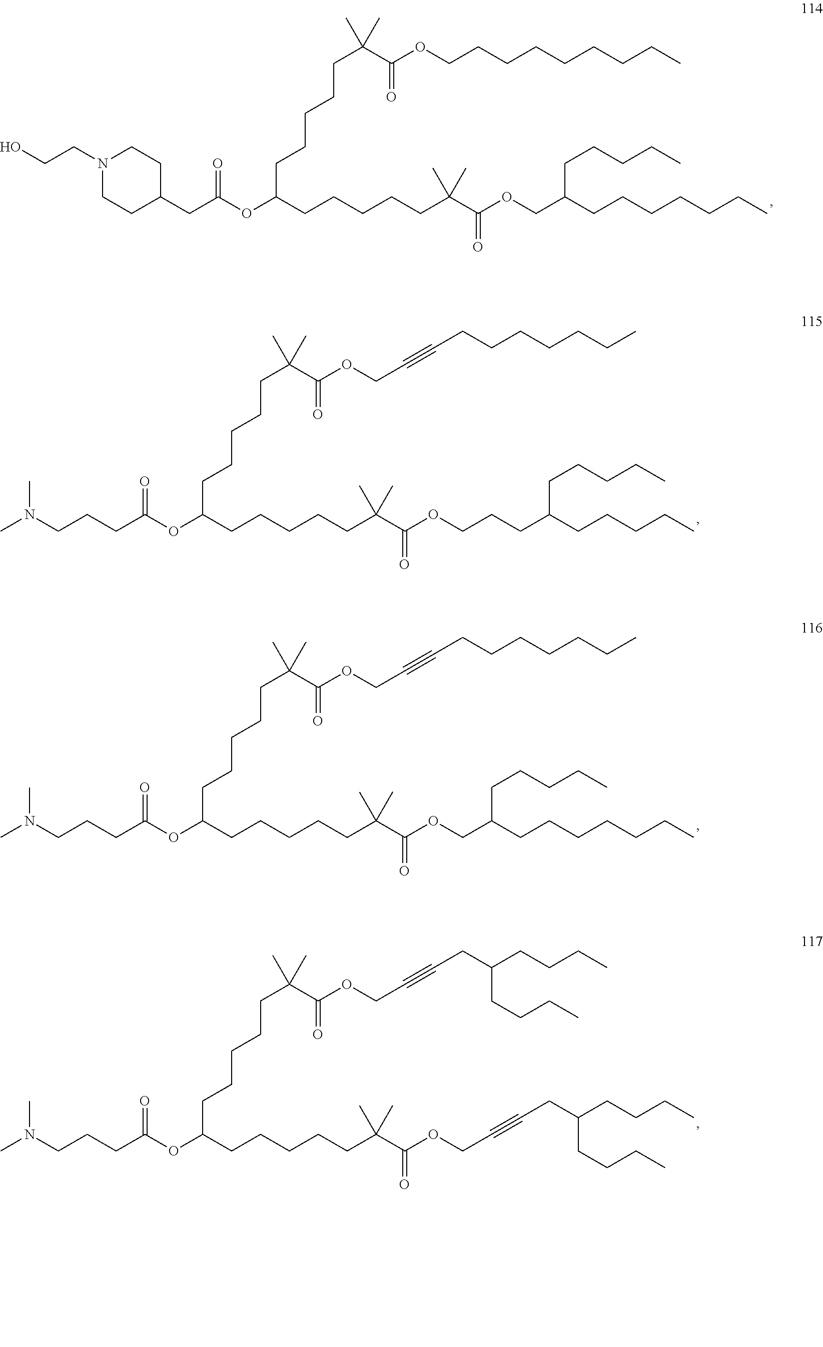

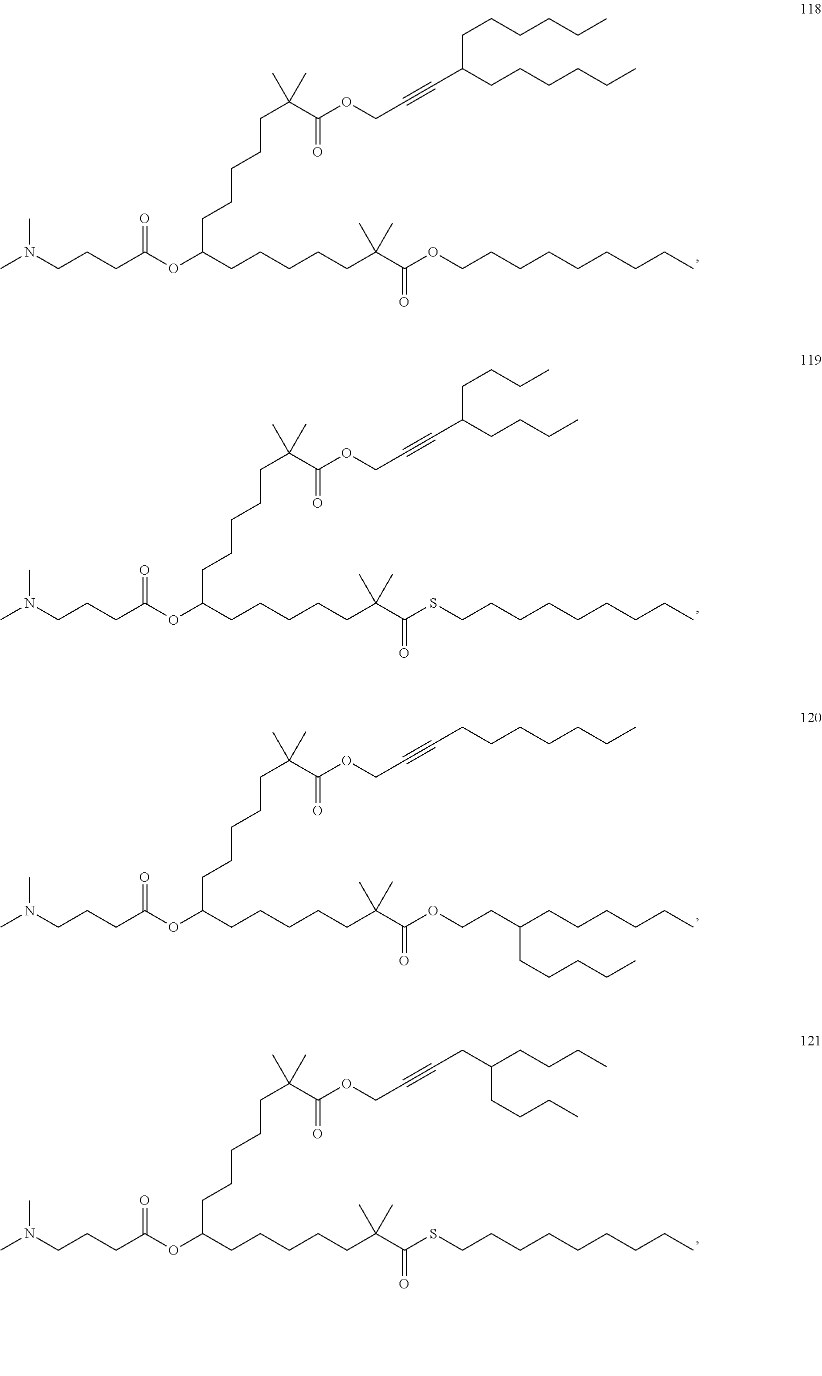

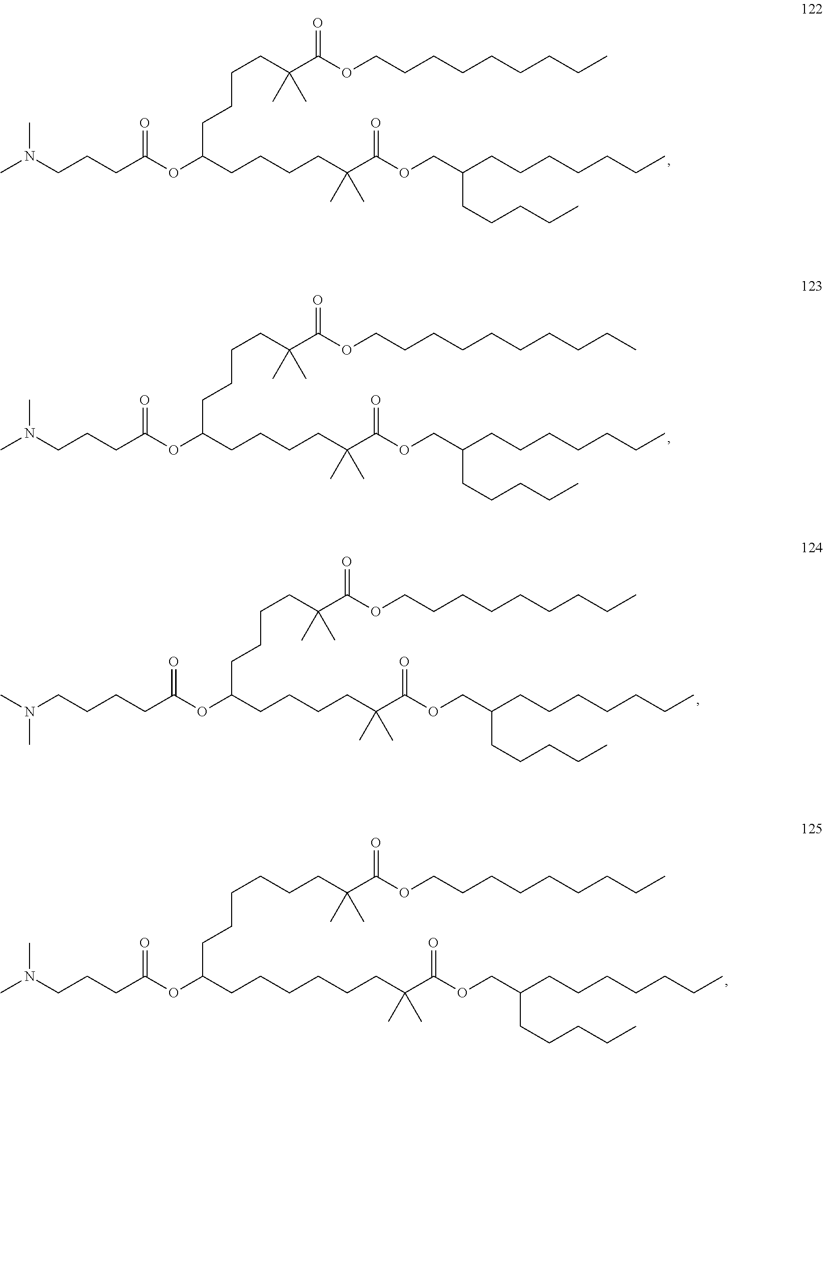

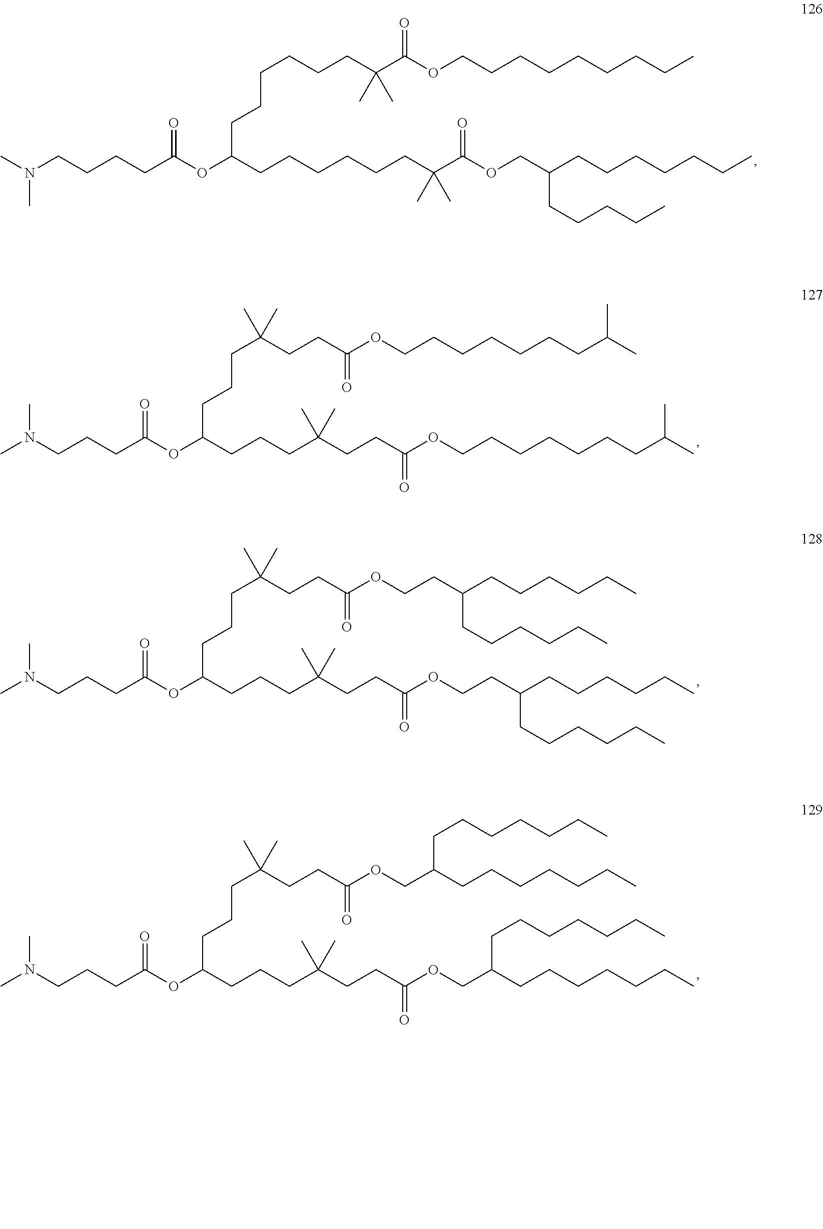

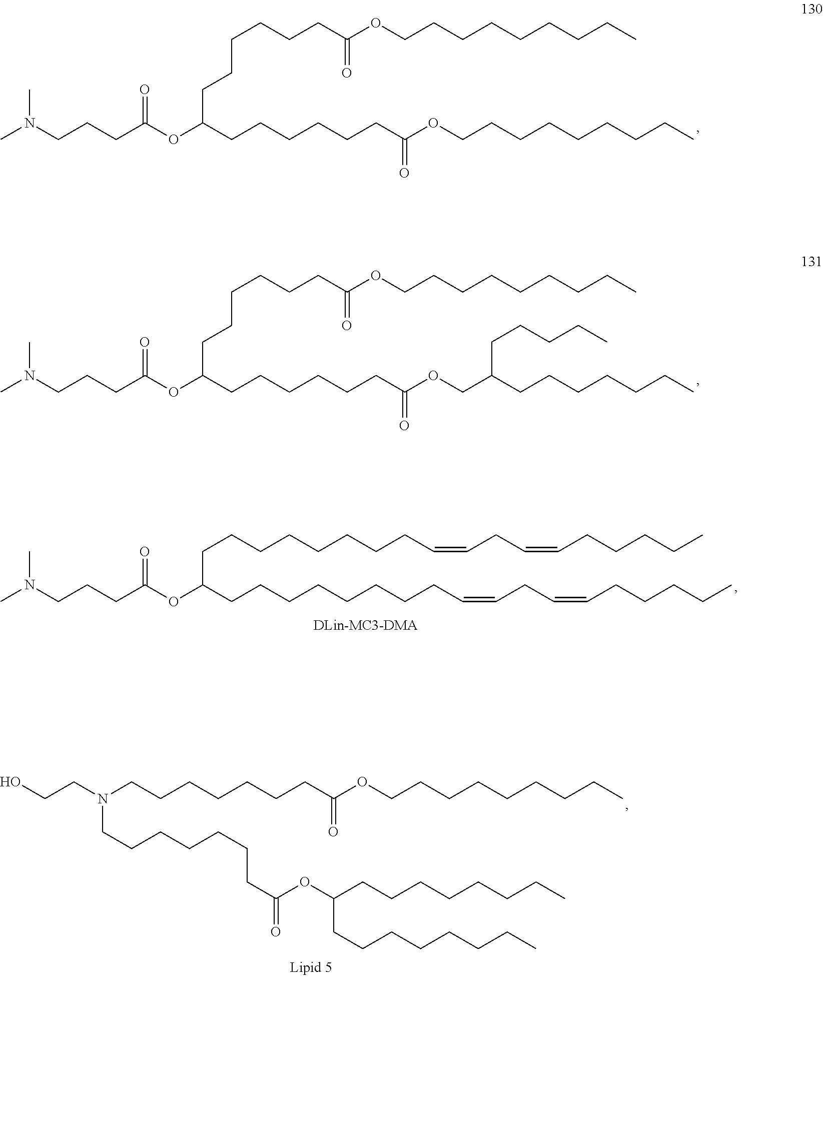

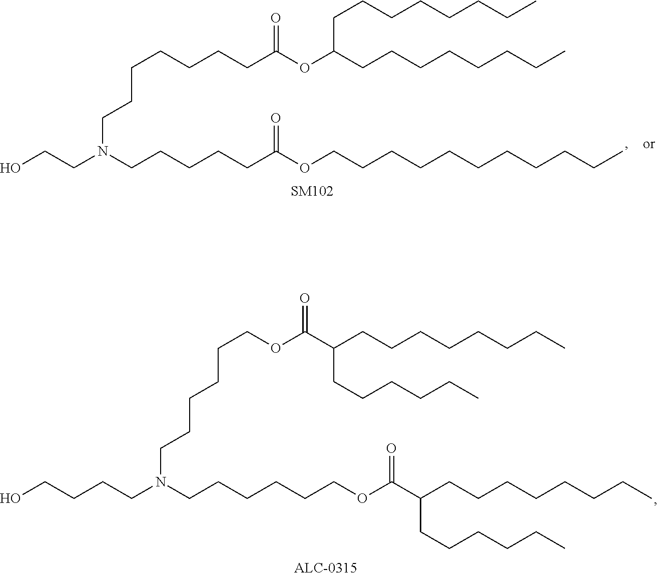 [SMILES: CCCCCCCCCCCOC(=O)CCCCCN(CCO)CCCCCCCC(=O)OC(CCCCCCCC)CCCCCCCC ; CCCCCCCCC(CCCCCC)C(=O)OCCCCCCN(CCCCO)CCCCCCOC(=O)C(CCCCCC)CCCCCCCC]

or a stereoisomer, a mixture of stereoisomers, or a pharmaceutically acceptable salt thereof.

In one embodiment, the ionizable lipid described herein is in free base form. In one embodiment, the ionizable lipid described herein is in a pharmaceutically acceptable salt form. In one embodiment, the pharmaceutically acceptable salt is a sulfate salt, a sulfite salt, a phosphate salt, a monohydrogen phosphate salt, a dihydrogen phosphates salt, a chloride salt, a bromide salt, an iodide salt, an acetate salt, an oxalate salt, an oleate salt, a palmitate salt, a stearate salt, a laurate salt, a borate salt, a benzoate salt, a lactate salt, a tosylate salt, a citrate salt, a maleate salt, a fumarate salt, a succinate salt, a tartrate salt, or a methanesulfonate salt. In one embodiment, the pharmaceutically acceptable salt is a chloride salt or a bromide salt. In one embodiment, the ionizable lipid described herein is in an ammonium chloride or an ammonium bromide form.

In one embodiment, the amount of the ionizable lipid is from about 15 mol % to about 60 mol % of the total lipid present in the lipid nanoparticle. In one embodiment, the amount of the ionizable lipid is from about 15 mol % to about 40 mol % of the total lipid present in the lipid nanoparticle. In one embodiment, the amount of the ionizable lipid is from about 20 mol % to about 30 mol % of the total lipid present in the lipid nanoparticle. In one embodiment, the amount of the ionizable lipid is about 50 mol % of the total lipid present in the lipid nanoparticle. In one embodiment, the amount of the ionizable lipid is 50 mol % of the total lipid present in the lipid nanoparticle. In one embodiment, the amount of the ionizable lipid is about 24 mol % of the total lipid present in the lipid nanoparticle. In one embodiment, the amount of the ionizable lipid is 24 mol % of the total lipid present in the lipid nanoparticle. In one embodiment, the amount of the ionizable lipid is about 29 mol %. In one embodiment, the amount of the ionizable lipid is 29 mol %. In one embodiment, the amount of the ionizable lipid is about 34 mol %. In one embodiment, the amount of the ionizable lipid is 34 mol %.

### 5.2.3. Phospholipid

In one embodiment, the lipid nanoparticle comprises a phospholipid. In one embodiment, phospholipid is distearoylphosphatidylcholine (DSPC). In one embodiment, phospholipid is dioleoylphosphatidylethanolamine (DOPE). In one embodiment, phospholipid is dimyristoylphosphatidylcholine (DMPC). In one embodiment, phospholipid is 1,2-Dioleoyl-sn-glycero-3-phosphocholine (DOPC). In one embodiment, phospholipid is dipalmitoylphosphatidylcholine (DPPC). In one embodiment, phospholipid is 1-palmitoyl-2-oleoyl-sn-glycero-3-phosphocholine (POPC). In one embodiment, phospholipid is 1,2-Dimyristoyl-sn-glycero-3-phosphoethanolamine (DMPE). In one embodiment, phospholipid is 1,2-dihexadecanoyl-sn-glycero-3-phosphoethanolamine (DPPE). In one embodiment, phospholipid is dipalmitoylphosphatidylcholine (DPPC). In one embodiment, phospholipid is hexadecanoyl-2-(9Z-Octadecenoyl)-sn-Glycero-3-Phosphoethanolamine (POPE).

In one embodiment, the amount of phospholipid is about 10 mol % of the total lipid present in the lipid nanoparticle. In one embodiment, the amount of phospholipid is 10 mol % of the total lipid present in the lipid nanoparticle. In one embodiment, the amount of phospholipid is less than 10 mol % of the total lipid present in the lipid nanoparticle. In one embodiment, the amount of phospholipid is about 5 mol % of the total lipid present in the lipid nanoparticle. In one embodiment, the amount of phospholipid is 5 mol % of the total lipid present in the lipid nanoparticle.

In one embodiment, the lipid nanoparticle is essentially free of phospholipid. In one embodiment, the lipid nanoparticle does not comprise a phospholipid. In one embodiment, the lipid nanoparticle comprises a phospholipid in an amount less than about 5 mol % of the total lipid present in the lipid nanoparticle. In one embodiment, the lipid nanoparticle comprises a phospholipid in an amount of about 4 mol % of the total lipid present in the lipid nanoparticle. In one embodiment, the lipid nanoparticle comprises a phospholipid in an amount of about 3 mol % of the total lipid present in the lipid nanoparticle. In one embodiment, the lipid nanoparticle comprises a phospholipid in an amount of about 2 mol % of the total lipid present in the lipid nanoparticle. In one embodiment, the lipid nanoparticle comprises a phospholipid in an amount of about 1 mol % of the total lipid present in the lipid nanoparticle. In one embodiment, the lipid nanoparticle comprises a phospholipid in an amount of about 0.5 mol % of the total lipid present in the lipid nanoparticle. In one embodiment, the lipid nanoparticle comprises a phospholipid in an amount of about 0 mol % of the total lipid present in the lipid nanoparticle.

### 5.2.4. Steroid

In one embodiment, the lipid nanoparticle comprises a steroid. In one embodiment, the steroid is a class of compounds with a four ring 17 carbon cyclic structure which can further comprises one or more substitutions, including alkyl groups, alkyne groups, alkynyl groups, alkoxy groups, hydroxy groups, oxo groups, acyl groups. In one embodiment, the steroid comprises three fused cyclohexyl rings and a fused cyclopentyl ring. In one embodiment, the steroid is a compound having a perhydrocyclopentanophenanthrene carbon core, which is optionally substituted. In one embodiment, the steroid is cholesterol. In one embodiment, the steroid is sitosterol or beta-sitosterol. In one embodiment, the steroid is coprosterol. In one embodiment, the steroid is fucosterol. In one embodiment, the steroid is brassicasterol. In one embodiment, the steroid is ergosterol. In one embodiment, the steroid is tomatine. In one embodiment, the steroid is ursolic acid. In one embodiment, the steroid is α-tocopherol. In one embodiment, the steroid is stigmasterol. In one embodiment, the steroid is avenasterol. In one embodiment, the steroid is ergocalciferol. In one embodiment, the steroid is campesterol. In one embodiment, the steroid is solanine. In one embodiment, the steroid is calciferol. In one embodiment, the steroid is sterol.

In one embodiment, the amount of the steroid is from about 5 mol % to about 60 mol % of the total lipid present in the lipid nanoparticle. In one embodiment, the amount of the steroid is from about 10 mol % to about 50 mol % of the total lipid present in the lipid nanoparticle. In one embodiment, the amount of the steroid is from about 10 mol % to about 40 mol % of the total lipid present in the lipid nanoparticle. In one embodiment, the amount of the steroid is from about 20 mol % to about 30 mol % of the total lipid present in the lipid nanoparticle. In one embodiment, the amount of the steroid is about 20 mol %, about 25 mol %, about 30 mol %, about 35 mol %, about 40 mol %, about 45 mol %, about 50 mol %, or about 55 mol % of the total lipid present in the lipid nanoparticle. In one embodiment, the amount of the steroid is about 38.5 mol % of the total lipid present in the lipid nanoparticle. In one embodiment, the amount of the steroid is 38.5 mol % of the total lipid present in the lipid nanoparticle. In one embodiment, the amount of the steroid is about 25 mol % of the total lipid present in the lipid nanoparticle. In one embodiment, the amount of the steroid is 25 mol % of the total lipid present in the lipid nanoparticle.

### 5.2.5. Pegylated Lipid

In one embodiment, the lipid nanoparticle comprises a pegylated lipid (PEG lipid). In one embodiment, the PEG lipid is a diglyceride which also comprises a PEG chain attached to the glycerol group. In one embodiment, the PEG lipid is a compound which contains one or more C₆-C₂₄ long chain alkyl or alkenyl group or a C₆-C₂₄ fatty acid group attached to a linker group with a PEG chain. Non-limiting examples of a PEG lipid includes a PEG modified phosphatidylethanolamine and phosphatidic acid, a PEG conjugated ceramide, PEG modified dialkylamines and PEG modified 1,2-diacryloxy propan-3-amines, PEG modified diacylglycerols and dialkylglycerols. In one embodiment, PEG modified diastearoylphosphatidylethanolamine or PEG modified dimyristoyl-sn-glycerol. In one embodiment, the PEG modification is measured by the molecular weight of PEG component of the lipid. In one embodiment, the pegylated lipid has a molecule weight of from about 1000 Da to about 10,000 Da. In one embodiment, In one embodiment, the pegylated lipid has a molecule weight of from about 1000 Da to about 5000 Da. In one embodiment, the pegylated lipid has a molecule weight of from about 1000 Da to about 2000 Da.

In one embodiment, the pegylated lipid is methoxypolyethyleneglycoloxy(2000)-N,N-ditetradecylacetamide (ALC-0159). In one embodiment, the pegylated lipid is 1,2-dimyristoyl-rac-glycero-3-methoxypolyethylene glycol-2000 (DMG-PEG2000). In one embodiment, the pegylated lipid is 1,2-Dimyristoyl-sn-glycero-3-phosphoethanolamine-polyethylene glycol 1000 (DMPE-PEG1000). In one embodiment, the pegylated lipid is 1,2-dipalmitoyl-sn-glycero-3-phosphoethanolamine-polyethylene glycol 1000 (DPPE-PEG1000). In one embodiment, the pegylated lipid is 1,2-distearoyl-sn-glycero-3-phosphoethanolamine-polyethylene glycol 1000 (DSPE-PEG1000). In one embodiment, the pegylated lipid is 1,2-dioleoyl-sn-glycero-3-phosphoethanolamine-polyethylene glycol 1000 (DOPE-PEG1000). In one embodiment, the pegylated lipid is 1,2-dimyristoyl-rac-glycero-3-methoxypolyethylene glycol-2000 (Ceramide-PEG2000). In one embodiment, the pegylated lipid is 1,2-Dimyristoyl-sn-glycero-3-phosphoethanolamine-polyethylene glycol 2000 (DMPE-PEG2000). In one embodiment, the pegylated lipid is 1,2-dipalmitoyl-sn-glycero-3-phosphoethanolamine-polyethylene glycol 2000 (DPPE-PEG2000). In one embodiment, the pegylated lipid is 1,2-distearoyl-sn-glycero-3-phosphoethanolamine-polyethylene glycol 2000 (DSPE-PEG2000). In one embodiment, the pegylated lipid is 1,2-distearoyl-sn-glycero-3-phosphoethanolamine-N-(polyethylene glycol 2000)-Mannose (DSPE-PEG2000-Mannose). In one embodiment, the pegylated lipid is 1,2-dimyristoyl-rac-glycero-3-methoxypolyethylene glycol-5000 (Ceramide-PEG5000). In one embodiment, the pegylated lipid is 1,2-distearoyl-sn-glycero-3-phosphoethanolamine-polyethylene glycol 5000 (DSPE-PEG5000). In one embodiment, the pegylated lipid is 1,2-distearoyl-sn-glycero-3-phosphoethanolamine-N-[amino(polyethylene glycol)-2000 (DSPE-PEG2000 amine).

In one embodiment, the amount of the pegylated lipid is from about 0.05 mol % to about 5 mol % of the total lipid present in the lipid nanoparticle. In one embodiment, the amount of the pegylated lipid is from about 0.1 mol % to about 3 mol % of the total lipid present in the lipid nanoparticle. In one embodiment, the amount of the pegylated lipid is from about 0.25 mol % to about 2 mol % of the total lipid present in the lipid nanoparticle. In one embodiment, the amount of the pegylated lipid is from about 0.5 mol % to about 1.5 mol % of the total lipid present in the lipid nanoparticle. In one embodiment, the amount of the pegylated lipid is about 0.1 mol %, about 0.5 mol %, about 1 mol %, about 1.5 mol %, about 2 mol %, about 2.5 mol %, or about 3 mol % of the total lipid present in the lipid nanoparticle. In one embodiment, the amount of the pegylated lipid is about 1.5 mol % of the total lipid present in the lipid nanoparticle. In one embodiment, the amount of the pegylated lipid is 1.5 mol % of the total lipid present in the lipid nanoparticle. In one embodiment, the amount of the pegylated lipid is about 1 mol % of the total lipid present in the lipid nanoparticle. In one embodiment, the amount of the pegylated lipid is 1 mol % of the total lipid present in the lipid nanoparticle. In one embodiment, the amount of the pegylated lipid is about 2 mol % of the total lipid present in the lipid nanoparticle. In one embodiment, the amount of the pegylated lipid is 2 mol % of the total lipid present in the lipid nanoparticle.

### 5.2.6. Therapeutic Agent

In one embodiment, the lipid nanoparticle comprises a therapeutic agent.

In one embodiment, the therapeutic agent is a small molecule.

In one embodiment, the therapeutic agent is an anticancer agent, antifungal agent, psychiatric agent such as analgesics, consciousness level-altering agent such as anesthetic agent or hypnotics, nonsteroidal anti-inflammatory drugs (NSAIDS), anthelminthics, antiacne agent, antianginal agent, antiarrhythmic agent, anti-asthma agent, antibacterial agent, anti-benign prostate hypertrophy agent, anticoagulant, antidepressant, antidiabetic, antiemetic, antiepileptic, antigout agent, antihypertensive agent, anti-inflammatory agent, antimalarial, antimigraine agent, antimuscarinic agent, antineoplastic agent, antiobesity agent, antiosteoporosis agent, antiparkinsonian agent, antiproliferative agent, antiprotozoal agent, antithyroid agent, antitussive agent, anti-urinary incontinence agent, antiviral agent, anxiolytic agent, appetite suppressant, beta-blockers, cardiac inotropic agent, chemotherapeutic drug, cognition enhancer, contraceptive, corticosteroid, Cox-2 inhibitor, diuretic, erectile dysfunction improvement agent, expectorant, gastrointestinal agent, histamine receptor antagonists, immunosuppressants, keratolytic, lipid regulating agent, leukotriene inhibitor, macrolide, muscle relaxant, neuroleptic, nutritional agent, opioid analgesics, protease inhibitor, or sedative, or a combination thereof.

In one embodiment, the therapeutic agent is a protein. In one embodiment, the therapeutic agent is a peptide. In one embodiment, the therapeutic agent is an antibody. In one embodiment, the therapeutic agent is a monoclonal antibody. In one embodiment, the therapeutic agent is a bispecific antibody.

In one embodiment, the therapeutic agent is antisense oligonucleotide (ASO). In one embodiment, the ASO comprises a naturally-occurring nucleoside. In one embodiment, the ASO comprises a modified nucleoside. In one embodiment, the ASO is capable of modulating expression of a target gene by hybridizing to a target nucleic acid, particularly a contiguous sequence on a target nucleic acid. In one embodiment, the ASO is singled stranded.

In one embodiment, the therapeutic agent is deoxyribonucleic acid (DNA). In one embodiment, the therapeutic agent is plasmid DNA (pDNA). In one embodiment, the therapeutic agent is double stranded DNA (dsDNA). In one embodiment, the therapeutic agent is single stranded DNA (ssDNA).

In one embodiment, the therapeutic agent is ribonucleic acid (RNA). In one embodiment, the therapeutic agent is RNA interference (RNAi). In one embodiment, the therapeutic agent is small interfering RNA (siRNA). In one embodiment, the therapeutic agent is short hairpin RNA (shRNA). In one embodiment, the therapeutic agent is antisense RNA (aRNA). In one embodiment, the therapeutic agent is messenger RNA (mRNA). In one embodiment, the therapeutic agent is modified messenger RNA (mmRNA). In one embodiment, the therapeutic agent is long noncoding RNA (lncRNA). In one embodiment, the therapeutic agent is microRNA (miRNA). In one embodiment, the therapeutic agent is small activating RNA (saRNA). In one embodiment, the therapeutic agent is multicoding nucleic acid (MCNA). In one embodiment, the therapeutic agent is polymer-coded nucleic acid (PCNA). In one embodiment, the therapeutic agent is any RNA in the ribozyme.

In one embodiment, the therapeutic agent is a clustered regularly interspaced short palindromic repeats (CRISPR) related nucleic acid. In one embodiment, the therapeutic agent is guide RNA (gRNA). In one embodiment, the therapeutic agent is CRISPR RNA (crRNA). In one embodiment, the therapeutic agent comprises a first nucleic acid and a second nucleic acid. In one embodiment, the first nucleic acid is a messenger RNA. In one embodiment, the second nucleic acid is a single guide RNA. In one embodiment, the first nucleic acid is a messenger RNA (mRNA) and the second nucleic acid is a single guide RNA (sgRNA).

In one embodiment, the ratio of (total number of nitrogen atoms in the permanently cationic lipid and ionizable lipid) and (total number of phosphorus atoms in the nucleic acid) is from about 1:1 to about 15:1 (N:P ratio). In one embodiment, the N:P ratio is from about 3:1 to about 12:1. In one embodiment, the N:P ratio is from about 4:1 to about 9:1. In one embodiment, the N:P ratio is about 4:1, about 5:1, about 6:1, about 7:1, about 8:1, or about 9:1. In one embodiment, the N:P ratio is about 6:1. In one embodiment, the N:P ratio is 6:1. In one embodiment, the N:P ratio is about 8:1. In one embodiment, the N:P ratio is 8:1.

In one embodiment, the amount of the therapeutic agent delivered or expressed in a non-hepatic tissued of a subject by the lipid nanoparticle is higher than the amount of the therapeutic agent delivered or expressed in the liver of the subject by the lipid nanoparticle, when the lipid nanoparticle is administered to the subject. In one embodiment, the amount of the therapeutic agent delivered or expressed in the lung of a subject by the lipid nanoparticle is higher than the amount of the therapeutic agent delivered or expressed in the liver of the subject by the lipid nanoparticle, when the lipid nanoparticle is administered to the subject.

In one embodiment, the amount of the therapeutic agent delivered or expressed in the lung of the subject is higher than the amount of the therapeutic agent delivered or expressed in the liver of the subject. In one embodiment, the amount of the therapeutic agent delivered or expressed in the lung of the subject is at least one time higher than the amount of the therapeutic agent delivered or expressed in the liver of the subject. In one embodiment, the amount of the therapeutic agent delivered or expressed in the lung of the subject is at least 2 times higher than the amount of the therapeutic agent delivered or expressed in the liver of the subject. In one embodiment, the amount of the therapeutic agent delivered or expressed in the lung of the subject is at least 3 times higher than the amount of the therapeutic agent delivered or expressed in the liver of the subject. In one embodiment, the amount of the therapeutic agent delivered or expressed in the lung of the subject is at least 4 times higher than the amount of the therapeutic agent delivered or expressed in the liver of the subject. In one embodiment, the amount of the therapeutic agent delivered or expressed in the lung of the subject is at least 5 times higher than the amount of the therapeutic agent delivered or expressed in the liver of the subject. In one embodiment, the amount of the therapeutic agent delivered or expressed in the lung of the subject is at least 10 times higher than the amount of the therapeutic agent delivered or expressed in the liver of the subject. In one embodiment, the amount of the therapeutic agent delivered or expressed in the lung of the subject is at least 20 times higher than the amount of the therapeutic agent delivered or expressed in the liver of the subject. In one embodiment, the amount of the therapeutic agent delivered or expressed in the lung of the subject is at least 40 times higher than the amount of the therapeutic agent delivered or expressed in the liver of the subject. In one embodiment, the amount of the therapeutic agent delivered or expressed in the lung of the subject is at least 60 times higher than the amount of the therapeutic agent delivered or expressed in the liver of the subject. In one embodiment, the amount of the therapeutic agent delivered or expressed in the lung of the subject is at least 100 times higher than the amount of the therapeutic agent delivered or expressed in the liver of the subject.

In one embodiment, the amount of protein expressed by a nucleic acid in the lung of a subject is higher than the amount of the protein expressed by the nucleic acid in the liver of the subject, when a lipid nanoparticle comprising the nucleic acid is administered to the subject. In one embodiment, the amount of protein expressed by an mRNA in the lung of a subject is higher than the amount of the protein expressed by the mRNA in the liver of the subject, when a lipid nanoparticle comprising the mRNA is administered to the subject.

In one embodiment, the amount of protein expressed in the lung of the subject is higher than the amount of protein expressed in the liver of the subject. In one embodiment, the amount of protein expressed in the lung of the subject is at least one time higher than the amount of protein expressed in the liver of the subject. In one embodiment, the amount of protein expressed in the lung of the subject is at least 2 times higher than the amount of protein expressed in the liver of the subject. In one embodiment, the amount of protein expressed in the lung of the subject is at least 3 times higher than the amount of protein expressed in the liver of the subject. In one embodiment, the amount of protein expressed in the lung of the subject is at least 4 times higher than the amount of protein expressed in the liver of the subject. In one embodiment, the amount of protein expressed in the lung of the subject is at least 5 times higher than the amount of protein expressed in the liver of the subject. In one embodiment, the amount of protein expressed in the lung of the subject is at least 10 times higher than the amount of protein expressed in the liver of the subject. In one embodiment, the amount of protein expressed in the lung of the subject is at least 20 times higher than the amount of protein expressed in the liver of the subject. In one embodiment, the amount of protein expressed in the lung of the subject is at least 40 times higher than the amount of protein expressed in the liver of the subject. In one embodiment, the amount of protein expressed in the lung of the subject is at least 60 times higher than the amount of protein expressed in the liver of the subject. In one embodiment, the amount of protein expressed in the lung of the subject is at least 100 times higher than the amount of protein expressed in the liver of the subject.

### 5.3 A Population of Lipid Nanoparticles

In one embodiment, provided herein is a population of lipid nanoparticles comprising the lipid nanoparticle described in Section 5.2. In one embodiment, the population of lipid nanoparticles described herein comprises the permanently cationic lipid described in Section 5.2.1. In one embodiment, the population of lipid nanoparticles described herein comprises the ionizable lipid described in Section 5.2.2. In one embodiment, the population of lipid nanoparticles described herein comprises the phospholipid described in Section 5.2.3. In one embodiment, the population of lipid nanoparticles described herein does not comprises phospholipid. In one embodiment, the population of lipid nanoparticles described herein comprises the steroid described in Section 5.2.4. In one embodiment, the population of lipid nanoparticles described herein comprises the pegylated lipid described in Section 5.2.5. In one embodiment, the population of lipid nanoparticles described herein comprises the therapeutic agent described in Section 5.2.6.

In one embodiment, the population of lipid nanoparticle have an average diameter of from about 160 nm to about 900 nm. In one embodiment, the population of lipid nanoparticle have an average diameter of from about 180 nm to about 900 nm. In one embodiment, the population of lipid nanoparticle have an average diameter of from about 300 nm to about 900 nm. In one embodiment, the population of lipid nanoparticle have an average diameter of from about 160 nm to about 600 nm. In one embodiment, the population of lipid nanoparticle have an average diameter of from about 160 nm to about 400 nm. In one embodiment, the population of lipid nanoparticle have an average diameter of from about 160 nm to about 350 nm. In one embodiment, the population of lipid nanoparticle have an average diameter of from about 160 nm to about 300 nm. In one embodiment, the population of lipid nanoparticle have an average diameter of from about 300 nm to about 400 nm. In one embodiment, the population of lipid nanoparticle have an average diameter of from about 180 nm, about 200 nm, about 220 nm, about 230 nm, about 240 nm, about 250 nm, about 260 nm, about 280 nm, about 300 nm, about 320 nm, about 340 nm, about 360 nm, about 380 nm, about 400 nm, about 450 nm, about 500 nm, about 600 nm, about 700 nm, about 800 nm, or about 900 nm. In one embodiment, the average diameter is determined Dynamic Light Scattering (DLS) described in Section 5.6.

In one embodiment, the population of lipid nanoparticle have a polydispersity index (PDI) of less than 0.2. In one embodiment, the population of lipid nanoparticle have a PDI of less than 0.1. In one embodiment, the population of lipid nanoparticle have a PDI of about 0.01, about 0.02, about 0.03, about 0.04, about 0.05, about 0.06, about 0.07, about 0.08, about 0.09, or about 0.10. In one embodiment, the PDI is determined by Dynamic Light Scattering (DLS) described in Section 5.6.

In one embodiment, the population of lipid nanoparticle have an apparent acid dissociation constant (pKa) of greater than 7. In one embodiment, the population of lipid nanoparticle have an apparent pKa of greater than 8. In one embodiment, the population of lipid nanoparticle have an apparent pKa of greater than 9. In one embodiment, the population of lipid nanoparticle have an apparent pKa of from about 7 to about 10. In one embodiment, the population of lipid nanoparticle have an apparent pKa of about 7, about 7.5, about 8, about 8.5, about 9, about 9.5, or about 10.

In one embodiment, the population of lipid nanoparticle have an apparent pKa between 6 and 7. In one embodiment, the population of lipid nanoparticle have an apparent pKa of about 6.1, about 6.2, about 6.3, about 6.4, about 6.5, about 6.6, about 6.7, about 6.8, about 6.9, about 7. In one embodiment, the population of lipid nanoparticle have an apparent pKa of less than 6. In one embodiment, the population of lipid nanoparticle have an apparent pKa of between 4 to 6.

In one embodiment, the apparent pKa is determined by 2-(p-toluidino)-6-naphthalene sulfonic acid (TNS) fluorescent methods. The TNS fluorescent method has been described in the art, such as Jayaraman M, et al., Maximizing the potency of siRNA lipid nanoparticles for hepatic gene silencing in vivo. Angew Chem Int Ed, 2012. 51, 8529-8533, which is incorporated herein by reference.

In one embodiment, the population of lipid nanoparticle has a positive surface charge. In one embodiment, the population of lipid nanoparticle has a positive surface charge at physiologic pH. In one embodiment, the population of lipid nanoparticle has a positive surface charge in vivo. In one embodiment, the population of lipid nanoparticle have a greater than neutral zeta potential at physiologic pH. In one embodiment, the zeta potential is from about 0 mV to about 50 mV. In one embodiment, the zeta potential is from about 0 mV to about 25 mV. In one embodiment, the zeta potential is from about 0 mV to about 20 mV, In one embodiment, the zeta potential from about 2 mV to about 15 mV. In one embodiment, the zeta potential is about 1 mV, about 5 mV, about 10 mV, about 15 mV, about 20 mV, about 25 mV, about 30 mV, about 35 mV, about 40 mV, or about 50 mV. Methods to measure zeta potential have been described (e.g. Clogston et al, Zeta potential measurement, Methods Mol. Biol., 2011:697:63-70). In one embodiment, the zeta potential is determined using Zetasizer Pro (From Malvern Instruments, Ltd). In one embodiment, zeta potential of LNPs is measured under the following conditions: LNPs were diluted to 1.0 ng/μL total mRNA in PBS buffer (pH=7.4) and loaded into a Disposable Folded Capillary Cell (DTS1070). The sample was equilibrated for 120 seconds, repeated for 3 times with 20 seconds between measurements.

### 5.4 Pharmaceutical Compositions

In one embodiment, provided herein are pharmaceutical compositions comprising the lipid nanoparticle described in Section 5.2 or the population of lipid nanoparticle described in Section 5.3. In one embodiment, the pharmaceutical composition further comprises a pharmaceutically acceptable carrier.

A pharmaceutically acceptable carrier for use in the present application includes a non-toxic carrier, adjuvant or vehicle which does not destroy the pharmacological activity of the compound formulated together. Pharmaceutically acceptable carriers that may be used in the compositions of the present disclosure include, but are not limited to, ion exchangers, alumina, aluminum stearate, lecithin, serum proteins (e.g., human serum albumin), buffer substances (such as phosphate), glycine, sorbic acid, potassium sorbate, a mixture of partial glycerides of saturated plant fatty acids, water, salt or electrolyte (such as protamine sulfate), disodium hydrogen phosphate, potassium hydrogen phosphate, sodium chloride, zinc salt, silica gel, magnesium trisilicate, polyvinyl pyrrolidone, cellulose-based materials, polyethylene glycol, sodium carboxymethyl cellulose, polyacrylate, wax, polyethylene-polyoxypropylene block polymers, polyethylene glycol and lanolin.

In one embodiment, the pharmaceutical compositions are formulated for oral administration. In one embodiment, the pharmaceutical compositions are formulated for intravenous administration. In one embodiment, the pharmaceutical compositions are formulated for intramuscular administration. In one embodiment, the pharmaceutical compositions are formulated for inhalation administration. In one embodiment, the administration is intraarterial administration. In one embodiment, the administration is intraperitoneal administration.

Generally, the pharmaceutical compositions provided herein are administered in an effective amount. The amount of the pharmaceutical composition actually administered will typically be determined by a physician, in the light of the relevant circumstances, including the condition to be treated or prevented, the chosen route of administration, the actual pharmaceutical composition administered, the age, weight, and response of the individual patient, the severity of the patient's symptoms, and the like.

When used to prevent the disorder of the present disclosure, the pharmaceutical compositions provided herein will be administered to a subject at risk for developing the condition, typically on the advice and under the supervision of a physician, at the dosage levels described above. Subjects at risk for developing a particular condition generally include those that have a family history of the condition, or those who have been identified by genetic testing or screening to be particularly susceptible to developing the condition.

The pharmaceutical compositions provided herein can also be administered chronically (“chronic administration”). Chronic administration refers to administration of a compound or pharmaceutical composition thereof over an extended period of time, e.g., for example, over 3 months, 6 months, 1 year, 2 years, 3 years, 5 years, etc., or may be continued indefinitely, for example, for the rest of the subject's life. In certain embodiments, the chronic administration is intended to provide a constant level of the compound in the blood, e.g., within the therapeutic window over the extended period of time.

The pharmaceutical compositions of the present disclosure may be further delivered using a variety of dosing methods. For example, in certain embodiments, the pharmaceutical composition may be given as a bolus, e.g., in order to raise the concentration of the compound in the blood to an effective level. The placement of the bolus dose depends on the systemic levels of the active ingredient desired throughout the body, e.g., an intramuscular or subcutaneous bolus dose allows a slow release of the active ingredient, while a bolus delivered directly to the veins (e.g., through an IV drip) allows a much faster delivery which quickly raises the concentration of the active ingredient in the blood to an effective level. In other embodiments, the pharmaceutical composition may be administered as a continuous infusion, e.g., by IV drip, to provide maintenance of a steady-state concentration of the active ingredient in the subject's body. Furthermore, in still yet other embodiments, the pharmaceutical composition may be administered as first as a bolus dose, followed by continuous infusion.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the active substance is usually a minor component (from about 0.1 to about 50% by weight or alternatively from about 1 to about 40% by weight) with the remainder being various vehicles or excipients and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable excipients known in the art. As before, the active compound in such compositions is typically a minor component, often being from about 0.05 to 10% by weight with the remainder being the injectable excipient and the like.

The above-described components for orally administrable, injectable or topically administrable compositions are merely representative. Other materials as well as processing techniques and the like are set forth in Part 8 of Remington's Pharmaceutical Sciences, 17^{th} edition, 1985, Mack Publishing Company, Easton, Pennsylvania, which is incorporated herein by reference.

Also provided herein is a kit (e.g., pharmaceutical packs) comprising the pharmaceutical composition described herein. A kits described herein may include the pharmaceutical composition and other therapeutic, or diagnostic, or prophylactic agents, and a first and a second containers (e.g., vials, ampoules, bottles, syringes, and/or dispersible packages or other materials) containing the pharmaceutical composition or other therapeutic, or diagnostic, or prophylactic agents. In some embodiments, kits provided can also optionally include a third container containing a pharmaceutically acceptable excipient for diluting or suspending the lipid nanoparticle composition of the present disclosure and/or other therapeutic, or diagnostic, or prophylactic agent. In some embodiments, the lipid nanoparticle composition of the present application provided in the first container and the other therapeutic, or diagnostic, or prophylactic agents provided in the second container is combined to form a unit dosage form.

5.5 Lipid Nanoparticle Produced from a Process

Also provided herein is a lipid nanoparticle produced by a process comprising the steps of:
(i) dissolving in a first solution a mixture comprising a permanently cationic lipid and an ionizable lipid to form a lipid solution, wherein the lipid solution is formed in an organic solvent;
(ii) dissolving in a second solution a therapeutic agent to form a therapeutic agent solution; and
(iii) mixing the lipid solution and the therapeutic agent solution at a mixing speed of about 1 mL/min to about 18 mL/min,
wherein the lipid nanoparticle has a diameter of from about 160 nm to about 900 nm.

In one embodiment, the organic solvent is a water-miscible organic solvent. In one embodiment, the organic solvent is an alcohol. In one embodiment, the organic solvent is ethanol.

In one embodiment, the second solution is an aqueous solution. In one embodiment, the second solution is an aqueous solution of pH below 7. In one embodiment, the second solution is an aqueous solution of pH between 3 and 6. In one embodiment, the second solution is an aqueous solution of pH about 3.5, about 4, about 4.5, about 5, about 5.5, or about 6. In one embodiment, the second solution is a sodium acetate buffer solution having a pH of about 4.5.

In one embodiment, the lipid solution and the therapeutic agent solution are mixed at a volumetric ratio of from about 1:1 to about 1:4. In one embodiment, the lipid solution and the therapeutic agent solution are mixed at a volumetric ratio of about 1:1. In one embodiment, the lipid solution and the therapeutic agent solution are mixed at a volumetric ratio of about 1:2. In one embodiment, the lipid solution and the therapeutic agent solution are mixed at a volumetric ratio of about 1:3. In one embodiment, the lipid solution and the therapeutic agent solution are mixed at a volumetric ratio of about 1:4.

In one embodiment, the mixing speed is from about 1 mL/min to about 18 mL/min. In one embodiment, the mixing speed is from about 1 mL/min to about 10 mL/min. In one embodiment, the mixing speed is from about 2 mL/min to about 6 mL/min. In one embodiment, the mixing speed is from about 10 mL/min to about 18 mL/min. In one embodiment, the mixing speed is from about 10 mL/min. In one embodiment, the mixing speed is from about 12 mL/min. In one embodiment, the mixing speed is from about 14 mL/min. In one embodiment, the mixing speed is from about 16 mL/min. In one embodiment, the mixing speed is from about 18 mL/min.

### 5.6 Size of a Lipid Nanoparticle

In one embodiment, provided herein is lipid nanoparticle having a diameter of from about 160 nm to about 900 nm. In one embodiment, provided herein is lipid nanoparticle having a diameter of from about 180 nm to about 900 nm. In one embodiment, the lipid nanoparticle having a diameter of from about 160 nm to about 600 nm. In one embodiment, provided herein is a lipid nanoparticle having a diameter of from about 160 nm to about 400 nm. In one embodiment, provided herein is a lipid nanoparticle having a diameter of from about 160 nm to about 350 nm. In one embodiment, provided herein is a lipid nanoparticle having a diameter of from about 180 nm to about 300 nm. In one embodiment, provided herein is a lipid nanoparticle having a diameter of from about 300 nm to about 400 nm. In one embodiment, the diameter is hydrodynamic diameter.

In one embodiment, a lipid nanoparticle of larger size has higher delivery efficiency to the lung compared with the lipid nanoparticle of smaller size. In one embodiment, the ratio of lipid nanoparticle delivered to lung and liver (lung/liver ratio) increases with the increase of the size of the lipid nanoparticle. In one embodiment, a lipid nanoparticle having a size between 160 to 900 nm has higher lung/liver ratio than the lipid nanoparticle having a size below 150 nm.

### 5.6.1 Method of Measurement of Lipid Nanoparticle Size

Many techniques known in the art can be used to measure the size of lipid nanoparticles. In one embodiment, the size is determined by dynamic light scattering (DLS). In one embodiment, the size is determined by laser diffraction. In one embodiment, the size is determined by size exclusion chromatography. In one embodiment, the size is determined by diffusion nuclear magnetic resonance. In one embodiment, the size is determined by nanoparticle tracking analysis (NTA). In one embodiment, the size is determined by centrifugal sedimentation. In one embodiment, the size is determined by atomic force microscopy (AFM). In one embodiment, the size is determined by transmission electron microscopy (TEM). In one embodiment, the size is determined by Cryo-electron microscopy (Cryo-TEM). In one embodiment, the size is determined by Small-Angle X-ray Scattering (SAXS).

### 5.6.2 Dynamic Light Scattering

In one embodiment, the size is determined by dynamic light scattering (DLS). DLS is a technique used to measure the size and size distribution of particles or molecules in solution. DLS is particularly useful for analyzing the hydrodynamic diameter of nanoparticles. Without bound by the theory, DLS is based on the Brownian motion of particles suspended in a liquid medium. The larger the particle, the slower the Brownian motion will be. Smaller particles are kicked further by the solvent molecules and move more rapidly. The velocity of the Brownian motion is defined by a property known as the translational diffusion coefficient

Without bound by the theory, in DLS, a laser beam is directed at the sample containing particles in suspension. These particles scatter the incident laser light in all directions. The scattered light is then collected at a specific angle using a detector. The fluctuations in the intensity of the scattered light are analyzed over time using a correlation function. The correlation function measures the intensity autocorrelation of the scattered light and provides information about the rate of fluctuations caused by Brownian motion. From the correlation function, the particle size information is obtained by applying the principles of autocorrelation analysis. The fluctuations in intensity due to Brownian motion result in a decay of the correlation function, and the decay rate is related to the particle's diffusion coefficient and, consequently, its hydrodynamic diameter.

DLS provides the hydrodynamic diameter of nanoparticles, which includes the lipid nanoparticle's core size and the surrounding solvent molecules that form the particle's dynamic hydration layer. DLS measures the effective size of nanoparticles as they behave in solution.

Periodical DLS measurements of a sample can show whether the particles aggregate over time by seeing whether the hydrodynamic radius of the particle increases. In one embodiment, the size of the lipid nanoparticle is determined by DLS within 7 days of preparation of the lipid nanoparticle. In one embodiment, the size of the lipid nanoparticle is determined by DLS within 48 hours of preparation of the lipid nanoparticle. In one embodiment, the size of the lipid nanoparticle is determined by DLS within 24 hours of preparation of the lipid nanoparticle. In one embodiment, the size of the lipid nanoparticle is determined by DLS within 3 hours of preparation of the lipid nanoparticle.

In one embodiment, the size of the lipid nanoparticle is determined after preparation without further processing. In one embodiment, the size of the lipid nanoparticle is determined after the lipid nanoparticle is passed through a filter membrane. In one embodiment, the filter membrane is a sterile membrane. In one embodiment, the filter membrane is a non-sterile membrane. In one embodiment, the filter membrane has a pore size of from about 0.2 μm to about 0.8 μm. In one embodiment, the filter membrane has a pore size of about 0.22 μm. In one embodiment, the filter membrane has a pore size of about 0.45 μm.

In one embodiment, the size of the lipid nanoparticle is measured by Zetasizer Pro (Malvern Panalytical). In one embodiment, the size of the lipid nanoparticle is measured in a disposable folded capillary cell. In one embodiment, the size of the lipid nanoparticle is measured at room temperature (e.g. 25° C.). In one embodiment, the size of the lipid nanoparticle is measured by diluting the lipid nanoparticle in a PBS buffer (1×, pH=7.4). In one embodiment, the lipid nanoparticle is diluted by a factor of at least 10, at least 20, at least 40, at least 60, at least 80, or at least 100 before DLS measurement. In one embodiment, the size of the lipid nanoparticle is measured at a refractive index of about 1.1.

### 5.7 Methods of Making or Producing a Lipid Nanoparticle

In one embodiment, provided herein is a method of producing a lipid nanoparticle having a diameter of from about 160 nm to about 900 nm comprising the steps of:
(i) dissolving in a first solution a mixture comprising a permanently cationic lipid and an ionizable lipid to form a lipid solution, wherein the lipid solution is formed in an organic solvent;
(ii) dissolving in a second solution a therapeutic agent to form a therapeutic agent solution; and
(iii) mixing the lipid solution and the therapeutic agent solution at a mixing speed of about 10 mL/min to about 18 mL/min.

In one embodiment, the organic solvent is a water-miscible organic solvent. In one embodiment, the organic solvent is an alcohol. In one embodiment, the organic solvent is ethanol.

In one embodiment, the second solution is an aqueous solution. In one embodiment, the second solution is an aqueous solution of pH below 7. In one embodiment, the second solution is an aqueous solution of pH between 3 and 6. In one embodiment, the second solution is an aqueous solution of pH about 3.5, about 4, about 4.5, about 5, about 5.5, or about 6. In one embodiment, the second solution is a sodium acetate buffer solution having a pH of about 4.5.

In one embodiment, the lipid solution and the therapeutic agent solution are mixed at a volumetric ratio of from about 1:1 to about 1:4. In one embodiment, the lipid solution and the therapeutic agent solution are mixed at a volumetric ratio of about 1:1. In one embodiment, the lipid solution and the therapeutic agent solution are mixed at a volumetric ratio of about 1:2. In one embodiment, the lipid solution and the therapeutic agent solution are mixed at a volumetric ratio of about 1:3. In one embodiment, the lipid solution and the therapeutic agent solution are mixed at a volumetric ratio of about 1:4.

In one embodiment, the diameter of the lipid nanoparticles produced is controlled by the mixing speed. In one embodiment, the mixing speed is from about 1 mL/min to about 18 mL/min. In one embodiment, the mixing speed is from about 1 mL/min to about 10 mL/min. In one embodiment, the mixing speed is from about 2 mL/min to about 6 mL/min. In one embodiment, the mixing speed is from about 10 mL/min to about 18 mL/min. In one embodiment, the mixing speed is about 10 mL/min. In one embodiment, the mixing speed is about 12 mL/min. In one embodiment, the mixing speed is about 14 mL/min. In one embodiment, the mixing speed is about 16 mL/min. In one embodiment, the mixing speed is about 18 mL/min.

### 5.8 Methods of Treatment

In one embodiment, provided herein is a method of treating or preventing a disease or disorder in a subject. In one embodiment, the method comprises administering to a subject the lipid nanoparticle described in Section 5.2 that comprises a therapeutic agent described in Section 5.2.6. In one embodiment, the method comprises administering to a subject the population of lipid nanoparticle described in Section 5.3 that comprises a therapeutic agent described in Section 5.2.6. In one embodiment, the method comprises administering to a subject the pharmaceutical composition described in Section 5.4 that comprises a therapeutic agent described in Section 5.2.6.

In one embodiment, provided herein is a method of treating or preventing a disease or disorder in a subject, comprising administering to the subject a therapeutically effective amount of a lipid nanoparticle, wherein the lipid nanoparticle comprises (i) a permanently cationic lipid in an amount of from about 15 mol % to about 90 mol % of the total lipid present in the lipid nanoparticle, and (ii) an ionizable lipid in an amount from about 20 mol % to about 60 mol % of the total lipid present in the lipid nanoparticle, and wherein the lipid nanoparticle has a diameter of from about 160 nm to about 900 nm.

In one embodiment, provided herein is a method of treating or preventing a disease or disorder in a subject, comprising administering to the subject having the disease or disorder a therapeutically effective amount of a lipid nanoparticle, wherein the lipid nanoparticle comprises a permanently cationic lipid and an ionizable lipid, and wherein the lipid nanoparticle has a diameter of from about 300 nm to about 900 nm.

In one embodiment, the administration is intravenous administration. In one embodiment, the administration is intraarterial administration. In one embodiment, the administration is intraperitoneal administration. In one embodiment, the administration is oral administration. In one embodiment, the administration is intramuscular administration. In one embodiment, the administration is inhalation administration.

In one embodiment, provided herein is a method of treating a lung disease in a subject. In one embodiment, provided herein is a method of treating lung cancer in a subject.

In one embodiment, provided herein is a method of delivering or expressing a therapeutic agent in a subject, comprising administration to the subject a therapeutically effective amount of a lipid nanoparticle, wherein the lipid nanoparticle comprises (i) a permanently cationic lipid in an amount from about 15 mol % to about 90 mol % of the total lipid present in the lipid nanoparticle, and (ii) an ionizable lipid in an amount from about 20 mol % to about 60 mol % of the total lipid present in the lipid nanoparticle, and wherein the lipid nanoparticle has a diameter of from about 160 nm to about 900 nm.

In one embodiment, the amount of the therapeutic agent delivered or expressed in a non-hepatic organ of the subject is higher than the amount of the therapeutic agent delivered or expressed in the liver of the subject. In one embodiment, the non-hepatic organ is lung. In one embodiment, the non-hepatic organ is spleen. In one embodiment, the non-hepatic organ is the lymph nodes.

In one embodiment, the amount of the therapeutic agent delivered or expressed in the lung of the subject is higher than the amount of the therapeutic agent delivered or expressed in the liver of the subject. In one embodiment, the amount of the therapeutic agent delivered or expressed in the lung of the subject is at least one time higher than the amount of the therapeutic agent delivered or expressed in the liver of the subject. In one embodiment, the amount of the therapeutic agent delivered or expressed in the lung of the subject is at least 2 times higher than the amount of the therapeutic agent delivered or expressed in the liver of the subject. In one embodiment, the amount of the therapeutic agent delivered or expressed in the lung of the subject is at least 3 times higher than the amount of the therapeutic agent delivered or expressed in the liver of the subject. In one embodiment, the amount of the therapeutic agent delivered or expressed in the lung of the subject is at least 5 times higher than the amount of therapeutic agent delivered or expressed in the liver of the subject. In one embodiment, the amount of the therapeutic agent delivered or expressed in the lung of the subject is at least 10 times higher than the amount of therapeutic agent delivered or expressed in the liver of the subject. In one embodiment, the amount of the therapeutic agent delivered or expressed in the lung of the subject is at least 20 times higher than the amount of therapeutic agent delivered or expressed in the liver of the subject. In one embodiment, the amount of the therapeutic agent delivered or expressed in the lung of the subject is at least 40 times higher than the amount of therapeutic agent delivered or expressed in the liver of the subject. In one embodiment, the amount of the therapeutic agent delivered or expressed in the lung of the subject is at least 60 times higher than the amount of therapeutic agent delivered or expressed in the liver of the subject. In one embodiment, the amount of the therapeutic agent delivered or expressed in the lung of the subject is at least 100 times higher than the amount of therapeutic agent delivered or expressed in the liver of the subject.

### 5.9 Embodiments

1. In one embodiment, provided herein is a lipid nanoparticle for use in delivering or expressing a therapeutic agent in the lung of a subject, wherein the lipid nanoparticle is administered intravenously, intraarterially, or intraperitoneally to the subject, wherein the lipid nanoparticle has a positive surface charge, and wherein the lipid nanoparticle has a diameter of from about 160 nm to about 900 nm.

2. The lipid nanoparticle for use of embodiment 1, wherein the lipid nanoparticle comprises a permanently cationic lipid and an ionizable lipid.

3. A lipid nanoparticle for use in delivering or expressing a therapeutic agent in the lung of a subject, wherein the lipid nanoparticle comprises a permanently cationic lipid and an ionizable lipid, and wherein the lipid nanoparticle has a diameter of from about 160 nm to about 900 nm.

4. The lipid nanoparticle for use of any one of embodiments 1 to 3, wherein the lipid nanoparticle has a diameter of from 180 nm to about 900 nm, from about 300 nm to about 900 nm, from about 180 nm to about 600 nm, from about 180 nm to about 400 nm, from about 180 nm to about 350 nm, or from about 180 nm to about 300 nm.

5. The lipid nanoparticle for use of any one of embodiments 1 to 4, wherein the lipid nanoparticle has a diameter of from about 180 nm to about 300 nm.

6. The lipid nanoparticle for use of any one of embodiments 1 to 5, wherein the lipid nanoparticle has a greater than neutral zeta potential at physiologic pH.

7. The lipid nanoparticle for use of any one of embodiments 1 to 5, wherein the lipid nanoparticle has a zeta potential of from about 0 mV to about 25 mV, from about 0 mV to about 20 mV, or from about 2 mV to about 15 mV.

8. The lipid nanoparticle for use of any one of embodiments 2 to 7, wherein the amount of the permanently cationic lipid is from about 15 mol % to about 90 mol %, from about 20 mol % to about 80 mol %, from about 30 mol % to about 70 mol %, from about 40 mol % to about 60 mol %, or from about 45 mol % to about 55 mol % of the total lipid present in the lipid nanoparticle.

9. The lipid nanoparticle for use of any one of embodiments 2 to 8, wherein the permanently cationic lipid has a pKa of greater than about 10, or greater than about 13.

10. The lipid nanoparticle for use of any one of embodiments 2 to 9, wherein the permanently cationic lipid comprises a quaternary ammonium group.

11. The lipid nanoparticle for use of any one of embodiments 2 to 10, wherein the permanently cationic lipid is a compound of formula (I):

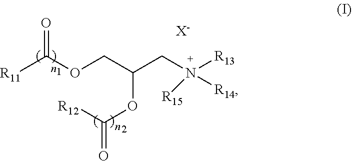

or a stereoisomer, a mixture of stereoisomers, or a pharmaceutically acceptable salt thereof, wherein R₁₁ and R₁₂ are each independently C₆₋₃₀ alkyl, C₆₋₃₀ alkenyl, or C₆₋₃₀ alkynyl, and wherein the alkyl, alkenyl and alkynyl are independently optionally substituted with one or more groups selected from hydroxyl, halogen, cyano, C₁₋₃₀ alkyl, C₁₋₃₀ haloalkyl, C₁₋₃₀ alkoxy, —S—C₁₋₃₀ alkyl, amino, —NH—C₁₋₃₀ alkyl, and —N(C₁₋₃₀ alkyl)₂;
R₁₃, R₁₄, and R₁₅ are each independently C₁₋₆ alkyl, C₁₋₆ haloalkyl, C₂₋₆ alkenyl, C₂₋₆ alkynyl, or any two of R₁₃, R₁₄, and R₁₅ together with the nitrogen atom they are attached to form a 4 to 8-membered ring, and wherein the alkyl, haloalkyl, alkenyl, alkynyl, and ring are optionally substituted;
X⁻ is an anion; and
n₁ and n₂ are each independently 0 or 1.

12. The lipid nanoparticle for use of embodiment 11, wherein R₁ and R₁₂ are each independently C₁₅₋₂₀ alkyl, C₁₅₋₂₀ alkenyl, or C₁₅₋₂₀ alkynyl, and wherein the alkyl, alkenyl and alkynyl are independently optionally substituted with one or more groups selected from hydroxyl, halogen, cyano, C₁₋₂₀ alkyl, C₁₋₂₀ haloalkyl, C₁₋₂₀ alkoxy, —S—C₁₋₂₀ alkyl, amino, —NH—C₁₋₂₀ alkyl, and —N(C₁₋₂₀ alkyl)₂.

13. The lipid nanoparticle for use of embodiment 11 or 12, wherein R₁₃, R₁₄, and R₁₅ are each independently C₁₋₆ alkyl optionally substituted with hydroxyl, halogen, cyano, C₁₋₆ alkoxy, —S—C₁₋₆ alkyl, amino, —NH—C₁₋₆ alkyl, or —N(C₁₋₆ alkyl)₂.

14. The lipid nanoparticle for use of any one of embodiments 2 to 10, wherein the permanently cationic lipid is a compound of formula (II):

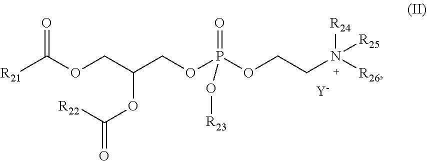

or a stereoisomer, a mixture of stereoisomers, or a pharmaceutically acceptable salt thereof, wherein R₂₁ and R₂₂ are each independently C₆₋₃₀ alkyl, C₆₋₃₀ alkenyl, or C₆₋₃₀ alkynyl, and wherein the alkyl, alkenyl and alkynyl are independently optionally substituted with one or more groups selected from hydroxyl, halogen, cyano, C₁₋₃₀ alkyl, C₁₋₃₀ haloalkyl, C₁₋₃₀ alkoxy, —S—C₁₋₃₀ alkyl, amino, —NH—C₁₋₃₀ alkyl, and —N(C₁₋₃₀ alkyl)₂;
R₂₃ is C₁₋₆ alkyl, C₁₋₆ haloalkyl, C₂₋₆ alkenyl, or C₂₋₆ alkynyl, and wherein R₂₃ is optionally substituted with one or more groups selected from halogen, hydroxyl, C₁₋₆ alkyl, C₁₋₆ haloalkyl, C₁₋₆ alkoxy, —OC(═O)R₂ₐ, —C(═O)OR₂ₐ, —C(═O)NHR₂ₐ, and —NHC(═O)R₂ₐ;
R₂ₐ is hydrogen, C₁₋₆ alkyl, or C₁₋₆ haloalkyl;
R₂₄, R₂₅, and R₂₆ are each independently C₁₋₆ alkyl, C₁₋₆ haloalkyl, C₂₋₆ alkenyl, C₂₋₆ alkynyl, or any two of R₂₄, R₃₅, and R₂₆ together with the nitrogen atom they are attached to form a 4 to 8-membered ring, and wherein the alkyl, haloalkyl, alkenyl, alkynyl, and ring are optionally substituted; and
Y⁻ is an anion.

15. The lipid nanoparticle for use of embodiment 14, wherein R₂₁ and R₂₂ are each independently C₁₀₋₂₅ alkyl, C₁₀₋₂₅ alkenyl, or C₁₀₋₂₅ alkynyl, and wherein the alkyl, alkenyl and alkynyl are independently optionally substituted with one or more groups selected from hydroxyl, halogen, cyano, C₁₋₂₅ alkyl, C₁₋₂₅ haloalkyl, C₁₋₂₅ alkoxy, —S—C₁₋₂₅ alkyl, amino, —NH—C₁₋₂₅ alkyl, and —N(C₁₋₂₅ alkyl)₂.

16. The lipid nanoparticle for use of embodiment 14 or 15, wherein R₂₃ is C₁₋₆ alkyl or C₁₋₆ haloalkyl.

17. The lipid nanoparticle for use of any one of embodiments 14 to 16, wherein R₂₄, R₂₅, and R₂₆ are each independently C₁₋₆ alkyl optionally substituted with hydroxyl, halogen, cyano, C₁₋₆ alkoxy, —S—C₁₋₆ alkyl, amino, —NH—C₁₋₆ alkyl, or —N(C₁₋₆ alkyl)₂, or any two of R₂₄, R₂₅, and R₂₆ together with the nitrogen atom they are attached to form a 5 to 6-membered ring.

18. The lipid nanoparticle for use of any one of embodiments 1 to 10, wherein the permanently cationic lipid is a pharmaceutically acceptable salt of

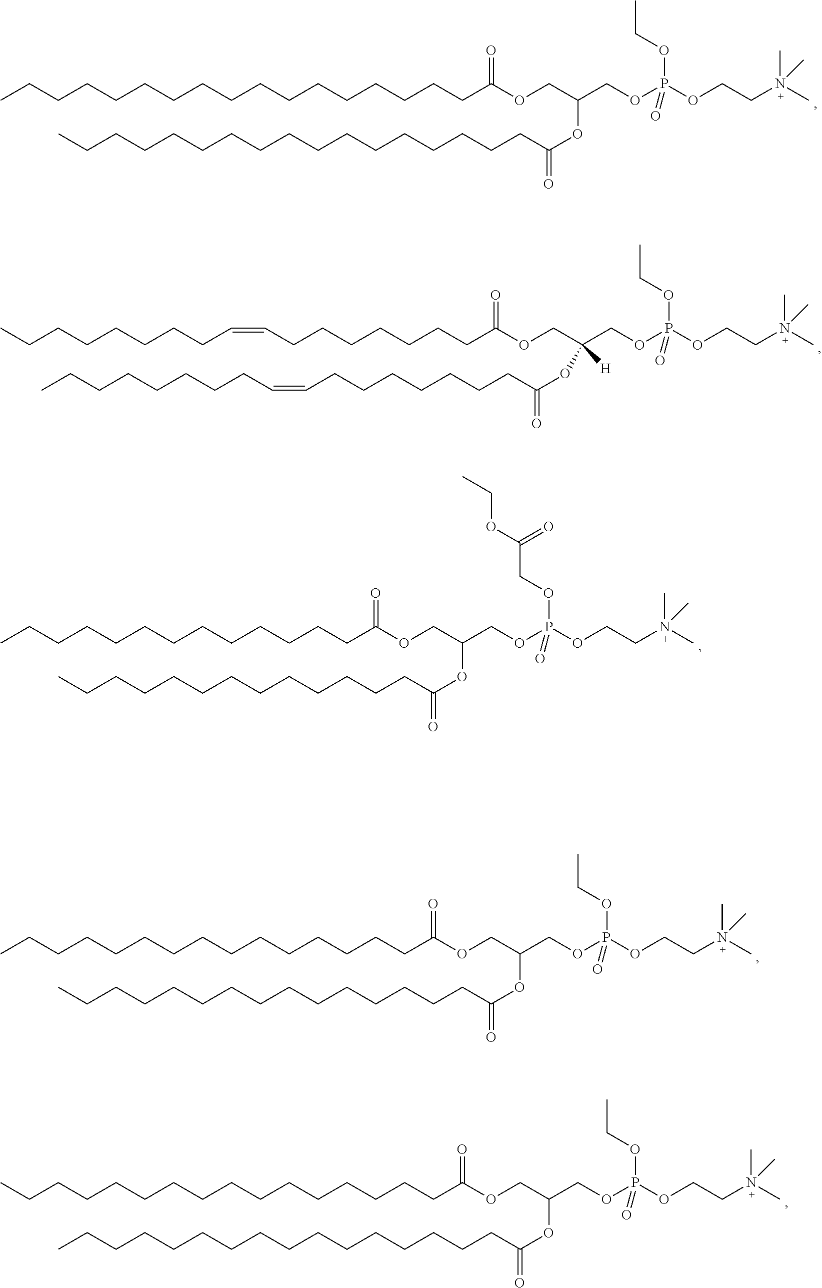

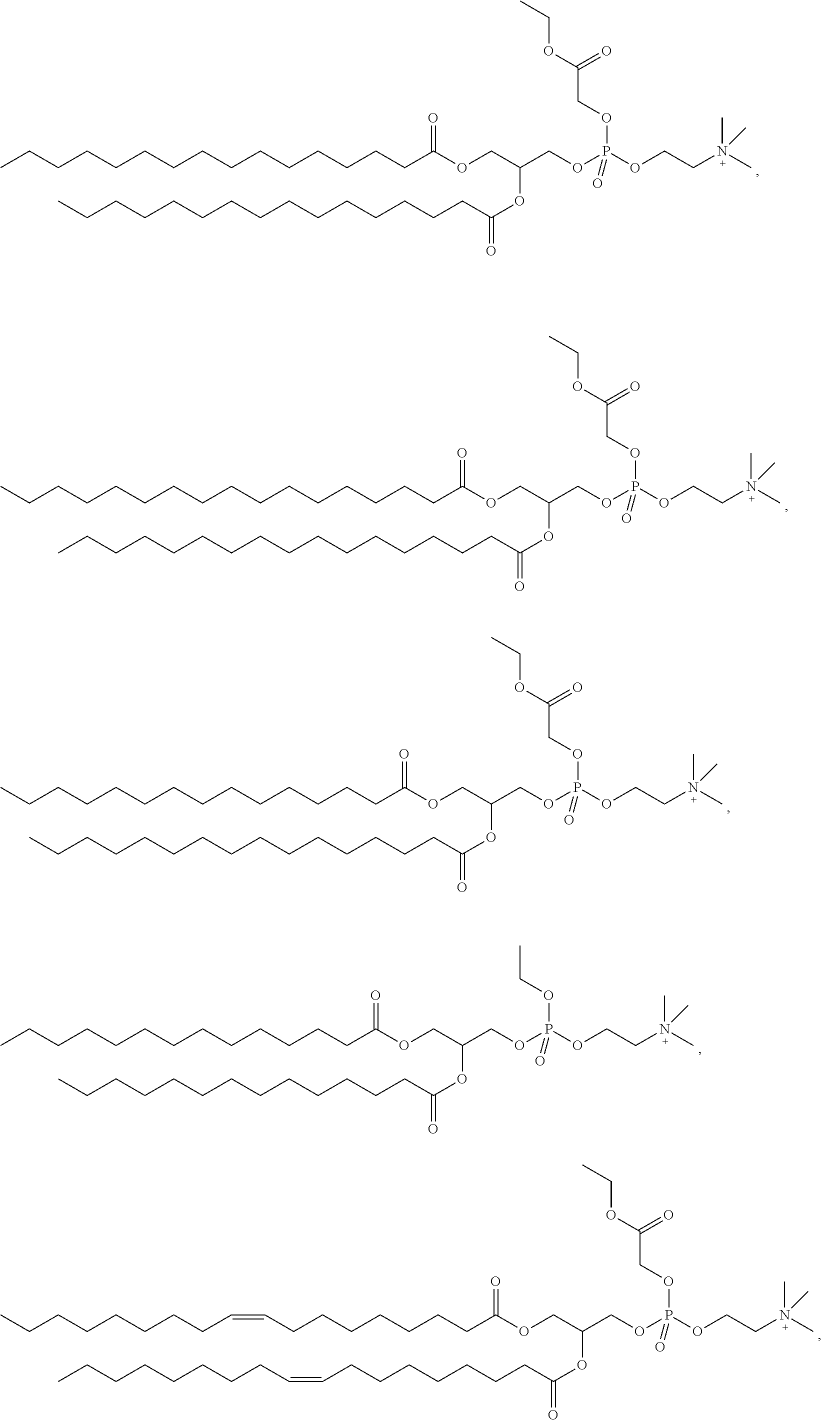

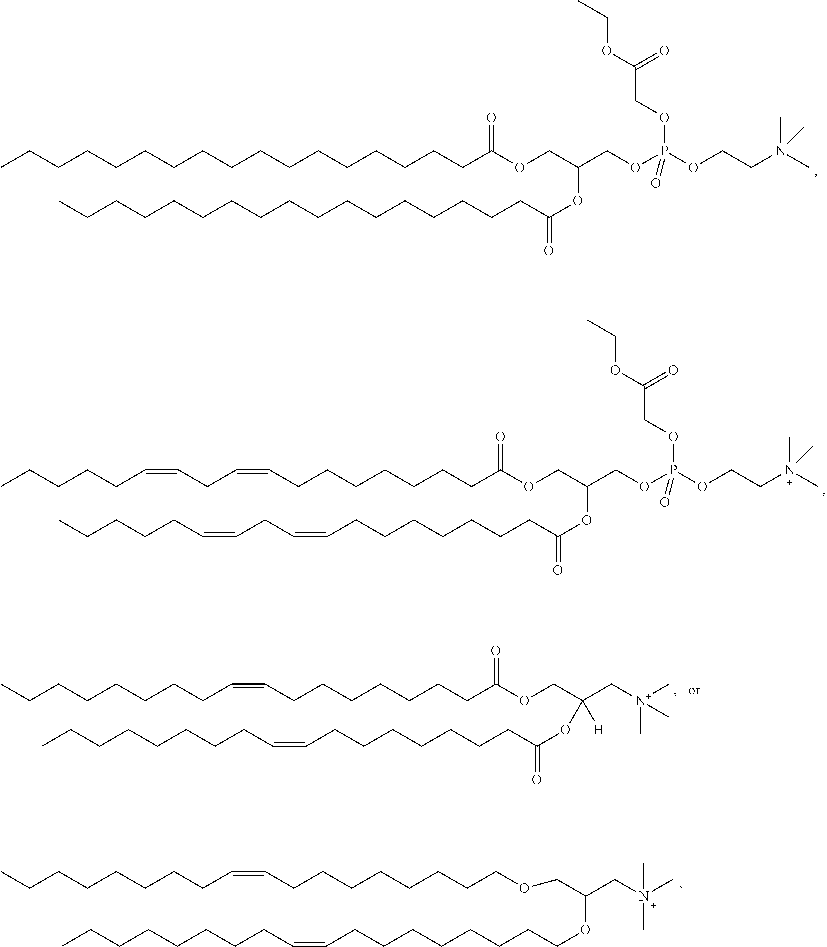

or a stereoisomer, or a mixture of stereoisomers thereof.

19. The lipid nanoparticle for use of any one of embodiments 1 to 10, wherein the permanently cationic lipid is DOTMA, DOTAP, MVL5, DOGS, DC-Chol, DDAB, EPC, or a mixture thereof.

20. The lipid nanoparticle for use of any one of embodiments 1 to 19, wherein the amount of the ionizable lipid is from about 15 mol % to about 60 mol % of the total lipid present in the lipid nanoparticle.

21. The lipid nanoparticle for use of embodiment 20, wherein the amount of the ionizable lipid is from about 15 mol % to about 40 mol %, or from about 20 mol % to about 30 mol % of the total lipid present in the lipid nanoparticle.

22. The lipid nanoparticle for use of any one of embodiments 1 to 21, wherein
(i) the amount of the permanently cationic lipid is from about 15 mol % to about 90 mol % of the total lipid present in the lipid nanoparticle, and the amount of the ionizable lipid is from about 15 mol % to about 60 mol % of the total lipid present in the lipid nanoparticle;
(ii) the amount of the permanently cationic lipid is from about 40 mol % to about 60 mol % of the total lipid present in the lipid nanoparticle, and the amount of the ionizable lipid is from about 15 mol % to about 40 mol % of the total lipid present in the lipid nanoparticle; or
(iii) the amount of the permanently cationic lipid is from about 45 mol % to about 55 mol % of the total lipid present in the lipid nanoparticle, and the amount of the ionizable lipid is from about 20 mol % to about 30 mol % of the total lipid present in the lipid nanoparticle.

23. The lipid nanoparticle for use of any one of embodiments 1 to 22, wherein the ionizable lipid has a pKa of from about 7 to about 13, from about 7 to about 11, or from about 7 to about 9.

24. The lipid nanoparticle for use of any one of embodiments 1 to 23, wherein the lipid nanoparticle further comprises a phospholipid.

25. The lipid nanoparticle for use of embodiment 24, wherein the phospholipid is DSPC, DMPC, DOPC, DPPC, POPC, DOPE, DMPE, POPOE, or DPPE, or a mixture thereof.

26. The lipid nanoparticle for use of any one of embodiments 1 to 23, wherein the lipid nanoparticle does not comprise a phospholipid or comprises a phospholipid in an amount less than about 15 mol %, less than about 10 mol %, less than about 8 mol %, less than about 5 mol %, less than about 3 mol %, or less than about 1 mol % of the total lipid present in the lipid nanoparticle.

27. The lipid nanoparticle for use of any one of embodiments 1 to 26, wherein the lipid nanoparticle further comprises a steroid.

28. The lipid nanoparticle for use of embodiment 27, wherein the steroid is cholesterol, campesterol, stigmasterol, sitosterol, brassicasterol, ergosterol, solanine, ursolic acid, alpha-tocopherol, beta-sitosterol, avenasterol, calciferol, or canola sterol.

29. The lipid nanoparticle for use of embodiment 27 or 28, wherein the amount of the steroid is from about 5 mol % to about 60 mol %, from about 10 mol % to about 50 mol %, from about 10 mol % to about 40 mol %, from about 20 mol % to about 30 mol %, or about 25 mol % of the total lipid present in the lipid nanoparticle.

30. The lipid nanoparticle for use of any one of embodiments 1 to 29, wherein the lipid nanoparticle further comprises a pegylated lipid.

31. The lipid nanoparticle for use of embodiment 30, wherein a pegylated moiety of the pegylated lipid has a molecule weight of from about 1000 Da to about 10,000 Da, from about 1000 Da to about 5000 Da, or from about 1000 Da to about 2000 Da.

32. The lipid nanoparticle for use of embodiment 30 or 31, wherein the pegylated lipid is ALC-0159, DMG-PEG2000, DMPE-PEG1000, DPPE-PEG1000, DSPE-PEG1000, DOPE-PEG1000, Ceramide-PEG2000, DMPE-PEG2000, DPPE-PEG2000, DSPE-PEG2000, DSPE-PEG2000-Mannose, Ceramide-PEG5000, DSPE-PEG5000, or DSPE-PEG2000 amine.

33. The lipid nanoparticle for use of any one of embodiments 30 to 32, wherein the amount of the pegylated lipid is from about 0.1 mol to about 5 mol %, from about 0.1 mol to about 3 mol %, from about 0.25 mol to about 2 mol %, from about 0.5 mol to about 1.5 mol %, or about 1 mol % of the total lipid present in the lipid nanoparticle.

34. The lipid nanoparticle for use of any one of embodiments 1 to 33, wherein:
(i) the lipid nanoparticle comprises a permanently cationic lipid in an amount from about 15 mol % to about 90 mol % of the total lipid present in the lipid nanoparticle, an ionizable lipid in an amount from about 15 mol % to about 60 mol % of the total lipid present in the lipid nanoparticle, a steroid in an amount from about 5 mol % to about 60 mol % of the total lipid present in the lipid nanoparticle and a pegylated lipid in an amount from about 0.1 mol % to about 5 mol % of the total lipid present in the lipid nanoparticle;
(ii) the lipid nanoparticle comprises a permanently cationic lipid in an amount from about 30 mol % to about 70 mol % of the total lipid present in the lipid nanoparticle, an ionizable lipid in an amount from about 15 mol % to about 40 mol % of the total lipid present in the lipid nanoparticle, a steroid in an amount from about 15 mol % to about 40 mol % of the total lipid present in the lipid nanoparticle, and a pegylated lipid in an amount from about 0.25 mol % to about 3 mol % of the total lipid present in the lipid nanoparticle; or
(iii) the lipid nanoparticle comprises a permanently cationic lipid in an amount from about 45 mol % to about 55 mol % of the total lipid present in the lipid nanoparticle, an ionizable lipid in an amount from about 20 mol % to about 30 mol % of the total lipid present in the lipid nanoparticle, a steroid in an amount from about 20 mol % to about 30 mol % of the total lipid present in the lipid nanoparticle, and a pegylated lipid in an amount from about 0.5 mol % to about 1.5 mol % of the total lipid present in the lipid nanoparticle.

35. The lipid nanoparticle for use of any one of embodiments 1 to 34, wherein the therapeutic agent is nucleic acid.

36. The lipid nanoparticle for use of embodiment 35, wherein the nucleic acid is antisense oligonucleotide (ASO), DNA, or RNA, optionally wherein the RNA is RNA interference (RNAi), small interfering RNA (siRNA), short hairpin RNA (shRNA), antisense RNA (aRNA), messenger RNA (mRNA), modified messenger RNA (mmRNA), long noncoding RNA (lncRNA), microRNA (miRNA), small activating RNA (saRNA), multicoding nucleic acid (MCNA), polymer-coded nucleic acid (PCNA), guide RNA (gRNA), CRISPR RNA (crRNA), or any other RNA in the ribozyme.

37. The lipid nanoparticle for use of embodiment 36, wherein the ratio of total number of nitrogen atoms in the permanently cationic lipid and ionizable lipid and total number of phosphate atoms in the nucleic acid is from about 1:1 to about 20:1, about 1:1 to about 15:1, from about 3:1 to about 12:1, or from about 4:1 to about 9:1.

38. The lipid nanoparticle for use of any one of embodiments 1 to 37, wherein the lipid nanoparticle has an apparent pKa of greater than about 7, greater than about 8, greater than about 9, greater than about 10, from about 7 to about 10, or greater than about 10.

39. The lipid nanoparticle for use of any one of embodiments 1 to 38, wherein the amount of the therapeutic agent delivered or expressed in the lung of the subject is higher than the amount of the therapeutic agent delivered or expressed in the liver of the subject.

40. The lipid nanoparticle for use of embodiment 39, wherein the amount of the therapeutic agent delivered or expressed in the lung of the subject is at least 1 time, at least 2 times, at least 3 times, at least 5 times, at least 10 times, at least 20 times, at least 40 times, at least 60 times, or at least 100 times higher than the amount of the therapeutic agent delivered or expressed in the liver of the subject.

41. The lipid nanoparticle for use of any one of embodiments 1 to 40, wherein the subject has a lung disease.

42. In embodiment, provided herein is a lipid nanoparticle comprising:
(i) a permanently cationic lipid in an amount from about 15 mol % to about 90 mol % of the total lipid present in the lipid nanoparticle; and
(ii) an ionizable lipid in an amount from about 15 mol % to about 60 mol % of the total lipid present in the lipid nanoparticle,
   wherein the lipid nanoparticle has a diameter of from about 160 nm to about 900 nm; and wherein the permanently cationic lipid is a compound of formula (I) or (II);

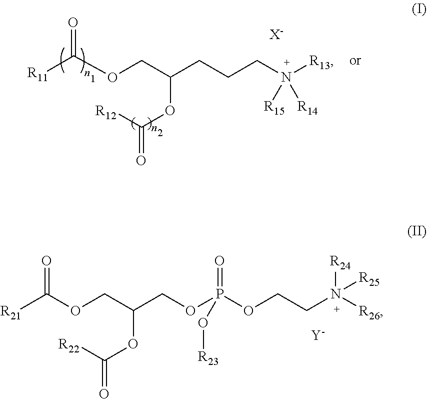

or a stereoisomer, a mixture of stereoisomers, or a pharmaceutically acceptable salt thereof, wherein Rn, R₁₂, R₂₁ and R₂₂ are each independently C₆₋₃₀ alkyl, C₆₋₃₀ alkenyl, or C₆₋₃₀ alkynyl, and wherein the alkyl, alkenyl and alkynyl are independently optionally substituted with one or more groups selected from hydroxyl, halogen, cyano, C₁₋₃₀ alkyl, C₁₋₃₀ haloalkyl, C₁₋₃₀ alkoxy, —S—C₁₋₃₀ alkyl, amino, —NH—C₁₋₃₀ alkyl, and —N(C₁₋₃₀ alkyl)₂;
   R₁₃, R₁₄, and R₁₅ are each independently C₁₋₆ alkyl, C₁₋₆ haloalkyl, C₂₋₆ alkenyl, C₂₋₆ alkynyl, or any two of R₁₃, R₁₄, and R₁₅ together with the nitrogen atom they are attached to form a 4 to 8-membered ring, and wherein the alkyl, haloalkyl, alkenyl, alkynyl, and ring are optionally substituted;
   R₂₄, R₂₅, and R₂₆ are each independently C₁₋₆ alkyl, C₁₋₆ haloalkyl, C₂₋₆ alkenyl, C₂₋₆ alkynyl, or any two of R₂₄, R₃₅, and R₂₆ together with the nitrogen atom they are attached to form a 4 to 8-membered ring, and wherein the alkyl, haloalkyl, alkenyl, alkynyl, and ring are optionally substituted;
   R₂₃ is C₁₋₆ alkyl, C₁₋₆ haloalkyl, C₂₋₆ alkenyl, or C₂₋₆ alkynyl, and wherein R₂₃ is optionally substituted with one or more groups selected from halogen, hydroxyl, C₁₋₆ alkyl, C₁₋₆ haloalkyl, C₁₋₆ alkoxy, —OC(═O)R₂ₐ, —C(═O)OR₂ₐ, —C(═O)NHR₂ₐ, and —NHC(═O)R₂ₐ;
   R₂ₐ is hydrogen, C₁₋₆ alkyl, or C₁₋₆ haloalkyl;
   X⁻ and Y⁻ are each independently an anion; and
   n₁ and n₂ are each independently 0 or 1.

43. The lipid nanoparticle of embodiment 42, wherein R₁₁ and R₁₂ are each independently C₁₅₋₂₀ alkyl, C₁₅₋₂₀ alkenyl, or C₁₅₋₂₀ alkynyl, and wherein the alkyl, alkenyl and alkynyl are independently optionally substituted with one or more groups selected from hydroxyl, halogen, cyano, C₁₋₂₀ alkyl, C₁₋₂₀ haloalkyl, C₁₋₂₀ alkoxy, —S—C₁₋₂₀ alkyl, amino, —NH—C₁₋₂₀ alkyl, and —N(C₁₋₂₀ alkyl)₂.

44. The lipid nanoparticle of embodiment 42 or 43, wherein R₁₃, R₁₄, and R₁₅ are each independently C₁₋₆ alkyl optionally substituted with hydroxyl, halogen, cyano, C₁₋₆ alkoxy, —S—C₁₋₆ alkyl, amino, —NH—C₁₋₆ alkyl, or —N(C₁₋₆ alkyl)₂.

45. The lipid nanoparticle of embodiment 42, wherein R₂₁ and R₂₂ are each independently C₁₀₋₂₅ alkyl, C₁₀₋₂₅ alkenyl, or C₁₀₋₂₅ alkynyl, and wherein the alkyl, alkenyl and alkynyl are independently optionally substituted with one or more groups selected from hydroxyl, halogen, cyano, C₁₋₂₅ alkyl, C₁₋₂₅ haloalkyl, C₁₋₂₅ alkoxy, —S—C₁₋₂₅ alkyl, amino, —NH—C₁₋₂₅ alkyl, and —N(C₁₋₂₅ alkyl)₂.

46. The lipid nanoparticle of embodiment 42, wherein R₂₃ is C₁₋₆ alkyl or C₁₋₆ haloalkyl.

47. The lipid nanoparticle of embodiment 42, wherein R₂₄, R₂₅, and R₂₆ are each independently C₁₋₆ alkyl optionally substituted with hydroxyl, halogen, cyano, C₁₋₆ alkoxy, —S—C₁₋₆ alkyl, amino, —NH—C₁₋₆ alkyl, or —N(C₁₋₆ alkyl)₂, or any two of R₂₄, R₂₅, and R₂₆ together with the nitrogen atom they are attached to form a 5 to 6-membered ring.

48. The lipid nanoparticle of embodiment 42, wherein the permanently cationic lipid is a pharmaceutically acceptable salt of:

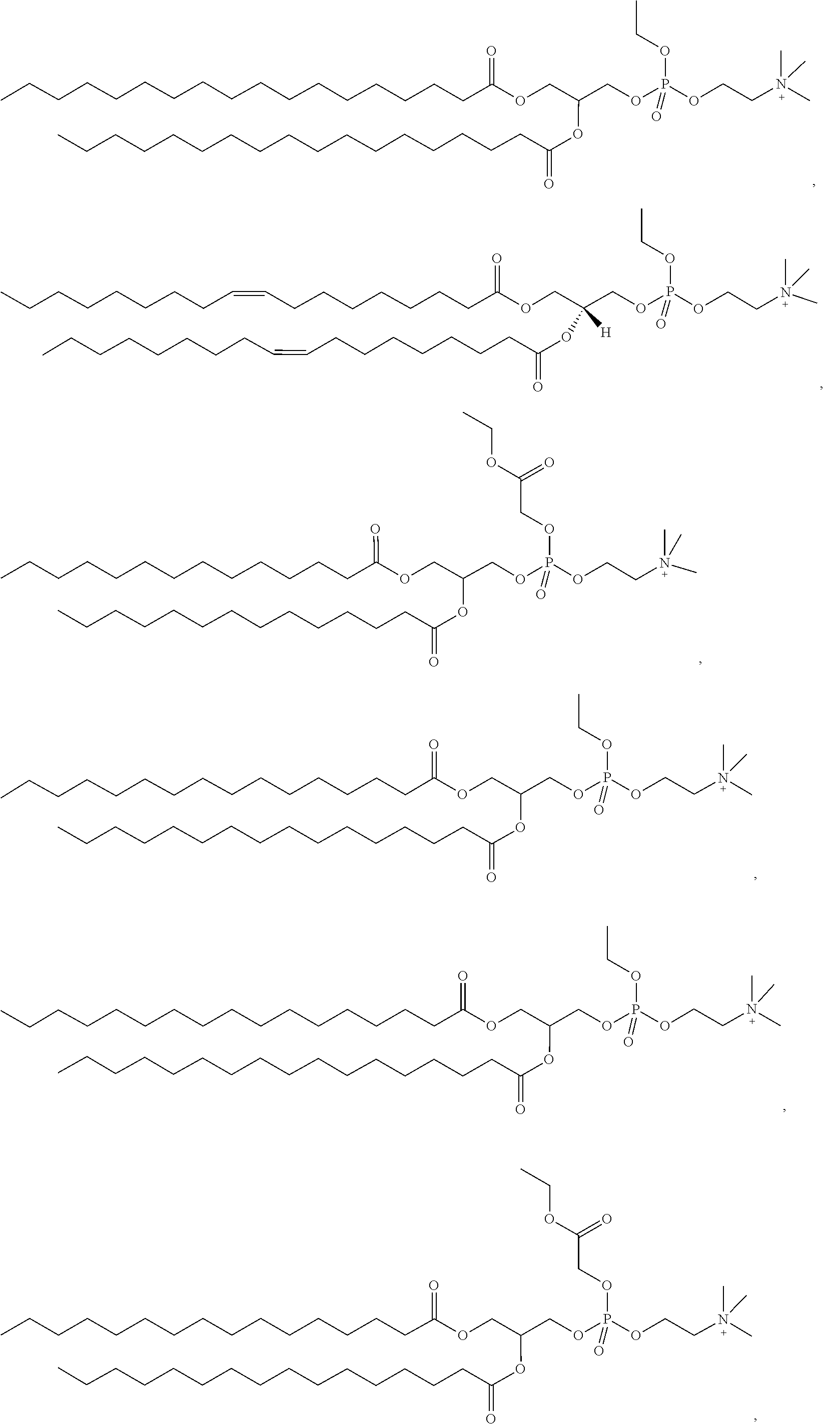

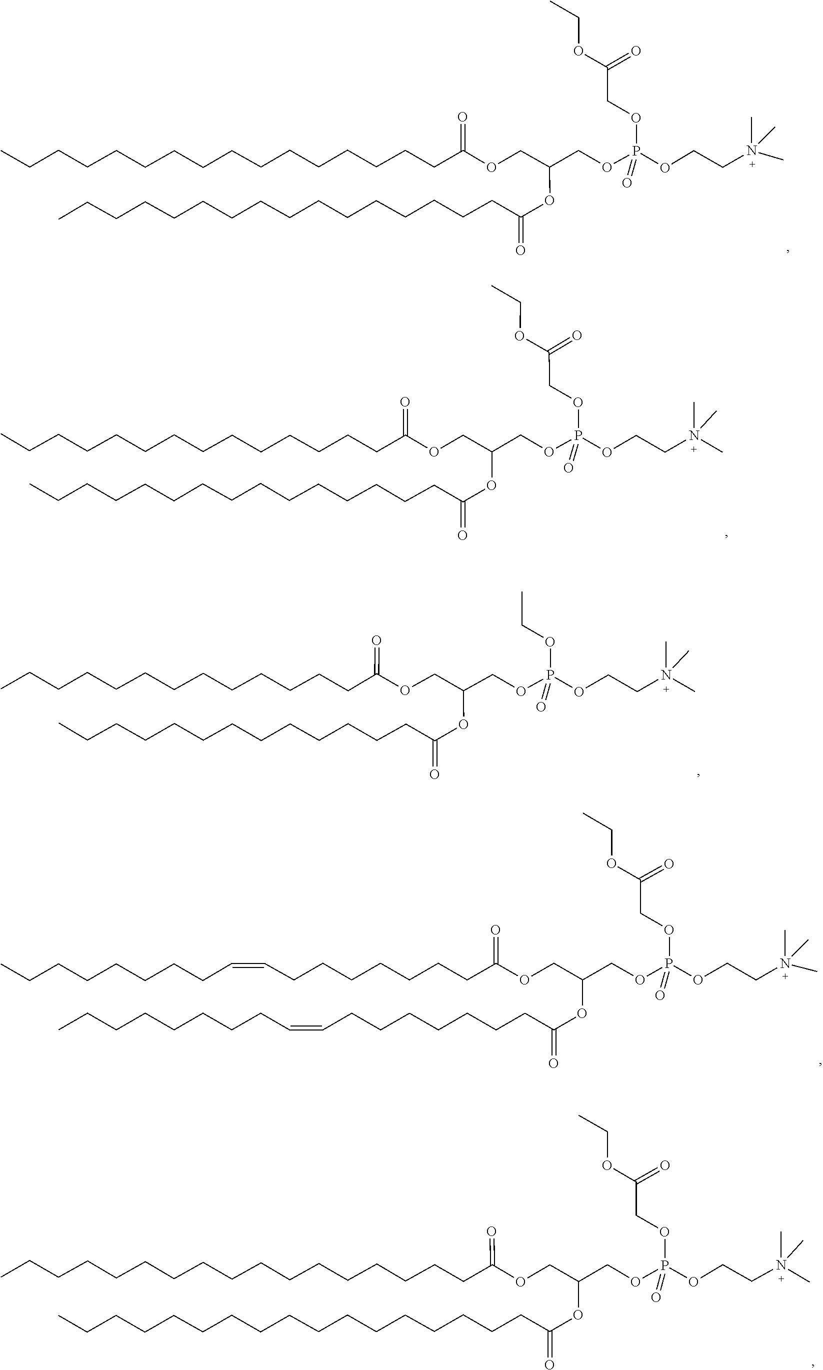

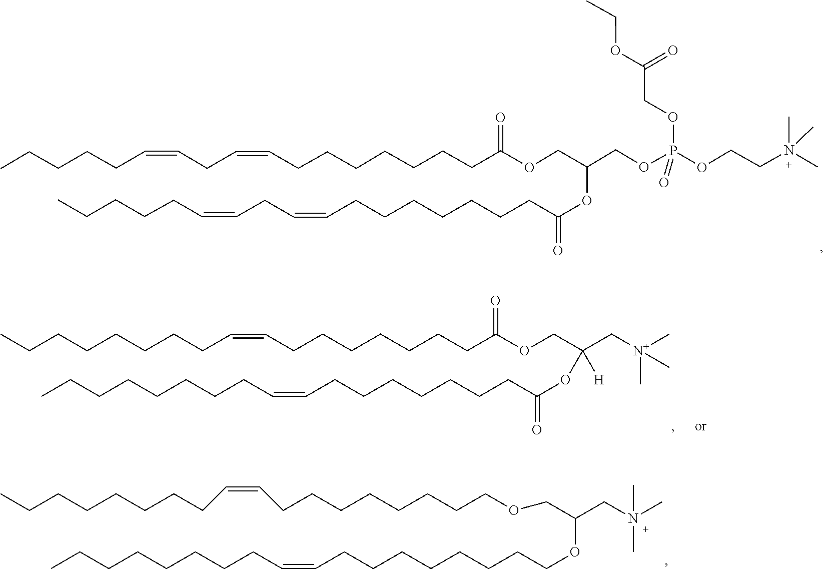

or a stereoisomer, or a mixture of stereoisomers thereof.

49. The lipid nanoparticle of embodiment 42, wherein the permanently cationic lipid is DOTMA, DOTAP, MVL5, DOGS, DC-Chol, DDAB, EPC, or a mixture thereof.

50. The lipid nanoparticle of any one of embodiments 42 to 49, wherein the lipid nanoparticle has a diameter of from 180 nm to about 900 nm, from about 300 nm to about 900 nm, from about 180 nm to about 600 nm, from about 180 nm to about 400 nm, from about 180 nm to about 350 nm, or from about 180 nm to about 300 nm.

51. The lipid nanoparticle of any one of embodiments 42 to 50, wherein the lipid nanoparticle has a diameter of from about 180 nm to about 300 nm.

52. The lipid nanoparticle of any one of embodiments 42 to 51, wherein the amount of permanently cationic lipid is from about 20 mol % to about 80 mol %, from about 30 mol % to about 70 mol %, from about 40 mol % to about 60 mol %, or from about 45 mol % to about 55 mol % of the total lipid present in the lipid nanoparticle.

53. The lipid nanoparticle of any one of embodiments 42 to 52, wherein the amount of the ionizable lipid is from about 15 mol % to about 40 mol %, or from about 20 mol % to about 30 mol % of the total lipid present in the lipid nanoparticle.

54. The lipid nanoparticle of any one of embodiments 42 to 53, wherein:
(i) the amount of the permanently cationic lipid is from about 40 mol % to about 60 mol % of the total lipid present in the lipid nanoparticle, and the amount of the ionizable lipid is from about 15 mol % to about 40 mol % of the total lipid present in the lipid nanoparticle; or
(ii) the amount of the permanently cationic lipid is from about 45 mol % to about 55 mol % of the total lipid present in the lipid nanoparticle, and the amount of the ionizable lipid is from about 20 mol % to about 30 mol % of the total lipid present in the lipid nanoparticle.

55. The lipid nanoparticle of any one of embodiments 42 to 54, wherein the ionizable lipid has a pKa of from about 7 to about 13, from about 7 to about 11, or from about 7 to about 9.

56. The lipid nanoparticle of any one of embodiments 42 to 55, further comprising phospholipid.

57. The lipid nanoparticle of embodiment 56, wherein the phospholipid is DSPC, DMPC, DOPC, DPPC, POPC, DOPE, DMPE, POPOE, or DPPE.

58. The lipid nanoparticle of any one of embodiments 42 to 55, wherein the lipid nanoparticle does not comprise a phospholipid or comprises a phospholipid in an amount less than about 15 mol 00 less than about 10 mol %, less than about 8 mol %, less than about 5 mol %, less than about 3 mol %, or less than about 1 mol % of the total lipid present in the lipid nanoparticle.

59. The lipid nanoparticle of any one of embodiments 42 to 58, further comprising a steroid.

60. The lipid nanoparticle of embodiment 59, wherein the steroid is cholesterol, campesterol, stigmasterol, sitosterol, brassicasterol, ergosterol, solanine, ursolic acid, alpha-tocopherol, beta-sitosterol, avenasterol, calciferol, or canola sterol.

61. The lipid nanoparticle of embodiment 59 or 60, wherein the amount of the steroid is from about 5 mol % to about 60 mol %, from about 10 mol % to about 50 mol %, from about 10 mol % to about 40 mol %, from about 20 mol % to about 30 mol %, or about 25 mol % of the total lipid present in the lipid nanoparticle.

62. The lipid nanoparticle of any one of embodiments 42 to 61, further comprising a pegylated lipid.

63. The lipid nanoparticle of embodiment 62, wherein a pegylated moiety of the pegylated lipid has a molecule weight of from about 1000 Da to about 10,000 Da, from about 1000 Da to about 5000 Da, or from about 1000 Da to about 2000 Da.

64. The lipid nanoparticle of embodiment 62 or 63, wherein the pegylated lipid is ALC-0159, DMG-PEG2000, DMPE-PEG1000, DPPE-PEG1000, DSPE-PEG1000, DOPE-PEG1000, Ceramide-PEG2000, DMPE-PEG2000, DPPE-PEG2000, DSPE-PEG2000, DSPE-PEG2000-Mannose, Ceramide-PEG5000, DSPE-PEG5000, or DSPE-PEG2000 amine.

65. The lipid nanoparticle of any one of embodiments 62 to 64, wherein the amount of the pegylated lipid is from about 0.1 mol to about 5 mol %, about 0.1 mol to about 3 mol %, from about 0.25 mol to about 2 mol %, from about 0.5 mol to about 1.5 mol %, or about 1 mol % of the total lipid present in the lipid nanoparticle.

66. The lipid nanoparticle of any one of embodiments 42 to 65, wherein the lipid nanoparticle comprises a therapeutic agent.

67. The lipid nanoparticle of embodiment 66, wherein a delivery efficiency of the therapeutic agent to a non-hepatic tissue by the lipid nanoparticle is higher than a delivery efficiency of the therapeutic agent to liver by the lipid nanoparticle, when the lipid nanoparticle is administered to a subject.

68. The lipid nanoparticle of embodiment 66 or 67, wherein the therapeutic agent is nucleic acid.

69. The lipid nanoparticle of embodiment 68, wherein the nucleic acid is antisense oligonucleotide (ASO), DNA, or RNA, optionally wherein the RNA is RNA interference (RNAi), small interfering RNA (siRNA), short hairpin RNA (shRNA), antisense RNA (aRNA), messenger RNA (mRNA), modified messenger RNA (mmRNA), long noncoding RNA (lncRNA), microRNA (miRNA), small activating RNA (saRNA), multicoding nucleic acid (MCNA), polymer-coded nucleic acid (PCNA), guide RNA (gRNA), CRISPR RNA (crRNA), or any other RNA in the ribozyme.

70. The lipid nanoparticle of embodiment 69, wherein the ratio of total number of nitrogen atoms in the permanently cationic lipid and ionizable lipid and total number of phosphate atoms in the nucleic acid is from about 1:1 to about 20:1, about 1:1 to about 15:1, from about 3:1 to about 12:1, or from about 4:1 to about 9:1.

71. The lipid nanoparticle of any one of embodiments 42 to 70, wherein the lipid nanoparticle has an apparent pKa of greater than 7, greater than 8, greater than 9, greater than 10, from about 7 to about 10, or greater than 10.

72. A population of lipid nanoparticles comprising the lipid nanoparticle of any one of embodiments 42 to 71, wherein the population of lipid nanoparticles have an average diameter of from about 160 nm to about 900 nm.

73. The population of lipid nanoparticles of embodiment 72, wherein the average diameter is determined by dynamic light scattering (DLS).

74. A pharmaceutical composition comprising the lipid nanoparticle of any one of embodiments 42 to 71 or the population of lipid nanoparticles of embodiment 72 or 73 and a pharmaceutically acceptable carrier.

75. A method of delivering or expressing a therapeutic agent in the lung of a subject or treating or preventing a lung disease in a subject, wherein the method comprises using a lipid nanoparticle comprising the therapeutic agent, wherein the lipid nanoparticle is administered intravenously, intraarterially, or intraperitoneally to the subject, wherein the lipid nanoparticle has a positive surface charge, and wherein the lipid nanoparticle has a diameter of from about 160 nm to about 900 nm.

76. A method of delivering or expressing a therapeutic agent in the lung of a subject or treating or preventing a lung disease in a subject, wherein the method comprises using a lipid nanoparticle comprising the therapeutic agent, wherein the lipid nanoparticle comprises a permanently cationic lipid and an ionizable lipid, and wherein the lipid nanoparticle has a diameter of from about 160 nm to about 900 nm.

77. A method of treating or preventing a lung disease in a subject, comprising administering to the subject a therapeutically effective amount of the lipid nanoparticle of any one of embodiments 42 to 71, the population of lipid nanoparticles of embodiment 72 or 73, or the pharmaceutical composition of embodiment 74.

78. The method of embodiment 77, wherein the administration is intravenous administration, intraarterial administration, or intraperitoneal administration.

79. A method of producing the lipid nanoparticle of any one of embodiments 42 to 71 or the population of lipid nanoparticles of embodiment 72 or 73 comprising the steps of:
(i) dissolving in a first solution a mixture comprising a permanently cationic lipid and an ionizable lipid to form a lipid solution, wherein the lipid solution is formed in an organic solvent;
(ii) dissolving in a second solution a therapeutic agent to form a therapeutic agent solution; and
(iii) mixing the lipid solution and the therapeutic agent solution at a mixing speed of about 1 mL/min to about 18 mL/min, about 1 mL/min to about 10 mL/min, or about 2 mL/min to about 6 mL/min.

80. The method of embodiment 79, wherein the organic solvent is ethanol.

81. The method of embodiment 79 or 80, wherein the second solution is a sodium acetate buffer having a pH of about 4.5.

82. The method of any one of embodiments 79 to 81, wherein the lipid solution and the therapeutic agent solution are mixed at a volumetric ratio of from about 1:1 to about 1:10, about 1:1 to about 1:6, or about 1:1 to about 1:4.

### 6. EXAMPLES

In order to make the technical solutions of the present disclosure clearer and more explicit, the present disclosure is further elaborated through the following examples. The following examples are used only to illustrate specific embodiments of the present disclosure so that a person skilled in the art can understand the present application, but are not intended to limit the scope of protection of the application. The technical means or methods, etc. not specifically described in the specific embodiments of the present disclosure are conventional technical means or methods, etc. in the art. The materials, reagents, etc. used in examples are commercially available if not otherwise specified.

TABLE 1

| Abbreviation | Full name |
|---|---|
| THF | Tetrahydrofuran |
| DCM | dichloromethane |
| MeOH | methanol |
| DMF | N, N-Dimethylformamide |
| DMSO | Dimethyl sulfoxide |
| DCE | 1, 2-Dichloroethane |
| CDCl₃ | Deuterated chloroform |
| TBAI | Tetrabutylammonium iodide |
| TsCH₂CN | 4-Toluenesulfonylacetonitrile |
| TMSOK | Potassium trimethylsiloxide |
| TBDMSCl | tert-Butyldimethylsilyl chloride |
| LDA | Lithium diisopropylamide |
| DMAP | 4-Dimethylaminopyridine |
| (COCl)₂ | Oxalyl chloride |
| SOCl₂ | Thionyl dichloride |
| NaBH₄ | Sodium borohydride |
| NaH | Sodium hydride |
| K₂CO₃ | Potassium carbonate |
| EDCI | 1-(3-Dimethylaminopropyl)-3-ethylcarbodiimide |
| DIPEA | N,N-Diisopropylethylamine |
| Et₃N | Triethylamine |
| AcOH | Acetic acid |
| NaBH₃CN | Sodium cyanoborohydride |
| Imidazole | Imidazole |
| NMO | 4-Methylmorpholine N-oxide |
| BDMEP | 2,6-di-tert-Butylpyridine |

### Example 1: Synthesis of Compound 1

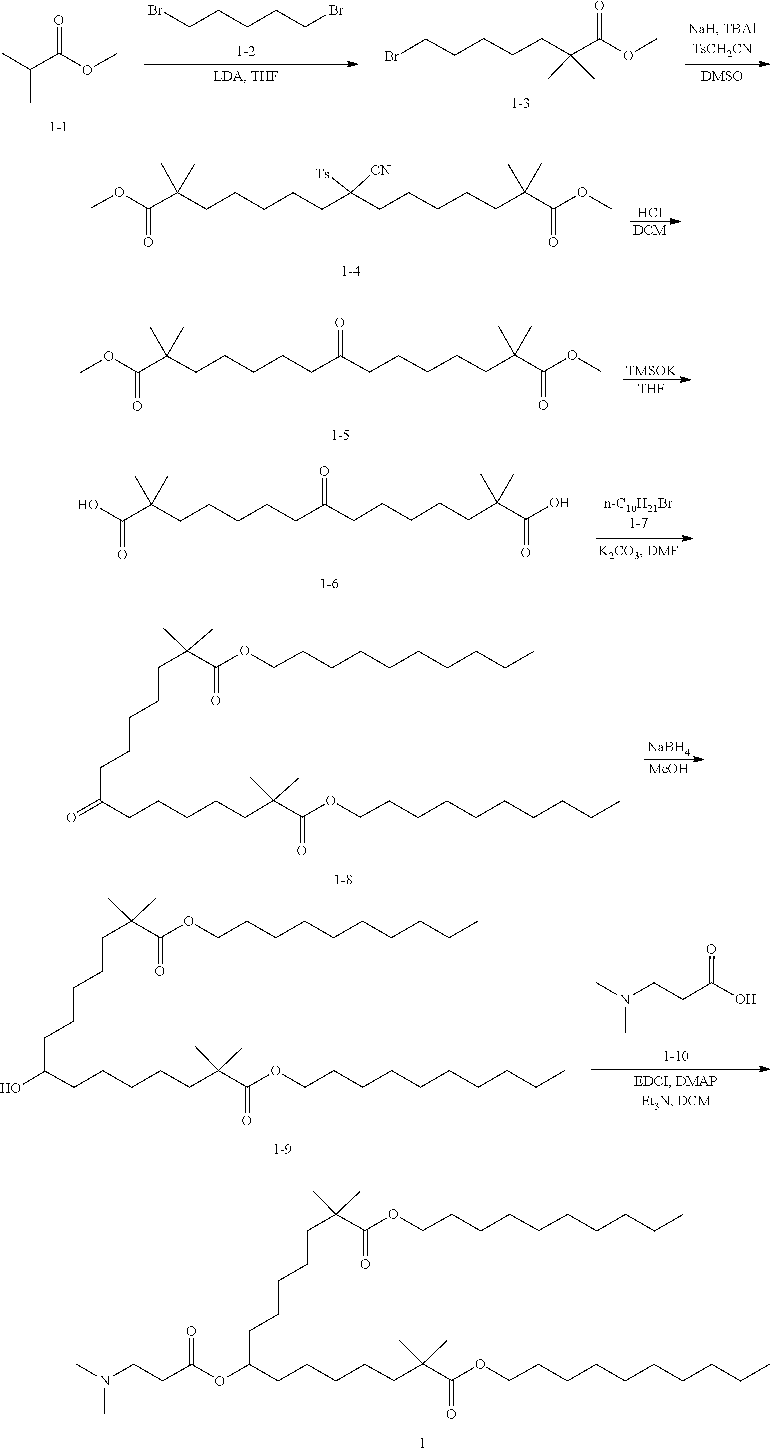

A solution of compound 1-1 (100 g, 979 mmol) in tetrahydrofuran (800 mL) was cooled to −40° C. LDA (2 M, 490 mL) was added slowly dropwise to the solution and the mixture was stirred for another 1 h after completion of the dropwise addition. A solution of 1-2 (315 g, 1.37 mol) in tetrahydrofuran (100 mL) was added dropwise to the reaction system at the same temperature and the reaction system was stirred overnight. The reaction system was quenched with saturated aqueous ammonium chloride, and extracted with ethyl acetate. The organic phases were combined, dried over anhydrous sodium sulfate, and filtered. The filtrate was concentrated to dryness to give the crude product. The crude product was purified by silica gel column to give compound 1-3 (115 g). ¹H NMR (400 MHz, CDCl₃): δ ppm 1.06-1.11 (m, 6H), 1.13-1.22 (m, 2H), 1.29-1.39 (m, 2H), 1.42-1.49 (m, 2H), 1.73-1.82 (m, 2H), 3.28-3.40 (m, 2H), 3.55-3.66 (m, 3H).

A solution of compound 1-3 (100 g, 398 mmol), TsCH₂CN (38.9 g, 199 mmol) and TBAI (14.7 g, 39.8 mmol) in dimethyl sulfoxide (800 mL) was cooled to 0° C., and sodium hydride (20.7 g, 517 mmol) was added slowly in batches. The mixture was reacted at room temperature overnight. The reaction system was quenched with saturated aqueous sodium chloride solution and extracted with ethyl acetate. The organic phases were combined, dried over anhydrous sodium sulfate, and filtered. The filtrate was concentrated to dryness to give 115 g of crude compound 1-4, which was used directly in the next reaction without isolation and purification.

To a solution of compound 1-4 crude (110 g, 205 mmol) in dichloromethane (880 mL) was added 330 mL of concentrated hydrochloric acid, and the mixture was reacted at room temperature for 2 h. The complete reaction of the substrate was monitored by TLC. The reaction system was quenched with saturated aqueous ammonium chloride solution and extracted with ethyl acetate. The organic phases were combined, dried over anhydrous sodium sulfate, and filtered. The filtrate was concentrated to dryness to give the crude product. The crude product was purified by silica gel column to give compound 1-5 (30.0 g, 80.9 mmol, yield 39.4%).

TMSOK (11.0 g, 86.4 mmol) was added to a solution of compound 1-5 (8.0 g, 21.6 mmol) in tetrahydrofuran (35.0 mL) at room temperature, and the reaction system was heated to 70° C. with stirring. The complete consumption of reaction materials was monitored by TLC. The reaction solution was cooled to room temperature, and the organic solvent was removed by rotary evaporation. The crude product was added to 20 mL of water and extracted with dichloromethane. The aqueous layer was collected, and the solution was adjusted to a pH of <5 with 1 M hydrochloric acid. The solution was extracted with dichloromethane. The organic phases were combined, dried over anhydrous sodium sulfate, and filtered. The filtrate was collected and concentrated to give compound 1-6 (7.0 g). ¹H NMR (400 MHz, CDCl₃): δ ppm 1.03 (s, 12H), 1.08-1.17 (m, 8H), 1.34-1.45 (m, 8H), 2.21 (t, J=7.2 Hz, 4H).

Potassium carbonate (482 mg, 3.48 mmol) was added to a solution of compound 1-6 (294 mg, 0.87 mmol) and 1-7 (771 mg, 3.48 mmol) in DMF, then the reaction was warmed up to 60° C. for 6 h. The complete disappearance of reactant 1-6 was monitored. The mixture was cooled to room temperature. The reaction system was quenched with saturated aqueous sodium chloride solution and extracted with ethyl acetate. The organic phases were combined, dried over anhydrous sodium sulfate, and filtered. The filtrate was concentrated to dryness to give the crude product. The crude was purified by silica gel column to give compound 1-8 (325 mg).

Compound 1-8 (325 mg) was dissolved in 4.0 mL of methanol and sodium borohydride (30 mg, 0.84 mmol) was added to the reaction system. The mixture was reacted at room temperature. The complete disappearance of the reactants was monitored by TLC. The reaction system was quenched with saturated aqueous sodium chloride solution and extracted with dichloromethane. The organic phases were combined, dried over anhydrous sodium sulfate, and filtered. The filtrate was concentrated to dryness to give crude compound 1-9 (260 mg), which was used directly in the next reaction without purification.

Crude compound 1-9 (260 mg, 0.42 mmol), 1-10 (73.1 mg, 0.63 mmol), EDCI (238 mg, 1.26 mmol), triethylamine (0.17 mL, 1.26 mmol) and DMAP (51 mg, 0.42 mmol) were dissolved in 5.0 mL of dichloromethane, and the reaction solution was stirred to react at room temperature for 12 h. The reaction solution was quenched with saturated aqueous sodium chloride and extracted with dichloromethane. The organic phases were combined, dried over anhydrous sodium sulfate, and filtered. The organic phase was collected and the organic solvent was removed using a rotary-evaporator to give the crude product, which was purified by preparative high performance liquid chromatography to give compound 1 (130 mg). ¹H NMR (400 MHz, CDCl₃): δ ppm 0.89 (t, J=7.2 Hz, 6H), 1.15 (s, 12H), 1.27 (m, 40H), 1.49 (m, 8H), 1.61 (m, 4H), 2.26 (s, 6H), 2.44-2.52 (t, J=7.2 Hz, 2H), 2.63 (t, J=7.2 Hz, 2H), 4.04 (t, J=6.8 Hz, 4H), 4.86 (m, 1H); ESI-MS m/z: 724.7 [M+H]⁺.

### Example 2: Synthesis of Compound 2

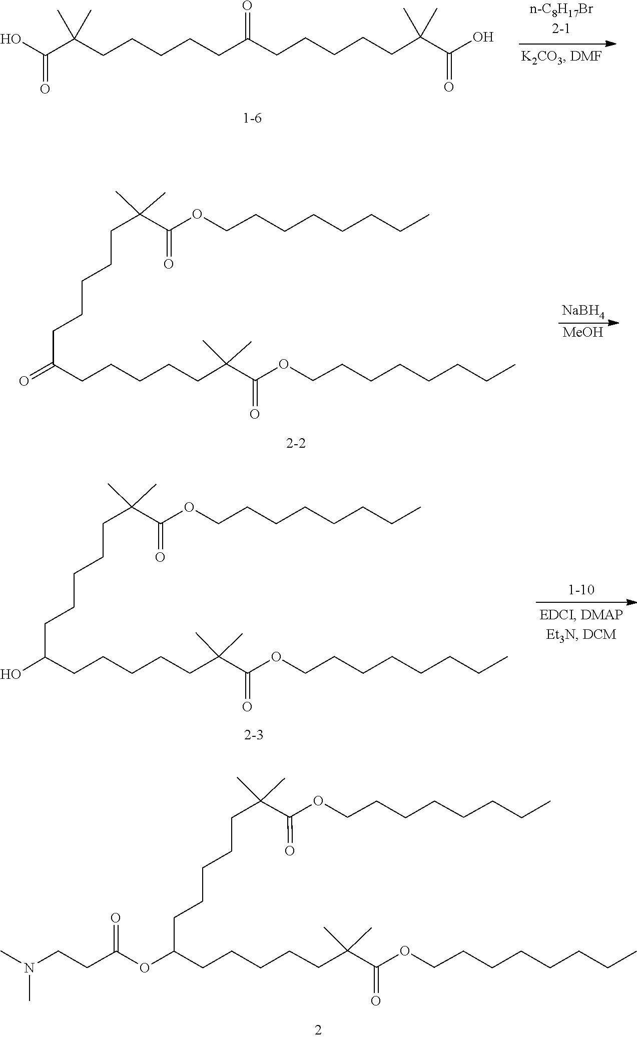

Referring to the method of Example 1, compound 2 was prepared as an oily product: 25.7 mg. ¹H NMR (400 MHz, CDCl₃): δ ppm 0.89 (t, J=6.8 Hz, 6H), 1.15 (s, 12H), 1.29 (m, 32H), 1.49 (m, 8H), 1.60 (m, 4H), 2.24 (s, 6H), 2.46 (t, J=7.2 Hz, 2H), 2.61 (t, J=7.2 Hz, 2H), 4.04 (t, J=6.8 Hz, 4H), 4.86 (m, 1H); ESI-MS m/z: 668.6 [M+H]⁺.

### Example 3: Synthesis of Compound 3

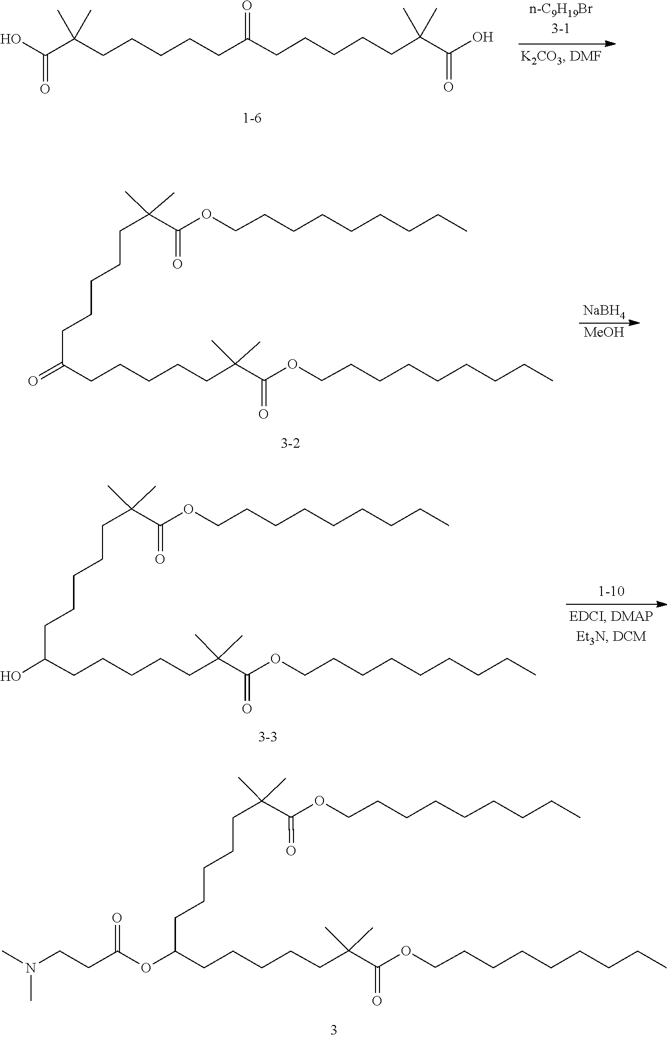

Referring to the method of Example 1, compound 3 was prepared as an oily product: 31.2 mg. ¹H NMR (400 MHz, CDCl₃): δ ppm 0.89 (t, J=6.8 Hz, 6H), 1.16 (s, 12H), 1.28 (m, 36H), 1.49 (m, 8H), 1.62 (m, 4H), 2.25 (s, 6H), 2.47 (t, J=7.2 Hz, 2H), 2.62 (t, J=7.2 Hz, 2H), 4.05 (t, J=6.8 Hz, 4H), 4.88 (m, 1H); ESI-MS m/z: 696.6 [M+H]⁺.

### Example 4: Synthesis of Compound 4

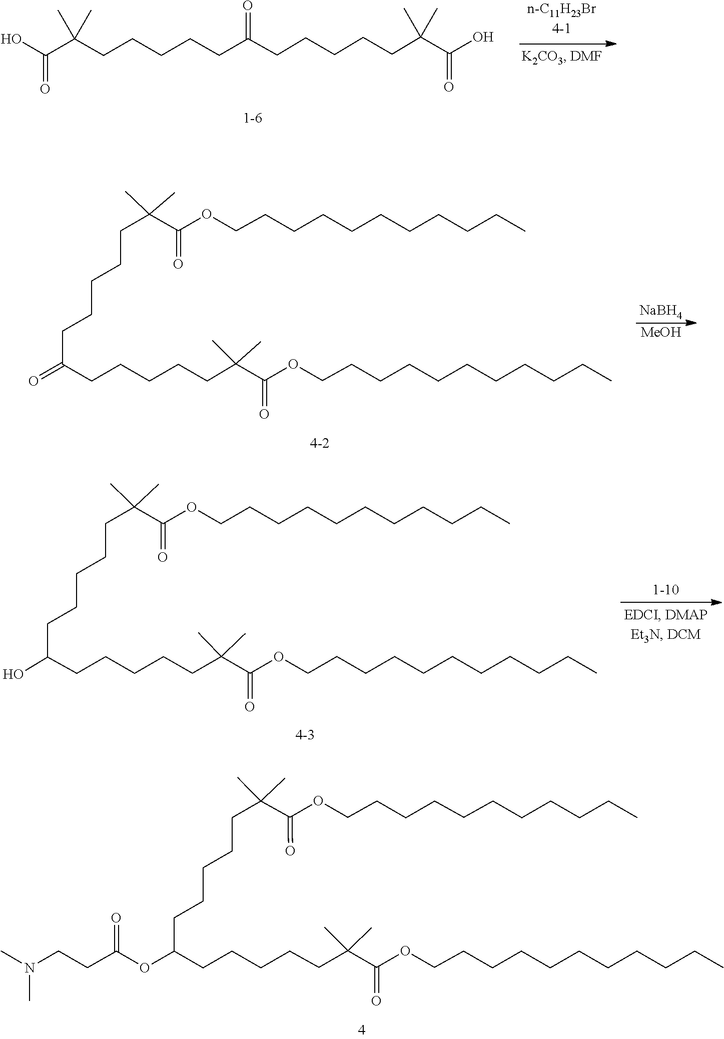

Referring to the method of Example 1, compound 4 was prepared as an oily product: 32 mg. H NMR (400 MHz, CDCl₃): δ ppm 0.89 (t, J=6.8 Hz, 6H), 1.16 (s, 12H), 1.28 (m, 44H), 1.49 (m, 8H), 1.52 (m, 4H), 2.51 (s, 6H), 2.53 (t, J=7.2 Hz, 2H), 3.12 (t, J=7.2 Hz, 2H), 3.91 (t, J=6.8 Hz, 4H), 4.82 (m, 1H); ESI-MS m/z: 752.7 [M+H]⁺.

### Example 5: Synthesis of Compound 5

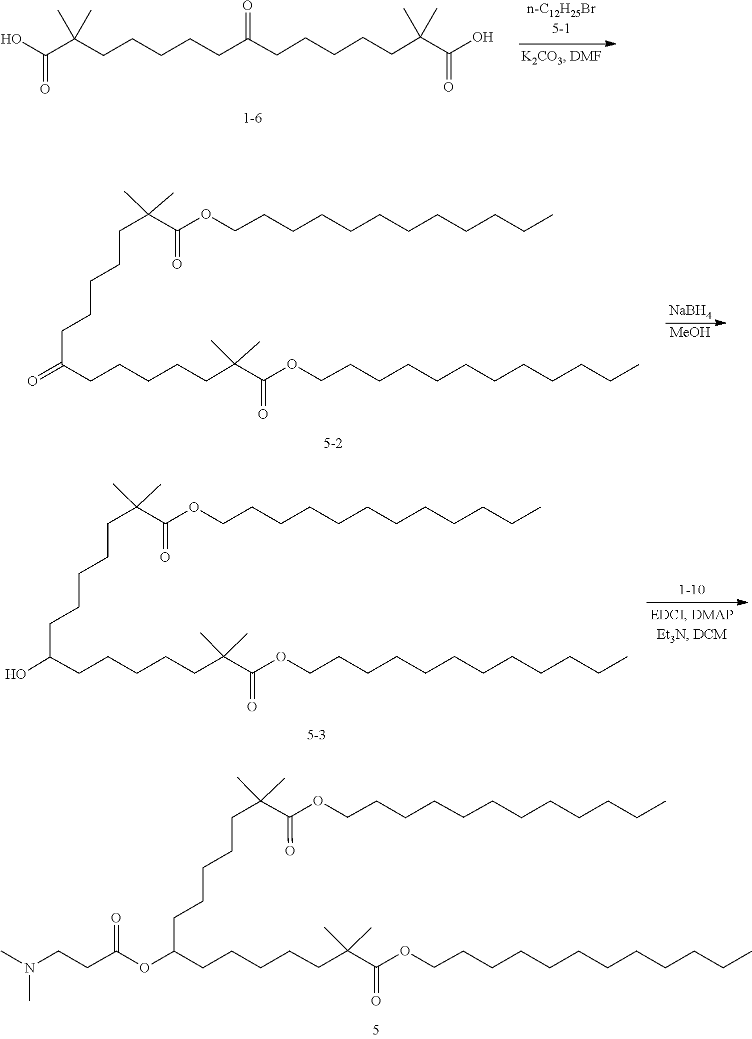

Referring to the method of Example 1, compound 5 was prepared as an oily product: 31.4 mg. ¹H NMR (400 MHz, CDCl₃): δ ppm 0.88 (t, J=6.8 Hz, 6H), 1.15 (s, 12H), 1.25 (m, 48H), 1.49 (m, 8H), 1.52 (m, 4H), 2.46 (s, 6H), 2.63 (m, 2H), 2.86 (m, 2H), 4.03 (t, J=6.8 Hz, 4H), 4.84 (m, 1H); ESI-MS m/z: 780.7 [M+H]⁺.

### Example 6: Synthesis of Compound 6

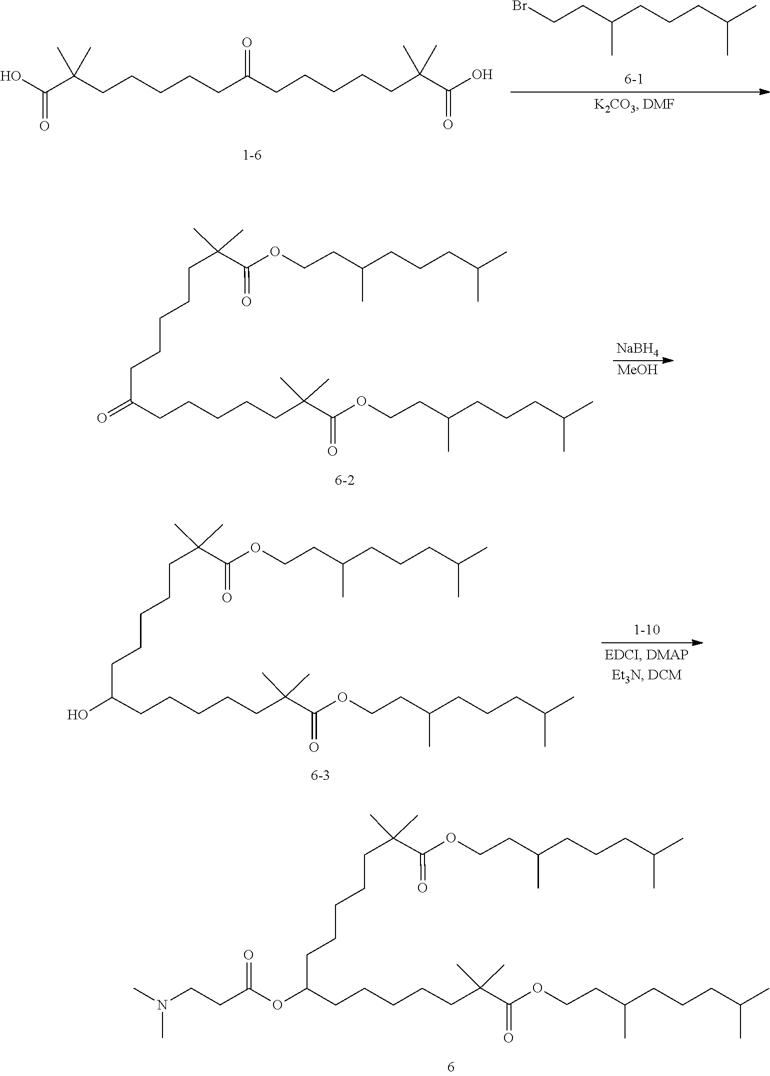

Referring to the method of Example 1, compound 6 was prepared as an oily product: 30.7 mg. ¹H NMR (400 MHz, CDCl₃): δ ppm 0.83 (t, J=6.8 Hz, 18H), 1.00-1.28 (m, 34H), 1.31-1.62 (m, 18H), 2.21 (s, 6H), 2.36-2.46 (m, 2H), 2.51-2.62 (m, 2H), 4.02 (t, J=6.8 Hz, 4H), 4.71-4.85 (m, 1H); ESI-MS m/z: 724.6 [M+H]⁺.

### Example 7: Synthesis of Compound 7

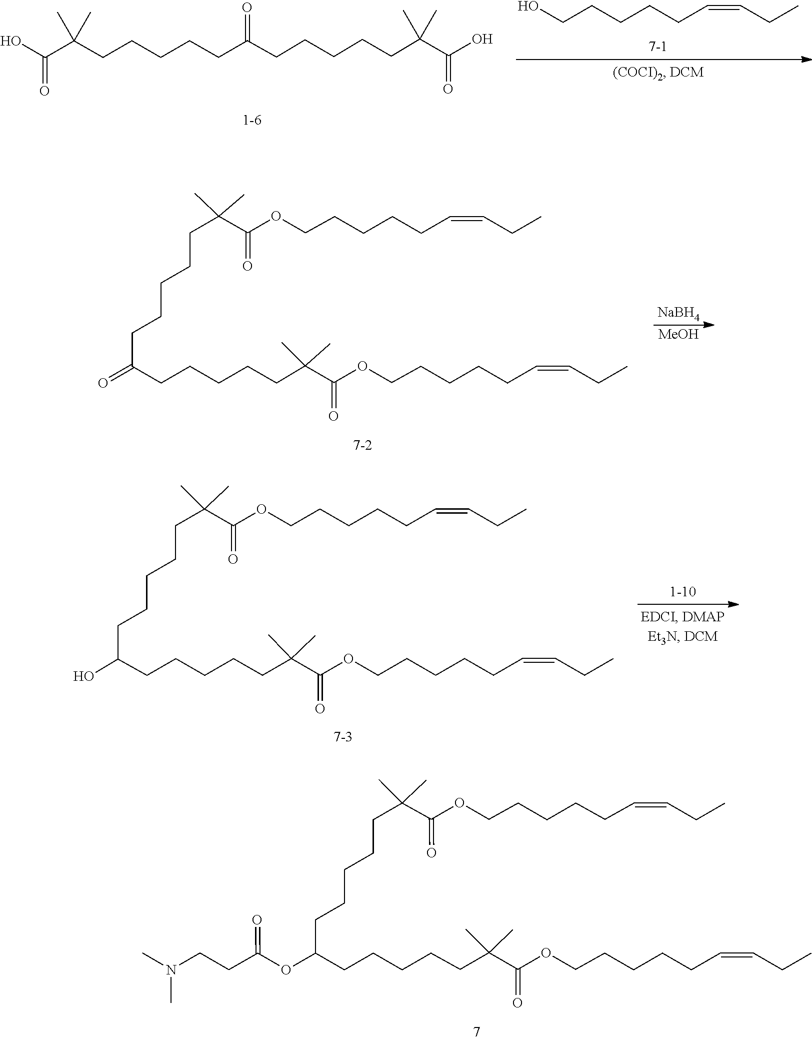

Compound 1-6 (548 mg, 1.5 mmol) was dissolved in 5.0 mL of dichloromethane, and the reaction system was cooled to 0° C. in an ice bath. DMF (12 μL, 0.15 mmol) was added and oxalyl chloride (0.47 mL, 6.0 mmol) was added dropwise to the reaction solution. The ice bath was removed and the mixture was stirred for 1 h at room temperature. The solvent was removed using a rotary-evaporator to give acyl chloride crude product (458 mg) as an oil, which was used directly in the next reaction step.

The above obtained acyl chloride crude product (458 mg) was dissolved in 3.0 mL of 1,2-dichloroethane, and then compound 7-1 (429 mg, 3.0 mmol) was added to the reaction solution. The mixture was stirred at room temperature until the substrate was reacted completely. The solvent was removed using a rotary-evaporator to give the crude product, which was purified by silica gel column to give compound 7-2 (540 mg).

Then referring to the method of Example 1, compound 7 was prepared as an oily product: 33.2 mg. ¹H NMR (400 MHz, CDCl₃): δ ppm 0.89 (t, J=6.8 Hz, 6H), 1.23 (s, 12H), 1.29-1.51 (m, 32H), 1.95 (m, 8H), 2.18 (s, 6H), 2.41 (m, 2H), 2.53 (m, 2H), 3.91 (t, J=6.8 Hz, 4H), 4.78 (m, 1H), 5.25 (m, 4H); ESI-MS m/z: 692.6 [M+H]⁺.

### Example 8: Synthesis of Compound 8

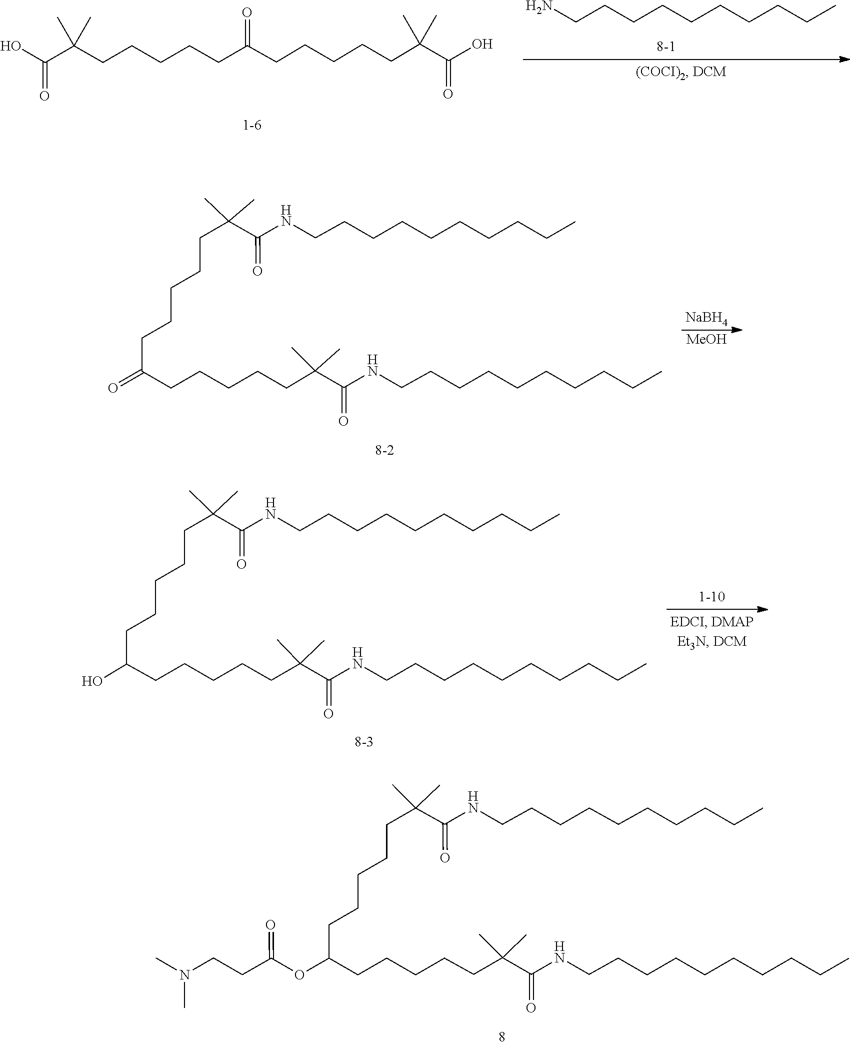

Compound 1-6 (548 mg, 1.5 mmol) was dissolved in 5.0 mL of dichloromethane, and the reaction system was cooled in an ice bath. DMF (12 μL, 0.15 mmol) was added and oxalyl chloride (0.47 mL, 6.0 mmol) was added dropwise to the reaction solution. The ice bath was removed and the mixture was stirred for 1 h at room temperature. The solvent was removed using a rotary-evaporator to give acyl chloride crude product (458 mg) as an oil, which was used directly in the next reaction step.

The above obtained 458 mg of acyl chloride crude product was dissolved in 3.0 mL of 1,2-dichloroethane, and then compound 8-1 (472 mg, 3.0 mmol) was added to the reaction solution. The mixture was stirred at room temperature until the substrate was reacted completely. The solvent was removed using a rotary-evaporator to give crude product, which was purified by silica gel column to give compound 8-2 (518 mg).

518 mg of compound 8-2 was dissolved in 5.0 mL of methanol, and sodium borohydride (48 mg, 1.25 mmol) was added to the reaction system. The mixture was reacted at room temperature. The complete disappearance of the reactants was monitored by TLC. The reaction system was quenched with saturated aqueous sodium chloride solution and extracted with dichloromethane. The organic phases were combined, dried over anhydrous sodium sulfate, and filtered. The filtrate was concentrated to dryness to give 473 mg of crude compound 8-3, which was used directly in the next reaction without purification.

Crude compound 8-3 (270 mg, 0.43 mmol), 1-10 (76.1 mg, 0.65 mmol), EDCI (248 mg, 1.3 mmol), triethylamine (0.18 mL, 1.3 mmol) and DMAP (53 mg, 0.43 mmol) were dissolved in 5.0 mL of dichloromethane, and the reaction solution was stirred to react at room temperature for 12 h. The reaction system was quenched with saturated aqueous sodium chloride and extracted with dichloromethane. The organic phases were combined, dried over anhydrous sodium sulfate, and filtered. The organic phase was collected and the organic solvent was removed using a rotary-evaporator to give the crude product, which was purified by preparative high performance liquid chromatography to give compound 8 (39 mg). ¹H NMR (400 MHz, CDCl₃): δ ppm 0.89 (t, J=6.8 Hz, 6H), 1.14 (s, 12H), 1.15-1.31 (m, 40H), 1.40-1.52 (m, 12H), 2.25 (s, 6H), 2.45 (m, 2H), 2.60 (m, 2H), 3.15 (t, J=6.8 Hz, 4H), 4.77-4.89 (m, 1H), 5.51-5.67 (m, 2H); ESI-MS m/z: 722.7 [M+H]⁺.

### Example 9: Synthesis of Compound 9

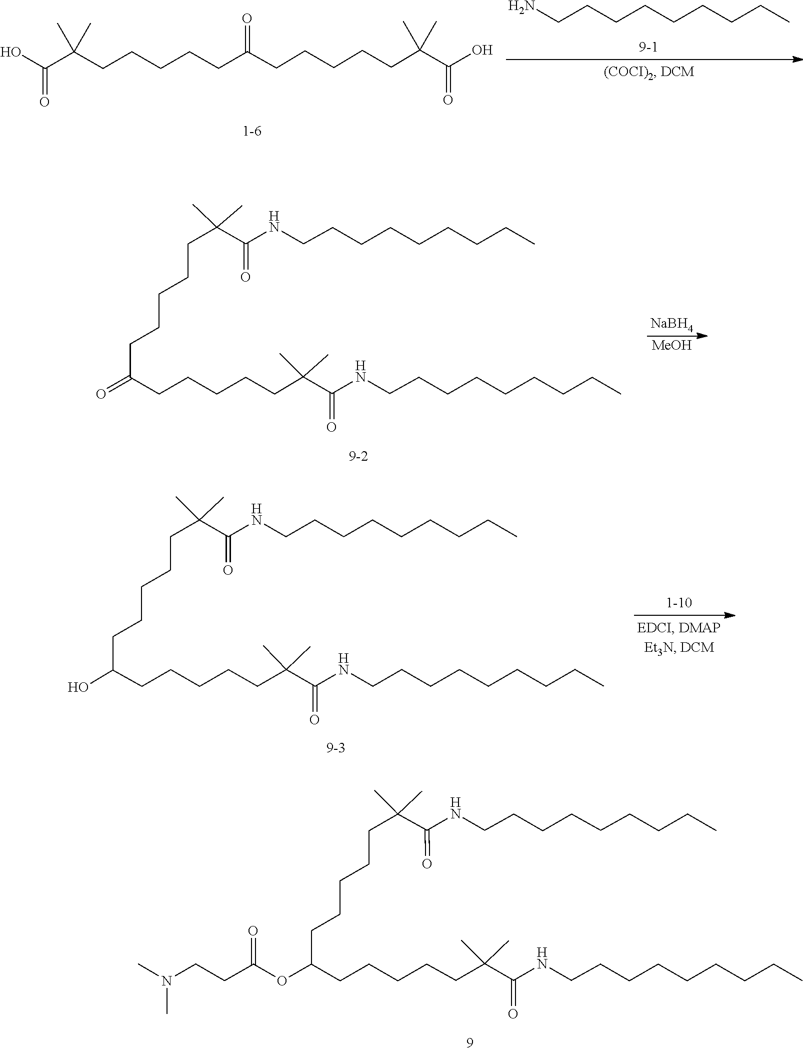

Referring to the method of Example 8, compound 9 (73 mg) was prepared. ¹H NMR (400 MHz, CDCl₃): δ ppm 0.89 (t, J=6.8 Hz, 6H), 1.15 (s, 12H), 1.27-1.49 (m, 48H), 2.25 (s, 6H), 2.46 (t, J=7.2 Hz, 2H), 2.62 (t, J=7.2 Hz, 2H), 3.24 (m, 4H), 4.85 (m, 1H), 5.58 (m, 2H); ESI-MS m/z: 694.6 [M+H]⁺.

### Example 10: Synthesis of Compound 10

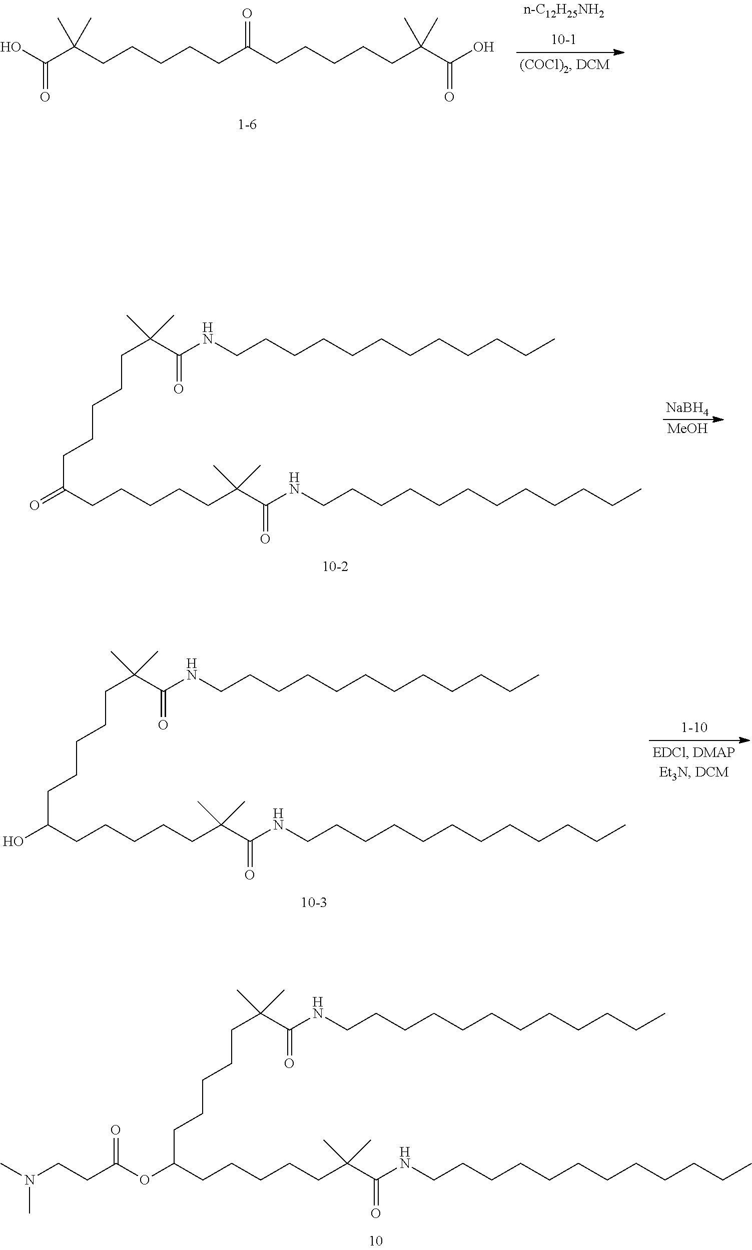

Referring to the method of Example 8, compound 10 (31.2 mg) was prepared. ¹H NMR (400 MHz, CDCl₃): δ ppm 0.79 (t, J=7.2 Hz, 6H), 1.07 (s, 12H), 1.27-1.49 (m, 48H), 1.41 (m, 12H), 2.18 (s, 6H), 2.41 (t, J=7.2 Hz, 2H), 2.55 (t, J=7.2 Hz, 2H), 3.16 (m, 4H), 4.78 (m, 1H), 5.51 (m, 2H); ESI-MS m/z: 778.8 [M+H]⁺.

### Example 11: Synthesis of Compound 11

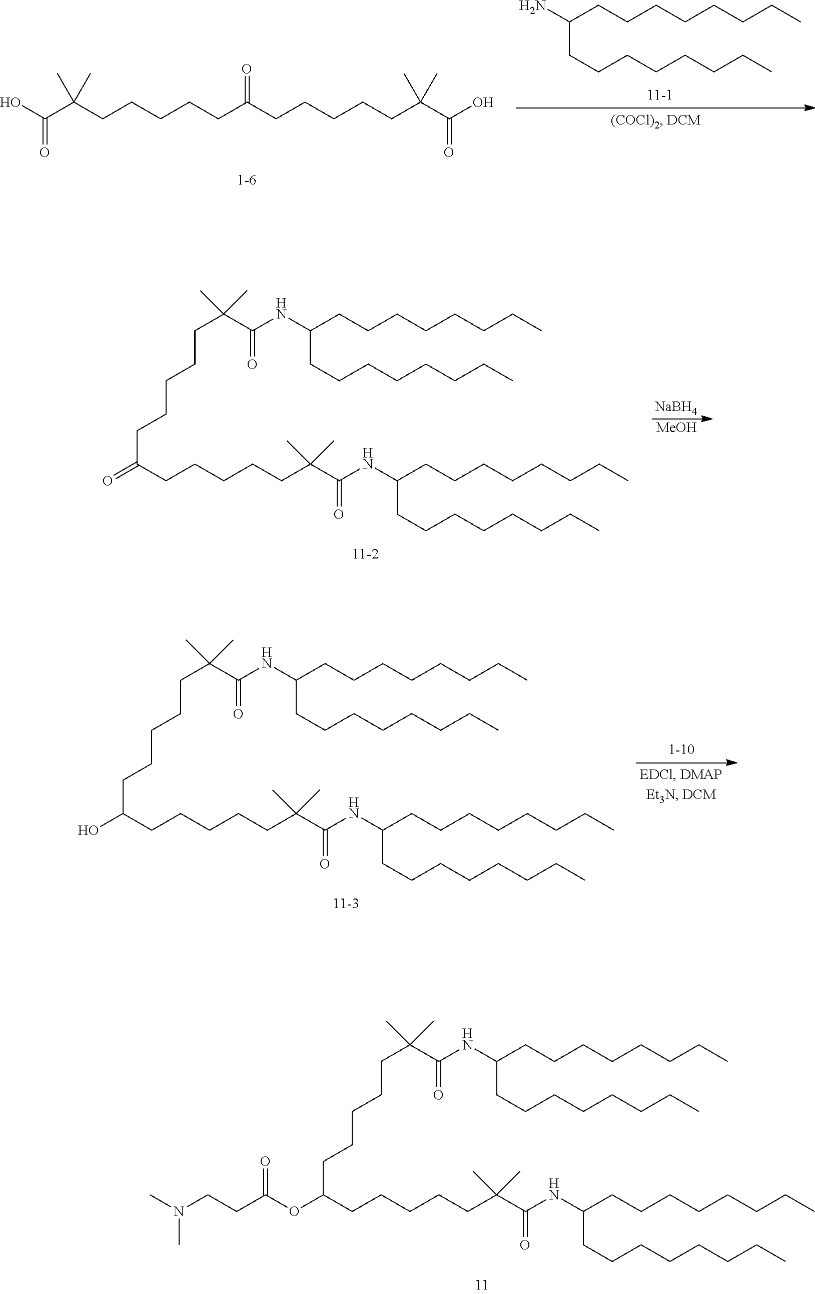

Referring to the method of Example 8, compound 11 (48.1 mg) was prepared. ¹H NMR (400 MHz, CDCl₃): δ ppm 0.78 (t, J=7.2 Hz, 12H), 1.07 (s, 12H), 1.14-1.19 (m, 60H), 1.40 (m, 16H), 2.18 (s, 6H), 2.36-2.47 (m, 2H), 2.49-2.68 (m, 2H), 3.76-3.88 (m, 2H), 4.74-4.83 (m, 1H), 5.10-5.19 (m, 2H); ESI-MS m/z: 918.9 [M+H]⁺.

### Example 12: Synthesis of Compound 12

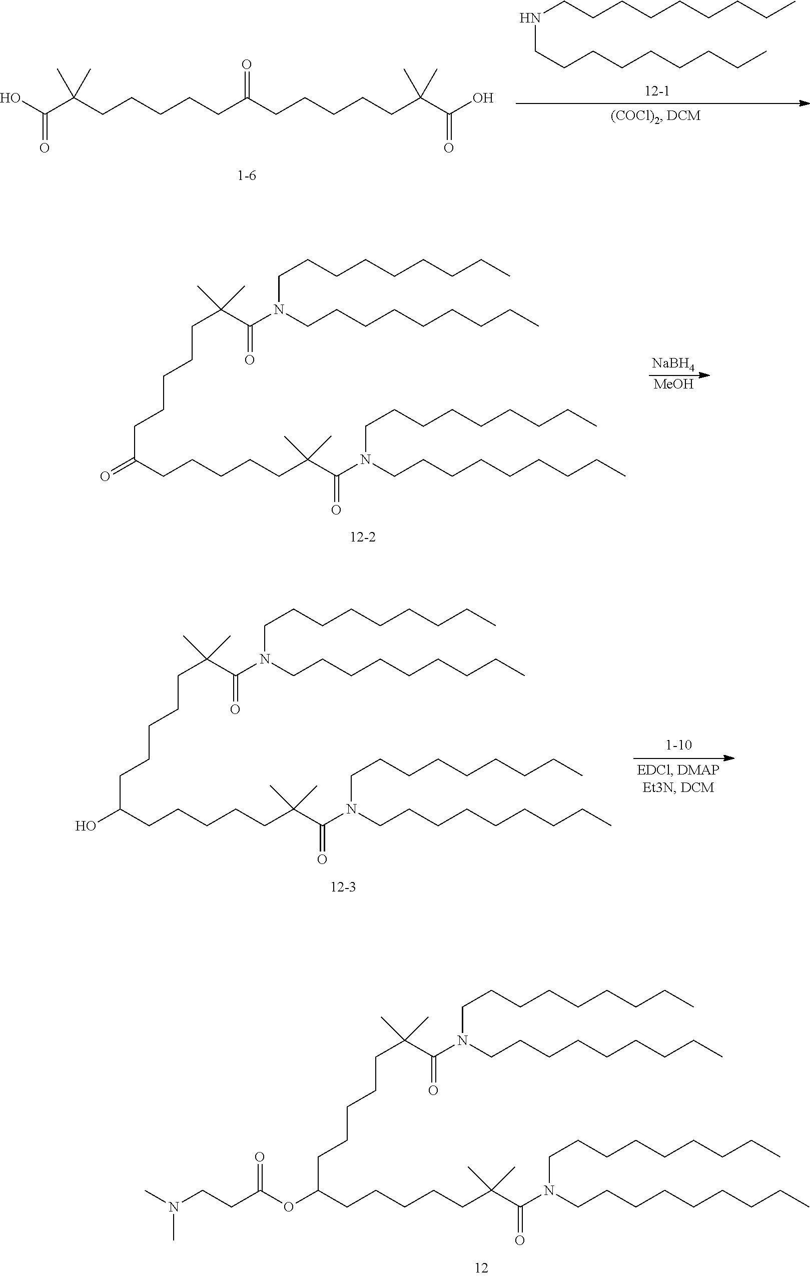

Referring to the method of Example 8, compound 12 (52 mg) was prepared. ¹H NMR (400 MHz, CDCl₃): δ ppm 0.82 (t, J=6.8 Hz, 12H), 1.15-1.32 (m, 72H), 1.54 (m, 16H), 2.31 (s, 6H), 2.51 (t, J=7.2 Hz, 2H), 2.60 (t, J=7.2 Hz, 2H), 3.14-3.33 (m, 8H), 4.75-4.83 (m, 1H); ESI-MS m/z: 946.9 [M+H]⁺.

### Example 13: Synthesis of Compound 13

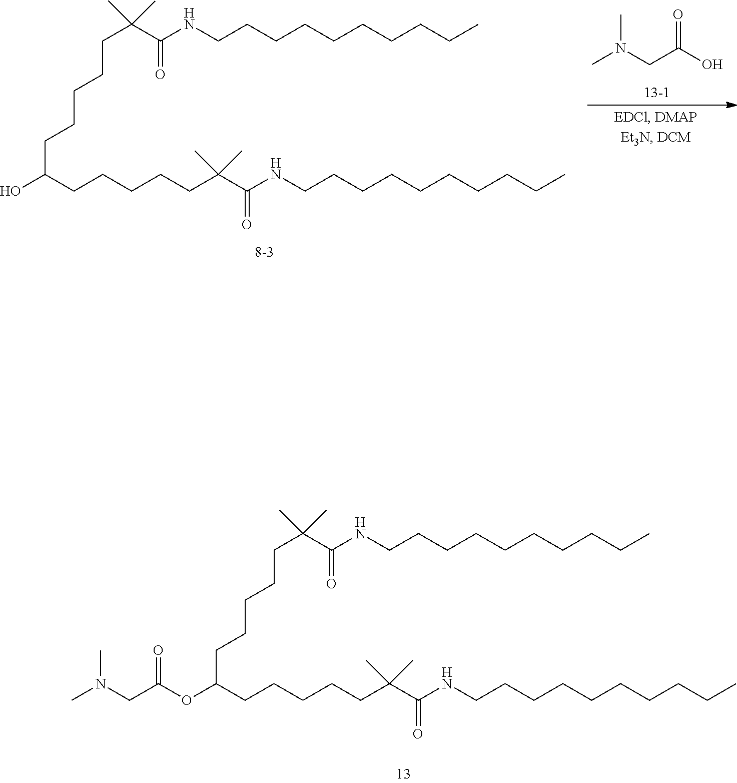

Referring to the method of Example 8, compound 13 (32 mg) was prepared. ¹H NMR (400 MHz, CDCl₃): δ ppm 0.81 (t, J=7.2 Hz, 6H), 1.19 (m, 52H), 1.41 (m, 12H), 2.26 (s, 6H), 3.05 (s, 2H), 3.16 (m, 4H), 4.83 (m, 1H), 5.51 (m, 2H); ESI-MS m/z: 708.7 [M+H]⁺.

### Example 14: Synthesis of Compound 14

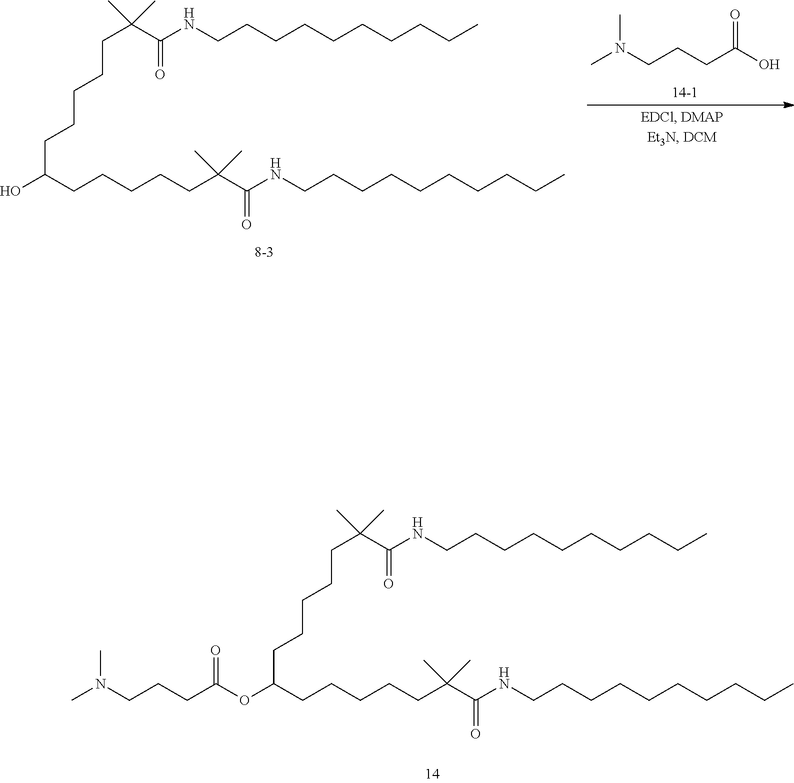

Referring to the method of Example 8, compound 14 (18 mg) was prepared. ¹H NMR (400 MHz, CDCl₃): δ ppm 0.81 (t, J=7.2 Hz, 6H), 1.07 (s, 12H), 1.08-1.31 (m, 44H), 1.35-1.47 (m, 8H), 1.71-1.84 (m, 2H), 2.09-2.38 (m, 10H), 3.12-3.27 (m, 4H), 4.70-4.82 (m, 1H), 5.49-5.63 (m, 2H); ESI-MS m/z: 736.7 [M+H]⁺.

### Example 15: Synthesis of Compound 15

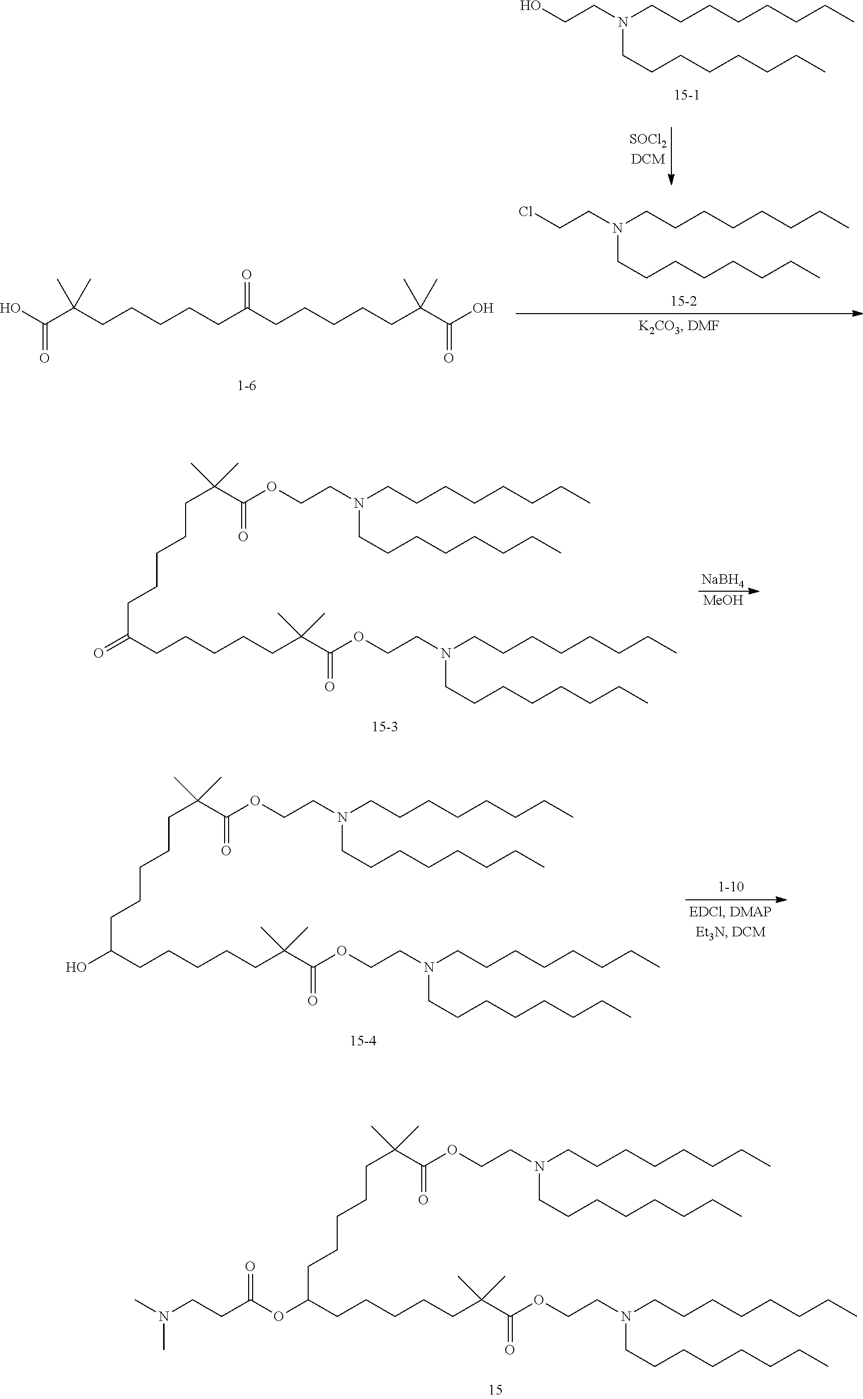

A solution of compound 15-1 (400 mg, 1.4 mmol) in dichloromethane (3.0 mL) was cooled to 0° C., then a solution of SOCl₂ (0.12 mL, 1.68 mmol) in dichloromethane (2.0 mL) was added dropwise. After the dropwise addition was completed, the mixture was stirred at 0° C. for another 1 h. After the reaction was completed, the reaction was quenched by adding saturated sodium bicarbonate solution to the reaction system, and the reaction system was extracted with dichloromethane. The organic phases were combined and the organic solvent was removed to give crude compound 15-2, which was used directly in the next reaction without purification.

Compound 1-6 (223, 0.65 mmol), 15-2 (496 mg, 1.63 mmol) and potassium carbonate (361 mg, 2.6 mmol) were dissolved in 5.0 mL of DMF and the reaction solution was heated to 70° C. to react for 6 hours. The reaction solution was cooled to room temperature, then the reaction was quenched by adding saturated sodium chloride solution to the reaction system, and the reaction system was extracted with dichloromethane. The organic phases were combined and the organic solvent was removed to give the crude product. The crude was purified by silica gel column to give compound 15-3.

Then referring to the method of Example 1, compound 15 (40 mg) was prepared. ¹H NMR (400 MHz, CDCl₃): δ ppm 0.89 (t, J=7.2 Hz, 12H), 1.08 (s, 12H), 1.12-1.35 (m, 46H), 1.38-1.58 (m, 22H), 2.35 (s, 6H), 2.41-2.52 (m, 10H), 2.57-2.65 (m, 2H), 2.62 (m, 4H), 4.10 (t, J=6.4 Hz, 4H), 4.86 (m, 1H); ESI-MS m/z: 978.9 [M+H]⁺.

### Example 16: Synthesis of Compound 16

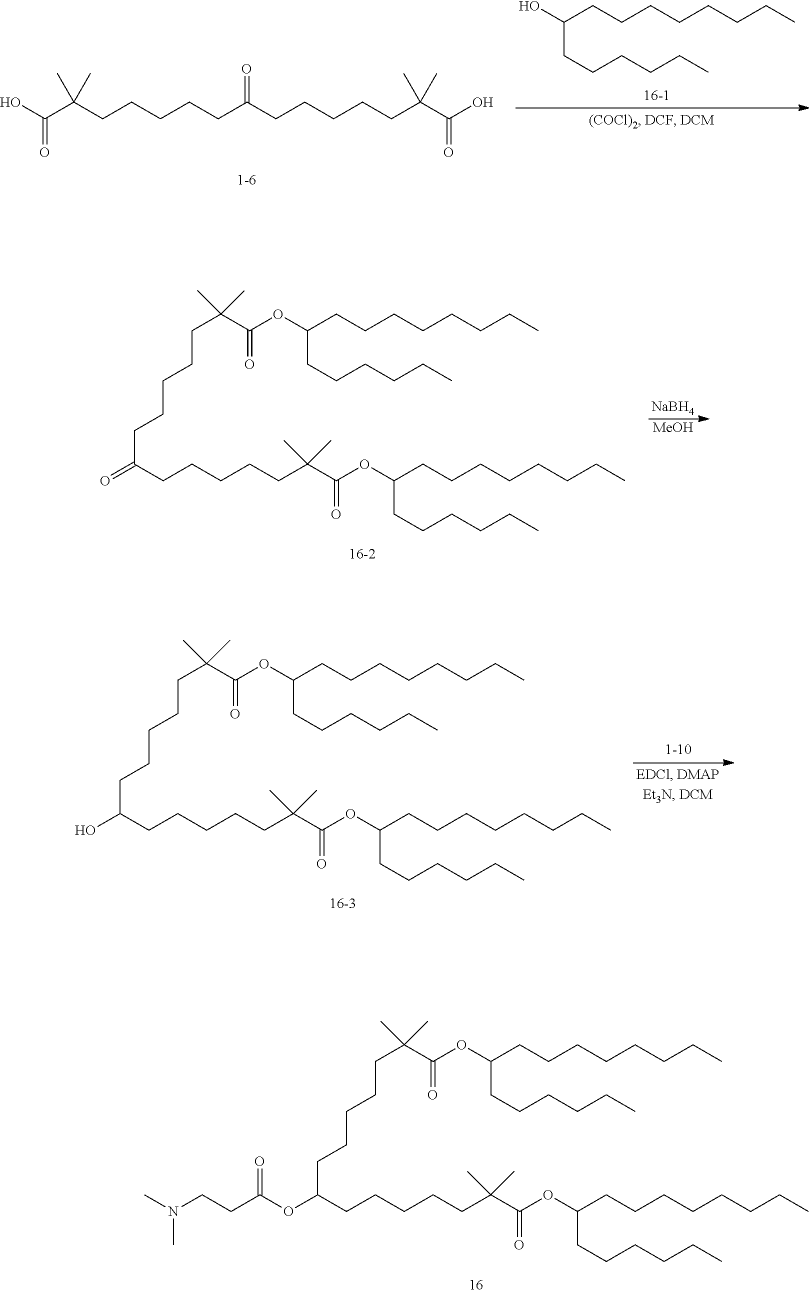

DMF (11 μL, 0.14 mmol) was added to a solution of compound 1-6 (460 mg, 1.34 mmol) in dichloromethane (5.0 mL) under ice bath conditions, and oxalyl chloride (0.47 mL, 5.37 mmol) was then added dropwise to the reaction solution. The ice bath was removed, and the mixture was stirred for 1 h at room temperature. The solvent was removed using a rotary-evaporator to give 255 mg of acyl chloride crude product as an oil, which was used directly in the next reaction step.

The above obtained acyl chloride crude product (255 mg, 0.67 mmol) was dissolved in 3.0 mL of 1,2-dichloroethane, and then compound 16-1 (384 mg, 1.68 mmol) was added to the reaction solution. The mixture was stirred at room temperature until the substrate was reacted completely. The solvent was removed using a rotary-evaporator to give the crude product, which was purified by silica gel column to give 300 mg of compound 16-2. ¹H NMR (400 MHz, CDCl₃): δ ppm 0.78-0.83 (m, 12H), 1.07 (s, 12H), 1.13-1.22 (m, 48H), 1.49 (br s, 16H), 2.29 (t, J=7.50 Hz, 4H), 4.76 (m, 2H).

Compound 16-2 (300 mg, 0.39 mmol) was dissolved in 4.0 mL of methanol. Then NaBH₄ (45 mg, 1.17 mmol) was slowly added to the reaction solution and the mixture was stirred at room temperature for 2 h. The reaction solution was quenched with saturated ammonium chloride solution, extracted with ethyl acetate. The organic phases were combined and the organic solvent was removed to give 300 mg of crude compound 16-3, which was used directly in the next reaction without purification.

Crude compound 16-3 (300 mg, 0.39 mmol) was dissolved in 2.0 mL DMF, and then 1-10 (69 mg, 0.59 mmol), EDCI (225 mg, 1.17 mmol), triethylamine (119 mg, 1.17 mmol) and DMAP (48 mg, 0.39 mmol) were added. The mixture was stirred at room temperature until the reactants was reacted completely. The reaction solution was quenched with saturated sodium chloride solution and extracted with ethyl acetate. The organic phases were combined, dried over anhydrous sodium sulfate, and filtered. The filtrate was concentrated to dryness to give the crude product. The crude product was purified by preparative high performance liquid chromatography to give compound 16 (32.5 mg). ¹H NMR (400 MHz, CDCl₃): δ ppm 0.79 (t, J=7.2 Hz, 12H), 1.07 (s, 12H), 1.19 (m, 52H), 1.40-1.46 (m, 16H), 2.15 (s, 6H), 2.34-2.58 (m, 4H), 4.74-4.81 (m, 3H); ESI-MS m/z: 864.8 [M+H]⁺.

### Example 17: Synthesis of Compound 17

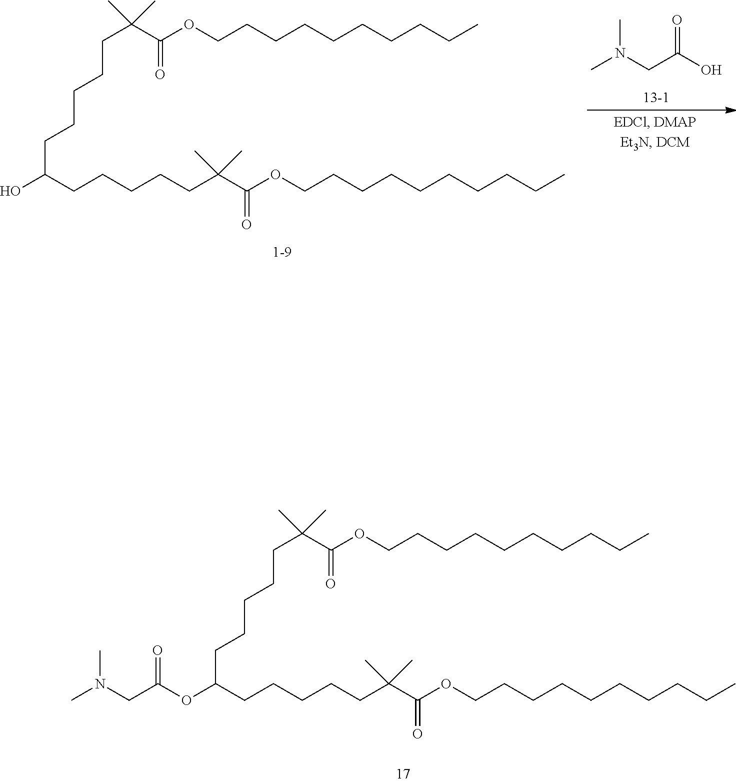

Referring to the method of Example 1, compound 17 was prepared as an oily product: 41.3 mg. ¹H NMR (400 MHz, CDCl₃): δ ppm 0.82 (t, J=7.2 Hz, 6H), 1.08 (s, 12H), 1.14-1.20 (m, 36H), 1.40-1.64 (m, 16H), 2.32 (s, 6H), 3.08-3.21 (m, 2H), 3.97 (t, J=7.2 Hz, 4H), 4.83-4.92 (m, 1H); ESI-MS m/z: 710.6 [M+H]⁺.

### Example 18: Synthesis of Compound 18

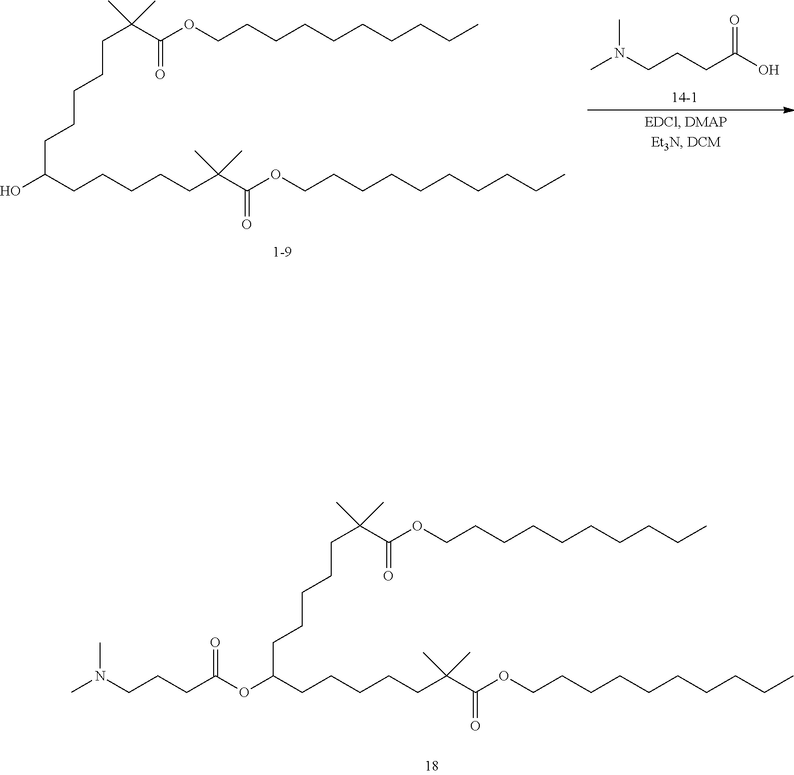

Referring to the method of Example 1, compound 18 was prepared as an oily product: 35.4 mg. ¹H NMR (400 MHz, CDCl₃): δ ppm 0.79 (t, J=7.2 Hz, 6H), 1.08 (s, 12H), 1.13-1.25 (m, 36H), 1.28-1.47 (m, 10H), 1.47-1.62 (m, 6H), 1.68-1.79 (m, 2H), 2.15 (s, 6H), 2.21-2.31 (m, 4H), 3.97 (t, J=7.2 Hz, 4H), 4.73-4.82 (m, 1H); ESI-MS m/z: 738.7 [M+H]⁺.

### Example 19: Synthesis of Compound 19

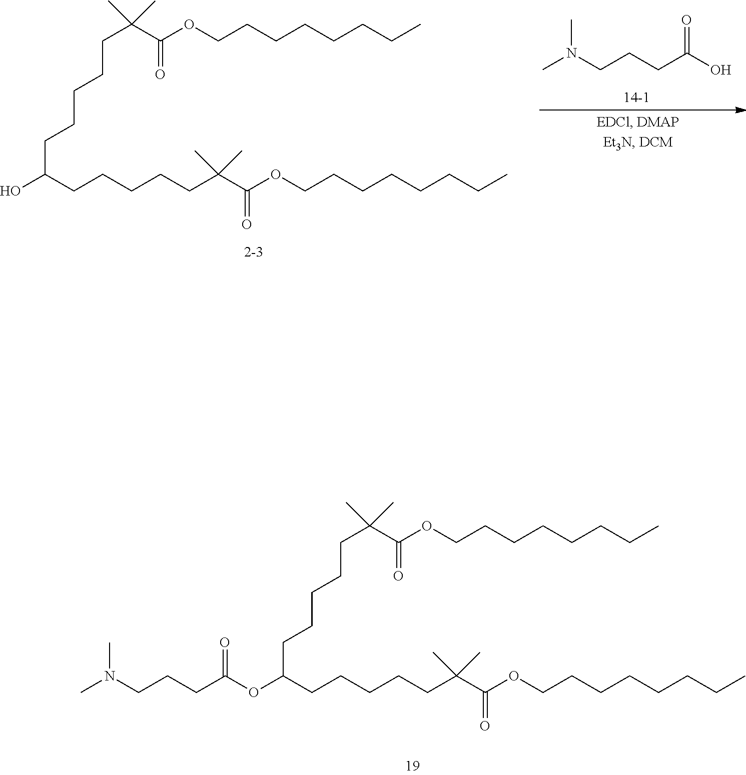

Referring to the method of Example 1, compound 19 was prepared as an oily product: 33.1 mg. ¹H NMR (400 MHz, CDCl₃): δ ppm 0.89 (t, J=7.2 Hz, 6H), 1.15 (s, 12H), 1.29 (m, 30H), 1.50 (m, 8H), 1.60 (m, 6H), 1.64 (m, 2H), 2.23 (s, 6H), 2.33 (m, 4H), 4.05 (t, J=6.8 Hz, 4H), 4.86 (m, 1H); ESI-MS m/z: 682.6 [M+H]⁺.

### Example 20: Synthesis of Compound 20

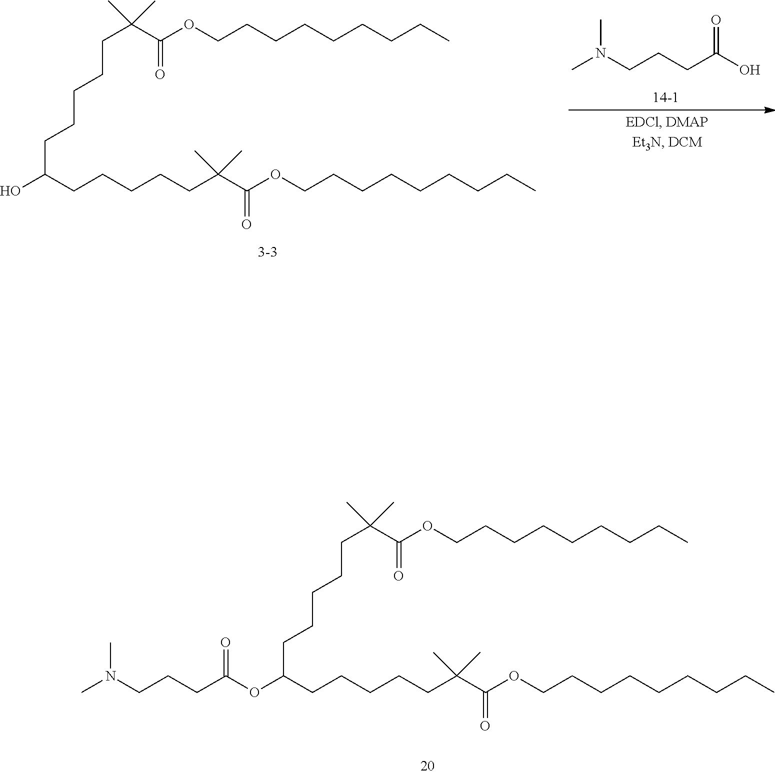

Referring to the method of Example 1, compound 20 was prepared as an oily product: 302 mg. ¹H NMR (400 MHz, CDCl₃): δ ppm 0.89 (t, J=7.2 Hz, 6H), 1.15 (s, 12H), 1.27 (m, 34H), 1.47 (m, 8H), 1.51 (m, 6H), 1.79 (m, 2H), 2.23 (s, 6H), 2.33 (m, 4H), 4.04 (t, J=6.8 Hz, 4H), 4.85 (m, 1H); ESI-MS m/z: 710.7 [M+H]⁺.

### Example 21: Synthesis of Compound 21

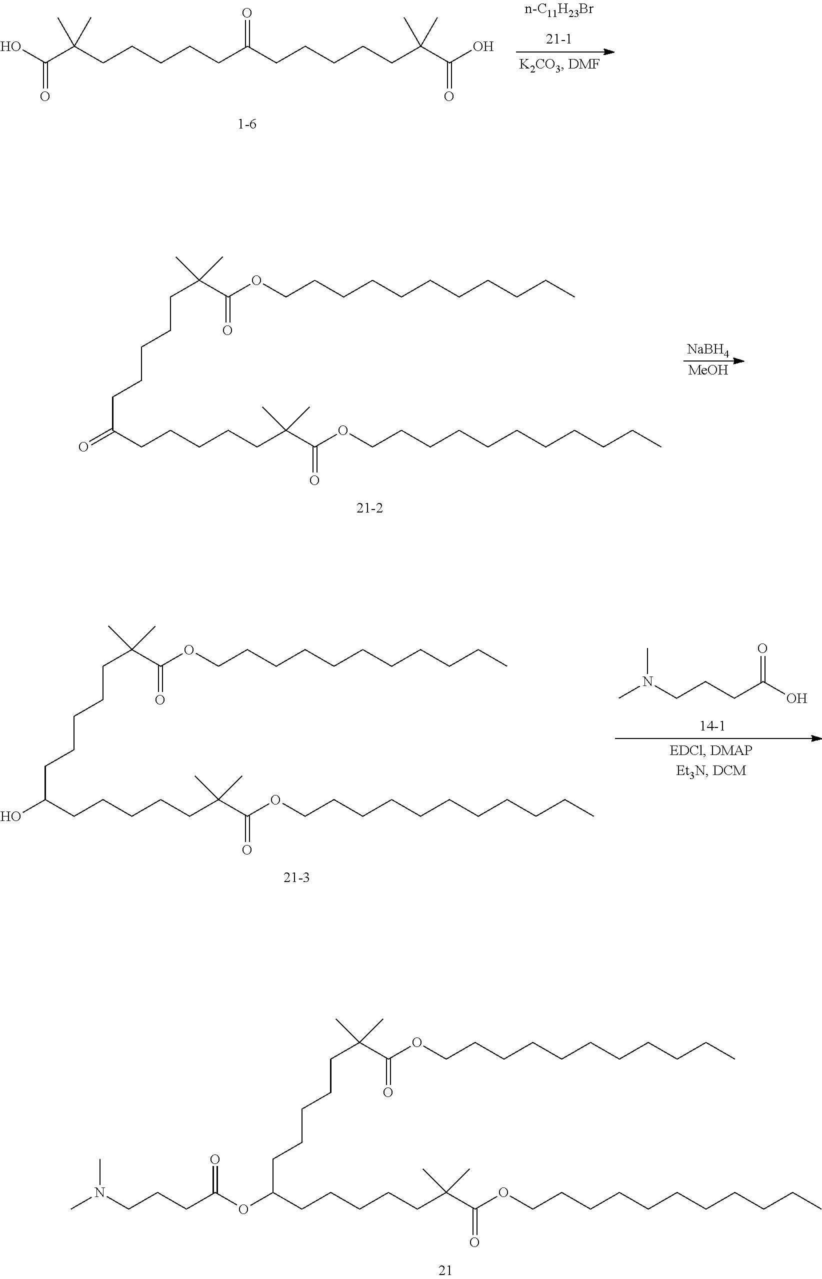

Referring to the method of Example 1, compound 21 was prepared as an oily product: 31.2 mg. ¹H NMR (400 MHz, CDCl₃): δ ppm 0.79 (t, J=7.2 Hz, 6H), 1.08 (s, 12H), 1.25 (m, 44H), 1.39 (m, 8H), 1.51 (m, 4H), 1.82 (m, 2H), 2.25 (t, J=7.2 Hz, 2H), 2.32 (s, 6H), 2.41 (m, 2H), 3.96 (t, J=6.8 Hz, 4H), 4.75 (m, 1H); ESI-MS m/z: 766.7 [M+H]⁺.

### Example 22: Synthesis of Compound 22

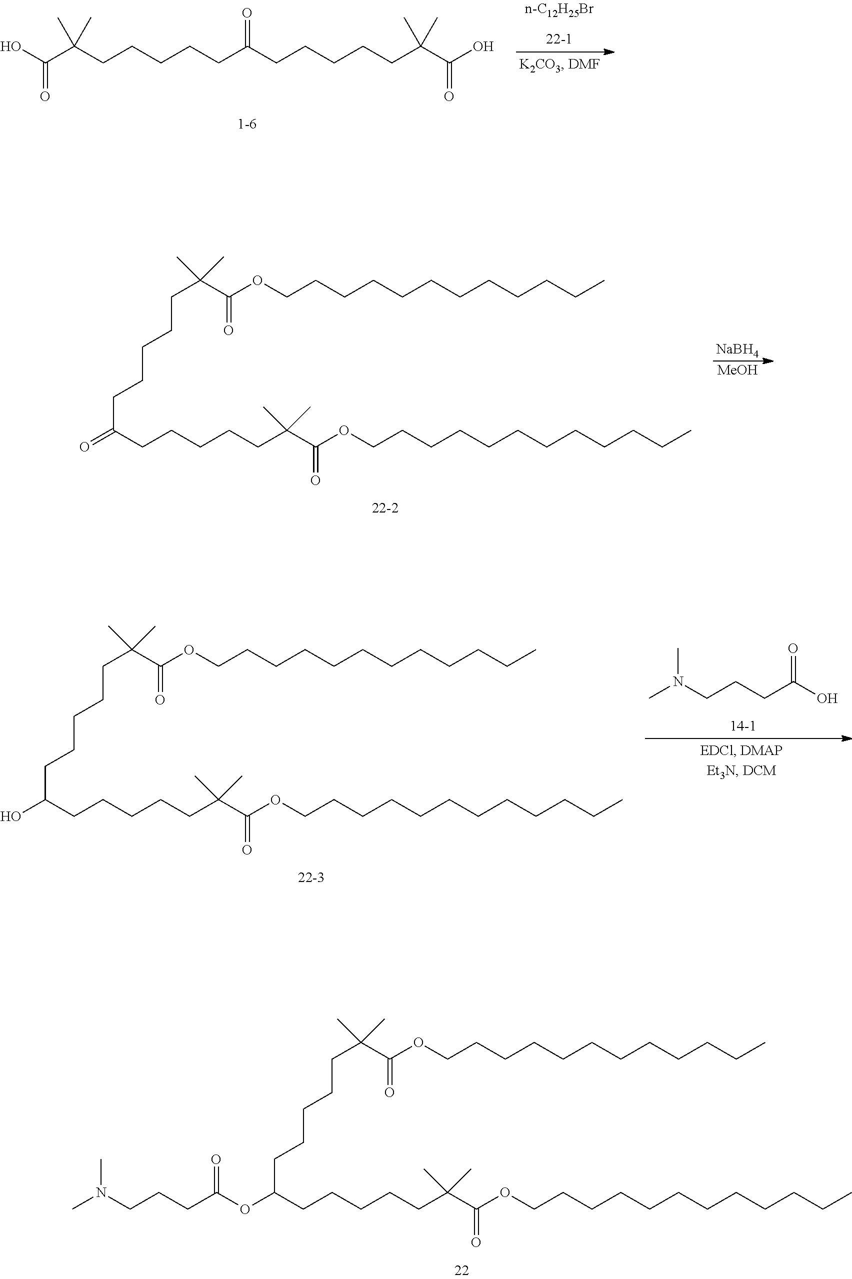

Referring to the method of Example 1, compound 22 was prepared as an oily product: 31.8 mg. ¹H NMR (400 MHz, CDCl₃): δ ppm 0.79 (t, J=7.2 Hz, 6H), 1.07 (s, 12H), 1.28 (m, 48H), 1.40 (m, 8H), 1.53 (m, 4H), 1.84 (m, 2H), 2.26 (t, J=7.2 Hz, 2H), 2.35 (s, 6H), 2.48 (m, 2H), 3.98 (t, J=6.8 Hz, 4H), 4.75 (m, 1H); ESI-MS m/z: 794.7 [M+H]⁺.

### Example 23: Synthesis of Compound 23

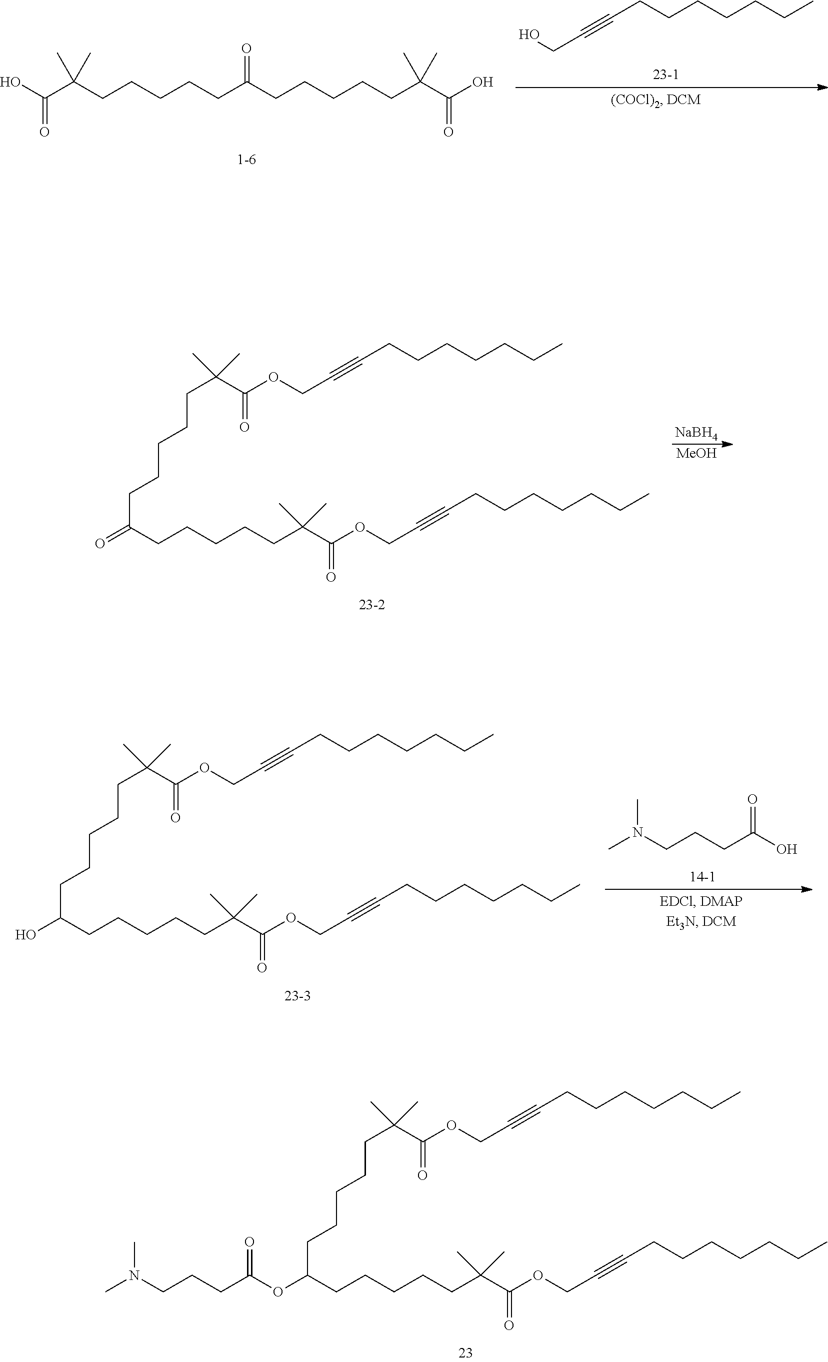

Referring to the method of Example 7, compound 23 was prepared as an oily product: 31.0 mg. ¹H NMR (400 MHz, CDCl₃): δ ppm 0.87 (t, J=7.2 Hz, 6H), 1.16 (s, 12H), 1.20-1.39 (m, 28H), 1.45-1.54 (m, 12H), 1.74-1.82 (m, 2H), 2.12-2.35 (m, 14), 4.63 (t, J=2.4 Hz, 4H), 4.79-4.88 (m, 1H); ESI-MS m/z: 730.6 [M+H]⁺.

### Example 24: Synthesis of Compound 24

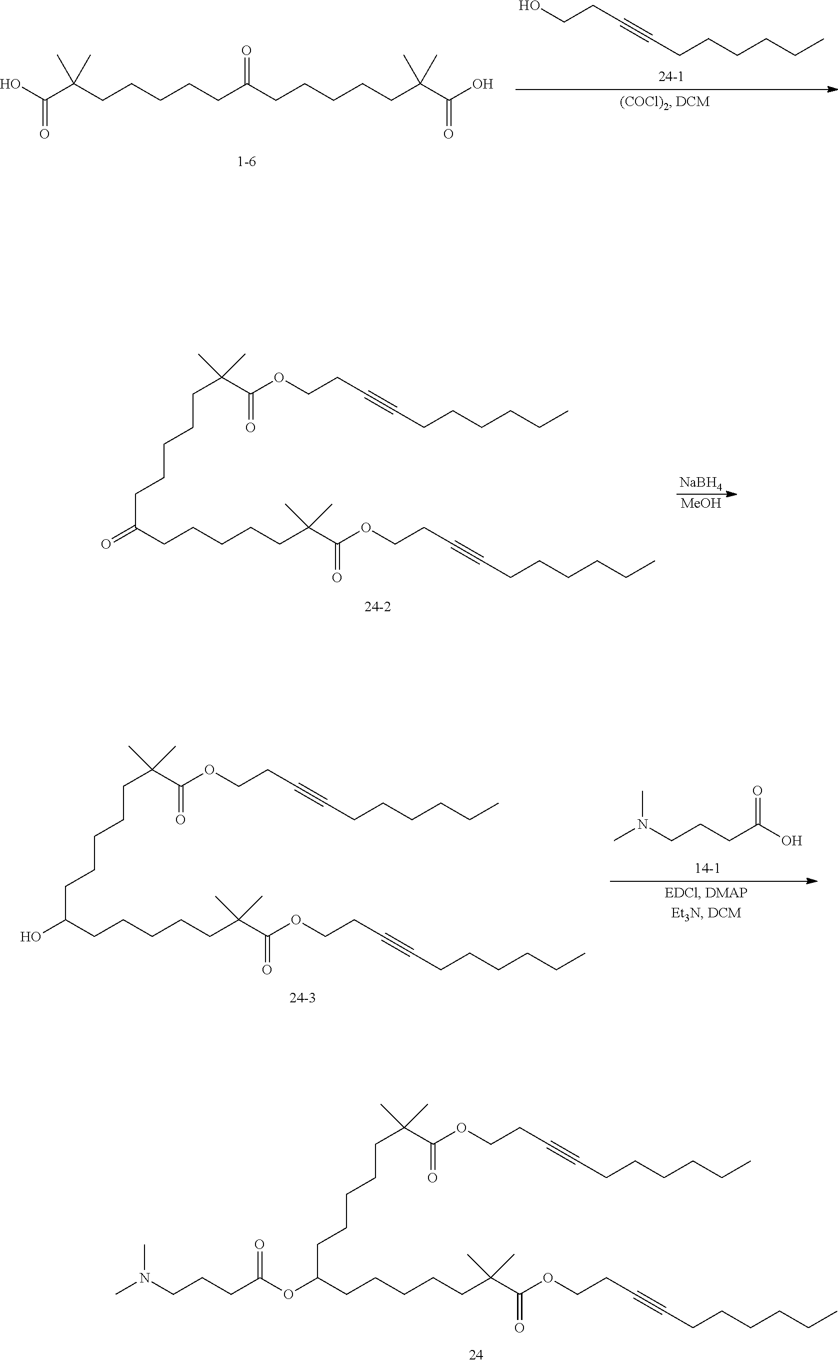

Referring to the method of Example 7, compound 24 was prepared as an oily product: 31.0 mg.

¹H NMR (400 MHz, CDCl₃): δ ppm 0.88 (t, J=7.2 Hz, 6H), 1.15 (s, 12H), 1.20-1.38 (m, 24H), 1.43-1.52 (m, 12H), 1.76-1.84 (m, 2H), 2.09-2.14 (m, 4H), 2.23 (s, 6H), 2.28-2.36 (m, 4H), 2.43-2.49 (m, 4H), 4.10 (t, J=7.2 Hz, 4H), 4.80-4.88 (m, 1H); ESI-MS m/z: 730.6 [M+H]⁺.

### Example 25: Synthesis of Compound 25

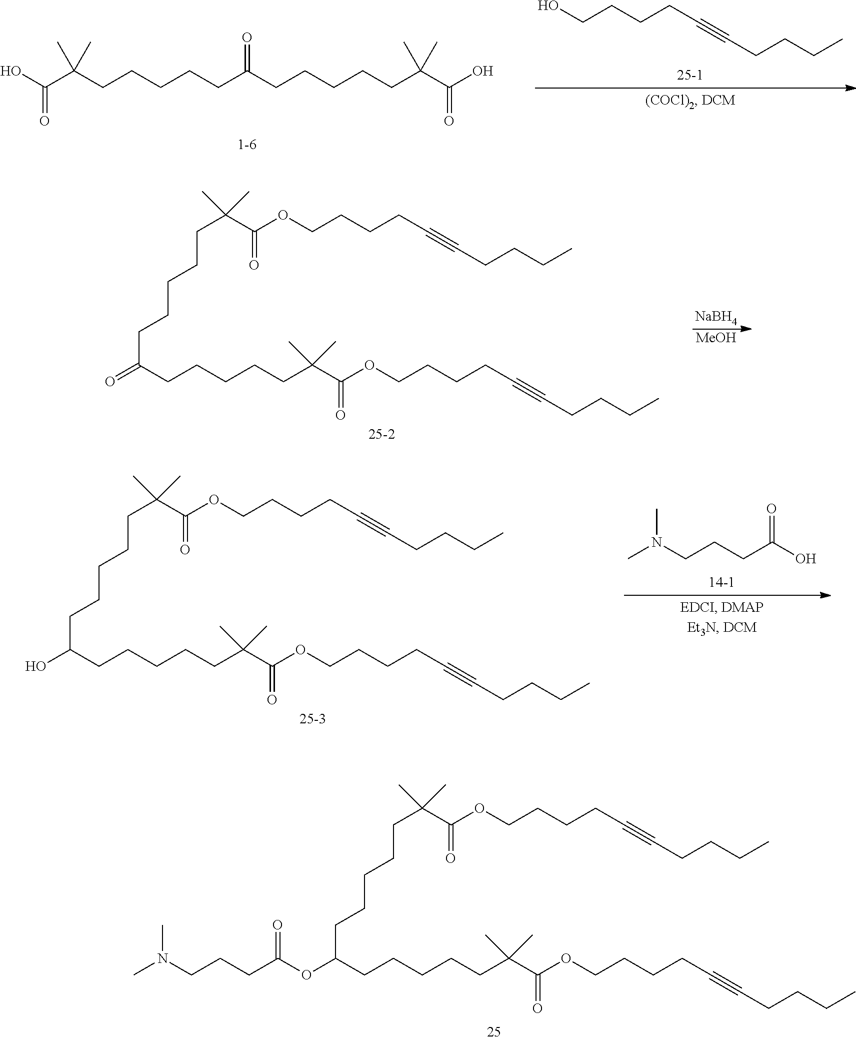

Referring to the method of Example 7, compound 25 was prepared as an oily product: 32.1 mg. ¹H NMR (400 MHz, CDCl₃): δ ppm 0.84 (t, J=7.2 Hz, 6H), 1.08 (s, 12H), 1.02-1.21 (m, 12H), 1.38-1.47 (m, 22H), 1.59-1.78 (m, 6H), 2.02-2.17 (m, 14H), 2.19-2.30 (m, 4H), 4.01 (t, J=6.8 Hz, 4H), 4.71-4.83 (m, 1H); ESI-MS m/z: 730.6 [M+H]⁺.

### Example 26: Synthesis of Compound 26

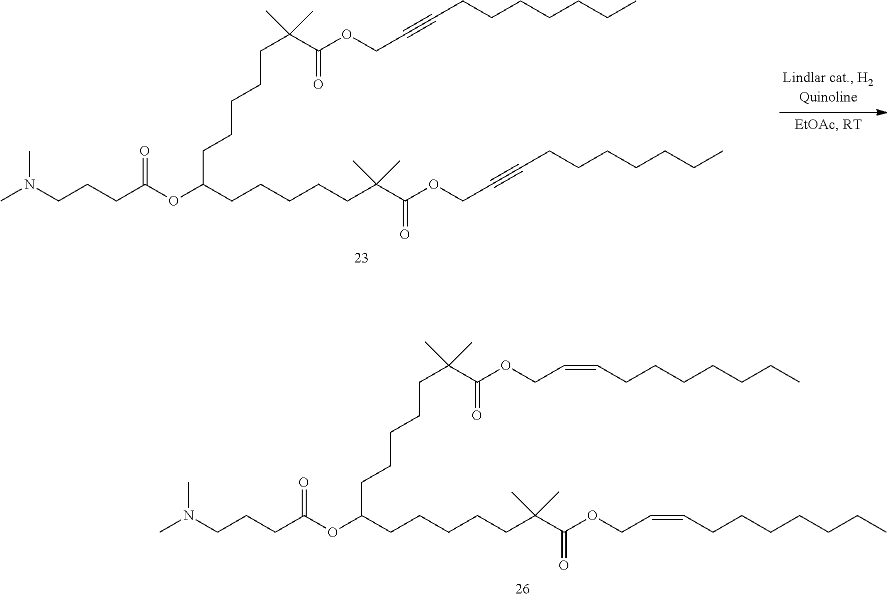

Compound 23 (300 mg, 0.41 mmol) and quinoline (106 mg, 0.82 mmol) were dissolved in 3.0 mL of ethyl acetate, and the air in the reaction system was replaced with nitrogen for 2-3 min at room temperature, then lindlar catalyst (16.9 mg) was added. Hydrogen gas was introduced to the reaction solution and the air was replaced with hydrogen for 2-3 min. The reaction system was kept under hydrogen atmosphere (15 psi) at room temperature for 30 min. The complete disappearance of the reactants was monitored by LC-MS. The reaction solution was filtered, and the filter cake was rinsed with ethyl acetate 3-4 times. The combined ethyl acetate was collected and the organic solvent was removed using a rotary-evaporator to give the crude product, which was purified by preparative high performance liquid chromatography to give compound 26 (31.3 mg). ¹H NMR (400 MHz, CDCl₃): δ ppm 0.81 (t, J=7.2 Hz, 6H), 1.08 (s, 12H), 1.15-1.28 (m, 32H), 1.38-1.44 (m, 8H), 1.70-1.79 (m, 2H), 2.01 (m, 4H), 2.15 (s, 6H), 2.16-2.28 (m, 4H), 4.54 (d, J=12.0 Hz, 4H), 4.75 (m, 1H), 5.39-5.59 (m, 4H); ESI-MS m/z: 734.6 [M+H]⁺.

### Example 27: Synthesis of Compound 27

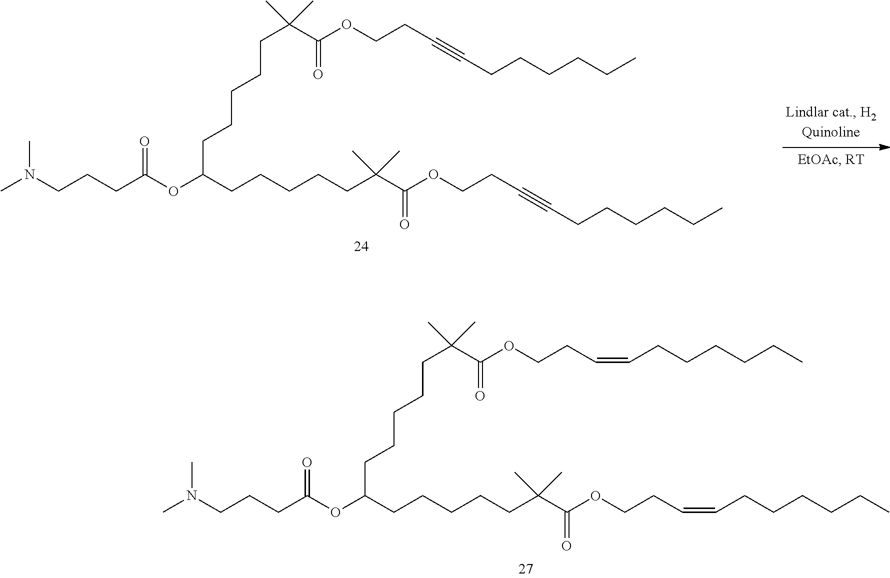

Referring to the method of Example 26, compound 27 was prepared as an oily product: 35.0 mg. ¹H NMR (400 MHz, CDCl₃): δ ppm 0.82 (m, 6H), 1.08 (s, 12H), 1.14-1.31 (m, 28H), 1.37-1.45 (m, 8H), 1.70-1.79 (m, 2H), 1.96 (m, 4H), 2.06-2.36 (m, 14H), 3.98 (t, J=7.2 Hz, 4H), 4.74-4.82 (m, 1H), 5.22-5.31 (m, 2H), 5.37-5.48 (m, 2H); ESI-MS m/z: 734.7 [M+H]⁺.

### Example 28: Synthesis of Compound 28

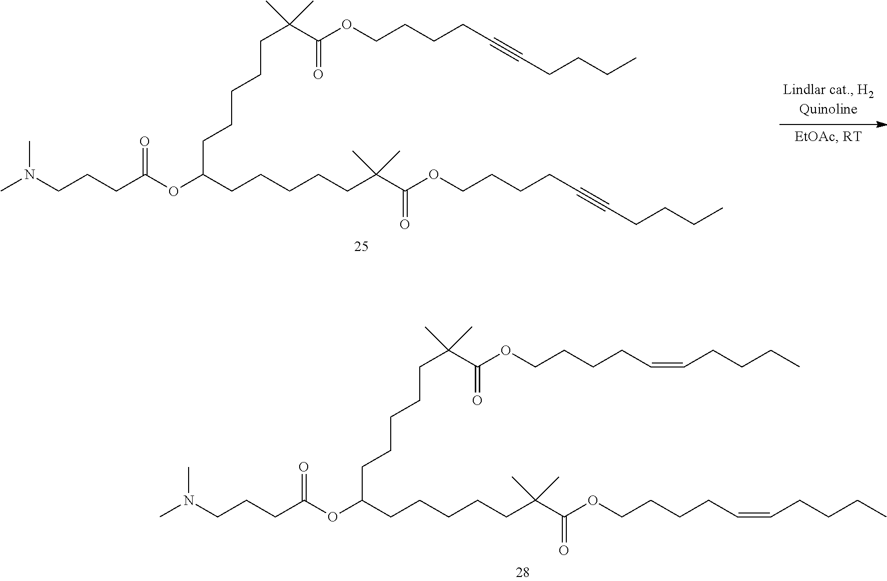

Referring to the method of Example 26, compound 28 was prepared as an oily product: 31.8 mg. ¹H NMR (400 MHz, CDCl₃): δ ppm 0.92 (t, J=6.8 Hz, 6H), 1.18 (s, 12H), 1.21-1.39 (m, 22H), 1.40-1.59 (m, 12H), 1.60-1.72 (m, 4H), 1.89-2.01 (m, 2H), 2.02-2.15 (m, 8H), 2.34-2.69 (m, 8H), 4.08 (t, J=6.4 Hz, 4H), 4.82-4.92 (m, 1H), 5.30-5.48 (m, 4H); ESI-MS m/z: 734.6 [M+H]⁺.

### Example 30: Synthesis of Compound 30

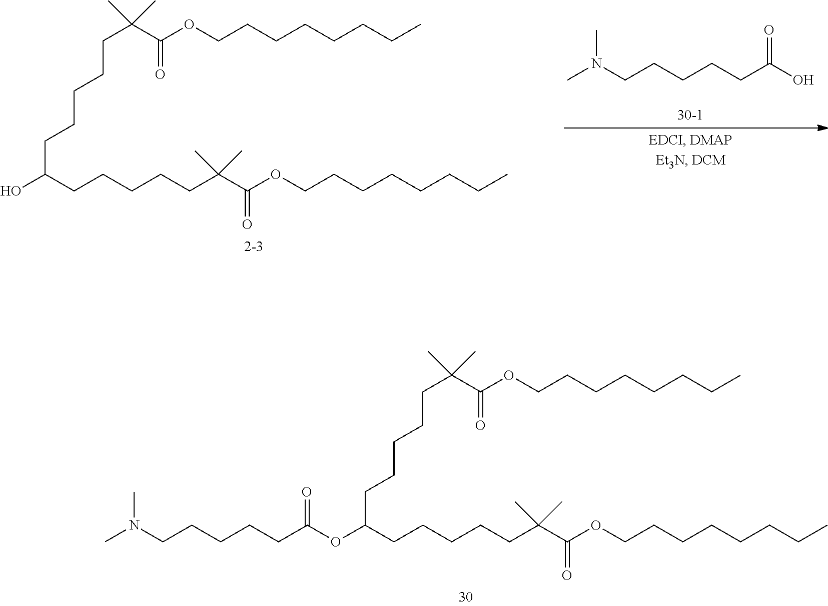

Referring to the method of Example 1, compound 30 was prepared as an oily product: 33.0 mg. ¹H NMR (400 MHz, CDCl₃): δ ppm 0.92 (t, J=6.8 Hz, 6H), 1.18 (s, 12H), 1.19-1.37 (m, 36H), 1.45-1.57 (m, 8H), 1.58-1.74 (m, 8H), 2.27-2.50 (m, 8H), 4.07 (t, J=6.8 Hz, 4H), 4.83-4.90 (m, 1H); ESI-MS m/z: 710.6 [M+H]⁺.

### Example 32: Synthesis of Compound 32

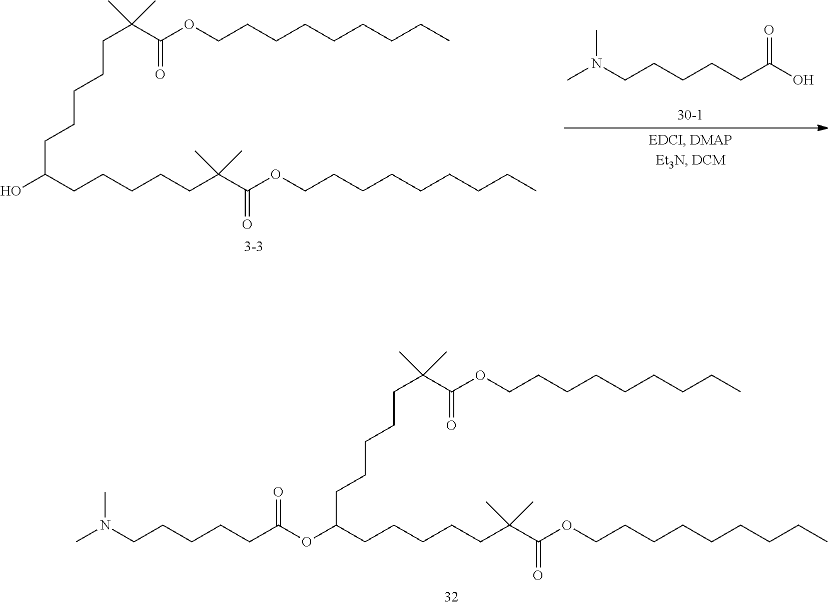

Referring to the method of Example 1, compound 32 was prepared as an oily product: 31.1 mg. ¹H NMR (400 MHz, CDCl₃): δ ppm 0.80 (t, J=6.8 Hz, 6H), 1.08 (s, 12H), 1.20-1.27 (m, 34H), 1.34-1.47 (m, 12H), 1.48-1.62 (m, 8H), 2.15 (s, 6H), 2.19-2.24 (m, 4H), 3.97 (t, J=6.8 Hz, 4H), 4.74-4.80 (m, 1H); ESI-MS m/z: 738.6 [M+H]⁺.

### Example 33: Synthesis of Compound 33

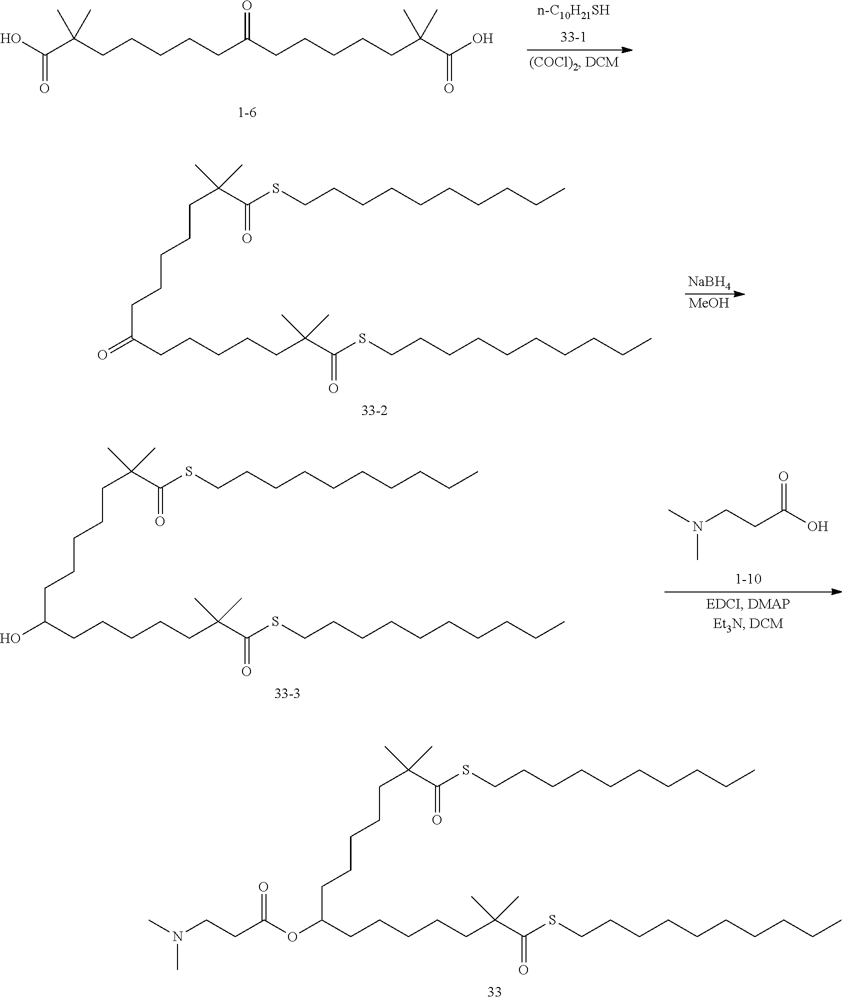

Compound 1-6 (448 mg, 1.3 mmol) was dissolved in 5.0 mL of dichloromethane, and the reaction system was cooled to 0° C. in an ice bath. DMF (10 μL, 0.13 mmol) was added, and oxalyl chloride (0.44 mL, 5.2 mmol) was then added dropwise to the reaction solution. The ice bath was removed after the dropwise addition was completed and the mixture was stirred for 1 h at room temperature. The solvent was removed using a rotary-evaporator to give acyl chloride crude product (330 mg) as an oil, which was used directly in the next reaction step.

1-Decanethiol 33-1 (455 mg, 2.61 mmol) was added to a solution of crude acyl chloride (330 mg, 0.87 mmol) in DCE (3.0 mL), and the reaction was heated to 70° C. to react overnight. The reaction solution was cooled to room temperature and the solvent was removed using a rotary-evaporator to give the crude product, which was purified by silica gel column to give compound 33-2 (400 mg). ¹H NMR (400 MHz, CDCl₃): δ ppm 0.84-0.87 (m, 6H), 1.14-1.18 (m, 12H), 1.20-1.28 (m, 36H), 1.48-1.55 (m, 12H), 2.33 (t, J=7.2 Hz, 4H), 2.79 (t, J=7.2 Hz, 4H).

Compound 33-2 (300 mg, 0.46 mmol) was dissolved in 3.0 mL of methanol and NaBH4 (52.5 mg, 1.38 mmol) was added in batches. The reaction solution was stirred under nitrogen atmosphere at room temperature for 2 h. The complete disappearance of the reaction material was monitored by TLC. The reaction solution was quenched by adding saturated ammonium chloride solution, and extracted with ethyl acetate. The organic phases were combined, dried over anhydrous sodium sulfate, and filtered. The filtrate was collected and concentrated to give 300 mg of crude compound 33-3, which was directly used in the next reaction step without further purification.

Crude compound 33-3 (150 mg, 0.23 mmol) was dissolved in 3.0 mL of dichloromethane, and 1-10 (80.2 mg, 0.69 mmol), EDCI (131 mg, 0.69 mmol), triethylamine (0.1 mL, 0.69 mmol) and DMAP (28 mg, 0.23 mmol) were added to the reaction system. The reaction solution was stirred at room temperature for 12 h. The reaction solution was then quenched by adding saturated ammonium chloride solution, and extracted with dichloromethane. The organic phases were combined, dried over anhydrous sodium sulfate, and filtered. The filtrate was collected and concentrated to give the crude product, which was passed through preparative high performance liquid chromatography to give compound 33 (28.6 mg). H NMR (400 MHz, CDCl₃): δ ppm 0.81 (t, J=7.2 Hz, 6H), 1.15 (s, 12H), 1.31 (m, 40H), 1.48 (m, 12H), 2.23 (s, 6H), 2.42 (m, 4H), 2.80 (t, J=7.2 Hz, 4H), 4.82 (m, 1H); ESI-MS m/z: 756.6 [M+H]⁺.

### Example 34: Synthesis of Compound 34

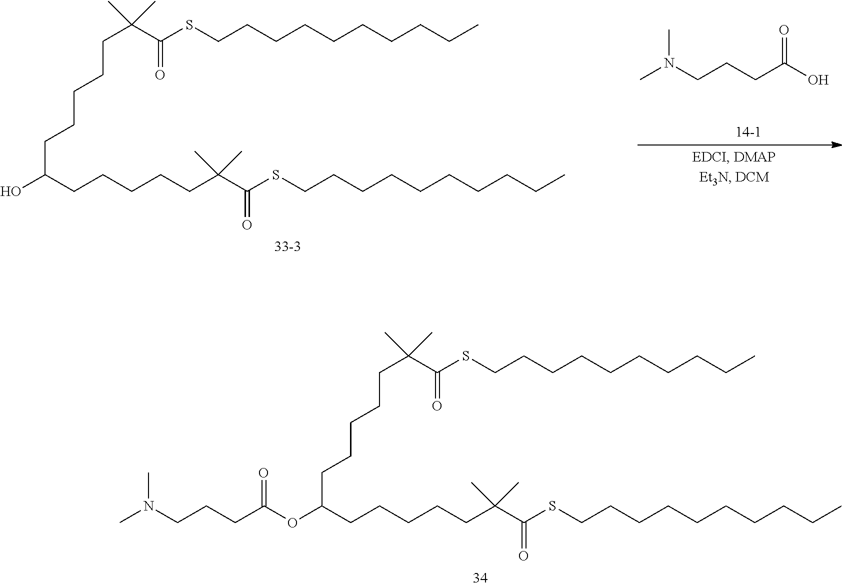

Referring to the method of Example 33, compound 34 was prepared as an oily product: 105.2 mg. ¹H NMR (400 MHz, CDCl₃): δ ppm 0.85 (t, J=7.2 Hz, 6H), 1.15 (s, 12H), 1.6-1.32 (m, 40H), 1.37-1.53 (m, 14H), 1.75 (m, 2H), 2.24-2.34 (m, 8H), 2.80 (t, J=7.2 Hz, 4H), 4.72-4.82 (m, 1H); ESI-MS m/z: 770.6 [M+H]⁺.

### Example 36: Synthesis of Compound 36

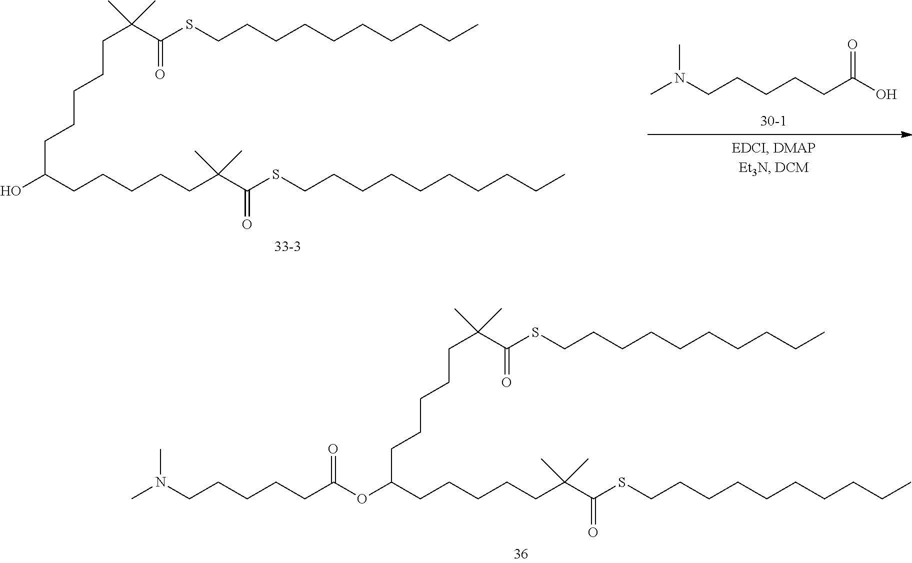

Referring to the method of Example 33, compound 36 was prepared as an oily product: 33.4 mg. ¹H NMR (400 MHz, CDCl₃): δ ppm 0.86 (t, J=6.8 Hz, 6H), 1.16 (s, 12H), 1.18-1.38 (m, 40H), 1.41-1.59 (m, 16H), 1.61-1.67 (m, 2H), 2.19-2.33 (m, 10H), 2.82 (t, J=7.2 Hz, 4H), 4.83 (m, H); ESI-MS m/z: 798.6 [M+H]⁺.

### Example 37: Synthesis of Compound 37

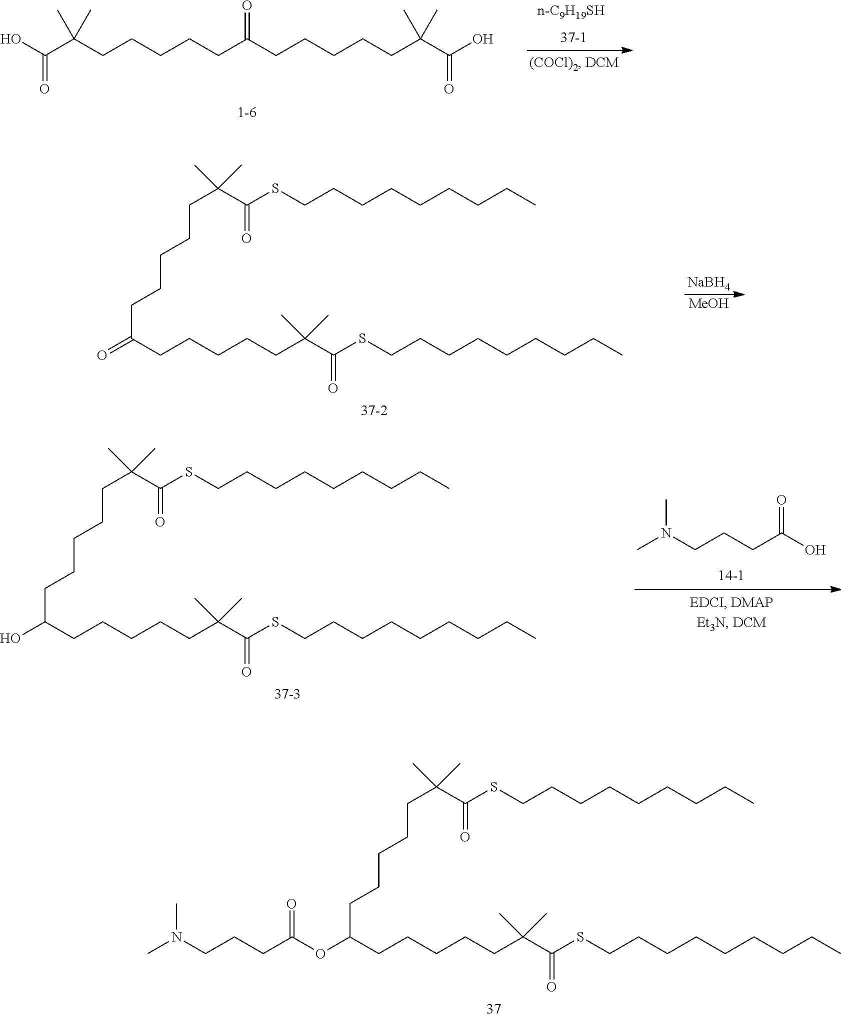

Referring to the method of Example 33, compound 37 was prepared as an oily product: 33.2 mg. ¹H NMR (400 MHz, CDCl₃): δ ppm 0.87 (t, J=6.8 Hz, 6H), 1.20 (s, 12H), 1.19-1.37 (m, 36H), 1.39-1.56 (m, 12H), 1.75-1.84 (m, 2H), 2.24 (s, 6H), 2.28-2.34 (m, 4H), 2.81 (t, J=7.2 Hz, 4H), 4.79-4.87 (m, 1H); ESI-MS m/z: 742.6 [M+H]⁺.

### Example 39: Synthesis of Compound 39

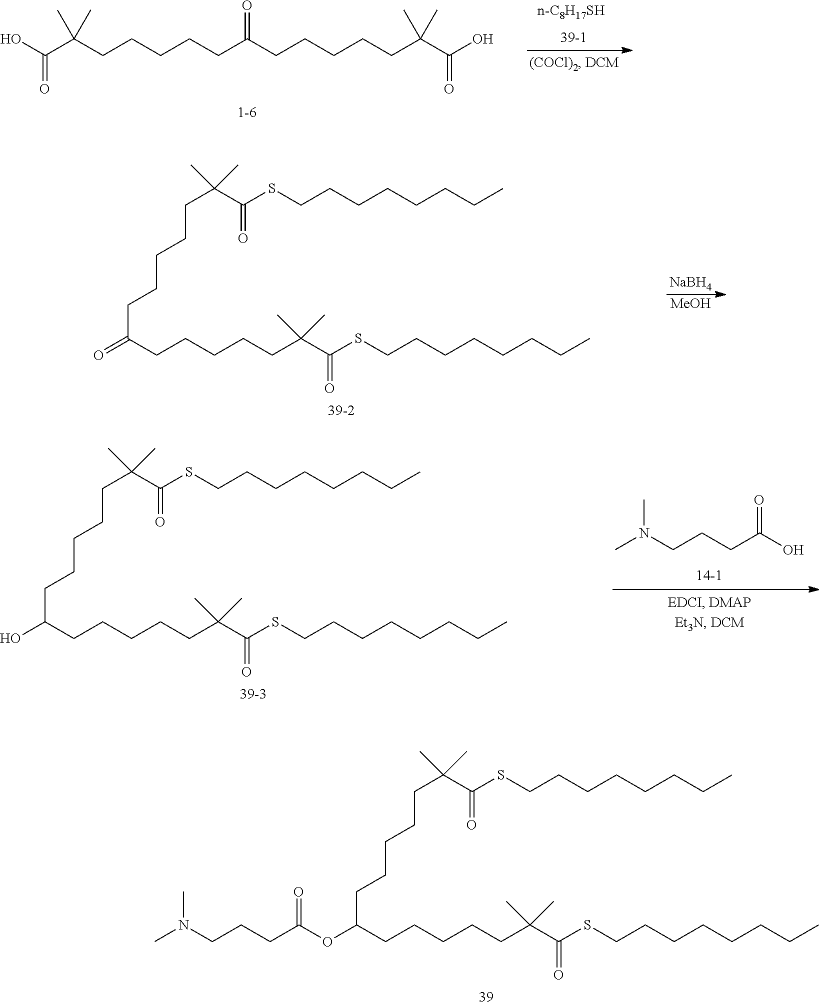

Referring to the method of Example 33, compound 39 was prepared as an oily product: 30.7 mg. ¹H NMR (400 MHz, CDCl₃): δ ppm 0.91 (t, J=7.2 Hz, 6H), 1.22 (s, 12H), 1.17-1.38 (m, 32H), 1.47-1.58 (m, 12H), 1.78-1.87 (m, 2H), 2.28 (s, 6H), 2.34-2.37 (m, 4H), 2.85 (t, J=7.2 Hz, 4H), 4.81-4.90 (m, 1H); ESI-MS m/z: 714.6 [M+H]⁺.

### Example 40: Synthesis of Compound 40

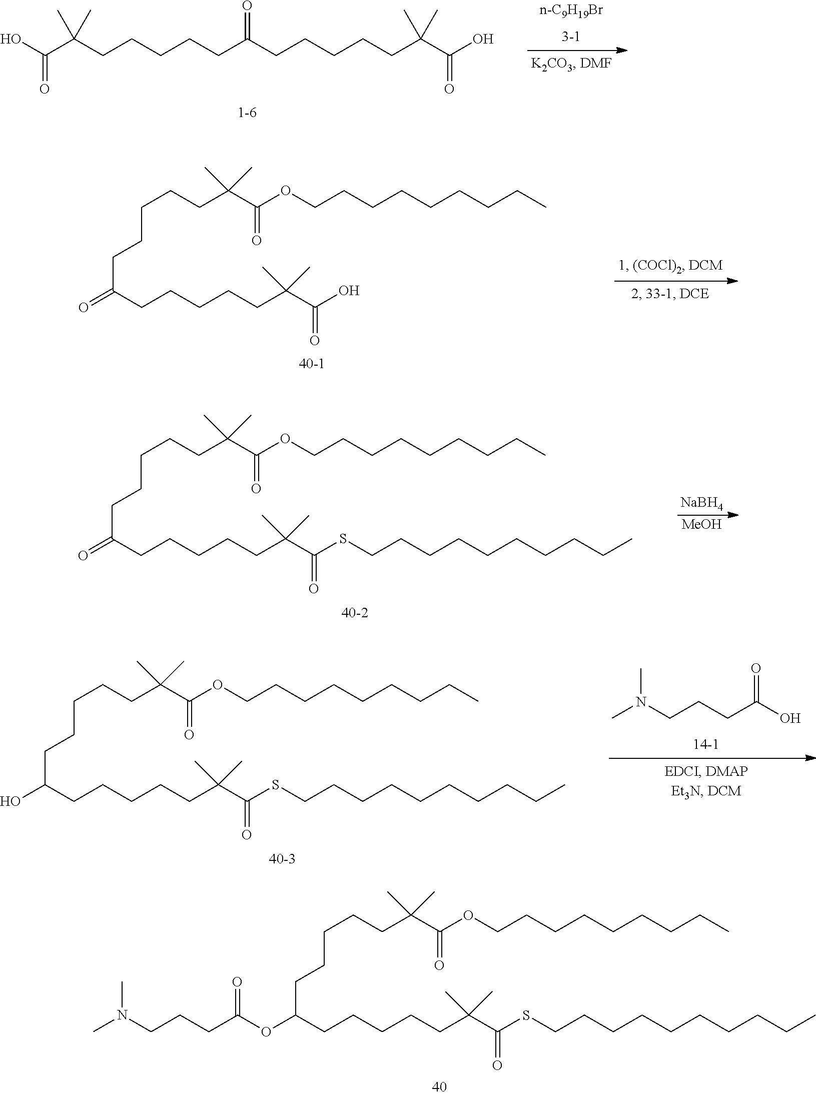

Potassium carbonate (1.55 g, 11.2 mmol, 4.0 eq.) was added to a solution of compound 1-6 (959 mg, 2.8 mmol, 1.0 eq.) and 3-1 (638 mg, 3.08 mmol, 1.1 eq.) in DMF. Then the reaction was warmed up to 60° C. for 4 h. The reaction was cooled to room temperature. The reaction system was quenched with saturated aqueous sodium chloride solution and extracted with ethyl acetate. The organic phases were combined, dried over anhydrous sodium sulfate, and filtered. The filtrate was concentrated to dryness to give the crude product, which was purified by silica gel column to give compound 40-1 (682 mg).

Compound 40-1 (324 mg, 0.69 mmol, 1.0 eq.) was dissolved in 5.0 mL of dichloromethane, and the reaction system was cooled to 0° C. in an ice bath. 2 drops of DMF were added and oxalyl chloride (0.24 mL, 2.8 mmol, 4.0 eq.) was then added dropwise to the reaction solution. The ice bath was removed after the dropwise addition was completed and the mixture was stirred for 1 h at room temperature. The solvent was removed using a rotary-evaporator to give acyl chloride crude product (309 mg) as an oil, which was used directly in the next reaction step.

1-Decanethiol 33-1 (331 mg, 1.9 mmol, 3.0 eq) was added to a solution of crude acyl chloride (309 mg) in DCE (3.0 mL), and the reaction was heated to 70° C. to react overnight. The reaction solution was cooled to room temperature and the solvent was removed using a rotary-evaporator to give the crude product, which was purified by silica gel column to give compound 40-2 (274 mg).

Then referring to the method of Example 1, compound 40 was prepared as an oily product: 34.2 mg. ¹H NMR (400 MHz, CDCl₃): δ ppm 0.81 (t, J=7.2 Hz, 6H), 1.05 (s, 6H), 1.12 (s, 6H), 1.08-1.28 (m, 36H), 1.37-1.57 (m, 14H), 1.71-1.76 (m, 2H), 2.22 (s, 6H), 2.25-2.31 (m, 4H), 2.75 (t, J=7.2 Hz, 2H), 3.97 (t, J=7.2 Hz, 2H), 4.75-4.84 (m, 1H); ESI-MS m/z: 740.6 [M+H]⁺.

### Example 41: Synthesis of Compound 41

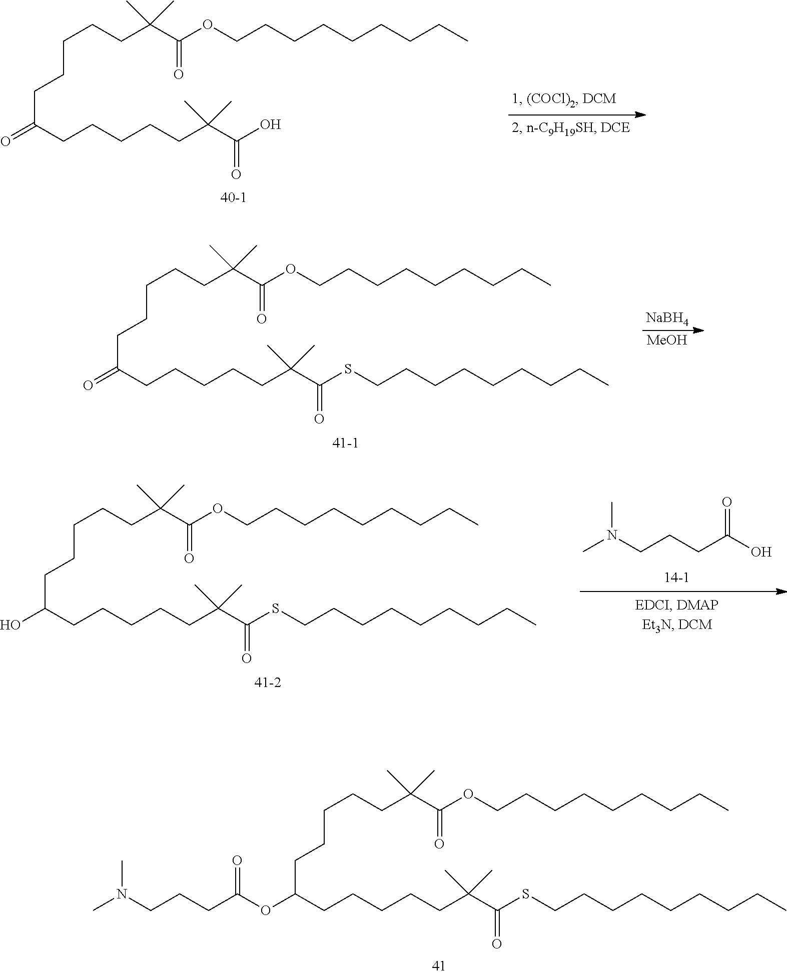

Referring to the method of Example 40, compound 41 was prepared as an oily product: 31.1 mg. ¹H NMR (400 MHz, CDCl₃): δ ppm 0.86-0.89 (m, 6H), 1.10 (s, 6H), 1.15 (s, 6H), 1.08-1.31 (m, 34H), 1.41-1.61 (m, 14H), 1.74-1.82 (m, 2H), 2.17-2.35 (m, 10H), 2.85 (t, J=7.2 Hz, 2H), 4.03 (t, J=7.2 Hz, 2H), 4.82-4.87 (m, 1H); ESI-MS m/z: 726.6 [M+H]⁺.

### Example 42: Synthesis of Compound 42

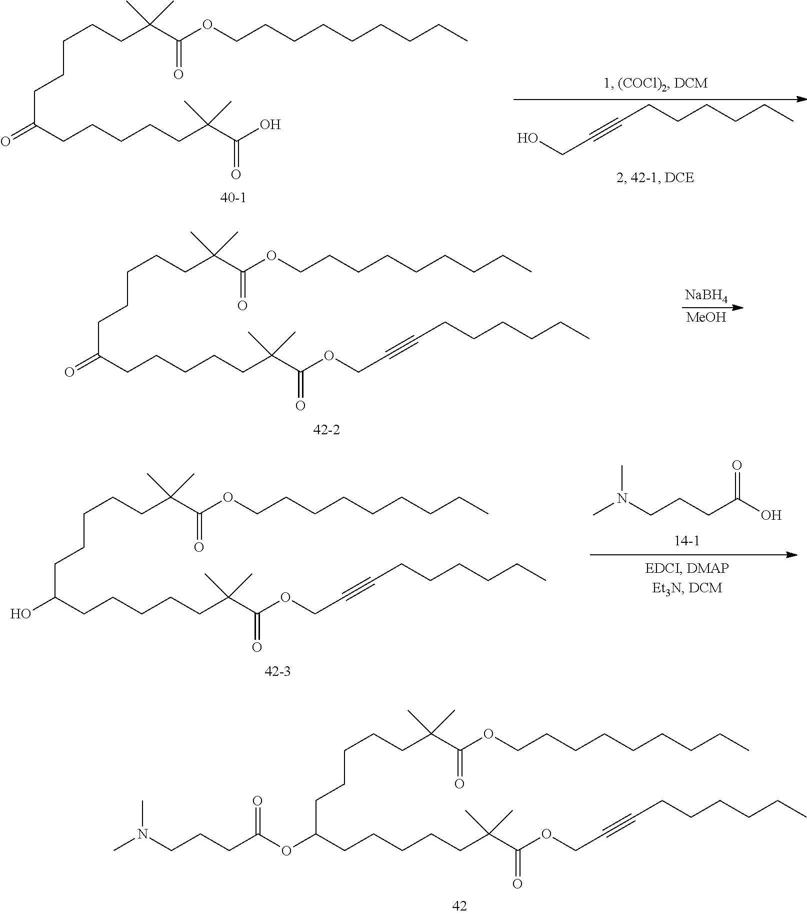

Referring to the method of Example 40, compound 42 was prepared as an oily product: 30.9 mg. ¹H NMR (400 MHz, CDCl₃): δ ppm 0.77-0.82 (m, 6H), 1.05 (s, 6H), 1.10 (s, 6H), 1.11-1.28 (m, 31H), 1.33-1.42 (m, 9H), 1.47-1.59 (m, 2H), 1.73-1.81 (m, 2H), 2.08-2.14 (m, 2H), 2.21-2.33 (m, 10H), 3.97 (t, J=7.2 Hz, 2H), 4.55 (m, 2H), 4.72-4.81 (m, 1H); ESI-MS m/z: 706.6 [M+H]⁺.

### Example 43: Synthesis of Compound 43

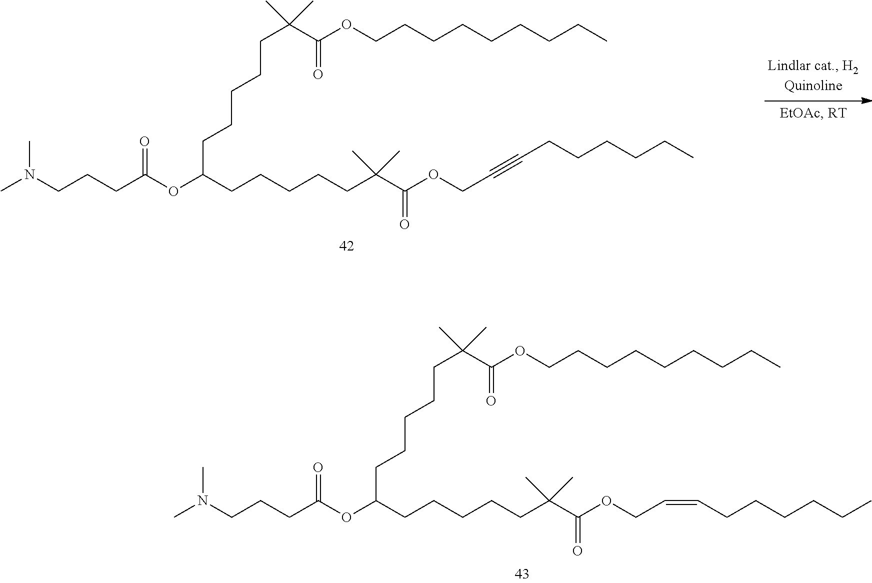

Referring to the method of Example 26, compound 43 was prepared as an oily product: 31.3 mg. ¹H NMR (400 MHz, CDCl₃): δ ppm 0.80 (m, 6H), 1.05 (s, 12H), 1.08-1.28 (m, 34H), 1.36-1.47 (m, 8H), 1.49-1.58 (m, 2H), 1.73-1.82 (m, 2H), 1.98-2.07 (m, 2H), 2.21-2.38 (m, 8H), 3.97 (t, J=7.2 Hz, 2H), 4.53 (d, J=7.2 Hz, 2H), 4.72-4.78 (m, 1H), 5.41-5.59 (m, 2H); ESI-MS m/z: 708.6 [M+H]⁺.

### Example 44: Synthesis of Compound 44

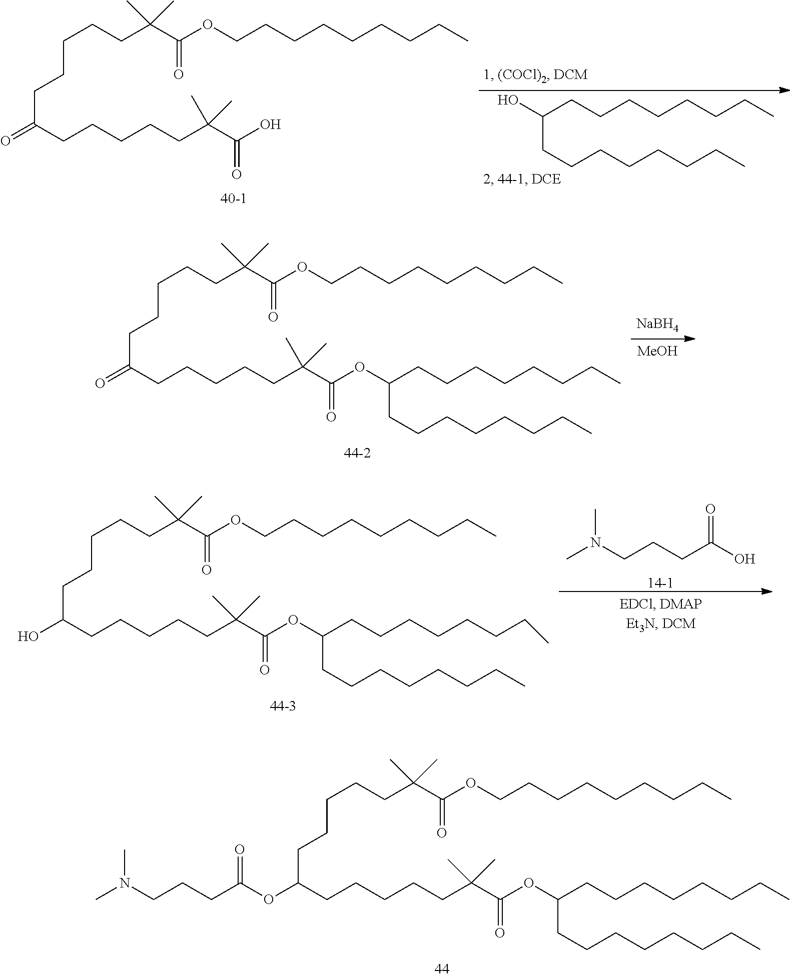

Referring to the method of Example 40, compound 44 was prepared as an oily product: 33.1 mg. ¹H NMR (400 MHz, CDCl₃): δ ppm 0.85 (m, 9H), 1.13 (s, 12H), 1.14-1.33 (m, 46H), 1.37-1.59 (m, 16H), 1.78-1.87 (m, 2H), 2.17-2.35 (m, 10H), 4.03 (t, J=6.8 Hz, 2H), 4.79-4.88 (m, 2H); ESI-MS m/z: 822.8 [M+H]⁺.

### Example 45: Synthesis of Compound 45

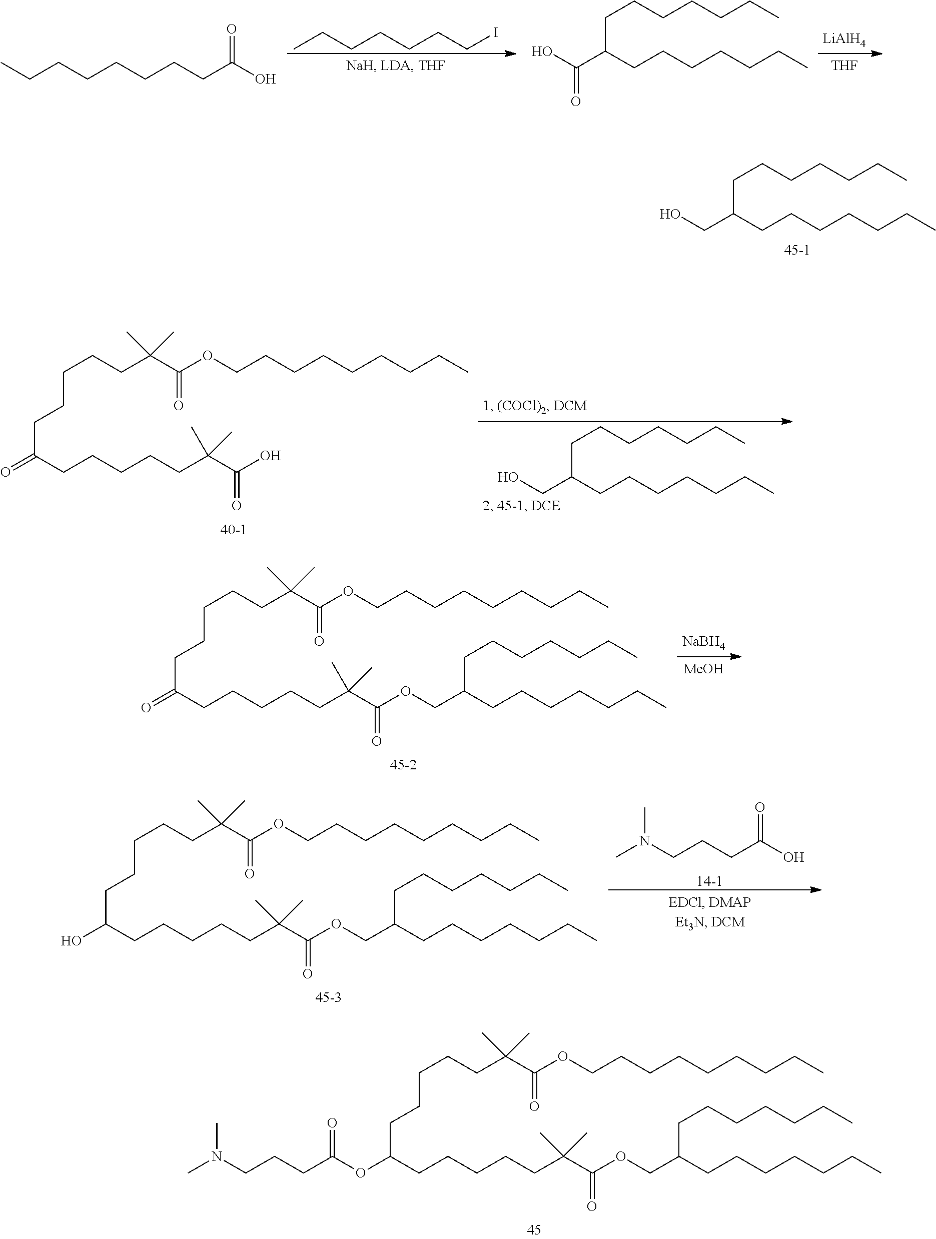

n-Nonanoic acid (3.0 g, 19 mmol) was added to 50 mL of anhydrous tetrahydrofuran and the reaction solution was cooled to 0° C. in an ice bath. Sodium hydride (836 mg, 20.9 mmol) and LDA (49.4 mL, 24.7 mmol) were added to the reaction solution, and the reaction solution was stirred at 0° C. for 1 hour. Then 1-iodoheptane was added dropwise to the reaction system. The ice bath was removed, then the mixture was reacted at room temperature for 12 h. The reaction solution was quenched by pouring the reaction solution into saturated ammonium chloride solution, and extracted with ethyl acetate. The organic phase was collected, dried over anhydrous sodium sulfate, and filtered. The filtrate was collected, and concentrated to remove the solvent to give the crude product, which was purified by silica gel column to give 2.0 g of compound 2-heptylnonanoic acid.

The 2-heptylnonanoic acid (2.0 g, 7.8 mmol) obtained in the previous step was dissolved in 30 mL of anhydrous tetrahydrofuran, and lithium tetrahydroaluminum (593 mg, 15.6 mmol) was added to the reaction solution. The reaction system was heated to 80° C. to react for 2 hours. The reaction solution was cooled to room temperature, quenched by pouring the reaction solution into saturated ammonium chloride solution, and extracted with ethyl acetate. The organic phase was collected, dried over anhydrous sodium sulfate, and filtered. The filtrate was collected, and concentrated to remove the solvent to give the crude product, which was purified by silica gel column to give 1.3 g of compound 45-1.

Then referring to the method of Example 40, compound 45 was prepared as an oily product: 31.6 mg. ¹H NMR (400 MHz, CDCl₃): δ ppm 0.88 (t, J=6.8 Hz, 9H), 1.14 (s, 12H), 1.15-1.26 (m, 47H), 1.47-1.50 (m, 8H), 1.57-1.62 (m, 4H), 1.79-1.81 (m, 2H), 2.25 (s, 6H), 2.32 (t, J=7.2 Hz, 4H), 3.93 (d, J=5.6 Hz, 2H), 4.03 (t, J=7.2 Hz, 2H), 4.81-4.87 (m, 1H); ESI-MS m/z: 808.7 [M+H]⁺.

### Example 46: Synthesis of Compound 46

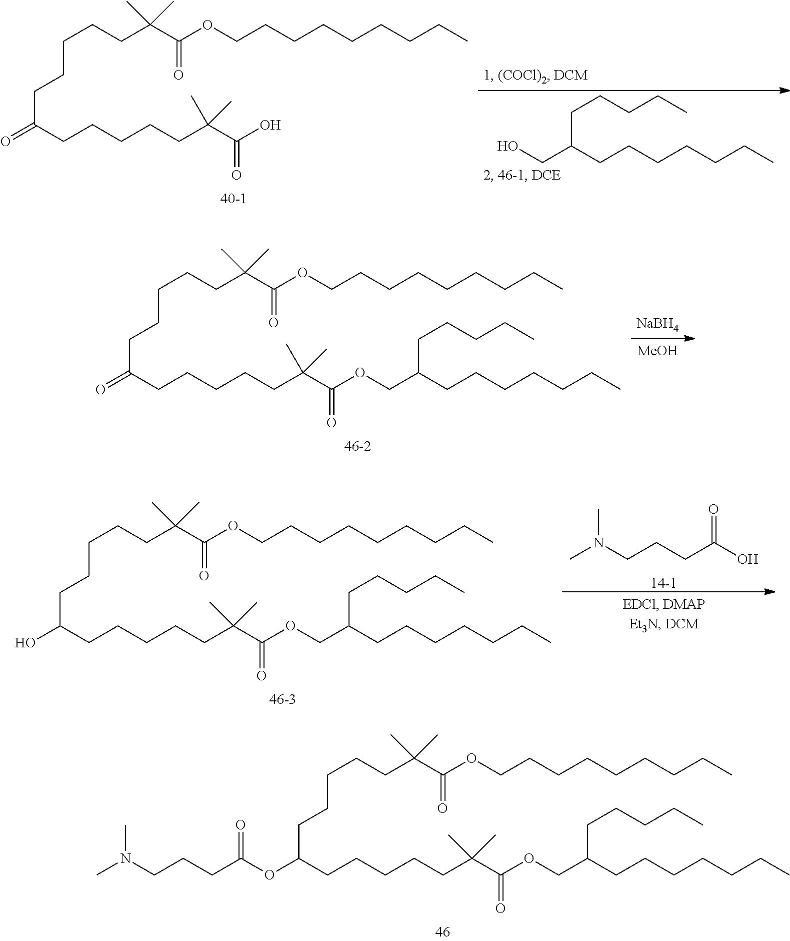

Referring to the method of Example 40, compound 46 was prepared as an oily product: 32.6 mg. ¹H NMR (400 MHz, CDCl₃): δ ppm 0.88 (t, J=7.2 Hz, 9H), 1.14 (s, 12H), 1.15-1.28 (m, 37H), 1.47-1.59 (m, 18H), 1.75-1.84 (m, 2H), 2.24-2.35 (m, 10H), 3.95 (d, J=5.6 Hz, 2H), 4.03 (t, J=6.8 Hz, 2H), 4.80-4.87 (m, 1H); ESI-MS m/z: 780.7 [M+H]⁺.

### Example 47: Synthesis of Compound 47

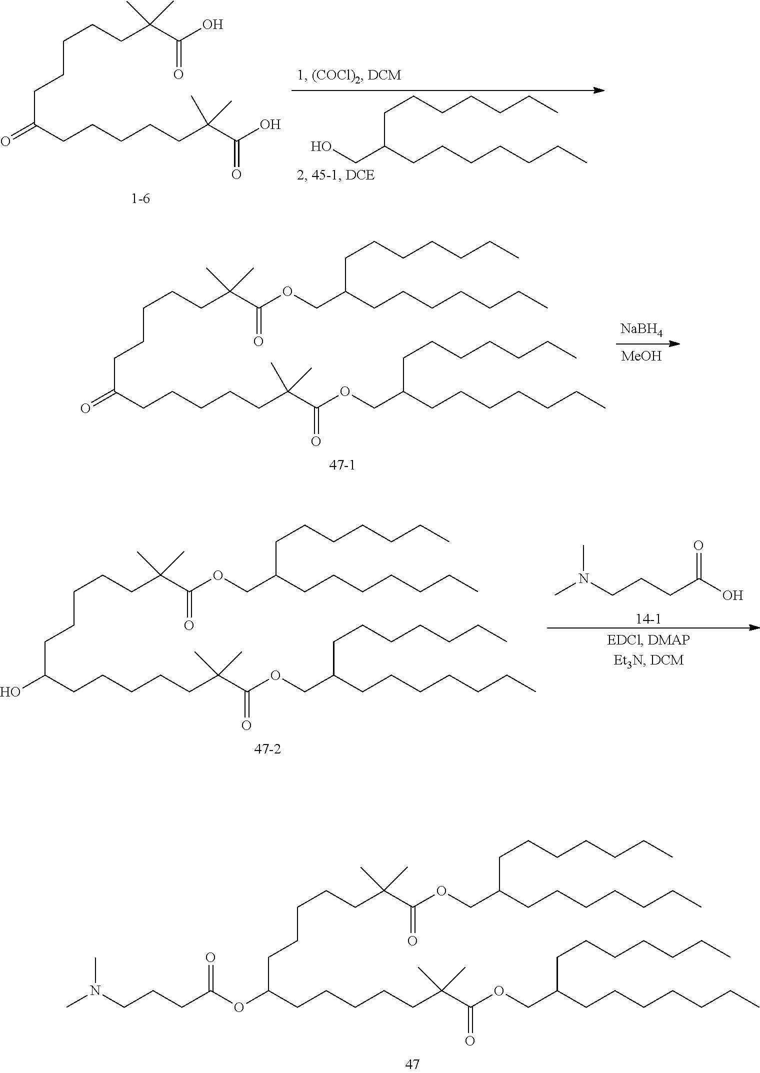

Referring to the method of Example 7, compound 47 was prepared as an oily product: 33.1 mg. ¹H NMR (400 MHz, CDCl₃): δ ppm 0.81 (t, J=6.8 Hz, 12H), 1.08 (s, 12H), 1.09-1.24 (m, 56H), 1.40-1.61 (m, 14H), 1.67-1.72 (m, 2H), 2.17 (s, 6H), 2.19-2.28 (m, 4H), 3.88 (d, J=5.6 Hz, 4H), 4.74-4.80 (m, 1H); ESI-MS m/z: 906.8 [M+H]⁺.

### Example 48: Synthesis of Compound 48

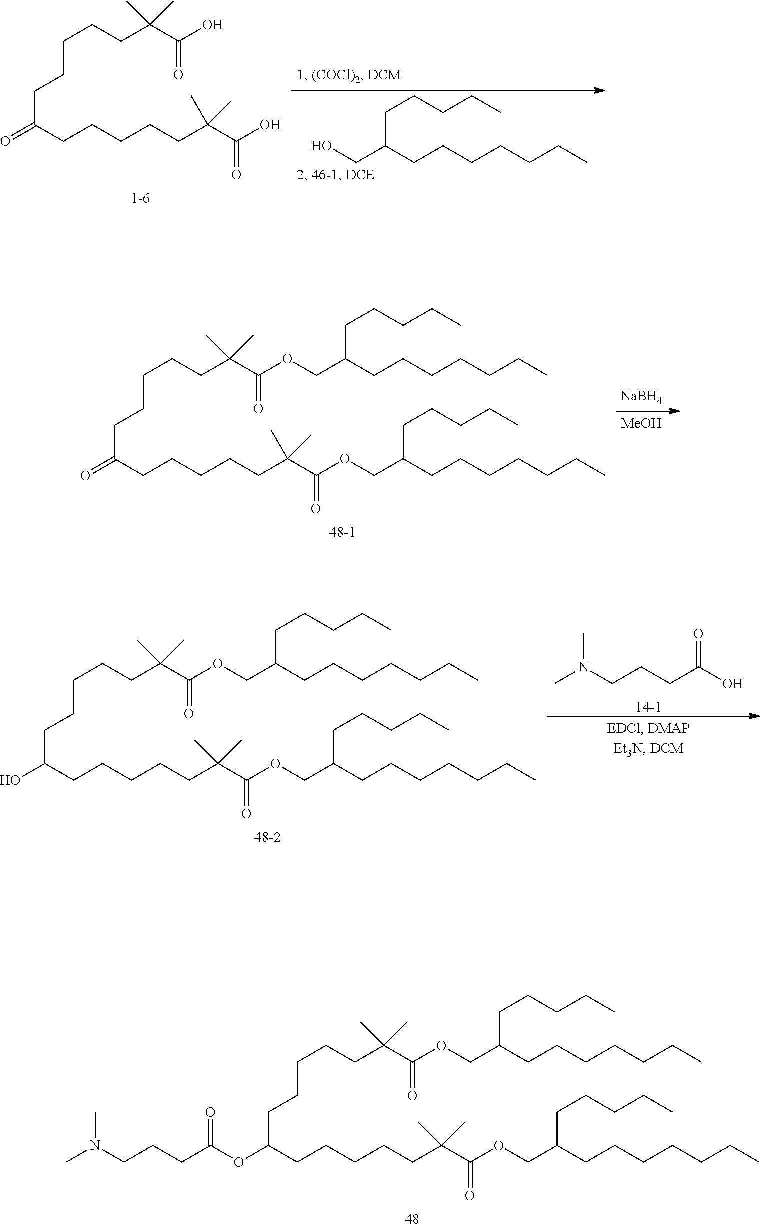

Referring to the method of Example 7, compound 48 was prepared as an oily product: 34.8 mg. ¹H NMR (400 MHz, CDCl₃): δ ppm 0.81 (t, J=7.2 Hz, 12H), 1.08 (s, 12H), 1.09-1.23 (m, 48H), 1.37-1.64 (m, 14H), 1.67-1.73 (m, 2H), 2.15 (s, 6H), 2.20-2.37 (m, 4H), 3.88 (d, J=5.6 Hz, 4H), 4.74-4.89 (m, 1H); ESI-MS m/z: 850.8 [M+H]⁺.

The compounds of Table 2 were synthesized using the methods of the above examples, or similar methods using the corresponding intermediates.

TABLE 2

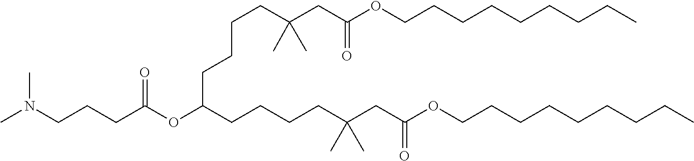 [SMILES: CCCCCCCCCOC(=O)CC(C)(C)CCCCC(CCCCC(C)(C)CC(=O)OCCCCCCCCC)OC(=O)CCCN(C)C]

Example 49: compound 49
[M + H]⁺: 710.6

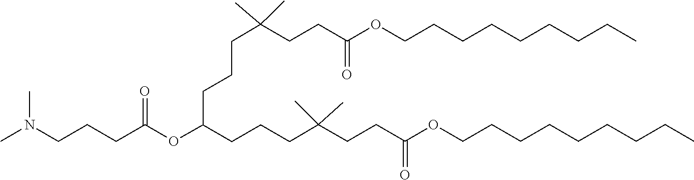 [SMILES: CCCCCCCCCOC(=O)CCC(C)(C)CCCC(CCCC(C)(C)CCC(=O)OCCCCCCCCC)OC(=O)CCCN(C)C]

Example 50: compound 50
[M + H]⁺: 710.6

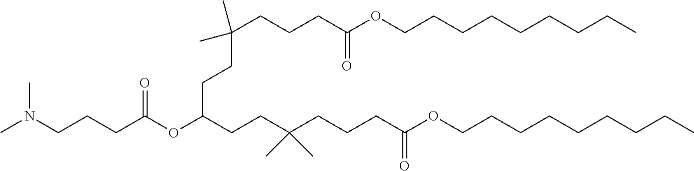 [SMILES: CCCCCCCCCOC(=O)CCCC(C)(C)CCC(CCC(C)(C)CCCC(=O)OCCCCCCCCC)OC(=O)CCCN(C)C]

Example 51: compound 51
[M + H]⁺: 710.6

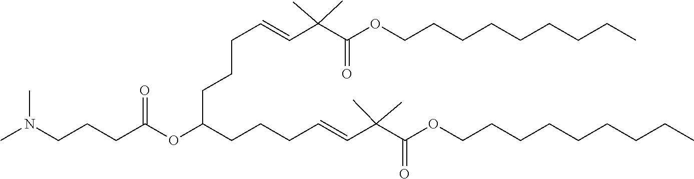 [SMILES: CCCCCCCCCOC(=O)C(C)(C)/C=C/CCCC(CCC/C=C/C(C)(C)C(=O)OCCCCCCCCC)OC(=O)CCCN(C)C]

Example 52: compound 52
[M + H]⁺: 706.6

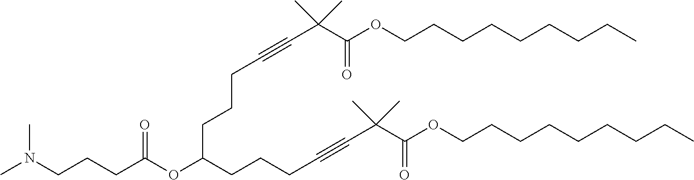 [SMILES: CCCCCCCCCOC(=O)C(C)(C)C#CCCCC(CCCC#CC(C)(C)C(=O)OCCCCCCCCC)OC(=O)CCCN(C)C]

Example 53: compound 53
[M + H]⁺: 702.6

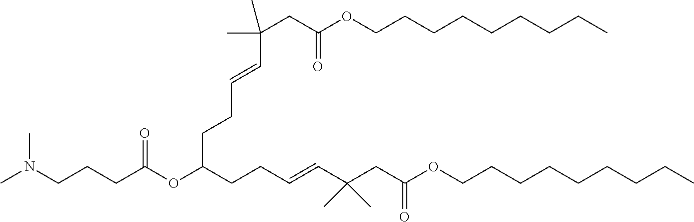 [SMILES: CCCCCCCCCOC(=O)CC(C)(C)/C=C/CCC(CC/C=C/C(C)(C)CC(=O)OCCCCCCCCC)OC(=O)CCCN(C)C]

Example 54: compound 54
[M + H]⁺: 706.6

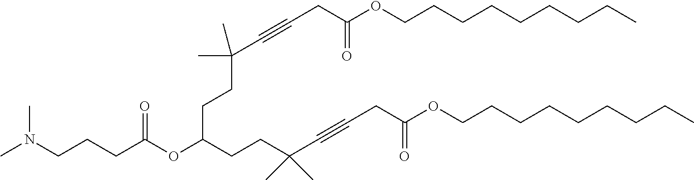 [SMILES: CCCCCCCCCOC(=O)CC#CC(C)(C)CCC(CCC(C)(C)C#CCC(=O)OCCCCCCCCC)OC(=O)CCCN(C)C]

Example 55: compound 55
[M + H]⁺: 702.6

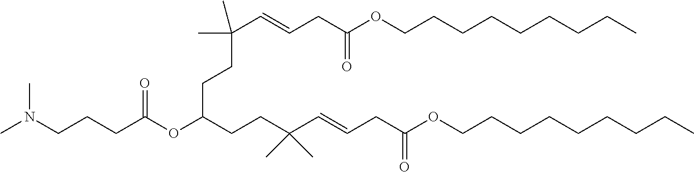 [SMILES: CCCCCCCCCOC(=O)C/C=C/C(C)(C)CCC(CCC(C)(C)/C=C/CC(=O)OCCCCCCCCC)OC(=O)CCCN(C)C]

Example 56: compound 56
[M + H]⁺: 706.6

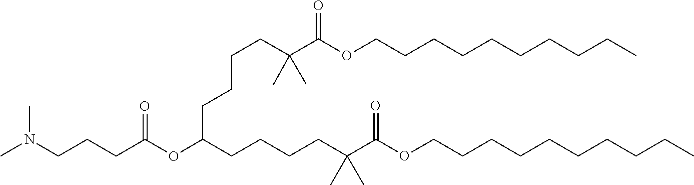 [SMILES: CCCCCCCCCCOC(=O)C(C)(C)CCCCC(CCCCC(C)(C)C(=O)OCCCCCCCCCC)OC(=O)CCCN(C)C]

Example 57: compound 57
[M + H]⁺: 710.6

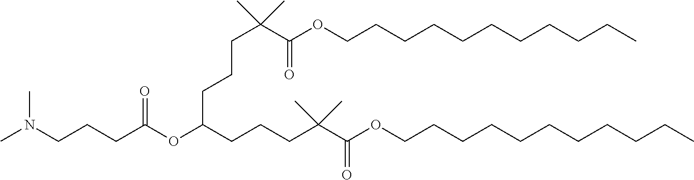 [SMILES: CCCCCCCCCCCOC(=O)C(C)(C)CCCC(CCCC(C)(C)C(=O)OCCCCCCCCCCC)OC(=O)CCCN(C)C]

Example 58: compound 58
[M + H]⁺: 710.6

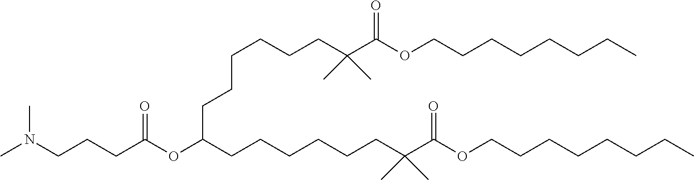 [SMILES: CCCCCCCCOC(=O)C(C)(C)CCCCCCC(CCCCCCC(C)(C)C(=O)OCCCCCCCC)OC(=O)CCCN(C)C]

Example 59: compound 59
[M + H]⁺: 710.6

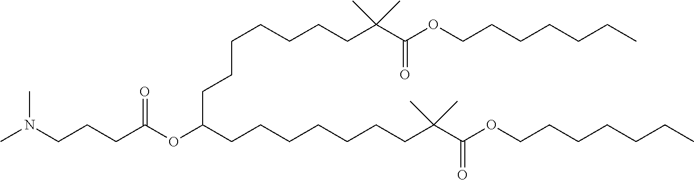 [SMILES: CCCCCCCOC(=O)C(C)(C)CCCCCCCC(CCCCCCCC(C)(C)C(=O)OCCCCCCC)OC(=O)CCCN(C)C]

Example 60: compound 60
[M + H]⁺: 710.6

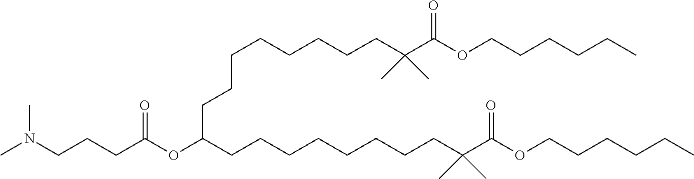 [SMILES: CCCCCCOC(=O)C(C)(C)CCCCCCCCC(CCCCCCCCC(C)(C)C(=O)OCCCCCC)OC(=O)CCCN(C)C]

Example 61: compound 61
[M + H]⁺: 710.6

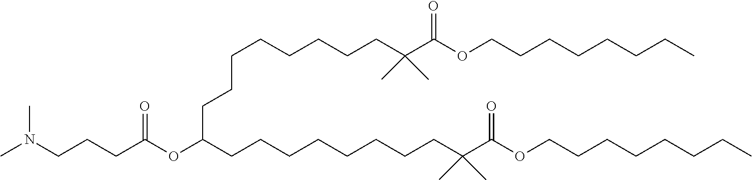 [SMILES: CCCCCCCCOC(=O)C(C)(C)CCCCCCCCC(CCCCCCCCC(C)(C)C(=O)OCCCCCCCC)OC(=O)CCCN(C)C]

Example 62: compound 62
[M + H]⁺: 766.7

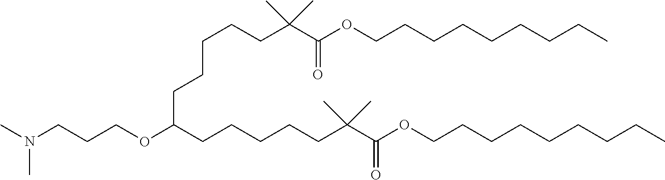 [SMILES: CCCCCCCCCOC(=O)C(C)(C)CCCCCC(CCCCCC(C)(C)C(=O)OCCCCCCCCC)OCCCN(C)C]

Example 63: compound 63
[M + H]⁺: 682.6

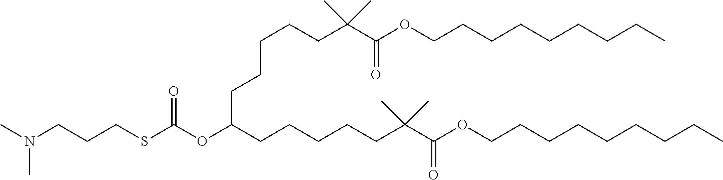 [SMILES: CCCCCCCCCOC(=O)C(C)(C)CCCCCC(CCCCCC(C)(C)C(=O)OCCCCCCCCC)OC(=O)SCCCN(C)C]

Example 64: compound 64
[M + H]⁺: 742.6

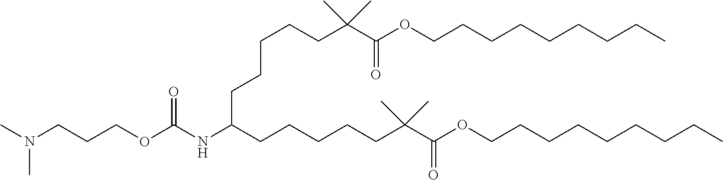 [SMILES: CCCCCCCCCOC(=O)C(C)(C)CCCCCC(CCCCCC(C)(C)C(=O)OCCCCCCCCC)NC(=O)OCCCN(C)C]

Example 65: compound 65
[M + H]⁺: 725.6

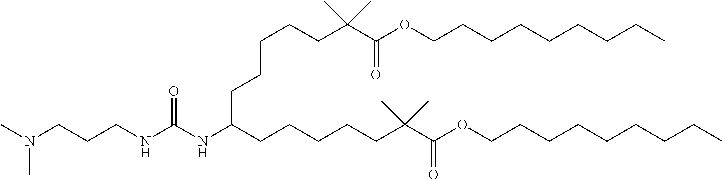 [SMILES: CCCCCCCCCOC(=O)C(C)(C)CCCCCC(CCCCCC(C)(C)C(=O)OCCCCCCCCC)NC(=O)NCCCN(C)C]

Example 66: compound 66
[M + H]⁺: 724.6

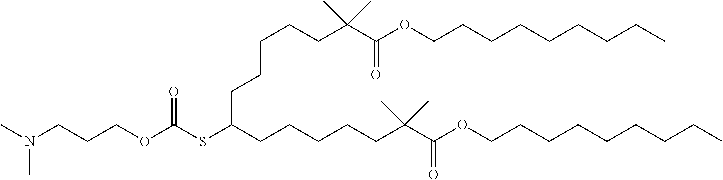 [SMILES: CCCCCCCCCOC(=O)C(C)(C)CCCCCC(CCCCCC(C)(C)C(=O)OCCCCCCCCC)SC(=O)OCCCN(C)C]

Example 67: compound 67
[M + H]⁺: 742.6

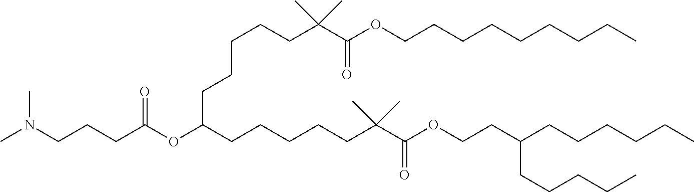 [SMILES: CCCCCCCCCOC(=O)C(C)(C)CCCCCC(CCCCCC(C)(C)C(=O)OCCC(CCCCC)CCCCCC)OC(=O)CCCN(C)C]

Example 68: compound 68
[M + H]⁺: 780.7

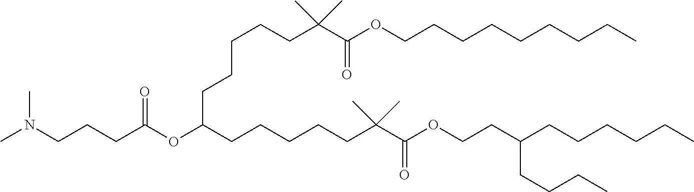 [SMILES: CCCCCCCCCOC(=O)C(C)(C)CCCCCC(CCCCCC(C)(C)C(=O)OCCC(CCCC)CCCCCC)OC(=O)CCCN(C)C]

Example 69: compound 69
[M + H]⁺: 766.7

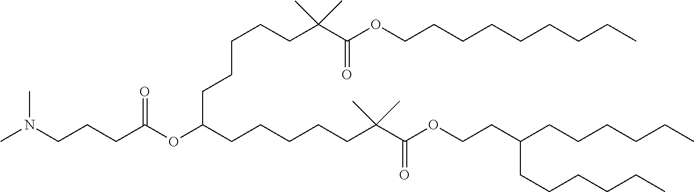 [SMILES: CCCCCCCCCOC(=O)C(C)(C)CCCCCC(CCCCCC(C)(C)C(=O)OCCC(CCCCCC)CCCCCC)OC(=O)CCCN(C)C]

Example 70: compound 70
[M + H]⁺: 794.7

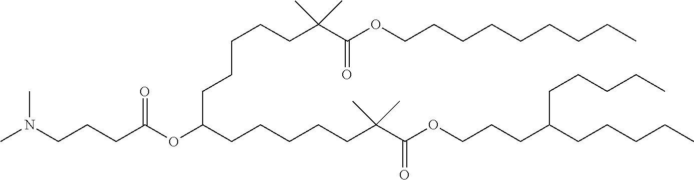 [SMILES: CCCCCCCCCOC(=O)C(C)(C)CCCCCC(CCCCCC(C)(C)C(=O)OCCCC(CCCCC)CCCCC)OC(=O)CCCN(C)C]

Example 71: compound 71
[M + H]⁺: 780.7

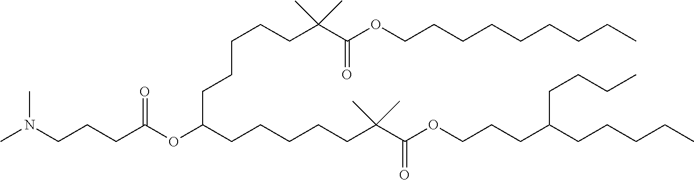 [SMILES: CCCCCCCCCOC(=O)C(C)(C)CCCCCC(CCCCCC(C)(C)C(=O)OCCCC(CCCC)CCCCC)OC(=O)CCCN(C)C]

Example 72: compound 72
[M + H]⁺: 766.7

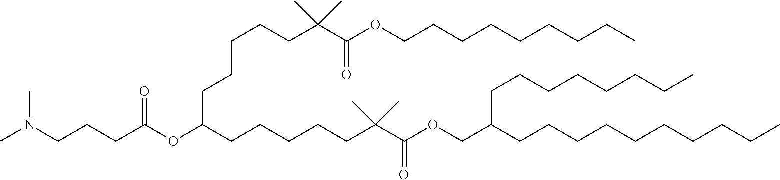 [SMILES: CCCCCCCCCCC(CCCCCCCC)COC(=O)C(C)(C)CCCCCC(CCCCCC(C)(C)C(=O)OCCCCCCCCC)OC(=O)CCCN(C)C]

Example 73: compound 73
[M + H]⁺: 864.8

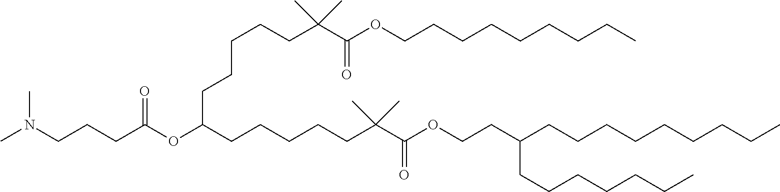 [SMILES: CCCCCCCCCOC(=O)C(C)(C)CCCCCC(CCCCCC(C)(C)C(=O)OCCC(CCCCCCC)CCCCCCCCC)OC(=O)CCCN(C)C]

Example 74: compound 74
[M + H]⁺: 850.8

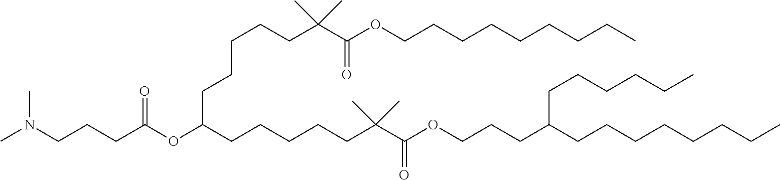 [SMILES: CCCCCCCCCOC(=O)C(C)(C)CCCCCC(CCCCCC(C)(C)C(=O)OCCCC(CCCCCC)CCCCCCCC)OC(=O)CCCN(C)C]

Example 75: compound 75
[M + H]⁺: 836.8

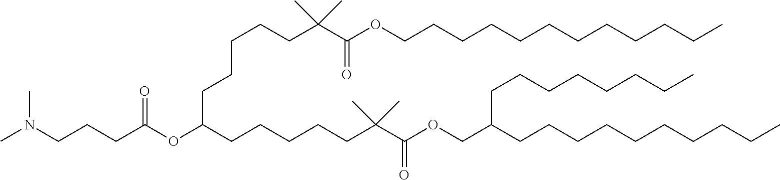 [SMILES: CCCCCCCCCCCCOC(=O)C(C)(C)CCCCCC(CCCCCC(C)(C)C(=O)OCC(CCCCCCCC)CCCCCCCCCC)OC(=O)CCCN(C)C]

Example 77: compound 77
[M + H]⁺: 906.8

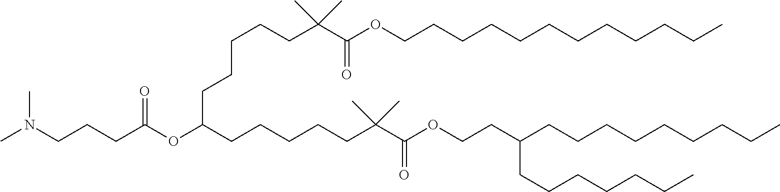 [SMILES: CCCCCCCCCCCCOC(=O)C(C)(C)CCCCCC(CCCCCC(C)(C)C(=O)OCCC(CCCCCCC)CCCCCCCCC)OC(=O)CCCN(C)C]

Example 78: compound 78
[M + H]⁺: 892.8

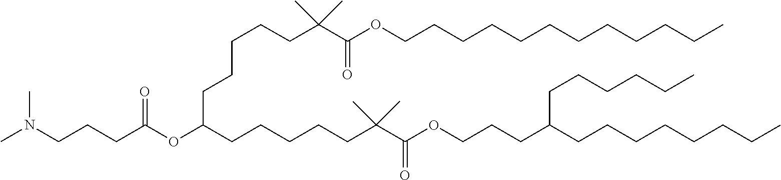 [SMILES: CCCCCCCCCCCCOC(=O)C(C)(C)CCCCCC(CCCCCC(C)(C)C(=O)OCCCC(CCCCCC)CCCCCCCC)OC(=O)CCCN(C)C]

Example 79: compound 79
[M + H]⁺: 878.8

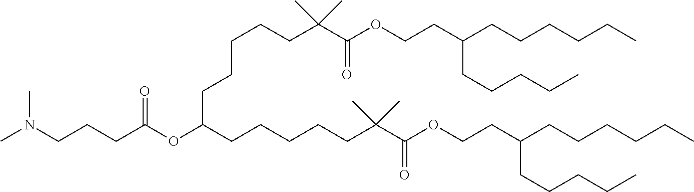 [SMILES: CCCCCCC(CCCCC)CCOC(=O)C(C)(C)CCCCCC(CCCCCC(C)(C)C(=O)OCCC(CCCCC)CCCCCC)OC(=O)CCCN(C)C]

Example 80: compound 80
[M + H]⁺: 850.8

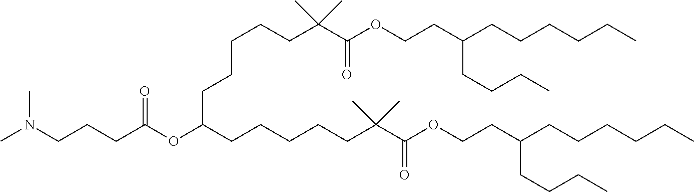 [SMILES: CCCCCCC(CCCC)CCOC(=O)C(C)(C)CCCCCC(CCCCCC(C)(C)C(=O)OCCC(CCCC)CCCCCC)OC(=O)CCCN(C)C]

Example 81: compound 81
[M + H]⁺: 822.8

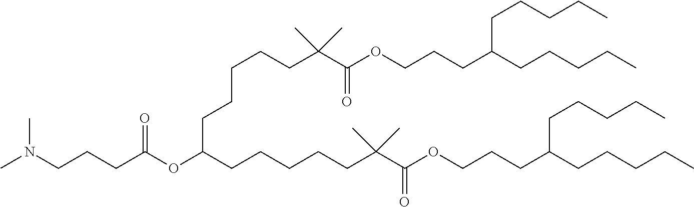 [SMILES: CCCCCC(CCCCC)CCCOC(=O)C(C)(C)CCCCCC(CCCCCC(C)(C)C(=O)OCCCC(CCCCC)CCCCC)OC(=O)CCCN(C)C]

Example 82: compound 82
[M + H]⁺: 850.8

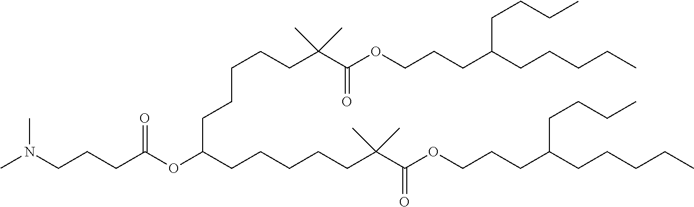 [SMILES: CCCCCC(CCCC)CCCOC(=O)C(C)(C)CCCCCC(CCCCCC(C)(C)C(=O)OCCCC(CCCC)CCCCC)OC(=O)CCCN(C)C]

Example 83: compound 83
[M + H]⁺: 822.8

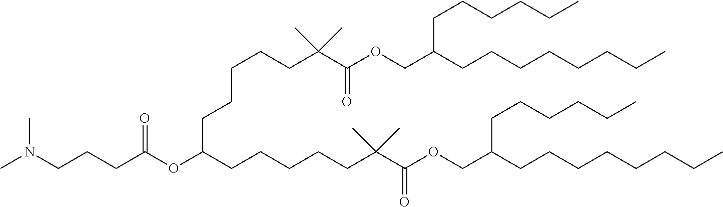 [SMILES: CCCCCCCCC(CCCCCC)COC(=O)C(C)(C)CCCCCC(CCCCCC(C)(C)C(=O)OCC(CCCCCC)CCCCCCCC)OC(=O)CCCN(C)C]

Example 84: compound 84
[M + H]⁺: 906.8

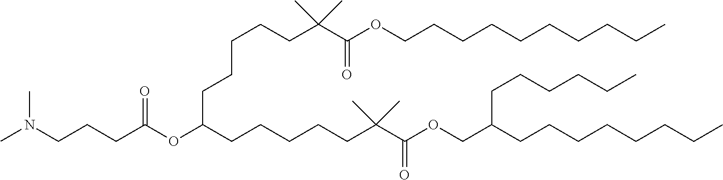 [SMILES: CCCCCCCCCCOC(=O)C(C)(C)CCCCCC(CCCCCC(C)(C)C(=O)OCC(CCCCCC)CCCCCCCC)OC(=O)CCCN(C)C]

Example 85: compound 85
[M + H]⁺: 822.8

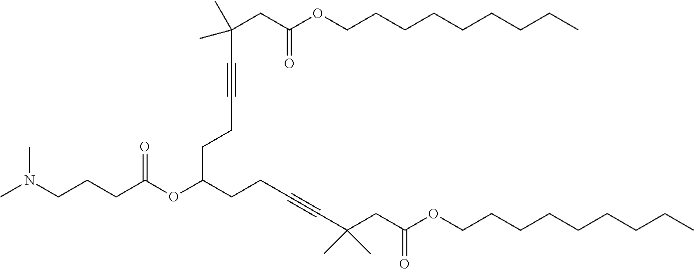 [SMILES: CCCCCCCCCOC(=O)CC(C)(C)C#CCCC(CCC#CC(C)(C)CC(=O)OCCCCCCCCC)OC(=O)CCCN(C)C]

Example 86: compound 86
[M + H]⁺: 702.6

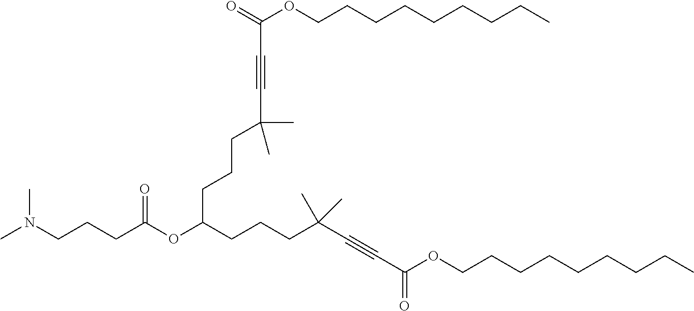 [SMILES: CCCCCCCCCOC(=O)C#CC(C)(C)CCCC(CCCC(C)(C)C#CC(=O)OCCCCCCCCC)OC(=O)CCCN(C)C]

Example 87: compound 87
[M + H]⁺: 702.6

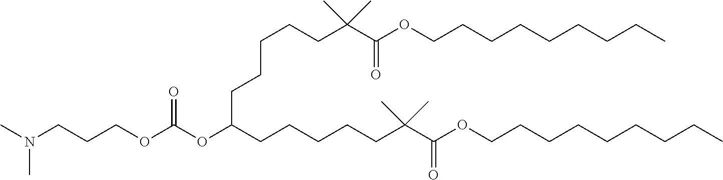 [SMILES: CCCCCCCCCOC(=O)C(C)(C)CCCCCC(CCCCCC(C)(C)C(=O)OCCCCCCCCC)OC(=O)OCCCN(C)C]

Example 88: compound 88
[M + H]⁺: 726.6

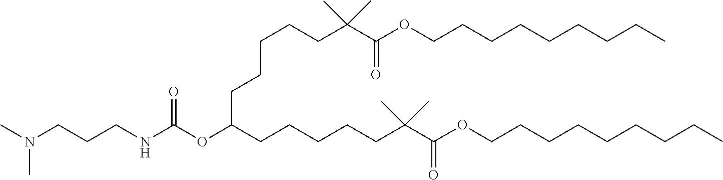 [SMILES: CCCCCCCCCOC(=O)C(C)(C)CCCCCC(CCCCCC(C)(C)C(=O)OCCCCCCCCC)OC(=O)NCCCN(C)C]

Example 89: compound 89
[M + H]⁺: 725.6

### Example 90: Synthesis of Compound 90

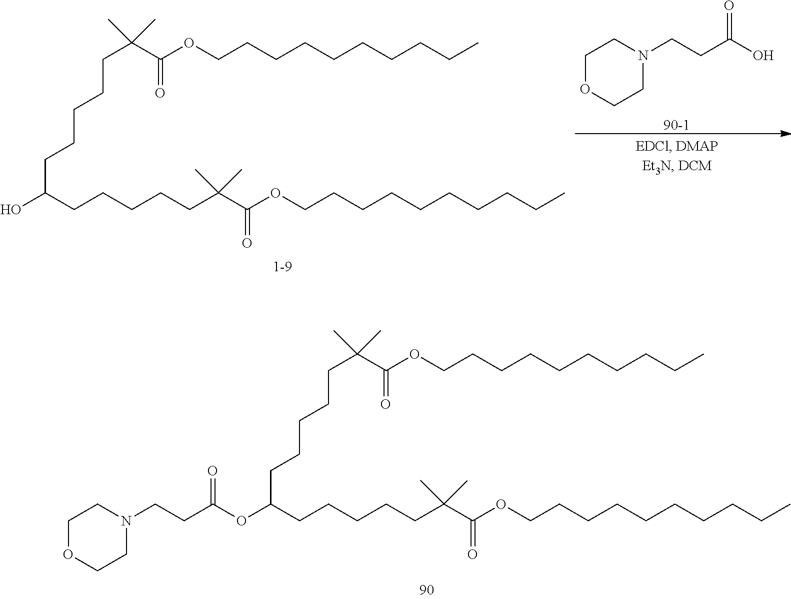

Referring to the method of Example 1, compound 90 was prepared as an oily product: 40.5 mg. ¹H NMR (400 MHz, CDCl₃): δ ppm 0.81 (t, J=6.8 Hz, 6H), 1.08 (s, 12H), 1.10-1.28 (m, 36H), 1.38-1.47 (m, 12H), 1.50-1.58 (m, 4H), 2.40 (m, 6H), 2.58 (t, J=6.8 Hz, 2H), 3.59-3.65 (m, 4H), 3.97 (t, J=6.8 Hz, 4H), 4.75-4.83 (m, 1H); ESI-MS m/z: 766.7 [M+H]⁺.

### Example 91: Synthesis of Compound 91

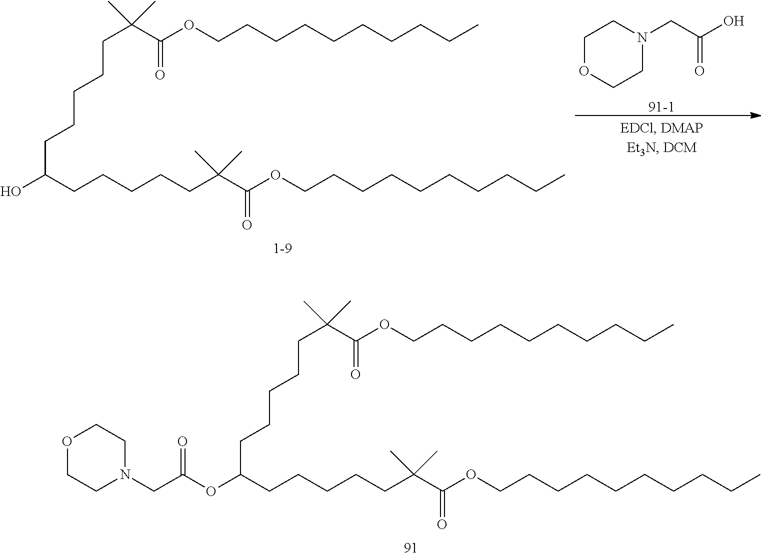

Referring to the method of Example 1, compound 91 was prepared as an oily product: 32.2 mg. ¹H NMR (400 MHz, CDCl₃): δ ppm 0.88 (t, J=6.8 Hz, 6H), 1.15 (s, 12H), 1.16-1.38 (m, 40H), 1.46 (m, 8H), 1.60 (m, 4H), 2.59 (m, 4H), 3.19 (s, 2H), 3.76 (t, J=4.8 Hz, 4H), 4.04 (t, J=6.8 Hz, 4H), 4.91 (m, 1H); ESI-MS m/z: 752.7 [M+H]⁺.

### Example 92: Synthesis of Compound 92

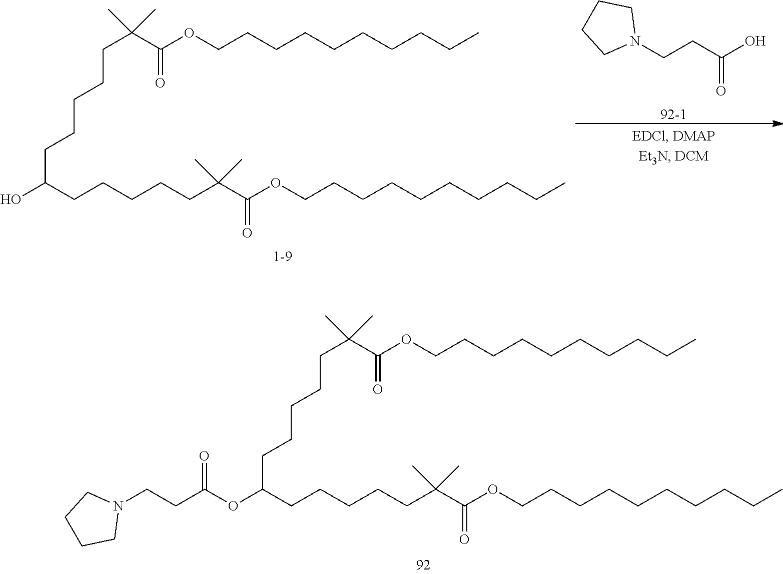 [SMILES: CCCCCCCCCCOC(=O)C(C)(C)CCCCCC(O)CCCCCC(C)(C)C(=O)OCCCCCCCCCC ; O=C(O)CCN1CCCC1 ; "92-1" ; "EDCl, DMAPEt3N, DCM" ; CCCCCCCCCCOC(=O)C(C)(C)CCCCCC(CCCCCC(C)(C)C(=O)OCCCCCCCCCC)OC(=O)CCN1CCCC1]

Referring to the method of Example 1, compound 92 was prepared as an oily product: 32 mg. H NMR (400 MHz, CDCl₃): δ ppm 0.87 (t, J=6.8 Hz, 6H), 1.13 (s, 12H), 1.28 (m, 42H), 1.46 (m, 8H), 1.45 (m, 4H), 1.76 (m, 4H), 2.50 (m, 4H), 2.76 (m, 2H), 4.01 (t, J=6.8 Hz, 4H), 4.84 (m, 1H); ESI-MS m/z: 750.9 [M+H]⁺.

### Example 93: Synthesis of Compound 93

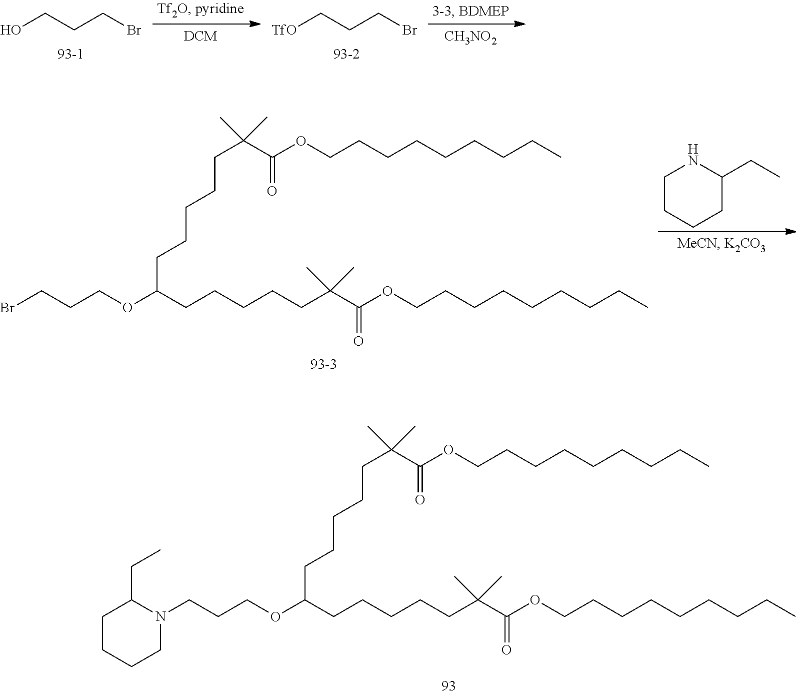

3-Bromopropanol (20 g, 144 mmol), trifluoromethanesulfonic anhydride (26.6 mL, 158 mmol) and pyridine (14.0 mL, 173 mmol) were added to a round bottom flask containing 500 mL of dichloromethane. The mixture was stirred at room temperature until the reaction materials were completely consumed by TLC monitoring. The reaction solution was quenched with 1 M hydrochloric acid solution, and extracted with dichloromethane. The organic phase were combined, dried over anhydrous sodium sulfate, and filtered to remove the sodium sulfate. The filtrate was collected. The solvent was removed using a rotary-evaporator to give 25 g of crude compound 93-2, which was used directly for subsequent reactions without further purification.

3-3 (6.0 g, 10 mmol) and crude compound 93-2 (3.0 g, 11 mmol) were added to a round bottom flask containing 50 mL of nitromethane, then 2,6-di-tert-butylpyridine (3.37 mL, 15 mmol) was added to the reaction solution. The reaction solution was warmed up to 95° C. to react overnight. The reaction solution was cooled to room temperature. The solvent was removed using a rotary-evaporator to give the crude product. The crude product was then dissolved in dichloromethane, extracted after adding saturated aqueous ammonium chloride. The organic phase were collected and combined, dried over anhydrous sodium sulfate, and filtered to remove the sodium sulfate. The filtrate was collected. The solvent was removed using a rotary-evaporator and then purified by silica gel column to give compound 93-3 (2.3 g).

Compound 93-3 (251 mg, 0.35 mmol) and 2-ethylpiperidine (71 μL, 0.53 mmol) were dissolved in 3.0 mL of anhydrous acetonitrile and anhydrous potassium carbonate (73 mg, 0.53 mmol) was added to the reaction solution. The mixture was warmed up to 80° C. to react for 6 hours. The reaction solution was cooled to room temperature, quenched by adding saturated aqueous ammonium chloride, and extracted with dichloromethane. The organic phase were collected and combined, dried over anhydrous sodium sulfate, and filtered to remove the sodium sulfate. The filtrate was collected. The solvent was removed using a rotary-evaporator and then purified by preparative high performance liquid chromatography to give compound 93 (82 mg). ¹H NMR (400 MHz, CDCl₃): δ ppm 0.72-0.91 (m, 9H), 1.08 (s, 12H), 1.11-1.75 (m, 60H), 1.87-2.25 (m, 3H), 2.57-2.93 (m, 4H), 3.04-3.15 (m, 2H), 3.32-3.45 (m, 2H), 3.98 (d, J=6.8 Hz, 4H); ESI-MS m/z: 750.6 [M+H]⁺.

### Example 94: Synthesis of Compound 94

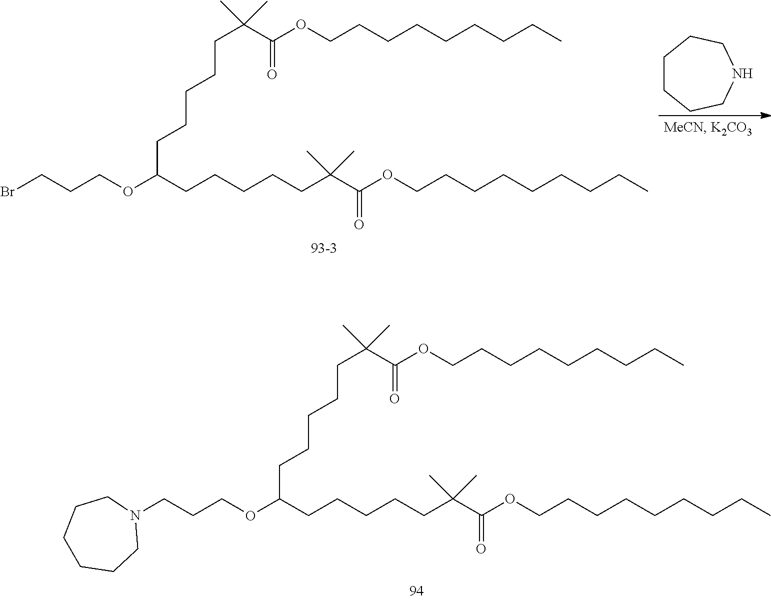 [SMILES: CCCCCCCCCOC(=O)C(C)(C)CCCCCC(CCCCCC(C)(C)C(=O)OCCCCCCCCC)OCCCBr ; C1CCCNCC1 ; CCCCCCCCCOC(=O)C(C)(C)CCCCCC(CCCCCC(C)(C)C(=O)OCCCCCCCCC)OCCCN1CCCCCC1]

Referring to the method of Example 93, compound 94 was prepared as an oily product: 79.2 mg. ¹H NMR (400 MHz, CDCl₃): δ ppm 0.82 (t, J=7.2 Hz, 6H), 1.09 (s, 12H), 1.11-1.34 (m, 44H), 1.52 (m, 14H), 2.45-2.74 (m, 6H), 3.07 (m, 1H), 3.38 (m, 2H), 3.94 (t, J=6.8 Hz, 4H); ESI-MS m/z: 736.6 [M+H]⁺.

The compounds of Table 3 were synthesized using the methods of the above examples, or similar methods using the corresponding intermediates.

TABLE 3

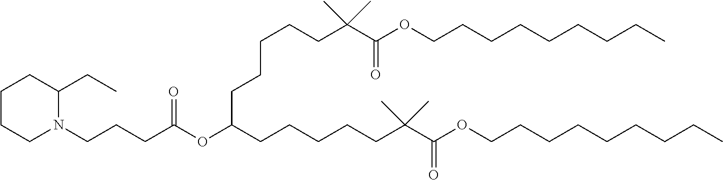 [SMILES: CCCCCCCCCOC(=O)C(C)(C)CCCCCC(CCCCCC(C)(C)C(=O)OCCCCCCCCC)OC(=O)CCCN1CCCCC1CC]

Example 95: compound 95
[M + H]⁺: 778.7

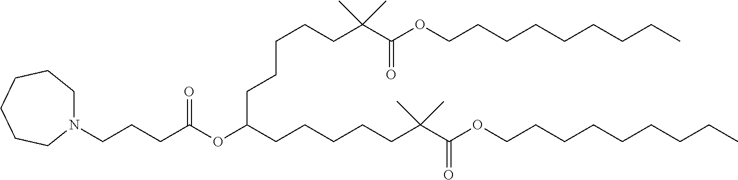 [SMILES: CCCCCCCCCOC(=O)C(C)(C)CCCCCC(CCCCCC(C)(C)C(=O)OCCCCCCCCC)OC(=O)CCCN1CCCCCC1]

Example 96: compound 96
[M + H]⁺: 764.7

### Example 97: Synthesis of Compound 97

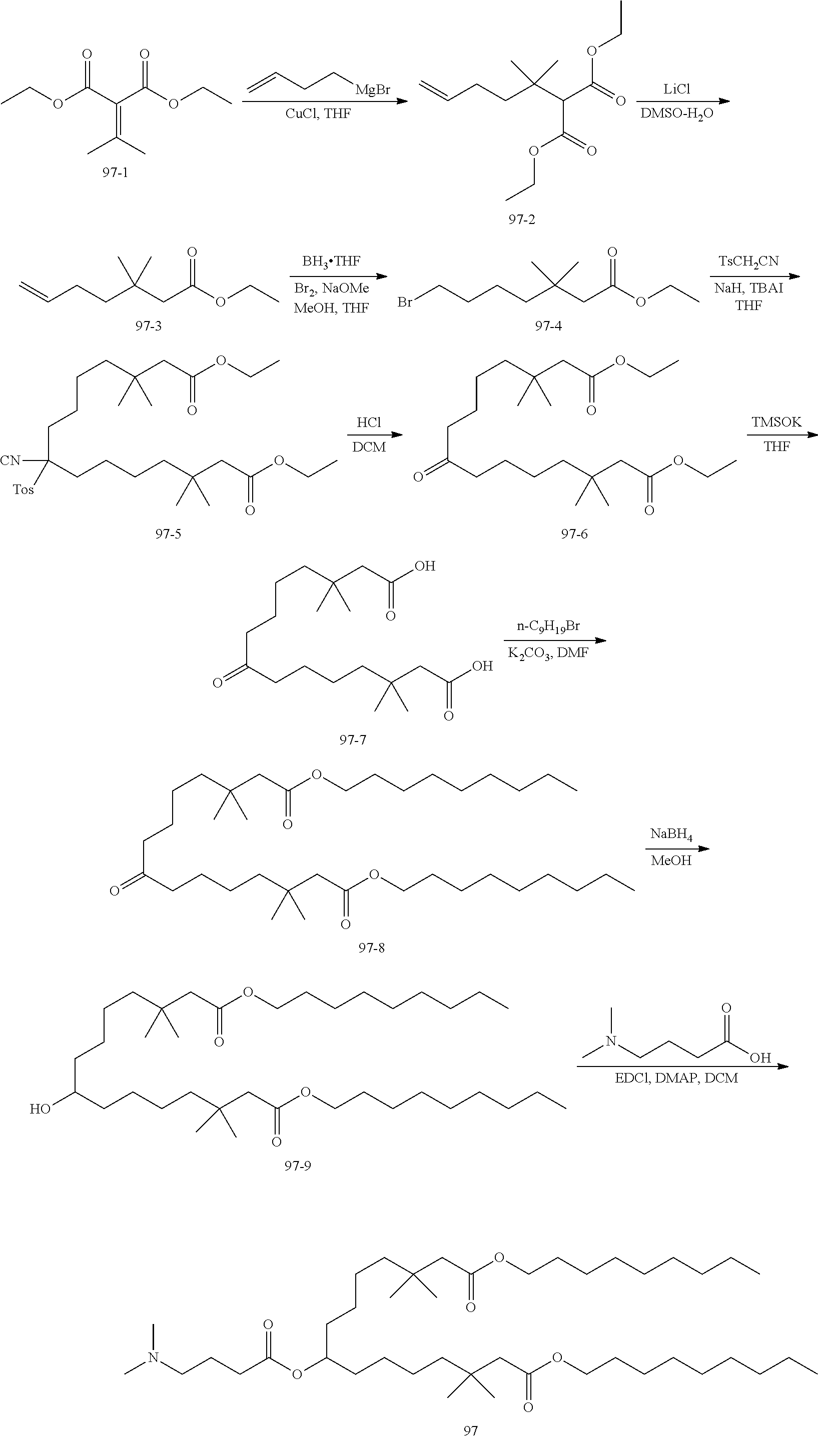

To a round bottom flask were added CuCl (989 mg, 9.99 mmol) and 160 mL THF, and the reaction system was cooled to −30° C. Then 3-butylmagnesium bromide (1 M, 299 mL) was added. 160 mL of solution of compound 97-1 (40.0 g, 199 mmol) in tetrahydrofuran was slowly added to the reaction system. After the dropwise addition was completed, the reaction system was warmed up to room temperature and stirred to react for another 2 hours. After the reaction material 97-1 was reacted completely by TLC monitoring, the reaction solution was quenched with 300 mL of saturated aqueous ammonium chloride, and extracted with ethyl acetate. The organic phases were combined, dried over anhydrous sodium sulfate, and filtered. The filtrate was concentrated to dryness to give the crude product. The crude was purified by silica gel column to give compound 97-2 (45.0 g).

Compound 97-2 (42.0 g, 164 mmol) was dissolved in 400 mL of DMSO, and 4 mL of water and LiCl (27.8 g, 655 mmol) were added to the reaction solution. Then the reaction system was heated to 180° C. and stirred until the reactant 97-2 was reacted completely by TLC monitoring. The reaction system was cooled to room temperature, then poured into water and extracted with ethyl acetate. The organic phases were combined, dried over anhydrous sodium sulfate, and filtered. The filtrate was concentrated to dryness to give the crude product 97-3 (31.0 g), which was used directly in the next reaction without further purification.

Crude product 97-3 (30.0 g, 163 mmol) was dissolved in 240 mL of tetrahydrofuran and BH₃·THF (1 M, 244 mL) was added dropwise to the reaction solution in an ice bath. Then the mixture was warmed up to room temperature and stirred for 2 h. The reaction system was then cooled to 0° C. in an ice bath and methanol (13.2 mL, 325 mmol), Br₂ (8.39 mL, 163 mmol) and sodium methoxide (43.9 g, 244 mmol) were added sequentially. The mixture was warmed up to room temperature and stirred for another 1 h. The reaction solution was quenched with cold saturated aqueous ammonium chloride solution and extracted with ethyl acetate. The organic phases were combined, dried over anhydrous sodium sulfate, and filtered. The filtrate was concentrated to dryness to give the crude product, which was purified by silica gel column to give compound 97-4 (14.0 g).

Then referring to the method of Example 1, compound 97 was prepared as an oily product: 31.6 mg. ¹H NMR (400 MHz, CDCl₃): δ ppm 0.84-0.90 (m, 6H), 0.93-1.01 (m, 12H), 1.20-1.31 (m, 32H), 1.45-1.62 (m, 16H), 2.17 (s, 4H), 2.19-2.44 (m, 8H), 3.99-4.08 (m, 4H), 4.81-4.91 (m, 1H); ESI-MS m/z: 710.7 [M+H]⁺.

### Example 98: Synthesis of Compound 98

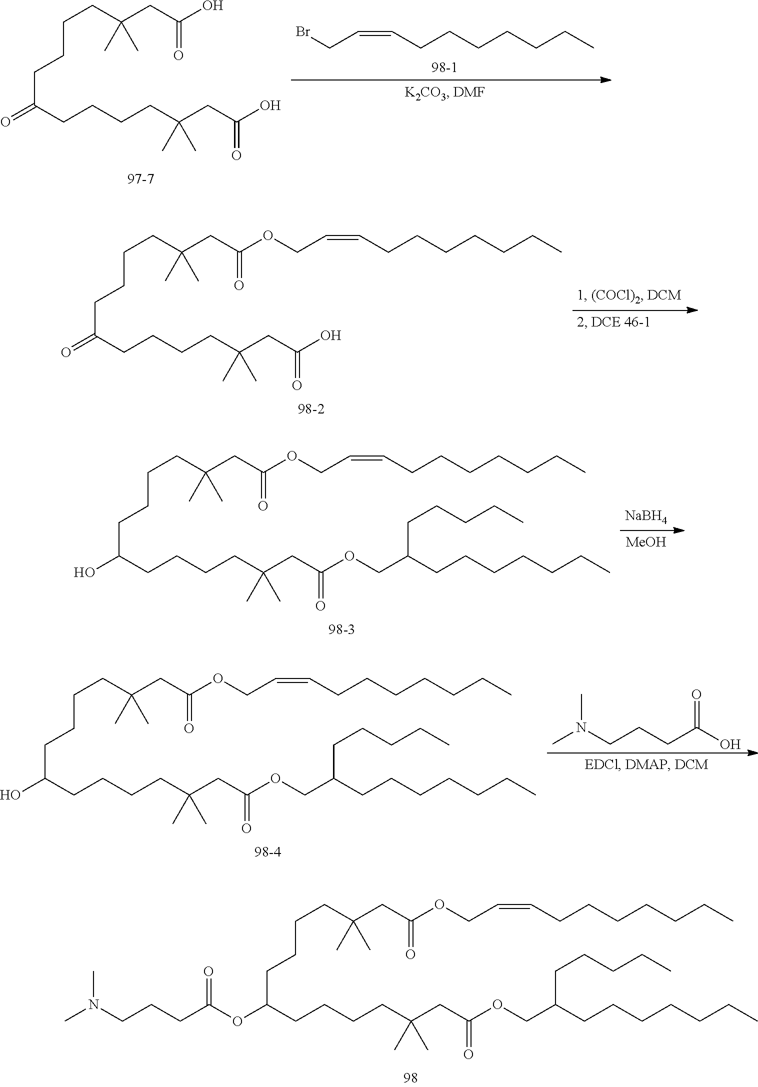

Referring to the method of Example 97, compound 98 was prepared as an oily product: 31.0 mg. ¹H NMR (400 MHz, CDCl₃): δ ppm 0.88 (t, J=6.8 Hz, 9H), 0.97 (s, 12H), 1.25-1.39 (m, 38H), 1.45-1.59 (m, 10H), 1.79 (m, 2H), 2.06-2.13 (m, 2H), 2.18 (m, 4H), 2.20-2.39 (m, 9H), 3.94 (d, J=5.6 Hz, 2H), 4.59 (d, J=6.8 Hz, 2H), 4.82-4.87 (m, 1H), 5.48-5.53 (m, 1H), 5.60-5.64 (m, 1H); ESI-MS m/z: 792.7 [M+H]⁺.

### Example 99: Synthesis of Compound 99

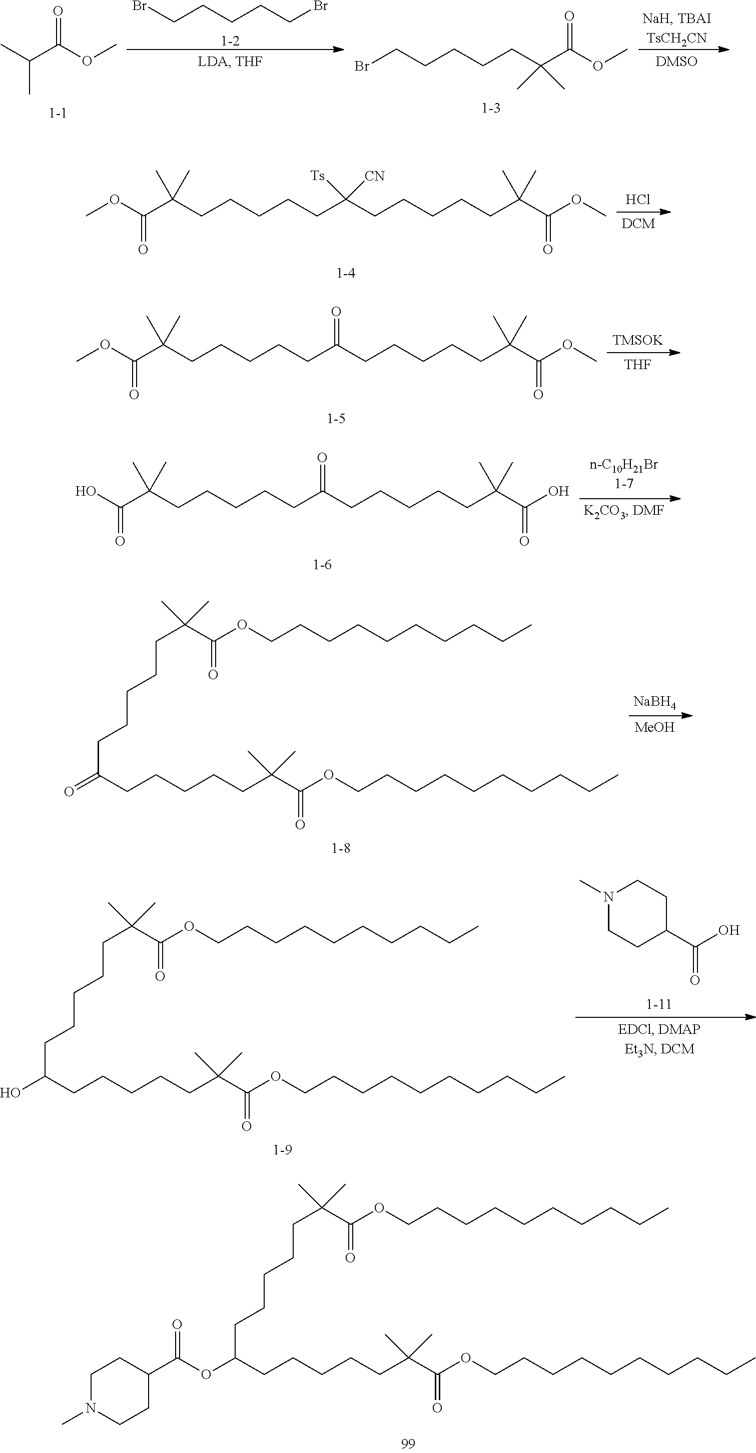

A solution of compound 1-1 (100 g, 979 mmol) in tetrahydrofuran (800 mL) was cooled to −40° C. LDA (2 M, 490 mL) was added slowly dropwise to the solution and the mixture was stirred for another 1 h after completion of the dropwise addition. A solution of 1-2 (315 g, 1.37 mol) in tetrahydrofuran (100 mL) was added dropwise to the reaction system at the same temperature and the reaction system was stirred overnight. The reaction system was quenched with saturated aqueous ammonium chloride, and extracted with ethyl acetate. The organic phases were combined, dried over anhydrous sodium sulfate, and filtered. The filtrate was concentrated to dryness to give the crude product. The crude product was purified by silica gel column to give compound 1-3 (115 g). ¹H NMR (400 MHz, CDCl₃): δ ppm 1.06-1.11 (m, 6H), 1.13-1.22 (m, 2H), 1.29-1.39 (m, 2H), 1.42-1.49 (m, 2H), 1.73-1.82 (m, 2H), 3.28-3.40 (m, 2H), 3.55-3.66 (m, 3H).

A solution of compound 1-3 (100 g, 398 mmol), TsCH₂CN (38.9 g, 199 mmol) and TBAI (14.7 g, 39.8 mmol) in dimethyl sulfoxide (800 mL) was cooled to 0° C., and sodium hydride (20.7 g, 517 mmol, 60% purity) was added slowly in batches. The mixture was reacted at room temperature overnight. The reaction system was quenched with saturated aqueous sodium chloride solution and extracted with ethyl acetate. The organic phases were combined, dried over anhydrous sodium sulfate, and filtered. The filtrate was concentrated to dryness to give 115 g of crude compound 1-4, which was used directly in the next reaction without isolation and purification.

To a solution of compound 1-4 crude (110 g, 205 mmol) in dichloromethane (880 mL) was added 330 mL of concentrated hydrochloric acid, and the mixture was reacted at room temperature for 2 h. The complete reaction of the substrate was monitored by TLC. The reaction system was quenched with saturated aqueous ammonium chloride solution and extracted with ethyl acetate. The organic phases were combined, dried over anhydrous sodium sulfate, and filtered. The filtrate was concentrated to dryness to give the crude product. The crude product was purified by silica gel column to give compound 1-5 (30.0 g, 80.9 mmol, 39.4%).

TMSOK (11.0 g, 86.4 mmol) was added to a solution of compound 1-5 (8.0 g, 21.6 mmol) in tetrahydrofuran (35.0 mL) at room temperature, and the reaction system was heated to 70° C. with stirring. The complete consumption of reaction materials was monitored by TLC. The reaction solution was cooled to room temperature, and the organic solvent was removed by rotary evaporation. The crude product was added to 20 mL of water and extracted with dichloromethane. The aqueous layer was collected, and the solution was adjusted to a pH of <5 with 1 M hydrochloric acid. The solution was extracted with dichloromethane. The organic phases were combined, dried over anhydrous sodium sulfate, and filtered. The filtrate was collected and concentrated to give compound 1-6 (7.0 g). ¹H NMR (400 MHz, CDCl₃): δ ppm 1.03 (s, 12H), 1.08-1.17 (m, 8H), 1.34-1.45 (m, 8H), 2.21 (t, J=7.2 Hz, 4H).

Potassium carbonate (482 mg, 3.48 mmol) was added to a solution of compound 1-6 (294 mg, 0.87 mmol) and 1-7 (771 mg, 3.48 mmol) in DMF, then the reaction was warmed up to 60° C. for 6 h. The complete disappearance of reactant 1-6 was monitored. The mixture was cooled to room temperature. The reaction system was quenched with saturated aqueous sodium chloride solution and extracted with ethyl acetate. The organic phases were combined, dried over anhydrous sodium sulfate, and filtered. The filtrate was concentrated to dryness to give the crude product. The crude was purified by silica gel column to give compound 1-8 (325 mg).

Compound 1-8 (325 mg) was dissolved in 4.0 mL of methanol and sodium borohydride (30 mg, 0.84 mmol) was added to the reaction system. The mixture was reacted at room temperature. The complete disappearance of the reactants was monitored by TLC. The reaction system was quenched with saturated aqueous sodium chloride solution and extracted with dichloromethane. The organic phases were combined, dried over anhydrous sodium sulfate, and filtered. The filtrate was concentrated to dryness to give crude compound 1-9 (260 mg), which was used directly in the next reaction without purification.

Crude compound 1-9 (250 mg, 0.40 mmol), 1-11 (35.9 mg, 0.60 mmol), EDCI (230 mg, 1.20 mmol), triethylamine (0.17 mL, 1.20 mmol) and DMAP (49 mg, 0.40 mmol) were dissolved in 5.0 mL of dichloromethane, and the reaction solution was stirred to react at room temperature for 12 h. The reaction solution was quenched with saturated aqueous sodium chloride and extracted with dichloromethane. The organic phases were combined, dried over anhydrous sodium sulfate, and filtered. The organic phase was collected and the organic solvent was removed using a rotary-evaporator to give the crude product, which was purified by preparative high performance liquid chromatography to give compound 99 (31.6 mg). ¹H NMR (400 MHz, CDCl₃): δ ppm 0.86 (t, J=6.8 Hz, 6H), 1.13 (s, 12H), 1.25 (m, 43H), 1.46 (m, 8H), 1.57 (m, 4H), 1.84 (m, 4H), 2.33 (s, 3H), 2.86 (m, 2H), 4.01 (m, 4H), 4.81 (m, 1H); ESI-MS m/z: 751.0 [M+H]⁺.

### Example 100: Synthesis of Compound 100

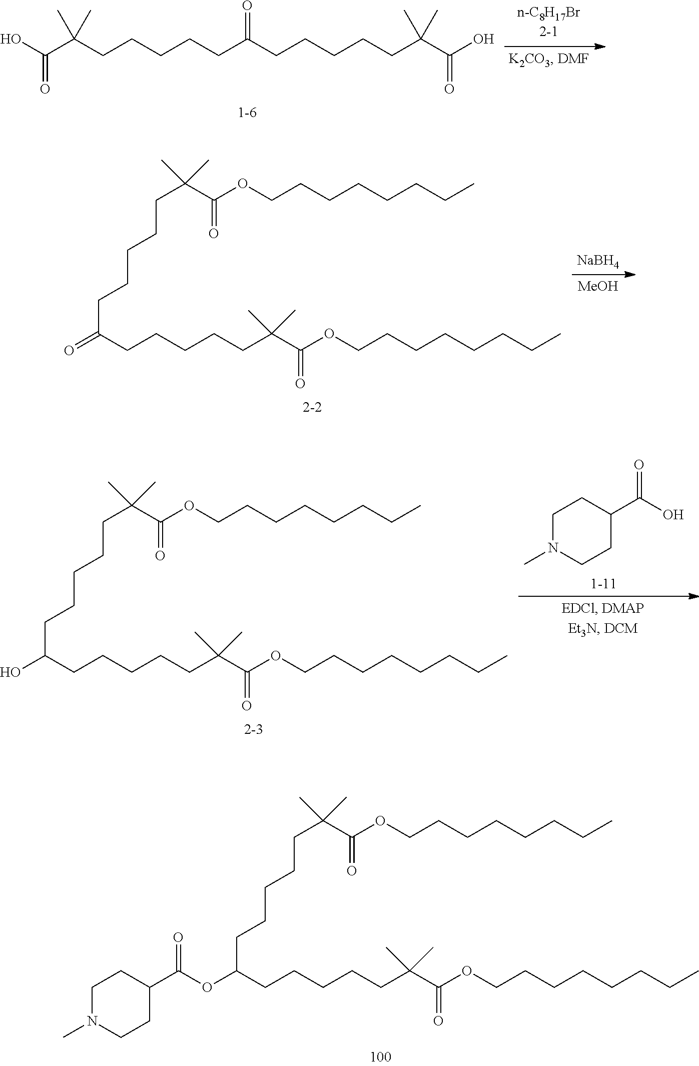

Referring to the method of Example 99, compound 100 was prepared as an oily product: 33.5 mg. ¹H NMR (400 MHz, CDCl₃): δ ppm 0.81 (t, J=6.8 Hz, 6H), 1.08 (s, 12H), 1.11-1.31 (m, 30H), 1.41 (m, 9H), 1.54 (m, 5H), 1.65-1.77 (m, 2H), 1.78-1.98 (m, 4H), 2.20 (m, 4H), 2.74 (m, 2H), 3.97 (t, J=6.8 Hz, 4H), 4.71-4.85 (m, 1H); ESI-MS m/z: 694.6 [M+H]⁺.

### Example 101: Synthesis of Compound 101

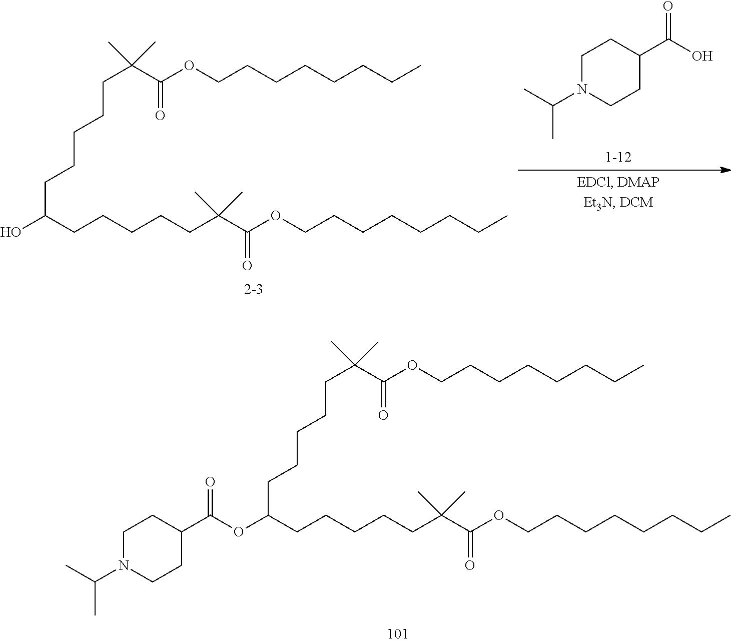

Referring to the method of Example 99, compound 101 was prepared as an oily product: 30.8 mg. ¹H NMR (400 MHz, CDCl₃): δ ppm 0.81 (t, J=6.8 Hz, 6H), 0.96 (d, J=6.8 Hz, 6H), 1.08 (s, 12H), 1.11-1.31 (m, 32H), 1.35-1.46 (m, 8H), 1.54 (m, 4H), 1.59-1.74 (m, 4H), 2.01-2.13 (m, 3H), 2.62 (m, 1H), 2.77 (m, 2H), 3.97 (t, J=6.8 Hz, 4H), 4.71-4.83 (m, 1H); ESI-MS m/z: 722.6 [M+H]⁺.

### Example 102: Synthesis of Compound 102

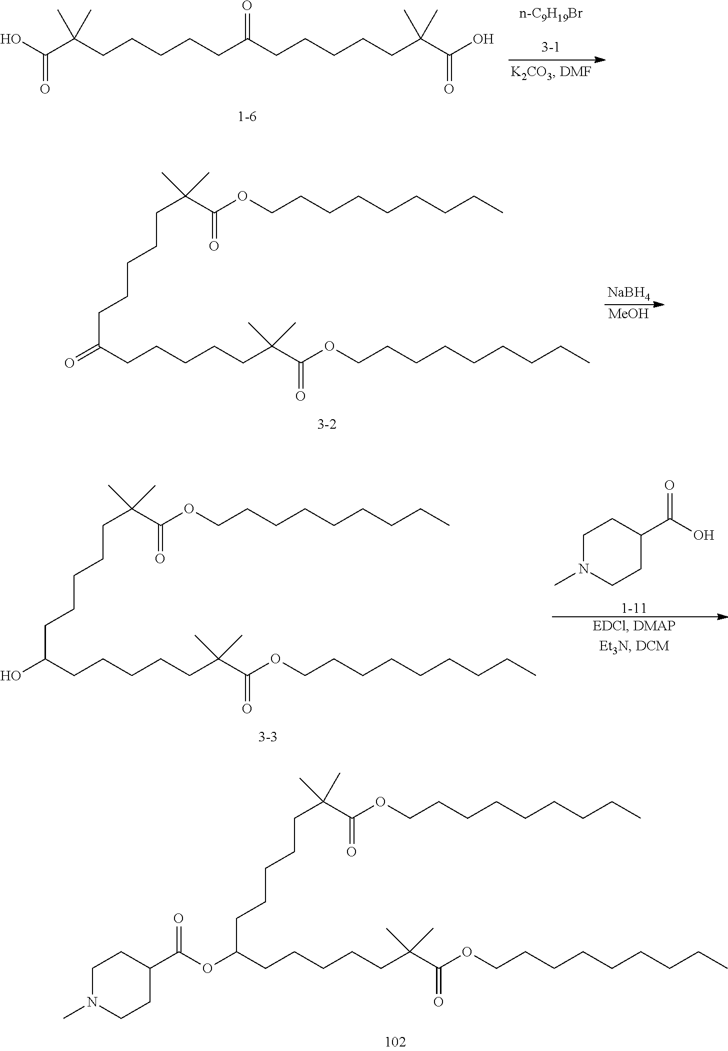

Referring to the method of Example 99, compound 102 was prepared as an oily product: 32.4 mg. ¹H NMR (400 MHz, CDCl₃): δ ppm 0.90 (t, J=6.8 Hz, 6H), 1.17 (s, 12H), 1.20-1.41 (m, 34H), 1.44-1.55 (m, 8H), 1.57-1.69 (m, 5H), 1.73-1.86 (m, 3H), 1.92 (m, 2H), 2.02 (m, 2H), 2.21-2.33 (m, 4H), 2.82-2.85 (m, 2H), 4.06 (t, J=6.8 Hz, 4H), 4.84-4.90 (m, 1H); ESI-MS m/z: 722.6 [M+H]⁺.

### Example 103: Synthesis of Compound 103

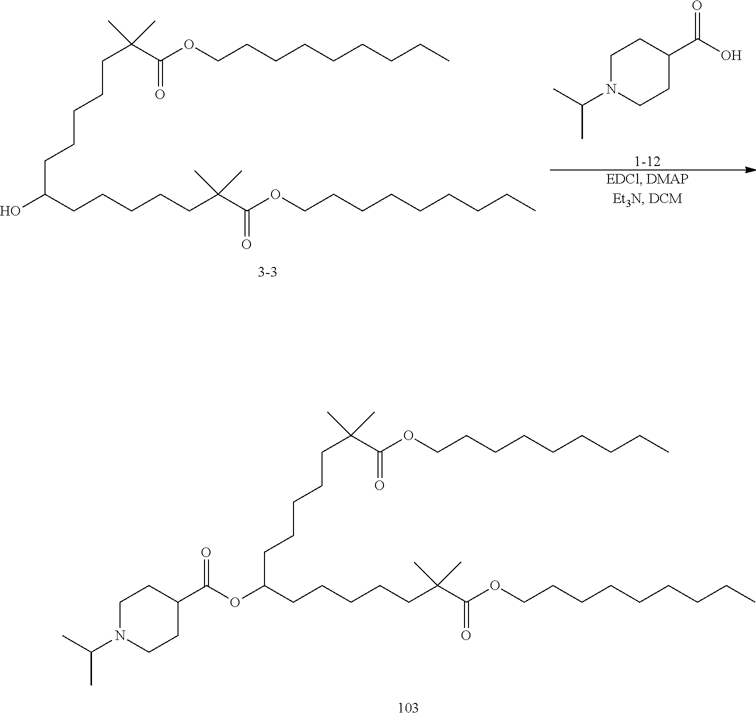

Referring to the method of Example 99, compound 103 was prepared as an oily product: 32.8 mg. ¹H NMR (400 MHz, CDCl₃): δ ppm 0.81 (t, J=6.8 Hz, 6H), 0.96 (d, J=6.8 Hz, 6H), 1.08 (s, 12H), 1.11-1.33 (m, 34H), 1.34-1.47 (m, 8H), 1.54 (m, 5H), 1.60-1.75 (m, 3H), 1.83 (m, 2H), 2.03-2.24 (m, 3H), 2.56-2.79 (m, 3H), 3.97 (t, J=6.8 Hz, 4H), 4.74-4.81 (m, 1H); ESI-MS m/z: 750.6 [M+H]⁺.

### Example 104: Synthesis of Compound 104

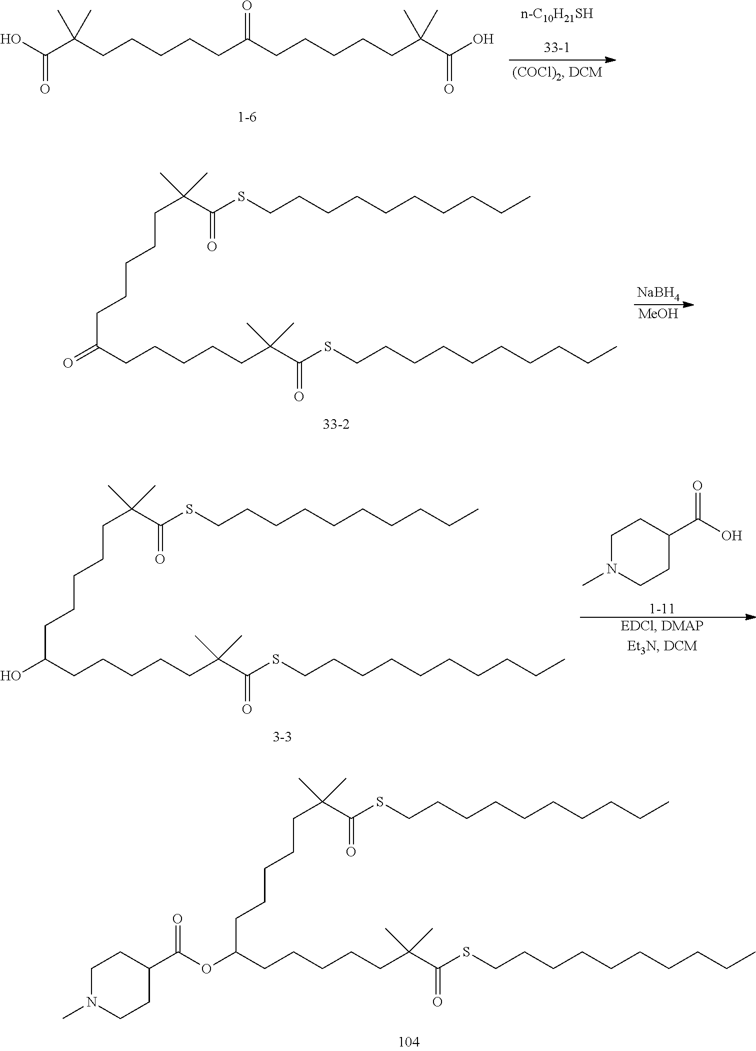

Compound 1-6 (448 mg, 1.3 mmol) was dissolved in 5.0 mL of dichloromethane, and the reaction system was cooled to 0° C. in an ice bath. DMF (10 μL, 0.13 mmol) was added and oxalyl chloride (0.44 mL, 5.2 mmol) was then added dropwise to the reaction solution. The ice bath was removed after the dropwise addition was completed and the mixture was stirred for 1 h at room temperature. The solvent was removed using a rotary-evaporator to give acyl chloride crude product (330 mg) as an oil, which was used directly in the next reaction step.

1-Decanethiol 33-1 (455 mg, 2.61 mmol) was added to a solution of crude acyl chloride (330 mg, 0.87 mmol) in DCE (3.0 mL), and the reaction was heated to 70° C. to react overnight. The reaction solution was cooled to room temperature and the solvent was removed using a rotary-evaporator to give the crude product, which was purified by silica gel column to give compound 33-2 (400 mg). ¹H NMR (400 MHz, CDCl₃): δ ppm 0.84-0.87 (m, 6H), 1.14-1.18 (m, 12H), 1.20-1.28 (m, 36H), 1.48-1.55 (m, 12H), 2.33 (t, J=7.2 Hz, 4H), 2.79 (t, J=7.2 Hz, 4H).

Compound 33-2 (300 mg, 0.46 mmol) was dissolved in 3.0 mL of methanol and NaBH₄ (52.5 mg, 1.38 mmol) was added in batches. The reaction solution was stirred under nitrogen atmosphere at room temperature for 2 h. The complete disappearance of the reaction material was monitored by TLC. The reaction solution was quenched by adding saturated ammonium chloride solution, and extracted with ethyl acetate. The organic phases were combined, dried over anhydrous sodium sulfate, and filtered. The filtrate was collected and concentrated to give 300 mg of crude compound 33-3, which was directly used in the next reaction step without further purification.

Crude compound 33-3 (300 mg, 0.46 mmol), 1-11 (98.8 mg, 0.69 mmol), EDCI (264.5 mg, 1.38 mmol), triethylamine (0.19 mL, 1.38 mmol) and DMAP (56.2 mg, 0.46 mmol) were dissolved in 8.0 mL of dichloromethane, and the reaction solution was stirred at room temperature until the reaction material 33-3 was completely consumed. The reaction solution was quenched with saturated aqueous sodium chloride and extracted with dichloromethane. The organic phases were combined, dried over anhydrous sodium sulfate, and filtered. The organic phase was collected, and the organic solvent was removed using a rotary-evaporator. The crude product was purified by preparative high performance liquid chromatography to give the compound 104 (67.3 mg). ¹H NMR (400 MHz, CDCl₃): δ ppm 0.81 (t, J=6.8 Hz, 6H), 1.08 (s, 12H), 1.09-1.31 (m, 42H), 1.35-1.51 (m, 14H), 1.61-2.25 (m, 8H), 2.73 (t, J=7.2 Hz, 4H), 4.77 (m, 1H); ESI-MS m/z: 782.7 [M+H]⁺.

### Example 105: Synthesis of Compound 105

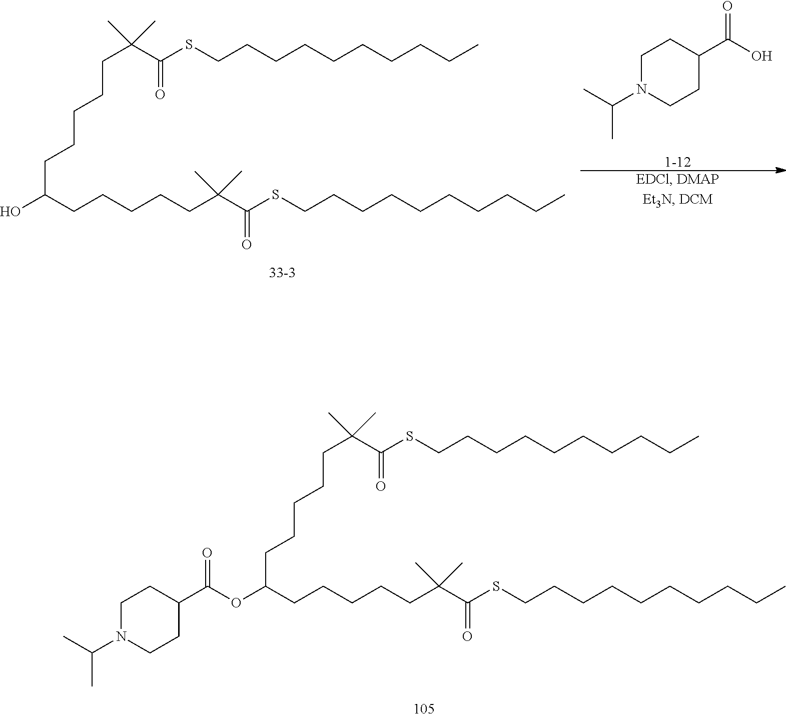

Referring to the method of Example 104, compound 105 was prepared as an oily product: 27.1 mg. ¹H NMR (400 MHz, CDCl₃): δ ppm 0.85-0.89 (m, 6H), 1.02 (br d, J=6.4 Hz, 6H), 1.18 (s, 12H), 1.20-1.40 (m, 40H), 1.42-1.59 (m, 12H), 1.64-1.83 (m, 3H), 1.87-1.93 (m, 2H), 2.11-2.23 (m, 3H), 2.66-2.94 (m, 6H), 4.72-4.94 (m, 1H); ESI-MS m/z: 810.6 [M+H]⁺.

### Example 106: Synthesis of Compound 106

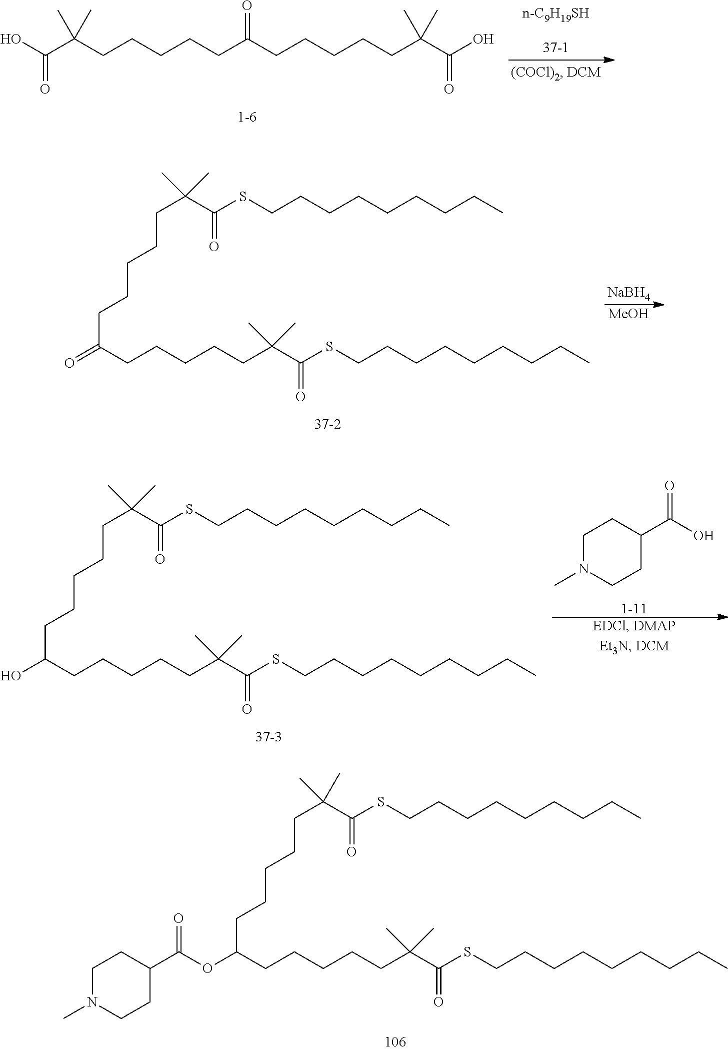

Referring to the method of Example 104, compound 106 was prepared as an oily product: 38.4 mg. ¹H NMR (400 MHz, CDCl₃): δ ppm 0.88 (t, J=7.2 Hz, 6H), 1.17 (s, 12H), 1.15-1.34 (m, 36H), 1.43-1.57 (m, 15H), 1.69-2.09 (m, 5H), 2.27-2.34 (m, 3H), 2.77-2.86 (m, 5H), 4.78-4.85 (m, 1H); ESI-MS m/z: 754.6 [M+H]⁺.

### Example 107: Synthesis of Compound 107

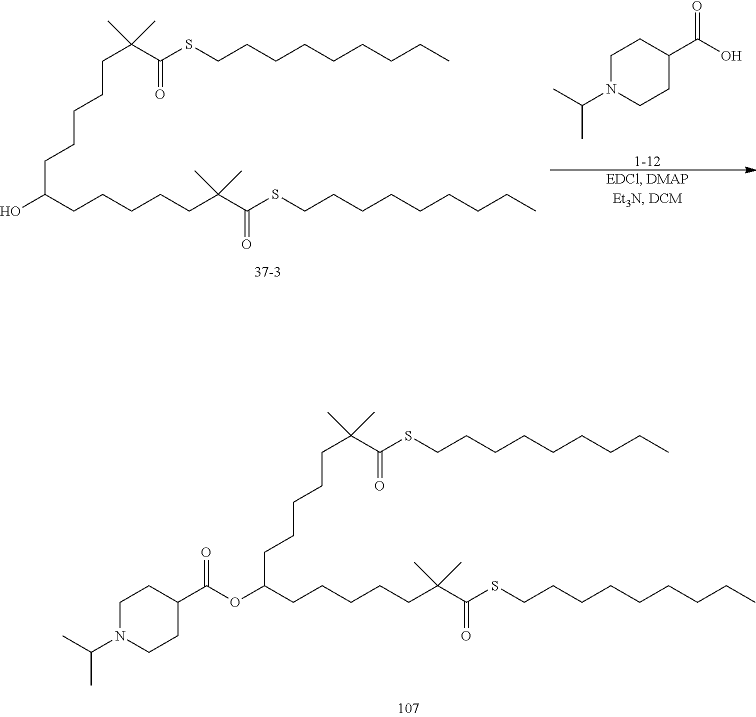

Referring to the method of Example 104, compound 107 was prepared as an oily product: 39 mg. ¹H NMR (400 MHz, CDCl₃): δ ppm 0.88 (t, J=6.8 Hz, 6H), 1.05 (d, J=6.8 Hz, 6H), 1.16 (s, 12H), 1.12-1.35 (m, 34H), 1.37-1.55 (m, 15H), 1.62-1.92 (m, 4H), 2.15-2.19 (m, 3H), 2.71-2.93 (m, 6H), 4.78-4.85 (m, 1H); ESI-MS m/z: 782.6 [M+H]⁺.

### Example 108: Synthesis of Compound 108

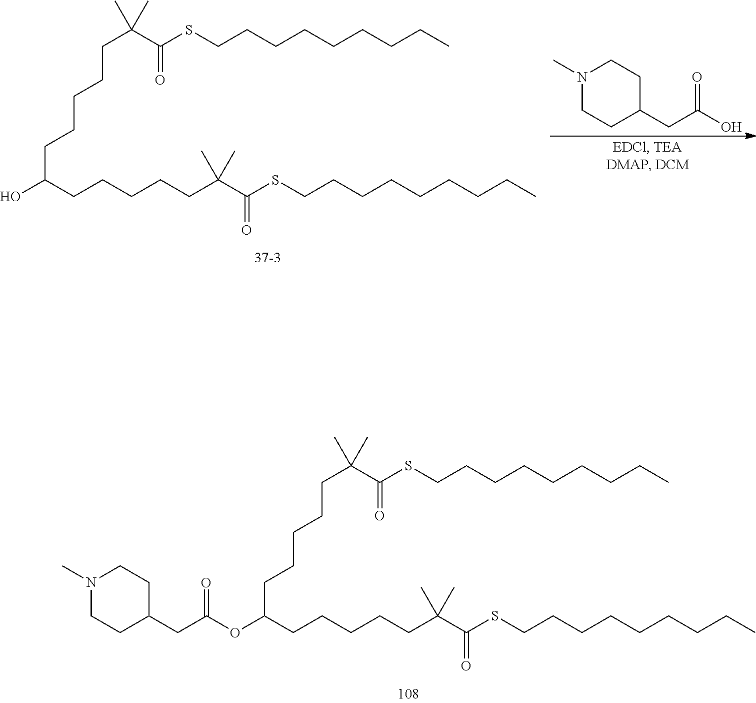

Referring to the method of Example 104, compound 108 was prepared as an oily product: 43.8 mg. ¹H NMR (400 MHz, CDCl₃): δ ppm 0.88 (t, J=6.80 Hz, 6H), 1.18 (s, 12H), 1.20-1.39 (m, 38H), 1.40-1.62 (m, 14H), 1.66-1.86 (m, 3H), 1.89-2.10 (m, 2H), 2.19-2.27 (m, 3H), 2.28 (br s, 2H), 2.79-2.83 (m, 4H), 4.79-4.88 (m, 1H); ESI-MS m/z: 768.5 [M+H]⁺.

### Example 109: Synthesis of Compound 109

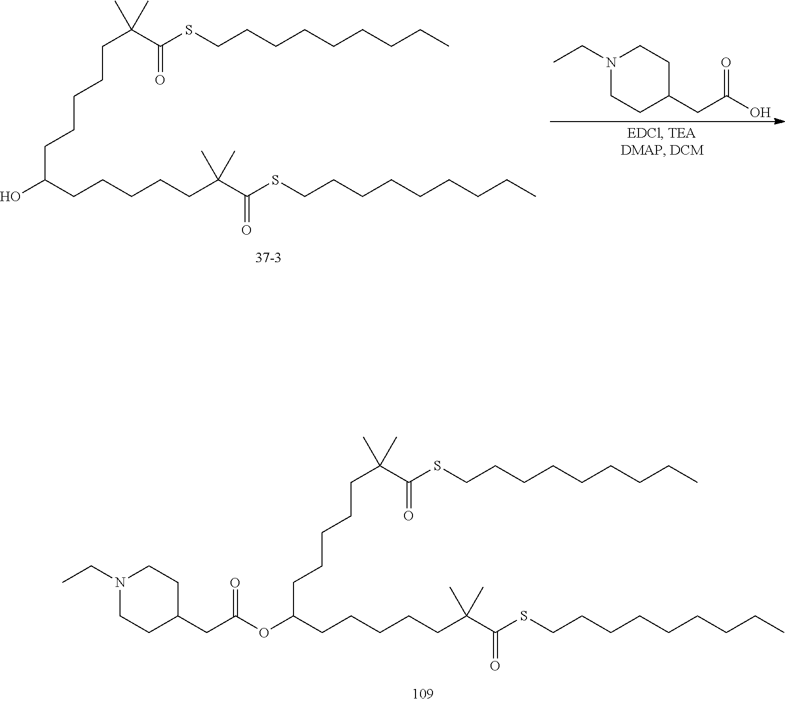

Referring to the method of Example 104, compound 109 was prepared as an oily product: 44.8 mg. ¹H NMR (400 MHz, CDCl₃): δ ppm 0.86-0.89 (m, 6H), 1.18 (s, 15H), 1.23-1.37 (m, 36H), 1.46-1.54 (m, 14H), 1.76-1.93 (m, 4H), 2.11-2.20 (m, 1H), 2.24-2.28 (m, 2H), 2.54-2.71 (m, 2H), 2.81 (d, J=7.2 Hz, 4H), 3.08-3.24 (m, 2H), 4.79-4.88 (m, 1H); ESI-MS m/z: 782.6 [M+H]⁺.

### Example 110: Synthesis of Compound 110

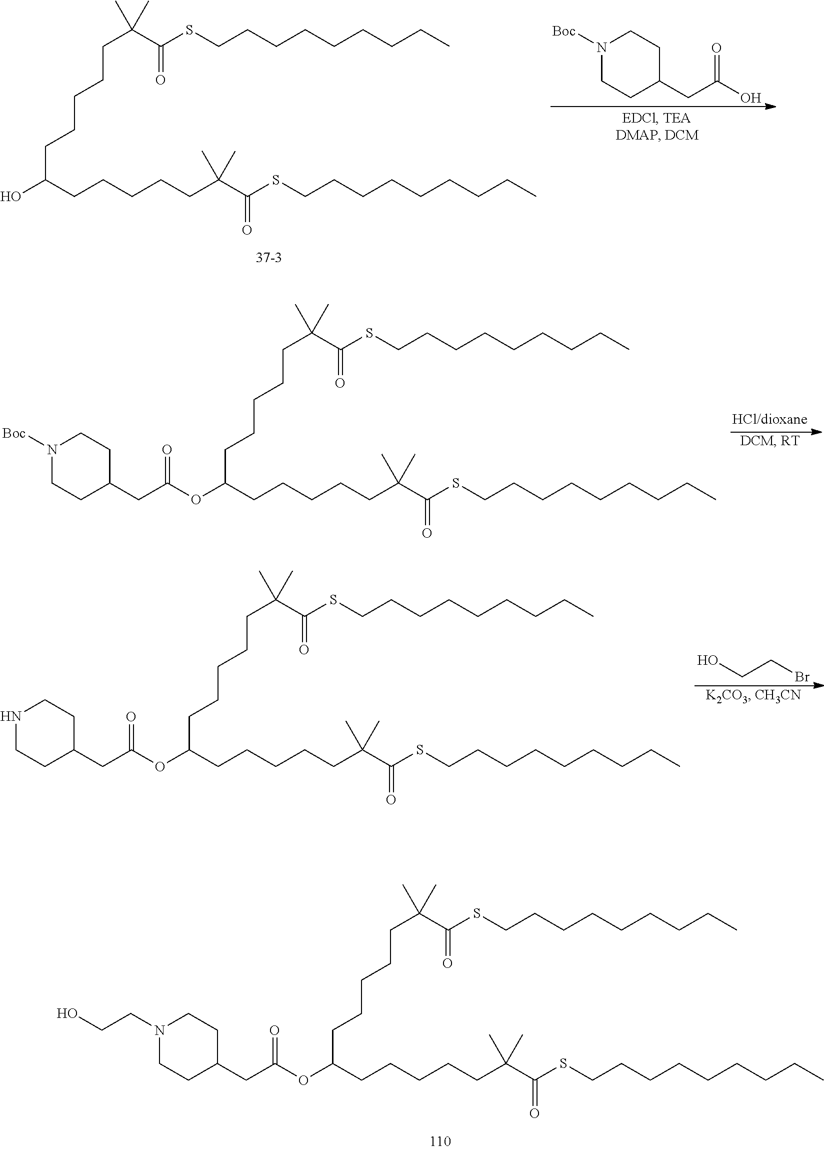

Referring to the method of Example 104, compound 110 was prepared as an oily product: 34.4 mg. ¹H NMR (400 MHz, CDCl₃): δ ppm 0.81 (t, J=7.2 Hz, 6H), 1.12 (s, 12H), 1.14-1.27 (m, 34H), 1.44-1.48 (m, 12H), 1.66-1.77 (m, 7H), 2.05-2.24 (m, 4H), 2.53 (m, 2H), 2.75 (t, J=7.2 Hz, 4H), 2.90-2.92 (m, 2H), 3.57 (t, J=5.2 Hz, 2H), 4.74-4.80 (m, 1H); ESI-MS m/z: 798.6 [M+H]⁺.

### Example 111: Synthesis of Compound 111

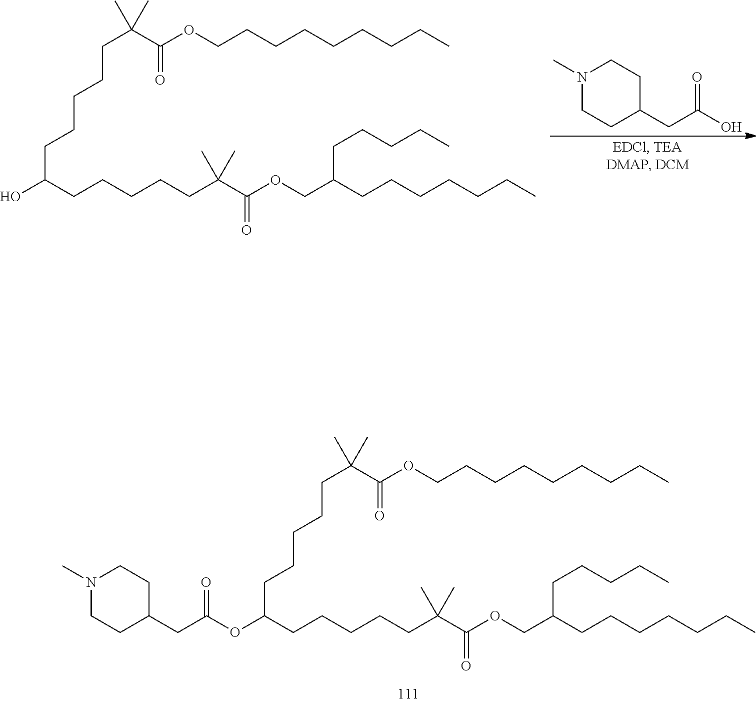

Referring to the method of Example 104, compound 111 was prepared as an oily product: 31.4 mg. ¹H NMR (400 MHz, CDCl₃): δ ppm 0.92 (t, J=6.8 Hz, 9H), 1.19 (s, 12H), 1.20-1.35 (m, 43H), 1.47-1.55 (m, 9H), 1.54-1.82 (m, 12H), 2.07-2.37 (m, 7H), 2.94-3.01 (m, 2H), 3.98 (d, J=6.8 Hz, 2H), 4.07 (t, J=6.8 Hz, 2H), 4.84-4.91 (m, 1H); ESI-MS m/z: 806.7 [M+H]⁺.

### Example 112: Synthesis of Compound 112

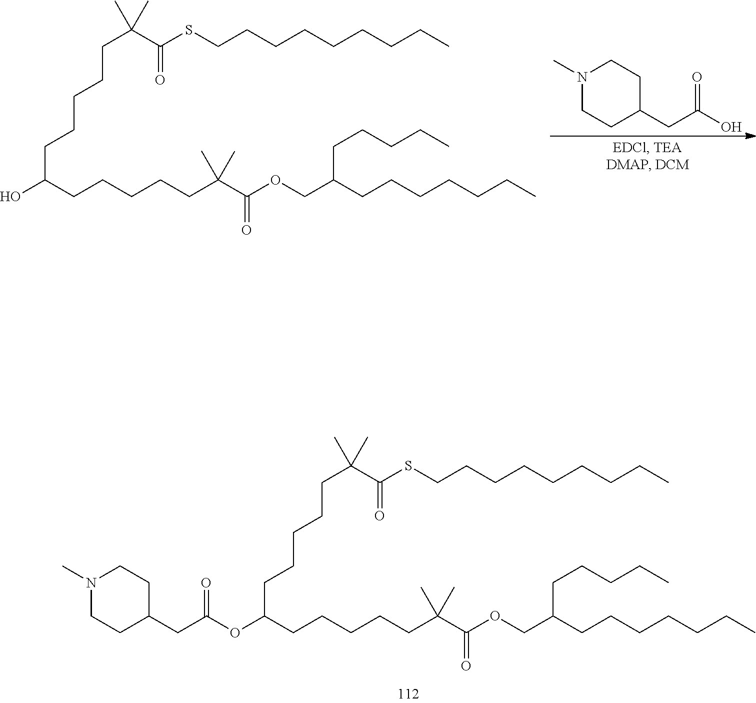

Referring to the method of Example 104, compound 112 was prepared as an oily product: 24.4 mg. ¹H NMR (400 MHz, CDCl₃): δ ppm 0.80-0.83 (m, 9H), 1.08 (s, 12H), 1.10-1.35 (m, 28H), 1.41-1.57 (m, 28H), 1.65-1.75 (m, 4H), 1.95-2.10 (m, 2H), 2.16 (d, J=6.4 Hz, 2H), 2.30 (s, 3H), 2.73-2.91 (m, 4H), 3.87 (d, J=5.6 Hz, 2H), 4.75-4.79 (m, 1H); ESI-MS m/z: 822.7 [M+H]⁺.

### Example 113: Synthesis of Compound 113

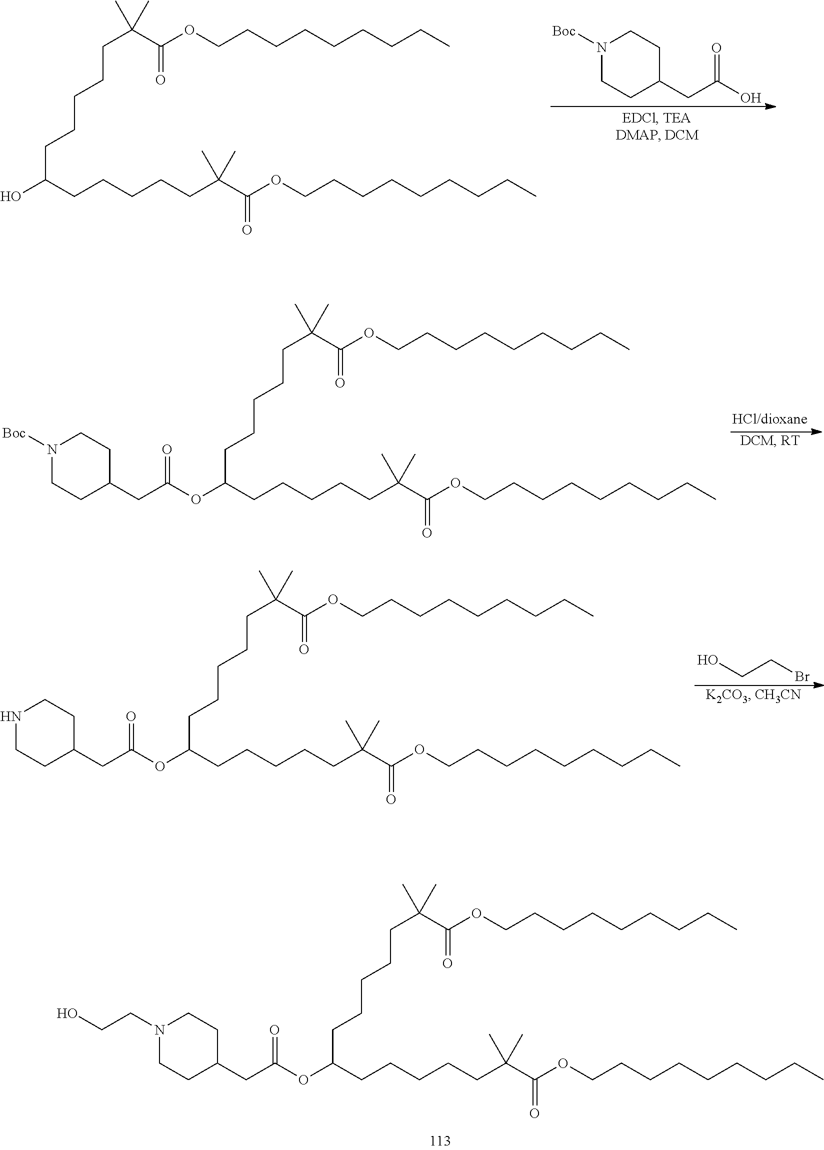

Referring to the method of Example 110, compound 113 was prepared as an oily product: 31.1 mg. ¹H NMR (400 MHz, CDCl₃): δ ppm 0.81 (t, J=7.2 Hz, 6H), 1.08 (s, 12H), 1.10-1.24 (m, 36H), 1.36-1.43 (m, 8H), 1.48-1.54 (m, 6H), 1.64-1.72 (m, 6H), 2.05 (t, J=6.8 Hz, 1H), 2.15 (d, J=6.8 Hz, 2H), 2.47 (t, J=5.6 Hz, 2H), 2.82-2.89 (m, 2H), 3.54 (t, J=5.6 Hz, 2H), 3.97 (t, J=6.8 Hz, 4H), 4.73-4.79 (m, 1H); ESI-MS m/z: 766.6 [M+H]⁺.

### Example 114: Synthesis of Compound 114

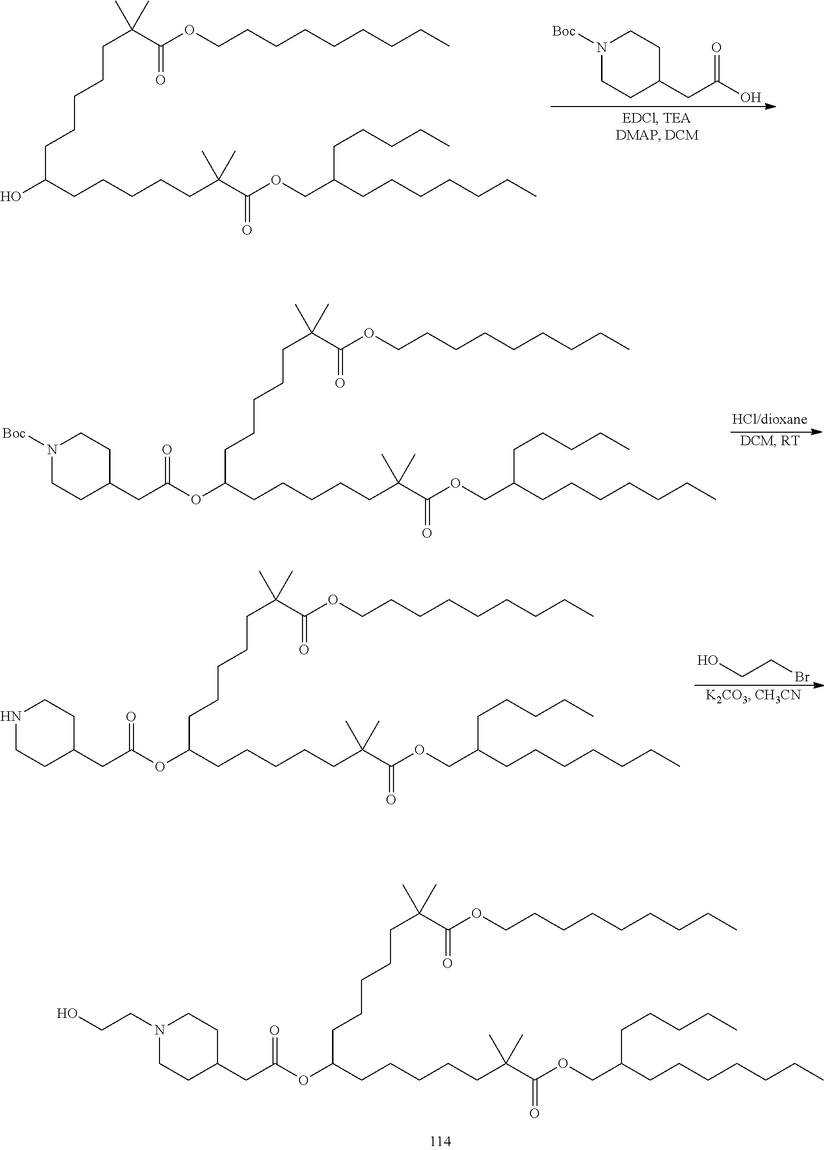

Referring to the method of Example 110, compound 114 was prepared as an oily product: 32.7 mg. ¹H NMR (400 MHz, CDCl₃): δ ppm 0.85-0.88 (m, 9H), 1.07 (s, 12H), 1.09-1.35 (m, 46H), 1.41-1.58 (m, 13H), 1.97-2.25 (m, 3H), 2.32 (d, J=5.6 Hz, 2H), 2.83-2.86 (m, 2H), 3.17-3.19 (m, 2H), 3.78-3.81 (d, J=7.2 Hz, 2H), 3.92 (d, J=5.6 Hz, 2H), 4.01 (t, J=6.4 Hz, 2H), 4.10 (m, 1H), 4.81-4.86 (m, 1H); ESI-MS m/z: 836.7 [M+H]⁺.

### Example 115: Synthesis of Compound 115

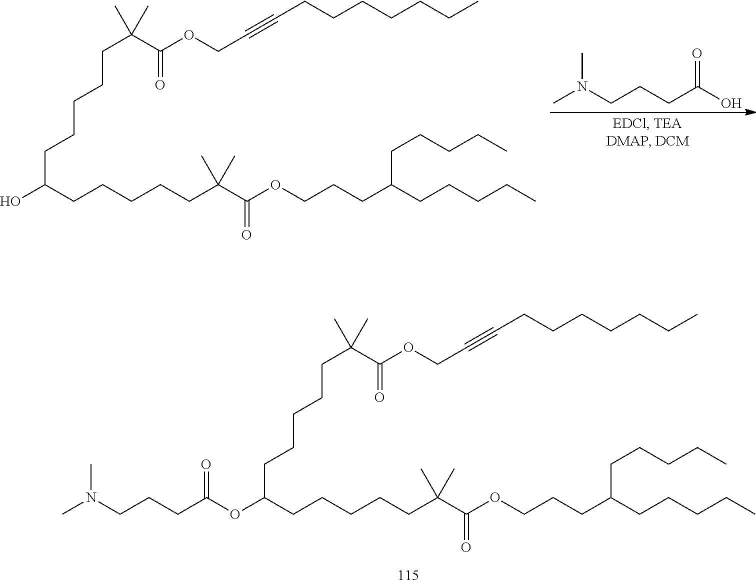

Referring to the method of Example 104, compound 115 was prepared as an oily product: 31.0 mg. ¹H NMR (400 MHz, CDCl₃): δ ppm 0.87-0.91 (m, 9H), 1.14-1.37 (m, 51H), 1.49-1.60 (m, 12H), 1.75-1.81 (m, 2H), 2.21-2.26 (m, 2H), 2.28 (s, 6H), 2.32-2.36 (m, 4H), 4.03 (t, J=6.4 Hz, 2H), 4.65 (s, 2H), 4.82-4.88 (m, 1H); ESI-MS m/z: 790.6 [M+H]⁺.

### Example 116: Synthesis of Compound 116

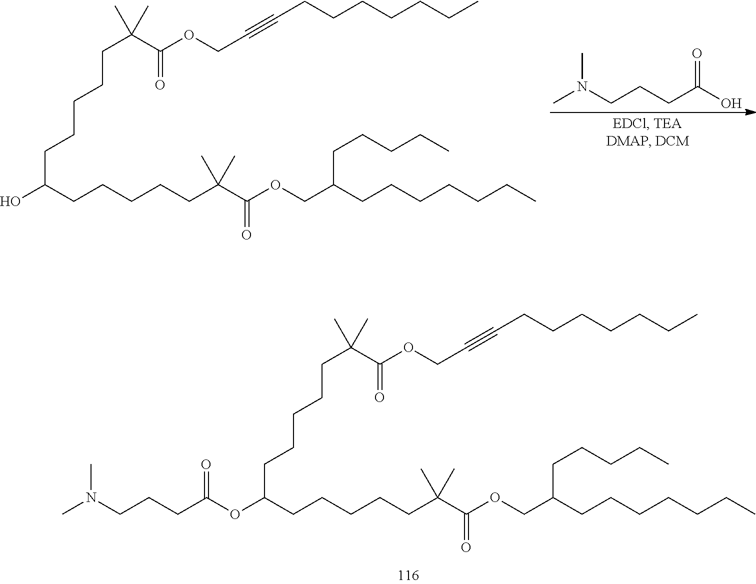

Referring to the method of Example 104, compound 116 was prepared as an oily product: 31.3 mg. ¹H NMR (400 MHz, CDCl₃): δ ppm 0.80-0.83 (m, 9H), 1.07-1.27 (m, 51H), 1.40-1.45 (m, 12H), 1.70-1.81 (m, 2H), 2.13-2.36 (m, 12H), 3.87 (d, J=5.6 Hz, 2H), 4.57 (s, 2H), 4.74-4.80 (m, 1H); ESI-MS m/z: 790.6 [M+H]⁺.

### Example 117: Synthesis of Compound 117

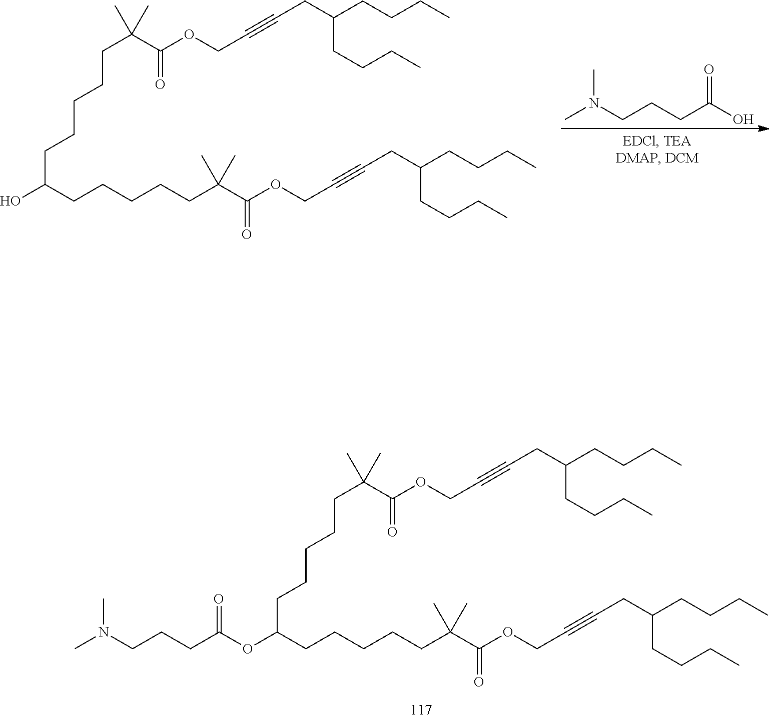

Referring to the method of Example 104, compound 117 was prepared as an oily product: 35.9 mg. ¹H NMR (400 MHz, CDCl₃): δ ppm 0.90 (t, J=6.8 Hz, 12H), 1.15 (s, 12H), 1.16-1.33 (m, 38H), 1.48-1.53 (m, 8H), 1.82-1.86 (m, 2H), 2.18-2.20 (m, 4H), 2.30-2.42 (m, 10H), 4.65 (m, 4H), 4.82-4.88 (m, 1H); ESI-MS m/z: 814.6 [M+H]⁺.

### Example 118: Synthesis of Compound 118

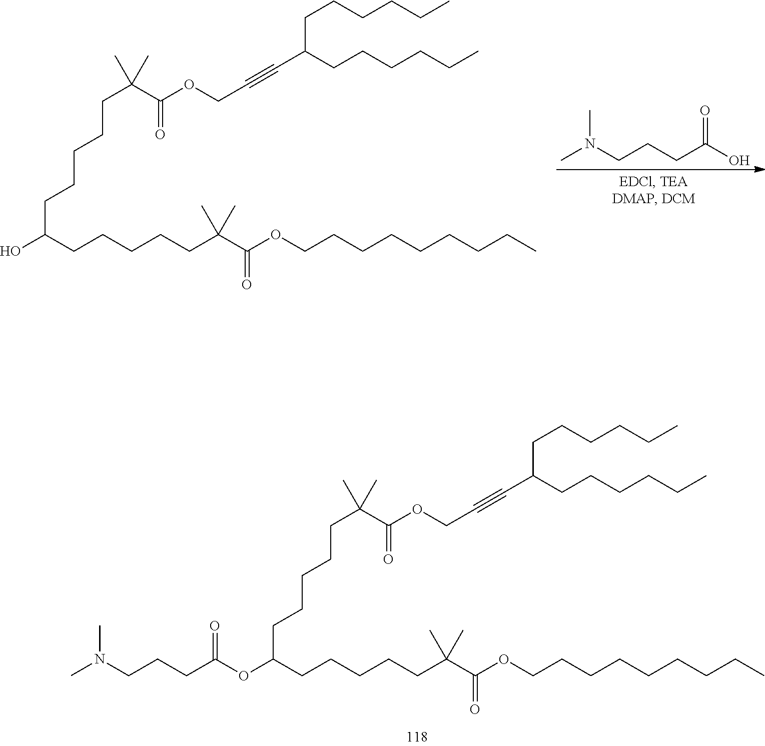

Referring to the method of Example 104, compound 118 was prepared as an oily product: 32.0 mg. ¹H NMR (400 MHz, CDCl₃): δ ppm 0.87-0.90 (m, 9H), 1.15-1.32 (m, 50H), 1.40-1.61 (m, 16H), 1.76-1.84 (m, 2H), 2.23 (s, 6H), 2.29-2.34 (m, 6H), 4.04 (t, J=6.8 Hz, 2H), 4.66 (d, J=2.0 Hz, 1H), 4.83-4.87 (m, 1H); ESI-MS m/z: 804.6 [M+H]⁺.

### Example 119: Synthesis of Compound 119

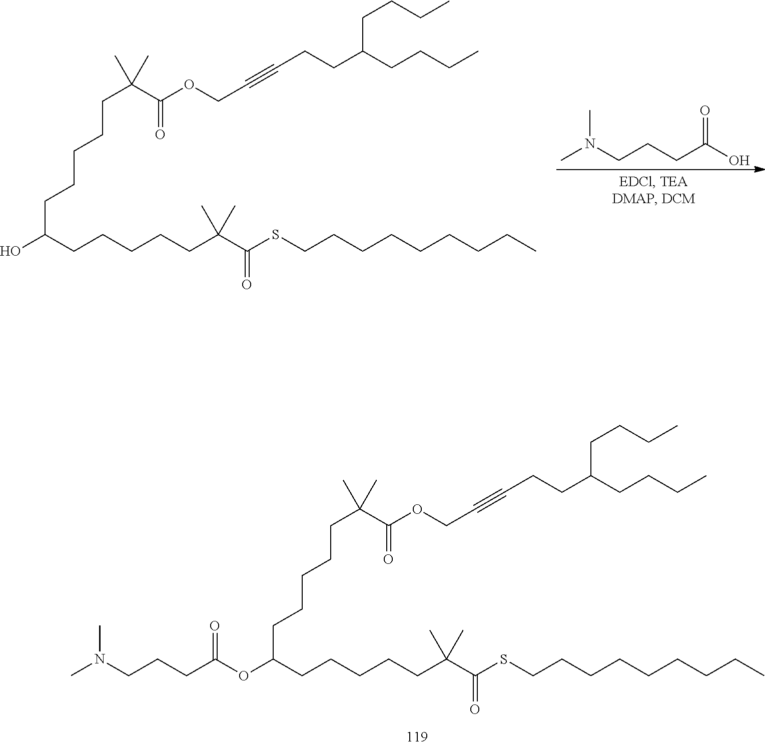

Referring to the method of Example 104, compound 119 was prepared as an oily product: 34.8 mg. ¹H NMR (400 MHz, CDCl₃): δ ppm 0.91-0.94 (m, 9H), 1.21-1.40 (m, 48H), 1.51-1.57 (m, 12H), 1.61-1.86 (m, 5H), 2.23 (m, 2H), 2.31 (s, 6H), 2.35-2.39 (m, 2H), 2.85 (t, J=7.2 Hz, 2H), 4.67 (t, J=2.0 Hz, 1H), 4.84-4.90 (m, 1H); ESI-MS m/z: 792.6 [M+H]⁺.

### Example 120: Synthesis of Compound 120

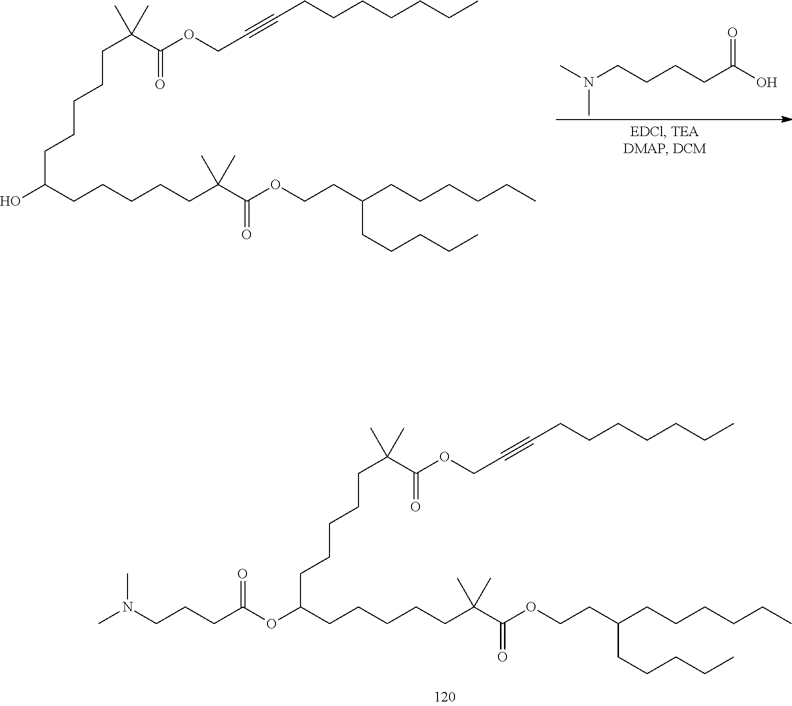

Referring to the method of Example 104, compound 120 was prepared as an oily product: 34.5 mg. ¹H NMR (400 MHz, CDCl₃): δ ppm 0.89 (t, J=6.8 Hz, 9H), 1.15-1.31 (m, 48H), 1.47-1.57 (m, 16H), 1.75-1.85 (m, 4H), 2.19-2.41 (m, 11H), 4.07 (t, J=6.8 Hz, 2H), 4.65 (t, J=2.0 Hz, 2H), 4.27-4.88 (m, 1H); ESI-MS m/z: 804.6 [M+H]⁺.

### Example 121: Synthesis of Compound 121

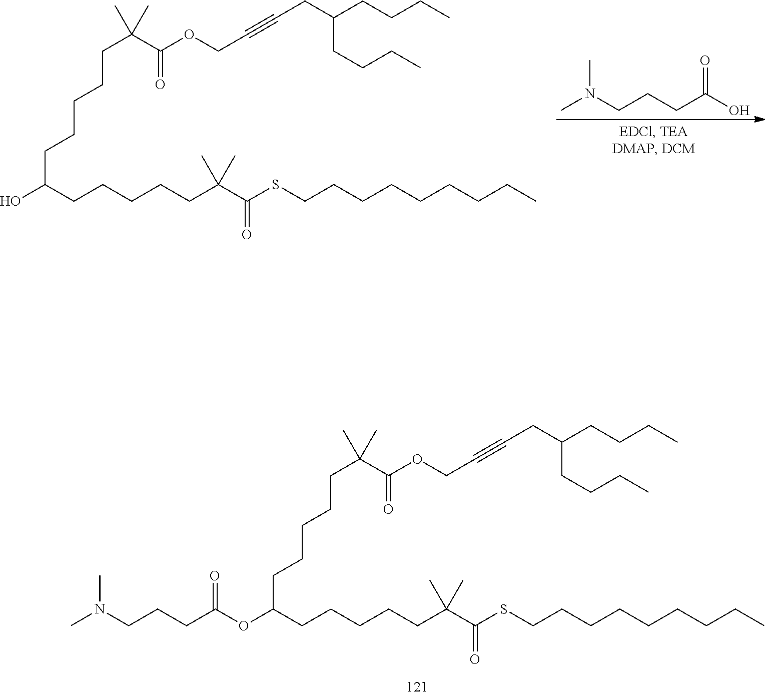

Referring to the method of Example 104, compound 121 was prepared as an oily product: 33.8 mg. ¹H NMR (400 MHz, CDCl₃): δ ppm 0.91-0.95 (m, 9H), 1.20-1.43 (m, 46H), 1.47-1.57 (m, 10H), 1.63-1.85 (m, 6H), 2.29-2.40 (m, 12H), 2.85 (t, J=7.2 Hz, 2H), 4.68 (s, 2H), 4.84-4.90 (m, 1H); ESI-MS m/z: 764.6 [M+H]⁺.

### Example 122: Synthesis of Compound 122

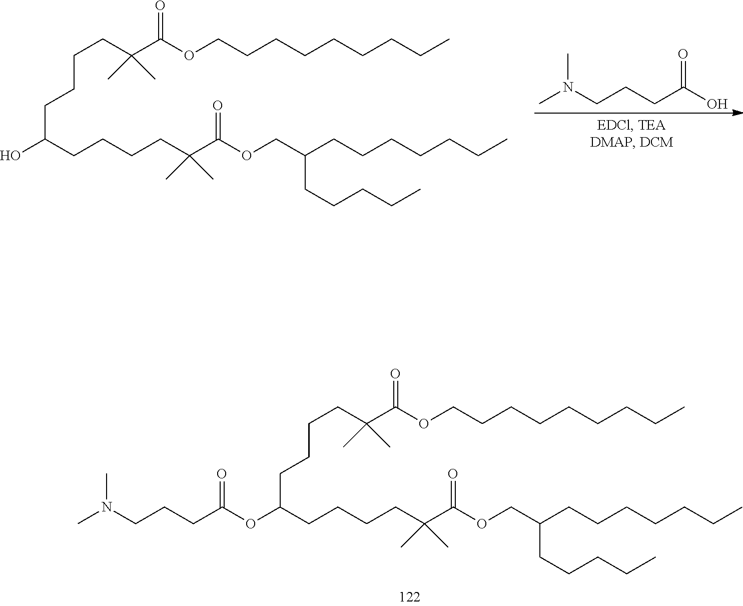

Referring to the method of Example 104, compound 122 was prepared as an oily product: 33.7 mg. ¹H NMR (400 MHz, CDCl₃): δ ppm 0.81 (t, J=6.8 Hz, 9H), 1.08-1.31 (m, 46H), 1.40-1.58 (m, 17H), 1.68-1.73 (m, 2H), 2.17 (s, 6H), 2.25 (t, J=7.2 Hz, 4H), 3.87 (d, J=5.6 Hz, 2H), 3.97 (t, J=6.8 Hz, 2H), 4.73-4.80 (m, 1H); ESI-MS m/z: 752.7 [M+H]⁺.

### Example 123: Synthesis of Compound 123

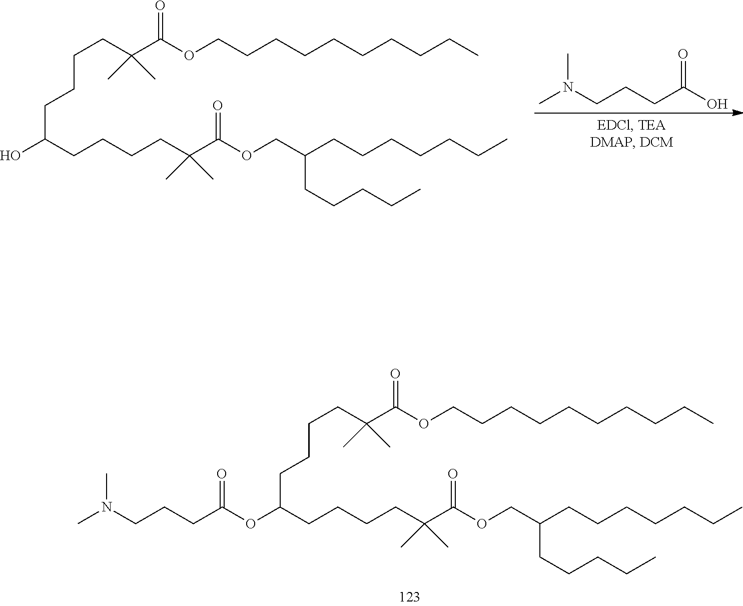

Referring to the method of Example 104, compound 123 was prepared as an oily product: 35.2 mg. ¹H NMR (400 MHz, CDCl₃): δ ppm 0.81 (t, J=6.8 Hz, 9H), 1.08 (s, 12H), 1.10-1.33 (m, 36H), 1.40-1.57 (m, 17H), 1.68-1.73 (m, 2H), 2.17 (s, 6H), 2.25 (t, J=7.2 Hz, 4H), 3.87 (d, J=5.6 Hz, 2H), 3.97 (t, J=6.8 Hz, 2H), 4.73-4.79 (m, 1H); ESI-MS m/z: 766.7 [M+H]⁺.

### Example 124: Synthesis of Compound 124

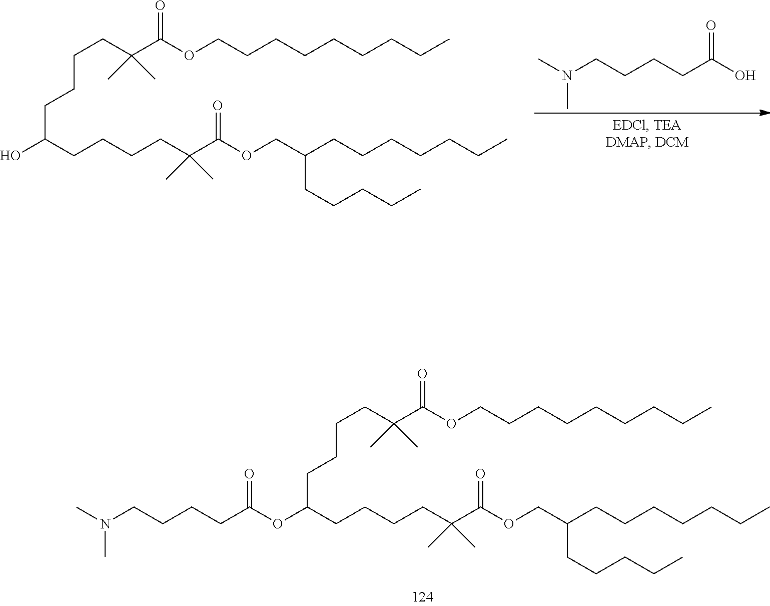

Referring to the method of Example 104, compound 124 was prepared as an oily product: 33.4 mg. ¹H NMR (400 MHz, CDCl₃): δ ppm 0.89 (t, J=6.8 Hz, 9H), 1.15 (s, 12H), 1.18-1.37 (m, 39H), 1.47-1.65 (m, 16H), 2.28 (s, 6H), 2.29-2.35 (m, 4H), 3.95 (d, J=5.6 Hz, 2H), 4.04 (t, J=6.8 Hz, 2H), 4.79-4.86 (m, 1H); ESI-MS m/z: 766.6 [M+H]⁺.

### Example 125: Synthesis of Compound 125

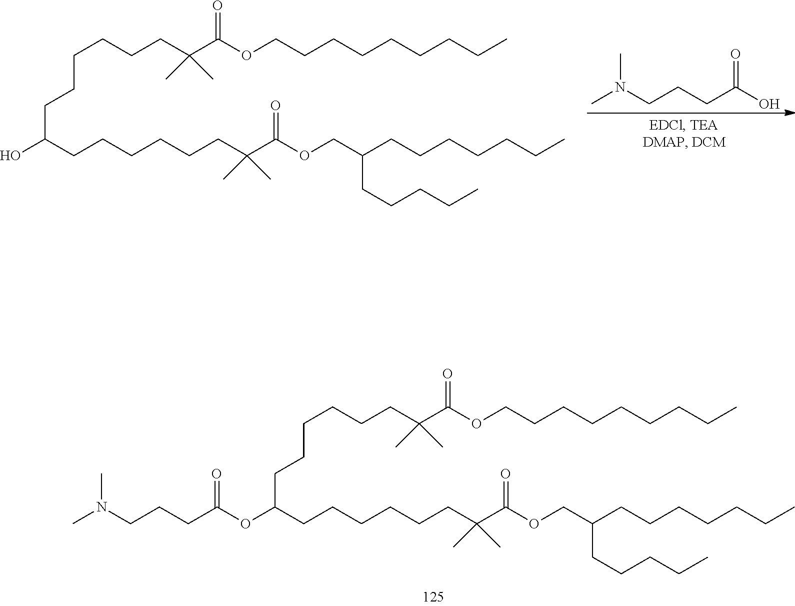

Referring to the method of Example 104, compound 125 was prepared as an oily product: 31.1 mg. ¹H NMR (400 MHz, CDCl₃): δ ppm 0.89 (t, J=6.8 Hz, 9H), 1.15 (s, 12H), 1.18-1.37 (m, 48H), 1.48-1.51 (m, 8H), 1.60-1.63 (m, 3H), 1.80-1.88 (m, 2H), 2.32-2.41 (m, 10H), 3.95 (d, J=5.6 Hz, 2H), 4.04 (t, J=6.8 Hz, 2H), 4.81-4.88 (m, 1H); ESI-MS m/z: 808.7 [M+H]⁺.

### Example 126: Synthesis of Compound 126

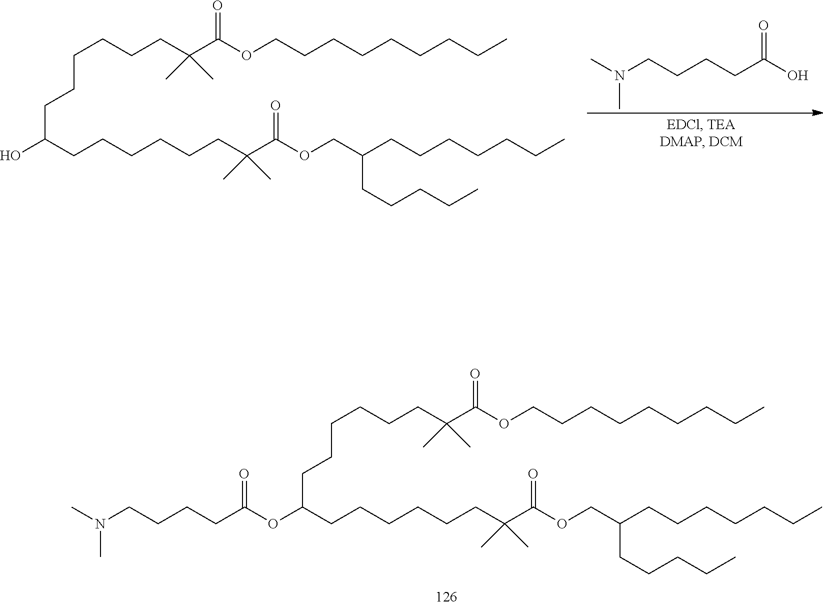

Referring to the method of Example 104, compound 126 was prepared as an oily product: 35.1 mg. ¹H NMR (400 MHz, CDCl₃): δ ppm 0.89 (t, J=6.8 Hz, 9H), 1.16 (s, 12H), 1.18-1.37 (m, 48H), 1.48-1.65 (m, 15H), 2.32-2.43 (m, 10H), 3.95 (d, J=5.6 Hz, 2H), 4.05 (t, J=6.8 Hz, 2H), 4.81-4.89 (m, 1H); ESI-MS m/z: 822.7 [M+H]⁺.

### Example 127: Synthesis of Compound 127

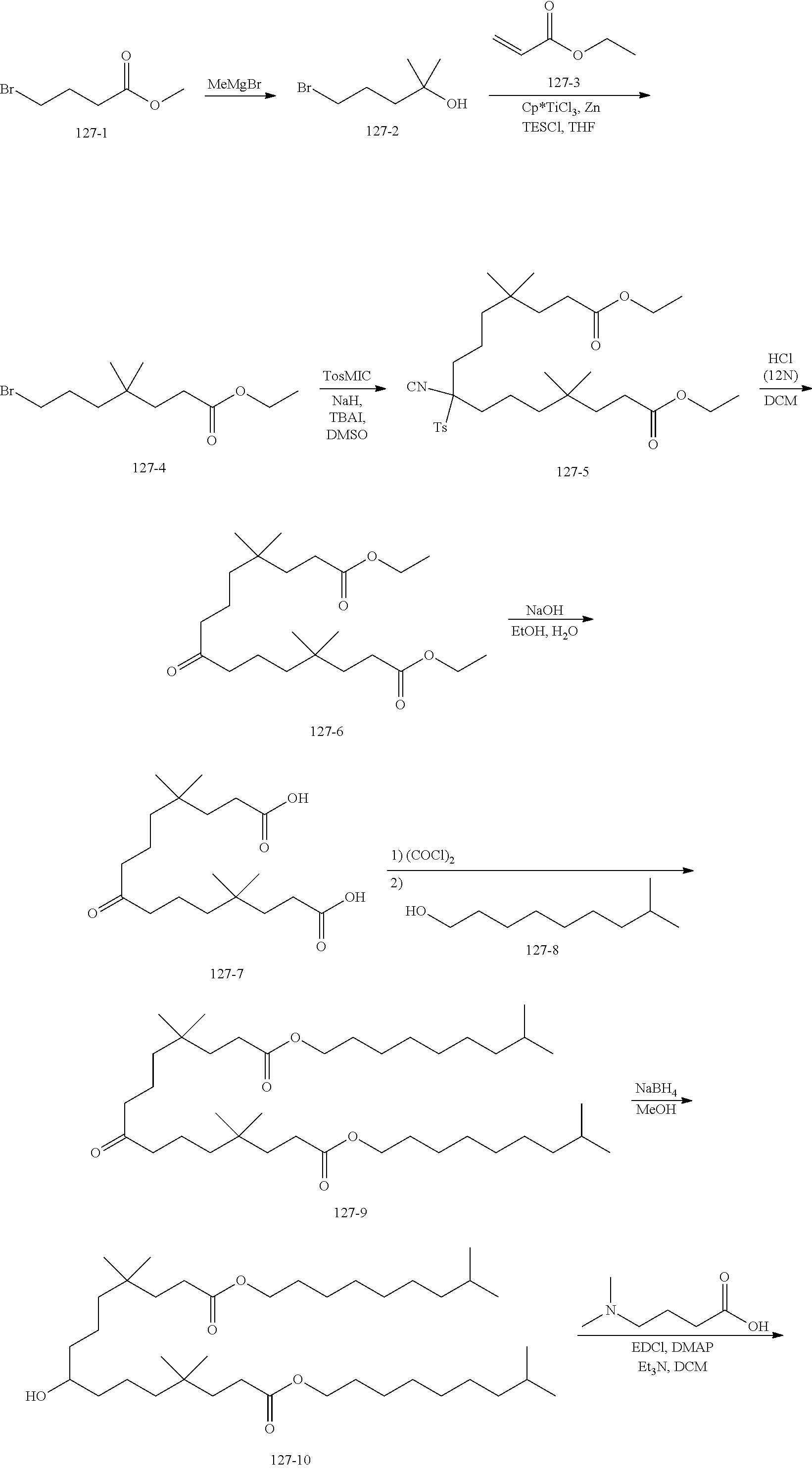

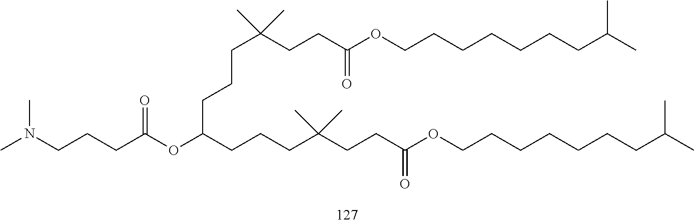 [SMILES: CC(C)CCCCCCCOC(=O)CCC(C)(C)CCCC(CCCC(C)(C)CCC(=O)OCCCCCCCC(C)C)OC(=O)CCCN(C)C]

A solution of compound 127-1 (100 g, 552.4 mmol) in anhydrous ether (800 mL) was cooled to 0° C. in an ice bath, and methylmagnesium bromide (3 M in ether, 737 mL) was slowly added dropwise to the solution. After the dropwise addition was completed, the ice bath was removed and the mixture was stirred to react for 4 h at room temperature. The reaction system was quenched with saturated ammonium chloride aqueous solution, and extracted with ether. The organic phases were combined, dried over anhydrous sodium sulfate, and filtered. The filtrate was concentrated to dryness to give the crude product. The crude product was purified by silica gel column to give compound 127-2 (100 g).

Compound 127-2 (42 g, 232 mmol), compound 127-3 (30.3 mL, 278 mmol), Cp*TiCl₃ (5.09 g, 23.2 mmol), zinc powder (45.5 g, 696 mmol), and triethylchlorosilane (116.8 mL, 696 mmol) were added to a round bottom flask. Then anhydrous tetrahydrofuran (1200 mL) was added to the reaction system and the reaction was carried out under the protection of argon gas. The reaction system was heated to 60° C. and stirred to react for 1 hour. The reaction system was quenched with saturated aqueous sodium chloride solution and extracted with ethyl acetate. The organic phases were combined, dried over anhydrous sodium sulfate, and filtered. The filtrate was concentrated to dryness to give crude product 1-4, which was purified by silica gel column to give compound 127-4 (21 g).

Compound TosMIC (7.03 g, 36 mmol) was dissolved in DMSO (200 mL), and NaH (4.32 g, 60%, 108 mmol) was added to the reaction system in batches under ice bath conditions. After the addition was completed, the ice bath was removed and the mixture was reacted at room temperature for another 1 h. Compound 127-4 (21 g, 79 mmol) and TBAI (1.33 g, 3.6 mmol) were added to the reaction system, and the mixture was stirred at room temperature overnight. The reaction system was quenched with saturated aqueous sodium chloride solution and extracted with ethyl acetate. The organic phases were combined, dried over anhydrous sodium sulfate, and filtered. The filtrate was concentrated to dryness to give crude compound 127-5 (21.9 g), which was used directly in the next reaction step without purification.

To a solution of crude compound 127-5 (21.9 g, 38.8 mmol) in dichloromethane (350 mL) was added 200 mL of concentrated hydrochloric acid, and the mixture was reacted at room temperature for 2 h. The complete reaction of the substrate was monitored by TLC. The reaction system was quenched with saturated aqueous ammonium chloride solution and extracted with ethyl acetate. The organic phases were combined, dried over anhydrous sodium sulfate, and filtered. The filtrate was concentrated to dryness to give the crude product, which was purified by silica gel column to give compound 127-6 (12.5 g).

Compound 127-6 (12.5 g, 31.4 mmol) was dissolved in ethanol (20 mL)-water (40 mL), and NaOH (3.77 g, 94.2 mmol) was added to the mixed solution in batches under ice bath conditions. After the addition was completed, the ice bath was removed and the mixture was stirred at room temperature. The complete consumption of the reaction materials was monitored by TLC. The organic solvent was removed by rotary evaporation, and the residue was extracted with dichloromethane. The aqueous layer was collected, and the solution was adjusted to a pH of <5 with 1 M hydrochloric acid. The solution was extracted with dichloromethane. The organic phases were combined, dried over anhydrous sodium sulfate, and filtered. The filtrate was collected and concentrated to give compound 127-7 (9.7 g).

DMF (17 μL, 0.22 mmol) was added to a solution of compound 127-7 (750 mg, 2.19 mmol) in dichloromethane (10.0 mL) under ice bath conditions, and oxalyl chloride (0.77 mL, 8.76 mmol) was then added dropwise to the reaction solution. The ice bath was removed, and the mixture was stirred for 1 h at room temperature. The solvent was removed using a rotary-evaporator to give acyl chloride crude product, which was used directly in the next reaction step.

The above obtained acyl chloride crude product was dissolved in 10.0 mL of 1,2-dichloroethane, and then compound 127-8 (693 mg, 4.38 mmol) was added to the reaction solution. The mixture was stirred at room temperature until the substrate was reacted completely. The solvent was removed using a rotary-evaporator. The crude was purified by silica gel column to give compound 127-9 (800 mg).

Compound 127-9 (800 mg, 1.29 mmol) was dissolved in 5.0 mL of methanol and sodium borohydride (146 mg, 3.87 mmol) was added to the reaction system. The mixture was reacted at room temperature. The complete disappearance of the reactants was monitored by TLC. The reaction system was quenched with saturated aqueous sodium chloride solution and extracted with dichloromethane. The organic phases were combined, dried over anhydrous sodium sulfate, and filtered. The filtrate was concentrated to dryness to give crude compound 127-10 (800 mg), which was used directly in the next reaction without purification.

Crude compound 127-10 (300 mg, 0.48 mmol), 4-dimethylaminobutyric acid (94.4 mg, 0.72 mmol), EDCI (276 mg, 1.44 mmol), triethylamine (0.21 mL, 1.44 mmol) and DMAP (59 mg, 0.48 mmol) were dissolved in 5.0 mL of dichloromethane, and the reaction solution was stirred to react at room temperature for 12 h. The reaction solution was quenched with saturated aqueous sodium chloride and extracted with dichloromethane. The organic phases were combined, dried over anhydrous sodium sulfate, and filtered. The organic phase was collected, and the organic solvent was removed using a rotary-evaporator. The crude product was purified by preparative high performance liquid chromatography to give the compound 127 (43.6 mg). ¹H NMR (400 MHz, CD₃OD): δ ppm 0.77-0.93 (m, 28H), 1.08-1.74 (m, 38H), 1.76-1.84 (m, 2H), 1.22-2.27 (m, 10H), 2.33-2.37 (m, 4H), 4.03-4.12 (m, 4H), 4.92-4.97 (m, 1H); ESI-MS m/z: 738.6 [M+H]⁺.

### Example 128: Synthesis of Compound 128

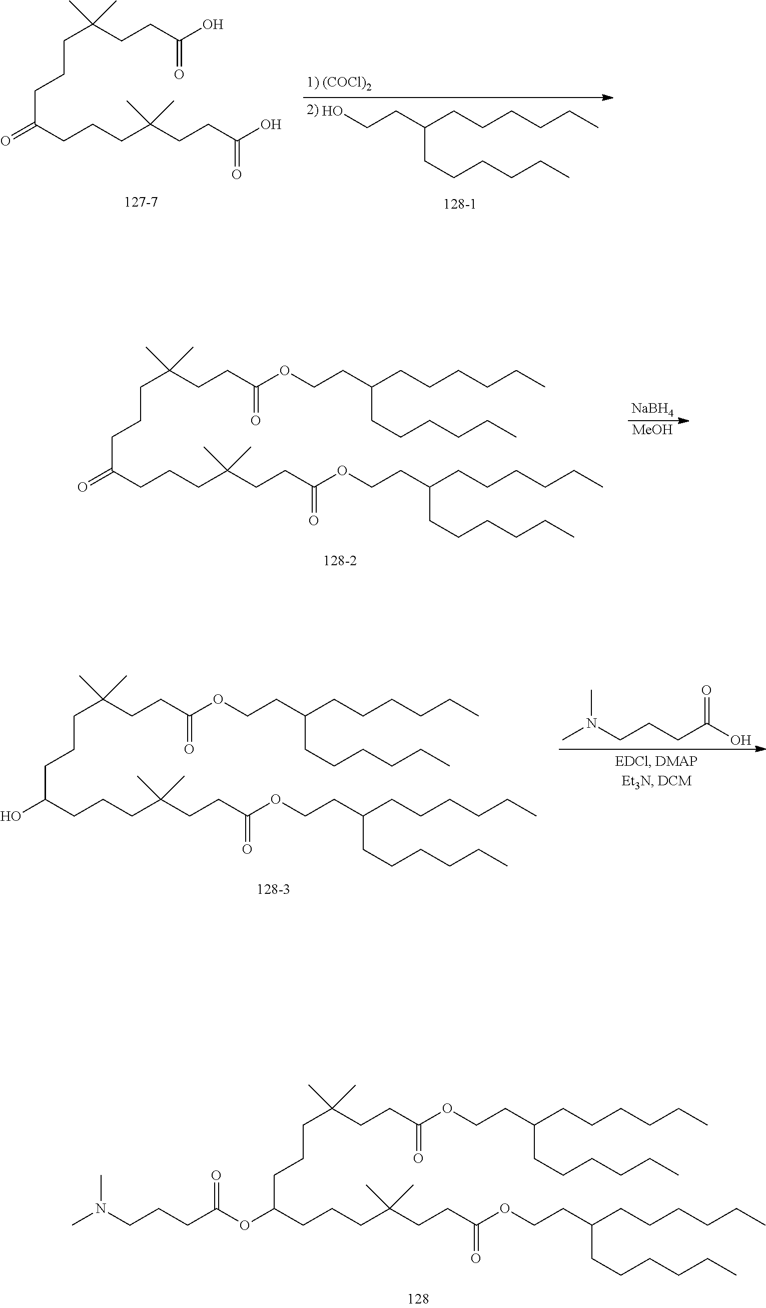

Referring to the method of Example 127, compound 128 was prepared as an oily product: 64.2 mg. ¹H NMR (400 MHz, CD₃OD): δ ppm 0.86 (s, 12H), 0.91 (t, J=6.8 Hz, 12H), 1.22-1.37 (m, 48H), 1.51-1.61 (m, 14H), 1.78-1.86 (m, 2H), 2.24 (t, J=8.0 Hz, 4H), 2.30 (s, 6H), 2.37 (t, J=7.2 Hz, 2H), 2.43 (t, J=8.0 Hz, 2H), 4.10 (t, J=6.8 Hz, 4H), 4.92-4.97 (m, 1H); ESI-MS m/z: 878.7 [M+H]⁺.

### Example 129: Synthesis of Compound 129

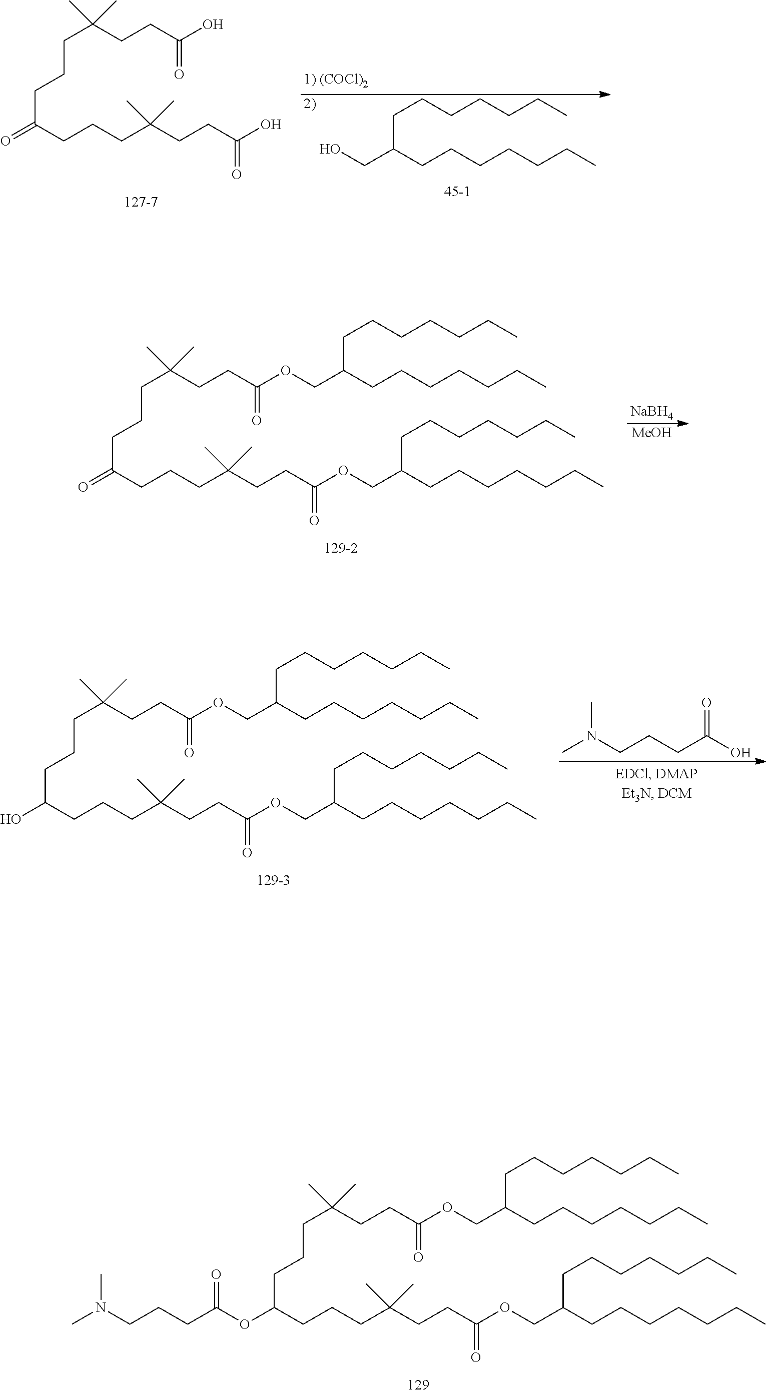

Referring to the method of Example 127, compound 129 was prepared as an oily product: 67.0 mg. ¹H NMR (400 MHz, CD₃OD): δ ppm 0.86 (s, 12H), 0.88-0.93 (m, 12H), 1.12-1.40 (m, 52H), 1.49-1.56 (m, 8H), 1.59-1.66 (m, 4H), 1.77-1.84 (m, 4H), 2.22-2.27 (m, 10H), 2.34-2.38 (m, 4H), 4.01 (t, J=6.8 Hz, 4H), 4.91-4.97 (m, 1H); ESI-MS m/z: 906.8 [M+H]⁺.

### Example 130: Synthesis of Compound 130

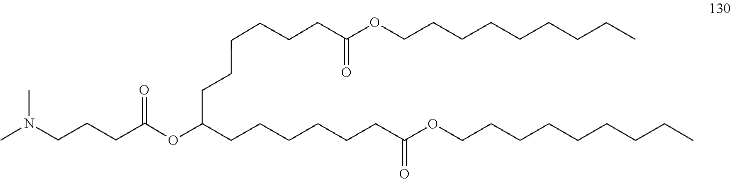 [SMILES: CCCCCCCCCOC(=O)CCCCCCC(CCCCCCC(=O)OCCCCCCCCC)OC(=O)CCCN(C)C]

Referring to the method of Example 20, compound 130 was prepared. 1H NMR (400 MHz, CDCl3): δ ppm 0.77-0.86 (t, J=7.2 Hz, 6H), 1.15-1.30 (m, 38H), 1.40-1.59 (m, 12H), 1.87-1.96 (m, 2H), 2.21 (t, J=7.2 Hz, 4H), 2.28-2.37 (m, 2H), 2.40-2.50 (m, 5H), 2.56-2.67 (m, 2H), 3.92-4.07 (m, 4H), 4.72-4.90 (m, 1H); ESI-MS m/z: [M+H]+: 654.6.

### Example 131: Synthesis of Compound 131

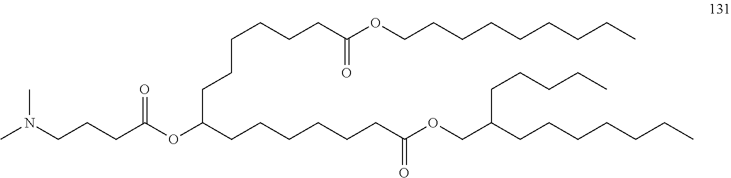 [SMILES: CCCCCCCCCOC(=O)CCCCCCC(CCCCCCC(=O)OCC(CCCCC)CCCCCCC)OC(=O)CCCN(C)C]

Referring to the method of Example 46, compound 131 was prepared. 1H NMR (400 MHz, CDCl3): δ ppm 0.89 (t, J=7.2 Hz, 9H), 1.21-1.30 (m, 44H), 1.50-1.63 (m, 11H), 1.77-1.92 (m, 2H), 2.27-2.36 (m, 14H), 3.97 (d, J=5.6 Hz, 2H), 4.06 (t, J=6.8 Hz, 2H), 4.85-4.92 (m, 1H); ESI-MS m/z: [M+H]+: 724.6.

### Example 132: Synthesis of Compound 132

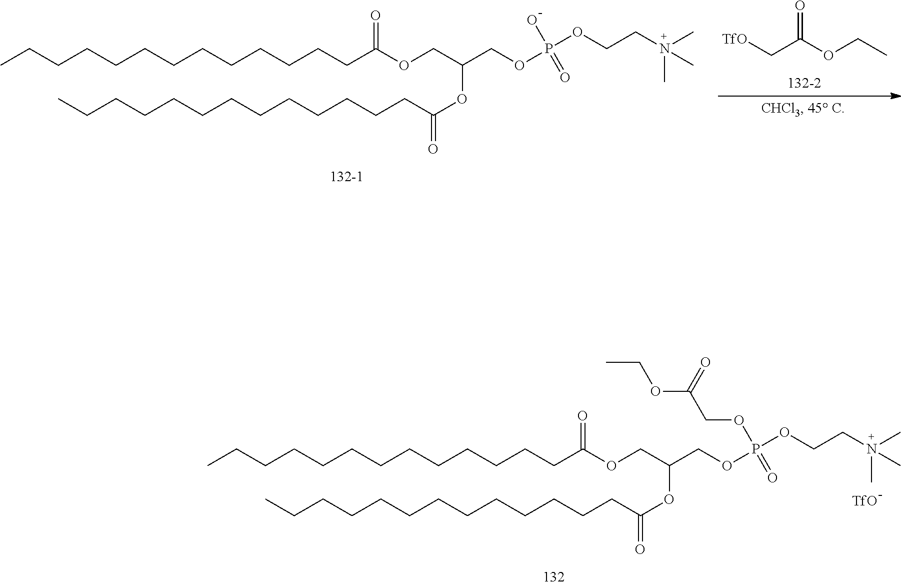

Compound 132-1 (101.7 mg, 0.15 mmol) and compound 132-2 (40.1 mg, 0.17 mmol) were added to a 5.0 mL chloroform solution, and the resulting mixture was heated at 45° C. with stirring. The reaction was monitored by LC-MS until compound 132-1 was completely consumed. The organic solvent was removed by nitrogen gas flow to obtain the crude product. The crude product was added to n-hexane (5.0 mL×3), stirred, filtered and dried to obtain Compound 132 (35.9 mg) as a white solid. ¹H NMR: (400 MHz, CDCl₃): δ 0.89 (t, J=7.2 Hz, 6H), 1.26-1.32 (m, 43H), 1.54-1.67 (m, 4H), 2.30-2.36 (m, 4H), 3.36 (s, 9H), 4.03 (br d, J=2.8 Hz, 2H), 4.23-4.32 (m, 6H), 4.57-4.74 (m, 4H), 5.25-5.29 (m, 1H); ESI-MS m/z: 764.5 [M]⁺.

### Example 133: Synthesis of Compound 133

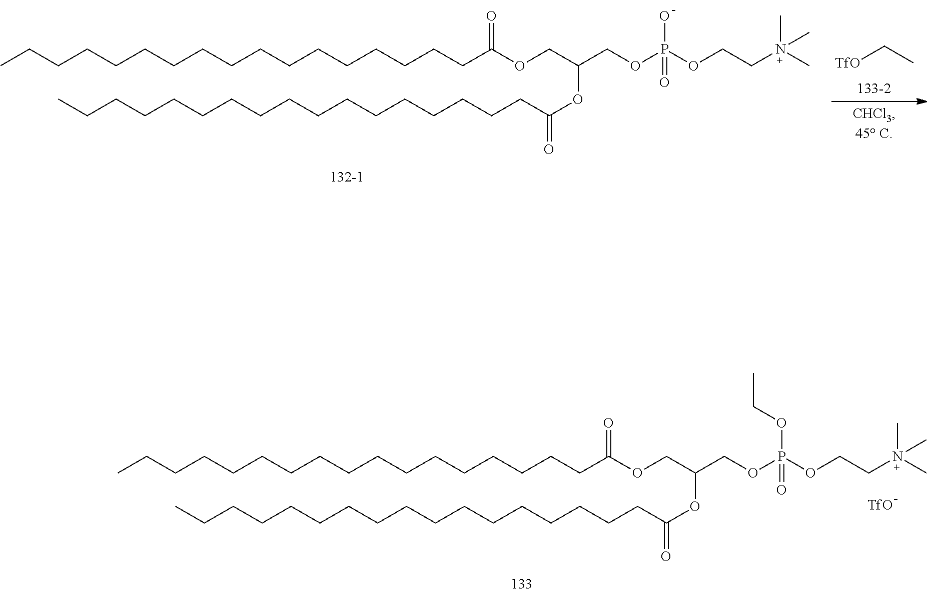

Compound 133-1 (250 mg, 0.32 mmol) and compound 133-2 (62.3 mg, 0.35 mmol) were added to a 10.0 mL chloroform solution, and the resulting mixture was heated at 45° C. with stirring for 2 hours. The reaction was monitored by LC-MS until compound 133-1 was completely consumed. The organic solvent was removed by nitrogen gas flow to obtain the crude product. The crude product was added to n-hexane (8.0 mL×3), stirred, filtered and dried to obtain Compound 133 (165 mg) as a white solid. ¹H NMR: (400 MHz, CDCl₃): δ 0.89 (t, J=7.2 Hz, 6H), 1.26-1.38 (m, 58H), 1.52-1.67 (m, 5H), 2.31-2.37 (m, 4H), 3.31 (s, 9H), 3.92 (br d, J=2.8 Hz, 2H), 4.16-4.34 (m, 6H), 4.47-4.63 (m, 2H), 5.24-5.28 (m, 1H); ESI-MS m/z: 818.7 [M]⁺.

### Formulation and Biological Studies

### Example 1A: Preparation of LNPs without Permanently Cationic Lipid

Materials used for lipid nanoparticle assembly include: (1) ionizable lipid compounds: e.g., ionizable lipids designed and synthesized in the present disclosure or Dlin-MC3-DMA (purchased from AVT) as a control; (2) steroid: e.g., Cholesterol (purchased from Sigma-Aldrich); (3) phospholipids: e.g., DSPC i.e., 1,2-distearoyl-SN-glycero-3-phosphocholine (Distearoylphosphatidylcholine, purchased from AVT); (4) polyethylene glycolated lipids: e.g. DMG-PEG2000 i.e., dimyristoylglycerol-polyethylene glycol 2000 (1,2-dimyristoyl-rac-glycero-3-methoxypolyethylene glycol-2000, purchased from AVT); (5) active ingredients of nucleic acid fragments: e.g. Luciferase mRNA, siRNA, CRISPR Cas 9 mRNA, etc. (manufactured in-house).

Lipid nanoparticles were prepared by (1) dissolving and mixing ionizable lipid compounds, cholesterol, phospholipids and polyethylene glycolated lipids in ethanol at (molar percentages) 50%, 38.5%, 10% and 1.5%, respectively; (2) dissolving the mRNA therapeutic agent in 25 mM sodium acetate solution (pH=4.5); (3) using an automated high-throughput microfluidic system to mix the organic phase containing the lipid mixture and the aqueous phase containing the mRNA component in the flow ratio range of 1:1 to 1:4 at a mixing speed of 10 mL/min to 18 mL/min; (4) the prepared lipid nanoparticles (N/P ratio of 6) were diluted with phosphate buffer solution and the lipid nanoparticle solutions were ultrafiltered to the original preparation volume using ultrafiltration tubes (purchased from Millipore) with a cut-off molecular weight of 30 kDa; and (5) the obtained nanoparticles were filtered through a sterile 0.2 μm filter membrane and then stored in a sealed glass vial at low temperature.

The preparation method of lipid nanoparticles includes microfluidic mixing systems, but is not limited to this method. Other methods include T-type mixers, and ethanol injection method, and the like.

### Example 2A: Characterization of LNPs without Permanently Cationic Lipid

The particle size and particle size dispersity index (PDI) of the prepared lipid nanoparticles were measured using Dynamic Light Scattering (DLS) with a Zetasizer Pro (purchased from Malvern Instruments Ltd) and a DynaPro NanoStar (purchased from Wyatt) instrument. The degree of RNA encapsulation by lipid nanoparticles was characterized by the Encapsulation Efficiency %, which reflects the degree of binding of lipid nanoparticles to RNA fragments. This parameter was measured by the method of Quant-it™ RiboGreen RNA Assay (purchased from Invitrogen). Lipid nanoparticle samples were diluted in TE buffer (10 mM Tris-HCl, 1 mM EDTA, pH=7.5). A portion of the sample solution was removed, to which 0.5% Triton (Triton X-100) was added, and then allowed to stand at 37° C. for 30 minutes. Immediately after the addition of RIBOGREEN® reaction solution, the fluorescence values were read on a Varioskan LUX multifunctional microplate reader (purchased from Thermo Fisher) at 485 nm for absorption and 528 nm for emission to give the encapsulation efficiency values.

### Example 3A: Animal Experiment Using LNPs without Permanently Cationic Lipid

The delivery effect and safety of nanoparticles encapsulated with luciferase mRNA (Trilink, L-7202) in mice were evaluated. The test mice were SPF-grade C57BL/6 mice, female, 6-8 weeks old, weighing 18-22 g, and were purchased from SPF (Beijing) Biotechnology Co., Ltd. All animals were acclimatized for more than 7 days prior to the experiment, and had free access to food and water during the experiment. The conditions include alternating light and dark for 12/12 h, the indoor temperature of 20-26° C. and the humidity of 40-70%. The mice were randomly grouped. The lipid nanoparticles encapsulated with luciferase mRNA prepared above were injected into mice by intravenous administration at a single dose of 0.5 mg/kg mRNA, and the mice were subjected to in vivo bioluminescence assay using a Small Animal In Vivo Imaging System (IVIS LUMINA III, purchased from PerkinElmer) at 6 h after administration. The assay was performed as follows: D-luciferin solution was prepared in saline at a concentration of 15 mg/mL, and each mouse was given the substrate by intraperitoneal injection. At ten minutes after administration of the substrate, the mice were anesthetized in an anesthesia chamber with isoflurane at a concentration of 2.5%. The anesthetized mice were placed in IVIS for luminescent imaging. Data acquisition and analysis were performed on the concentrated distribution area of luminescence.

The in vivo delivery efficiency of lipid nanoparticle carriers was expressed as the mean values of fluorescence intensity and total photon count in different animals measured at 6 hours after administration of the lipid nanoparticle within the same subject group, as shown in Table 4. Higher values of fluorescence intensity and total photon count indicate higher in vivo delivery efficiency of this mRNA fragment by the lipid nanoparticles. These lipid nanoparticles containing ionizable lipids exhibit excellent in vivo delivery efficiency and mainly target the liver.

TABLE 4

| | Particle | Particle size | | Total photon |
|---|---|---|---|---|
| Ionizable | size | dispersity | Encapsulation | count in vivo |
| Lipid | (nm) | (PDI) | efficiency (%) | (Total Flux) |
| 1 | 80.07 | 0.06 | 91.54 | 6.43E+08 |
| 2 | 229.35 | 0.03 | 79.14 | 4.23E+08 |
| 3 | 133.80 | 0.04 | 86.88 | 1.75E+09 |
| 6 | 106.18 | 0.05 | 90.26 | 2.25E+09 |
| 7 | 203.67 | 0.04 | 69.51 | 1.16E+08 |
| 10 | 118.59 | 0.07 | 53.83 | 1.26E+09 |
| 12 | 89.34 | 0.07 | 85.02 | 1.10E+09 |
| 15 | 91.68 | 0.06 | 84.78 | 4.89E+08 |
| 18 | 161.29 | 0.05 | 87.86 | 1.14E+10 |
| 19 | 135.15 | 0.19 | 67.96 | 3.20E+09 |
| 20 | 99.61 | 0.10 | 71.93 | 2.40E+10 |
| 23 | 151.35 | 0.07 | 70.22 | 1.43E+10 |
| 24 | 133.72 | 0.04 | 60.98 | 5.56E+09 |
| 25 | 236.90 | 0.06 | 32.43 | 1.89E+08 |
| 26 | 96.69 | 0.05 | 81.29 | >4.00E+10 |
| 27 | 105.70 | 0.05 | 88.99 | 2.20E+10 |
| 28 | 138.16 | 0.06 | 80.36 | 3.21E+09 |
| 32 | 116.61 | 0.07 | 99.02 | 7.30E+09 |
| 33 | 105.84 | 0.04 | 88.67 | 2.12E+08 |
| 34 | 104.71 | 0.04 | 90.29 | 1.26E+10 |
| 36 | 88.33 | 0.07 | 97.52 | 4.84E+09 |
| 37 | 92.18 | 0.03 | 89.07 | 7.88E+09 |
| 40 | 83.90 | 0.05 | 97.06 | 4.22E+09 |
| 41 | 104.27 | 0.05 | 93.91 | 2.19E+10 |
| 42 | 152.04 | 0.07 | 71.95 | 5.32E+09 |
| 46 | 97.30 | 0.09 | 95.68 | >4.00E+10 |
| 92 | 107.56 | 0.05 | 91.66 | 2.13E+09 |
| 97 | 157.00 | 0.25 | 93.72 | 3.37E+09 |
| 98 | 107.87 | 0.13 | 93.28 | 2.12E+10 |
| 99 | 139.99 | 0.05 | 90.69 | 5.30E+09 |
| 100 | 118.19 | 0.07 | 83.29 | 1.18E+10 |
| 101 | 152.45 | 0.06 | 76.09 | 5.19E+09 |
| 102 | 102.18 | 0.03 | 90.03 | 2.03E+10 |
| 103 | 148.67 | 0.04 | 90.97 | 5.69E+09 |
| 104 | 71.14 | 0.06 | 85.54 | 2.61E+10 |
| 105 | 78.80 | 0.05 | 94.67 | 3.33E+09 |
| 106 | 83.09 | 0.05 | 85.65 | 1.85E+10 |
| 107 | 99.54 | 0.05 | 83.74 | 1.36E+10 |
| 108 | 121.41 | 0.05 | 92.63 | 1.21E+10 |
| 109 | 101.04 | 0.06 | 87.11 | 1.32E+10 |
| 110 | 97.59 | 0.01 | 92.65 | 2.06E+08 |
| 111 | 141.30 | 0.06 | 95.12 | 1.79E+10 |
| 112 | 121.05 | 0.08 | 94.41 | 3.53E+10 |
| 113 | 112.65 | 0.03 | 94.77 | 3.59E+08 |
| 114 | 72.32 | 0.04 | 93.41 | 1.06E+08 |
| 115 | 77.12 | 0.04 | 91.76 | 2.56E+10 |
| 116 | 88.62 | 0.05 | 89.36 | >4.00E+10 |
| 117 | 127.31 | 0.06 | 95.29 | 4.48E+09 |
| 118 | 88.33 | 0.07 | 93.87 | 3.14E+10 |
| 119 | 80.98 | 0.05 | 86.92 | 3.25E+10 |
| 120 | 98.99 | 0.07 | 95.55 | 3.15E+10 |
| 121 | 103.57 | 0.06 | 93.62 | >4.00E+10 |
| 122 | 96.72 | 0.07 | 91.76 | >4.00E+10 |
| 123 | 84.98 | 0.05 | 92.27 | >4.00E+10 |
| 125 | 95.69 | 0.07 | 89.45 | 1.99E+10 |
| 126 | 133.76 | 0.06 | 94.35 | 1.77E+10 |
| 127 | 83.01 | 0.05 | 62.35 | 3.59E+10 |
| 128 | 83.10 | 0.05 | 79.88 | 1.00E+10 |
| 129 | 112.08 | 0.06 | 57.87 | 7.82E+09 |
| 131 | 245.33 | 0.20 | 79.63 | 1.57E+09 |
| Dlin-MC3-DMA | 96.83 | 0.04 | 95.62 | 8.04E+09 |

### Example 4A: LNPs Comprising Permanently Cationic Lipid

Materials used for lipid nanoparticle assembly include: (1) ionizable lipid compounds, such as ionizable lipids 20, 26, 46, MC3, SM102, ALC0315, or Lipid 5 used herein; (2) steroids: e.g., Cholesterol (purchased from Sigma-Aldrich); (3) permanently cationic lipid, such as DOTMA (1,2-di-O-octadecenyl-3-trimethylammonium propane (chloride salt), purchased from AVT), DOTAP (1,2-dioleoyl-3-trimethylammonium-propane (chloride salt)), (4) polyethylene glycolated (pegylated) lipid, such as DMG-PEG2000 (1,2-dimyristoyl-rac-glycero-3-methoxypolyethylene glycol-2000, purchased from AVT); (5) Phospholipid, such as DSPC or DOPE; (6) therapeutic agent, such as nucleic acid (e.g., Luciferase mRNA). Materials used for the manufacturing of the LNPs are listed in Table 5A. Exemplary formulations are listed in Table 5B.

TABLE 5A

| LNP | |
|---|---|
| Compositions | Details |
| Luciferase | Commercially available from Trilink |
| mRNA | |
| (Therapeutic | |
| Agent) | |
| Compound 46 (Ionizable Lipid) | 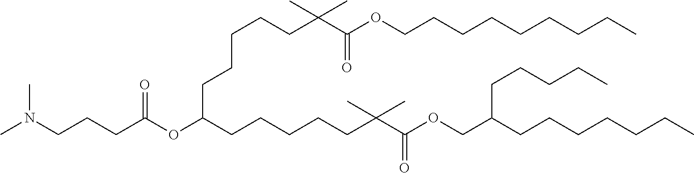 [SMILES: CCCCCCCCCOC(=O)C(C)(C)CCCCCC(CCCCCC(C)(C)C(=O)OCC(CCCCC)CCCCCCC)OC(=O)CCCN(C)C] |
| Compound 20 (Ionizable Lipid) | 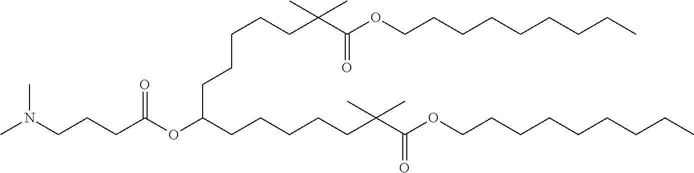 [SMILES: CCCCCCCCCOC(=O)C(C)(C)CCCCCC(CCCCCC(C)(C)C(=O)OCCCCCCCCC)OC(=O)CCCN(C)C] |
| Compound 26 (Ionizable Lipid) | 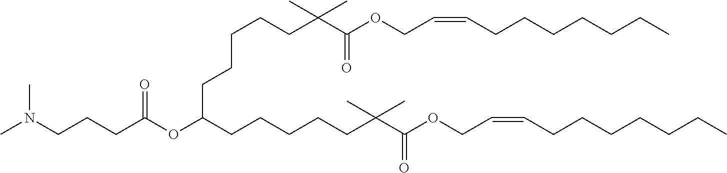 [SMILES: CCCCCCC/C=C\COC(=O)C(C)(C)CCCCCC(CCCCCC(C)(C)C(=O)OC/C=C\CCCCCCC)OC(=O)CCCN(C)C] |
| MC3 (Ionizable Lipid) | 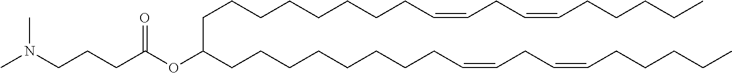 [SMILES: CCCCC/C=C\C/C=C\CCCCCCCCC(CCCCCCCC/C=C\C/C=C\CCCCC)OC(=O)CCCN(C)C] |
| SM102 (Ionizable Lipid) | 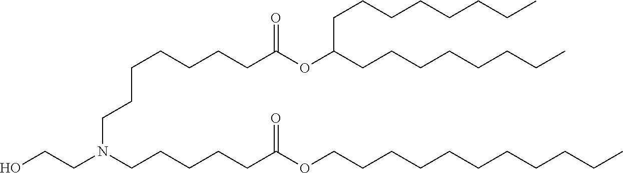 [SMILES: CCCCCCCCCCCOC(=O)CCCCCN(CCO)CCCCCCCC(=O)OC(CCCCCCCC)CCCCCCCC] |
| ALC0315 (Ionizable Lipid) | 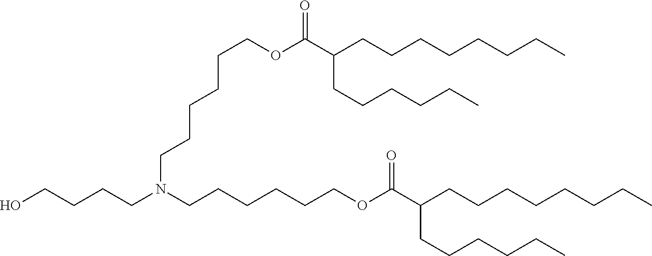 [SMILES: CCCCCCCCC(CCCCCC)C(=O)OCCCCCCN(CCCCO)CCCCCCOC(=O)C(CCCCCC)CCCCCCCC] |
| Lipid 5 (Ionizable Lipid) | 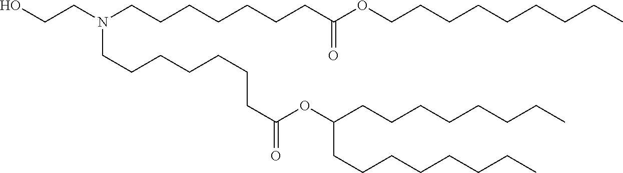 [SMILES: CCCCCCCCCOC(=O)CCCCCCCN(CCO)CCCCCCCC(=O)OC(CCCCCCCC)CCCCCCCC] |
| DOTAP (Permanently Cationic Lipid) | 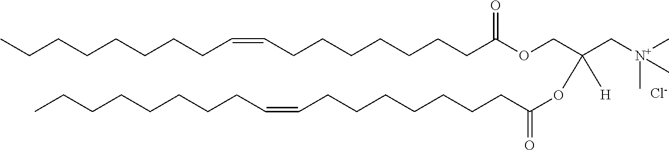 [SMILES: CCCCCCCC/C=C\CCCCCCCC(=O)OCC(C[N+](C)(C)C)OC(=O)CCCCCCC/C=C\CCCCCCCC ; [Cl-]] |
| DOTMA (Permanently Cationic Lipid) | 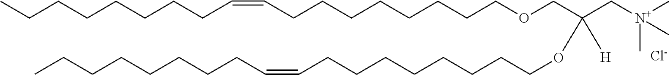 [SMILES: CCCCCCCC/C=C\CCCCCCCCOCC(C[N+](C)(C)C)OCCCCCCCC/C=C\CCCCCCCC ; [Cl-]] |
| Cholesterol (Structural Lipid) | 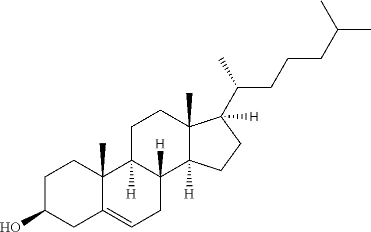 |
| DMG-PEG2K (Pegylated Lipid) | 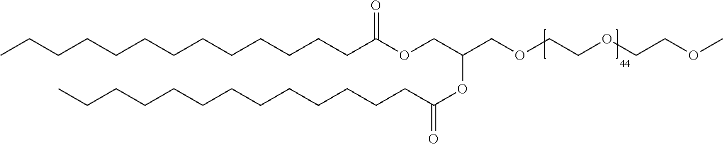 |
| ALC-0159 (Pegylated Lipid) | 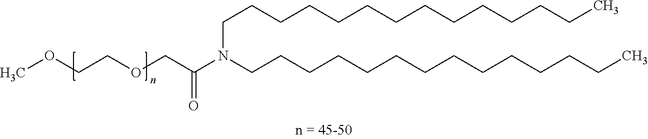 |
| DSPC | 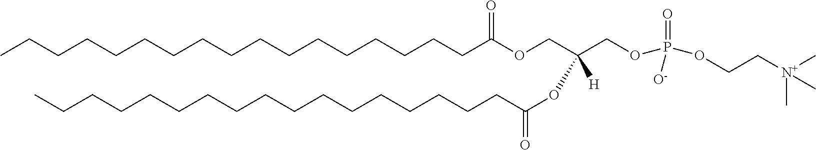 [SMILES: CCCCCCCCCCCCCCCCCC(=O)OC[C@H](COP(=O)([O-])OCC[N+](C)(C)C)OC(=O)CCCCCCCCCCCCCCCCC] |
| DOPE | 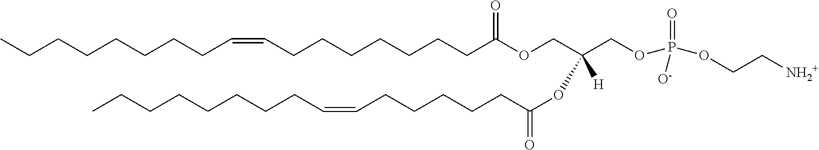 [SMILES: CCCCCCCC/C=C\CCCCCCCC(=O)OC[C@H](COP(=O)([O-])OCC[NH3+])OC(=O)CCCCC/C=C\CCCCCCCC] |

TABLE 5B

| Amount | |
|---|---|
| (MOL %) | Components |
| 50:24:0:25:1 | Permanently Cationic Lipid:Ionizable |
| | Lipid:Phospholipid:Chol:PEG-lipid |
| 50:29:0:20:1 | Permanently Cationic Lipid:Ionizable |
| | Lipid:Phospholipid:Chol:PEG-lipid |
| 50:34:0:15:1 | Permanently Cationic Lipid:Ionizable |
| | Lipid:Phospholipid:Chol:PEG-lipid |
| 45:24:0:30:1 | Permanently Cationic Lipid:Ionizable |
| | Lipid:Phospholipid:Chol:PEG-lipid |
| 45:29:0:25:1 | Permanently Cationic Lipid:Ionizable |
| | Lipid:Phospholipid:Chol:PEG-lipid |
| 45:34:0:20:1 | Permanently Cationic Lipid:Ionizable |
| | Lipid:Phospholipid:Chol:PEG-lipid |
| 55:24:0:20:1 | Permanently Cationic Lipid:Ionizable |
| | Lipid:Phospholipid:Chol:PEG-lipid |
| 55:29:0:15:1 | Permanently Cationic Lipid:Ionizable |
| | Lipid:Phospholipid:Chol:PEG-lipid |
| 55:34:0:10:1 | Permanently Cationic Lipid:Ionizable |
| | Lipid:Phospholipid:Chol:PEG-lipid |
| 50:24:0:24:2 | Permanently Cationic Lipid:Ionizable |
| | Lipid:Phospholipid:Chol:PEG-lipid |
| 50:29:0:19:2 | Permanently Cationic Lipid:Ionizable |
| | Lipid:Phospholipid:Chol:PEG-lipid |
| 50:34:0:14:2 | Permanently Cationic Lipid:Ionizable |
| | Lipid:Phospholipid:Chol:PEG-lipid |
| 45:24:0:29:2 | Permanently Cationic Lipid:Ionizable |
| | Lipid:Phospholipid:Chol:PEG-lipid |
| 45:29:0:24:2 | Permanently Cationic Lipid:Ionizable |
| | Lipid:Phospholipid:Chol:PEG-lipid |
| 45:34:0:19:2 | Permanently Cationic Lipid:Ionizable |
| | Lipid:Phospholipid:Chol:PEG-lipid |
| 55:24:0:19:2 | Permanently Cationic Lipid:Ionizable |
| | Lipid:Phospholipid:Chol:PEG-lipid |
| 55:29:0:14:2 | Permanently Cationic Lipid:Ionizable |
| | Lipid:Phospholipid:Chol:PEG-lipid |
| 55:34:0:9:2 | Permanently Cationic Lipid:Ionizable |
| | Lipid:Phospholipid:Chol:PEG-lipid |
| 45:24:5:25:1 | Permanently Cationic Lipid:Ionizable |
| | Lipid:Phospholipid:Chol:PEG-lipid |
| 45:29:5:20:1 | Permanently Cationic Lipid:Ionizable |
| | Lipid:Phospholipid:Chol:PEG-lipid |
| 45:34:5:15:1 | Permanently Cationic Lipid:Ionizable |
| | Lipid:Phospholipid:Chol:PEG-lipid |
| 40:24:10:30:1 | Permanently Cationic Lipid:Ionizable |
| | Lipid:Phospholipid:Chol:PEG-lipid |
| 40:29:10:25:1 | Permanently Cationic Lipid:Ionizable |
| | Lipid:Phospholipid:Chol:PEG-lipid |
| 40:34:10:20:1 | Permanently Cationic Lipid:Ionizable |
| | Lipid:Phospholipid:Chol:PEG-lipid |
| 45:24:5:24:2 | Permanently Cationic Lipid:Ionizable |
| | Lipid:Phospholipid:Chol:PEG-lipid |
| 45:29:5:19:2 | Permanently Cationic Lipid:Ionizable |
| | Lipid:Phospholipid:Chol:PEG-lipid |
| 45:34:5:14:2 | Permanently Cationic Lipid:Ionizable |
| | Lipid:Phospholipid:Chol:PEG-lipid |
| 40:24:10:29:2 | Permanently Cationic Lipid:Ionizable |
| | Lipid:Phospholipid:Chol:PEG-lipid |
| 40:29:10:24:2 | Permanently Cationic Lipid:Ionizable |
| | Lipid:Phospholipid:Chol:PEG-lipid |
| 40:34:10:19:2 | Permanently Cationic Lipid:Ionizable |
| | Lipid:Phospholipid:Chol:PEG-lipid |

### Example 5A: Preparation of LNPs Comprising Permanently Cationic Lipid

Lipid nanoparticles were prepared by (1) dissolving and mixing ionizable lipid, permanently cationic lipid, cholesterol, and pegylated lipids in ethanol at molar ratios listed below; (2) dissolving the mRNA therapeutic agent in 25 mM sodium acetate solution (pH=4.5); (3) using an automated high-throughput microfluidic system to mix the organic phase containing the lipids and the aqueous phase containing the mRNA at a volumetric ratio of from about 1:1 to about 1:4 and at a flow rate of from 2 mL/min to 20 mL/min. LNPs having diameters at about 100 nm, 200 nm, 300 nm, and over 300 nm were produced by controlling the flow rate during the mixing process (4) The LNPs prepared were diluted with a phosphate-buffered saline (PBS) solution and filtered through a 30 kDa molecular weight cut-off ultrafiltration membrane (purchased from Millipore) to the original volume of the preparation. The resulting LNP solution was filtered through a 0.2 μm filter membrane for sterilization to remove any bacteria, and stored in a sealed glass vial at low temperature. Exemplary formulations are shown in Table 6A and 6B. Table 7 shows the size of the LNPs prepared and encapsulation efficiency of mRNA. The LNPs prepared have positive surface charge.

TABLE 6A

| Formulation 1 Comprising DOTAP | | Formulation 2 Comprising DOTMA | |
|---|---|---|---|
| Lipids | Molar Ratio % | Lipids | Molar Ratio % |
| Compound 46 | 24 | Compound 46 | 24 |
| CHO | 25 | CHO | 25 |
| DOTAP | 50 | DOTMA | 50 |
| PEG2K-DMG | 1 | PEG2K-DMG | 1 |

| | |
|---|---|
| Nitrogen/Phosphate (N/P) Ratio = 8 | Nitrogen/Phosphate (N/P) Ratio = 8 |

TABLE 6B

| Formulation 3 Comprising DOTAP | | Formulation 4 Comprising DOTAP | |
|---|---|---|---|
| Lipids | Molar Ratio % | Lipids | Molar Ratio % |
| SM102 | 24 | MC3 | 24 |
| CHO | 25 | CHO | 25 |
| DOTAP | 50 | DOTAP | 50 |
| PEG2K-DMG | 1 | PEG2K-DMG | 1 |

| | |
|---|---|
| Nitrogen/Phosphate (N/P) Ratio = 8 | Nitrogen/Phosphate (N/P) Ratio = 8 |

TABLE 7

| | | Permanently | | |
|---|---|---|---|---|
| Formulation | Entry | cationic lipid | Diameter (nm) | EE % |
| 1 | 1-1 | DOTAP | 98.74 ± 0.52 | 99.28 |
| | 1-2 | DOTAP | 203.63 ± 5.44 | 98.85 |
| | 1-3 | DOTAP | 300.67 ± 10.49 | 96.90 |
| | 1-4 | DOTAP | 372.80 ± 29.38 | 93.59 |
| 2 | 2-1 | DOTMA | 119.60 ± 1.55 | 99.76 |
| | 2-2 | DOTMA | 173.5 ± 12.83 | 101.39 |
| | 2-3 | DOTMA | 258.07 ± 18.13 | 99.76 |
| | 2-4 | DOTMA | 337.23 ± 99.68 | 96.29 |
| 3 | 3-1 | DOTAP | 110.63 ± 2.93 | 98.94 |
| | 3-2 | DOTAP | 202.13 ± 2.15 | 99.41 |
| 4 | 4-1 | DOTAP | 95.38 ± 1.23 | 99.11 |
| | 4-2 | DOTAP | 219.03 ± 0.49 | 99.15 |

### Example 6A: Animal Experiment Using LNPs Comprising Permanently Cationic Lipid

The delivery efficiency, as well as the impact of particle size on delivery to non-hepatic organs (e.g. lung) of LNPs of different sizes loaded with firefly luciferase mRNA are evaluated in mice. SPF-grade female C57BL/6 mice aged 6-8 weeks and weighted 18-22 g were purchased from Beijing Sibeifu Biotechnology Co., Ltd. All animals were adaptively fed for at least 7 days before the experiment, and are given free access to food and water during the experiment. The light/dark cycle was set to 12/12 h, and the indoor temperature and humidity were maintained at 20-26° C. and 40-70%, respectively. The prepared LNPs loaded with firefly luciferase mRNA were injected into the mice via tail vein injection at a single dose of 0.5 mg/kg of mRNA. At 6 hours after administration, the mice were dissected and the lungs and livers were removed for in vivo bioluminescent imaging using a small animal imaging system (IVIS LUMINA III, purchased from PerkinElmer). The specific steps of the detection are as follows: a 15 mg/mL concentration of D-luciferin solution was prepared with physiological saline, and each mouse was given the substrate by intraperitoneal injection. After 9 minutes of substrate administration, the mice were dissected, and the harvested organs were placed in the IVIS for fluorescence imaging. Data acquisition and analysis were performed on the concentrated distribution area of luminescence.

The in vivo delivery efficiency of LNPs and load mRNA was represented by the average luminescence intensity and total photon counts of different animals or organs within the same test group. A higher value of luminescence intensity and total photon counts represents higher in vivo delivery efficiency of the LNPs and loaded mRNA.

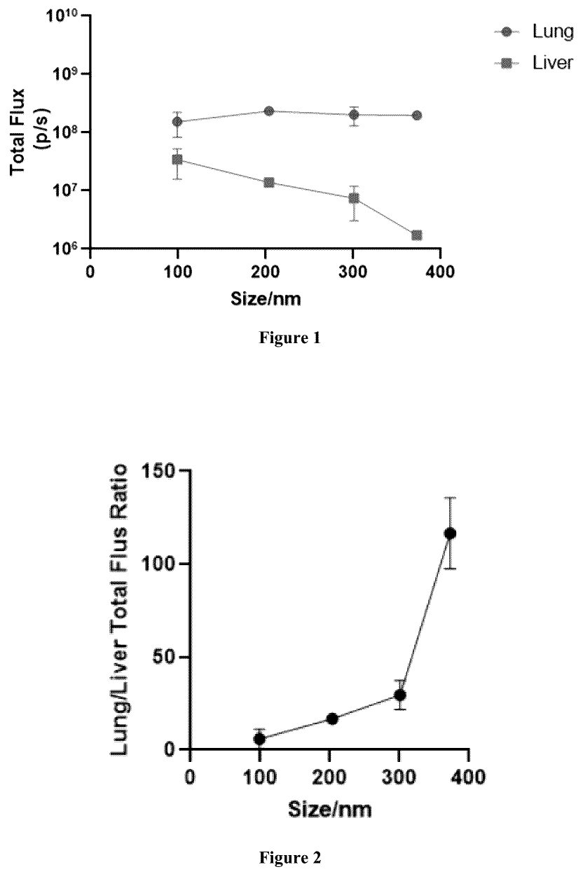

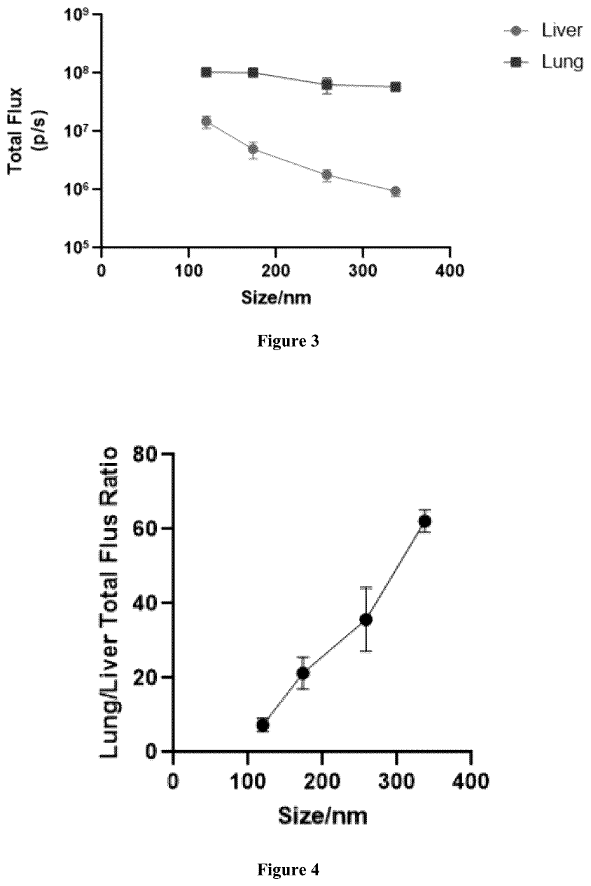

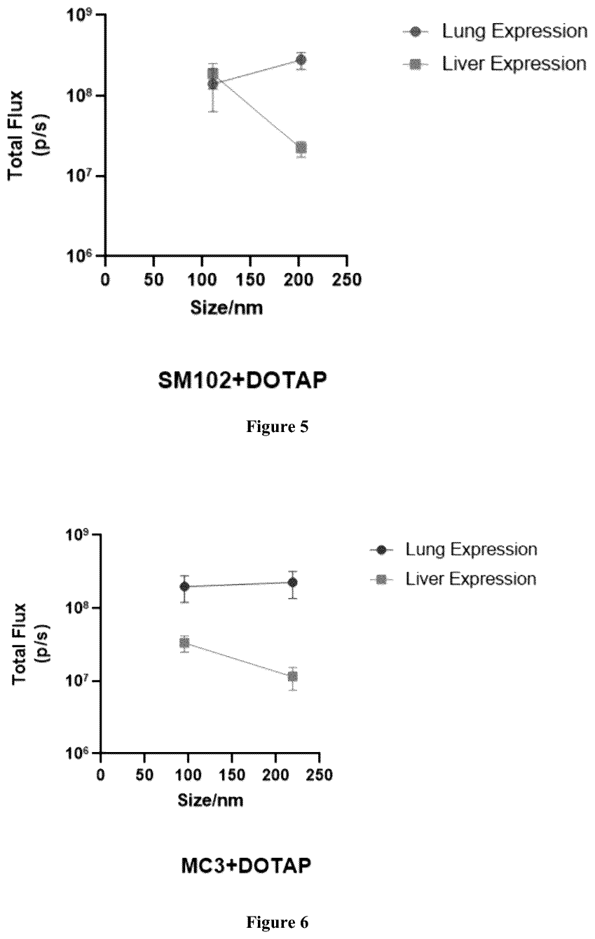

The results show that with the increase of LNP particle size, the expression level of protein (luciferase) in the liver decreased significantly, while the expression level of fluorescent protein in the lungs remain almost unchanged (FIG. 1 and FIG. 3). With the increase of LNP particle size, the ratio of protein expression in the lung to that in the liver in both the DOTAP group (Formulation 1) and the DOTMA group (Formulation 2) significantly increased, exceeding 100-fold and 60-fold, respectively (FIGS. 2 and 4). The effect of LNP size on lung/liver expression ratio is summarized in Table 8. As shown in Table 8, for LNPs of the same composition, LNPs of larger particle size exhibited better lung-specific delivery or expression of therapeutic agents, such as mRNA.

TABLE 8

| | | | | | Ratio of |
|---|---|---|---|---|---|
| | | Permanently | | | luminescence |
| Formu- | | cationic | Ionizable | | in lung |
| lation | Entry | lipid | Lipid | Diameter (nm) | and liver |
| 1 | 1-1 | DOTAP | Compound | 98.74 ± 0.52 | 5.93 |
| | | | 46 | | |
| | 1-2 | DOTAP | Compound | 203.63 ± 5.44 | 16.85 |
| | | | 46 | | |
| | 1-3 | DOTAP | Compound | 300.67 ± 10.49 | 29.55 |
| | | | 46 | | |
| | 1-4 | DOTAP | Compound | 372.80 ± 29.38 | 116.5 |
| | | | 46 | | |
| 2 | 2-1 | DOTMA | Compound | 119.60 ± 1.55 | 7.30 |
| | | | 46 | | |
| | 2-2 | DOTMA | Compound | 173.5 ± 12.83 | 15.67 |
| | | | 46 | | |
| | 2-3 | DOTMA | Compound | 258.07 ± 18.13 | 35.70 |
| | | | 46 | | |
| | 2-4 | DOTMA | Compound | 337.23 ± 99.68 | 62.15 |
| | | | 46 | | |
| 3 | 3-1 | DOTAP | SM102 | 110.63 ± 2.93 | 0.72 |
| | 3-2 | DOTAP | SM102 | 202.13 ± 2.15 | 13.20 |
| 4 | 4-1 | DOTAP | MC3 | 95.38 ± 1.23 | 6.61 |
| | 4-2 | DOTAP | MC3 | 219.03 ± 0.49 | 20.76 |

### Example 7A: Study on Lung Targeting of siRNA/LNP with Different Particle Sizes

The delivery effect of LNP loaded with Cy3 fluorescently labeled siRNA in mice with different particle sizes and the effect of particle size on lung-specific expression are evaluated. Formulation of the LNPs used is the same as Formulation 1 in Table 6A, the preparation process is the same as described in Example 5A, and the characterization method of the obtained LNP is described in Example 2. The size range of LNP is from about 100 nm to about 300 nm. The physical properties of the LNPs encapsulating Cy3-siRNA (sequence shown below) are characterized in Table 9.

Cy3-siRNA sequence:
Sense
5′-UUCUCCGAACGUGUGUCACGUTT-3′
antisense,
5′-ACGUGACACGUUCGGAGAATT-3′

The test mice were SPF grade C57BL/6 mice, female, 6-8 weeks old, weighing 18-22 g, purchased from Beijing Speifu Biotechnology Co., Ltd. All animals were fed adaptively for more than 7 days before the experiment. During the experiment, they had free access to food and water, 12/12 hours of light and dark alternately, the indoor temperature was 20-26° C., and the humidity was 40-70%. The above-prepared lipid nanoparticles loaded with Cy3 fluorescently labeled siRNA were injected into the mice with a single dose of 1 mg/kg mRNA by tail vein injection, and the mice were dissected 2 hours after the administration to take out the lungs and liver, the fluorescence distribution of the mouse organs was detected with a small animal in vivo imaging system (IVIS LUMINA III, purchased from PerkinElmer). Data collection and data analysis were performed on the lungs and liver.

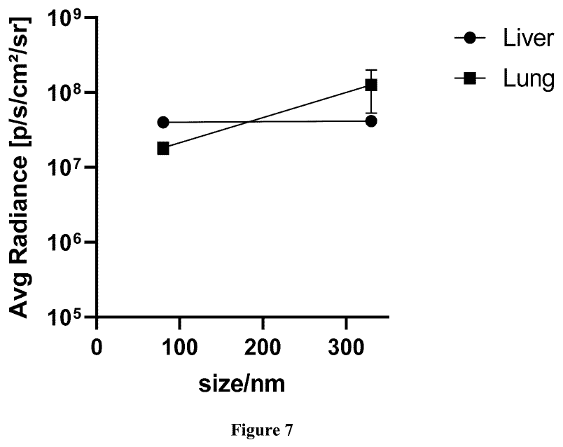

The experimental results showed that with the increase of LNP particle size, the amount of Cy3 fluorescence signal distributed in the lung increased significantly, while the distribution of Cy3 fluorescence signal in the liver remained almost unchanged (FIG. 7). As shown in Table 9, The lung/liver fluorescence expression ratio of about 80 nm LNP is 0.62. As the size of LNPs increased to about 330 nm, the Lung/Liver ratio is increased to 3.04, which is about 4.8 times higher than LNPs of smaller particle size. The above data shows that under the same formulation, the delivery efficiency of siRNA in the lungs by large particle size LNP is better than the LNP of smaller size.

TABLE 9

| | | | | | Ratio of |
|---|---|---|---|---|---|
| | | Permanently | | | luminescence |
| Formu- | | Cationic | Ionizable | Diameter | in lung |
| lation | Entry | Lipid | Lipid | (nm) | and liver |
| 5 | 5-1 | DOTAP | Compound | 79.67 ± 1.04 | 0.62 |
| | | | 46 | | |
| | 5-2 | DOTAP | Compound | 330.17 ± 1.64 | 3.04 |
| | | | 46 | | |

What is claimed is:

1. A lipid nanoparticle for use in delivering or expressing a therapeutic agent in the lung of a subject, wherein the lipid nanoparticle is administered intravenously, intraarterially, or intraperitoneally to the subject, wherein the lipid nanoparticle has a positive surface charge, and wherein the lipid nanoparticle has a diameter of from about 160 nm to about 900 nm.

2. The lipid nanoparticle for use of claim 1, wherein the lipid nanoparticle comprises a permanently cationic lipid and an ionizable lipid.

3. A lipid nanoparticle for use in delivering or expressing a therapeutic agent in the lung of a subject, wherein the lipid nanoparticle comprises a permanently cationic lipid and an ionizable lipid, and wherein the lipid nanoparticle has a diameter of from about 160 nm to about 900 nm.

4. The lipid nanoparticle for use of any one of claims 1 to 3, wherein the lipid nanoparticle has a diameter of from 180 nm to about 900 nm, from about 300 nm to about 900 nm, from about 180 nm to about 600 nm, from about 180 nm to about 400 nm, from about 180 nm to about 350 nm, or from about 180 nm to about 300 nm; optionally wherein the lipid nanoparticle has a diameter of from about 180 nm to about 300 nm.

5. The lipid nanoparticle for use of any one of claims 1 to 4, wherein the lipid nanoparticle has:
(i) a greater than neutral zeta potential at physiologic pH, or
(ii) a zeta potential of from about 0 mV to about 25 mV, from about 0 mV to about 20 mV, or from about 2 mV to about 15 mV.

6. The lipid nanoparticle for use of any one of claims 2 to 5, wherein the amount of the permanently cationic lipid is from about 15 mol % to about 90 mol %, from about 20 mol % to about 80 mol %, from about 30 mol % to about 70 mol %, from about 40 mol % to about 60 mol %, or from about 45 mol % to about 55 mol % of the total lipid present in the lipid nanoparticle.

7. The lipid nanoparticle for use of any one of claims 2 to 6, wherein the permanently cationic lipid has a pKa of greater than about 10, or greater than about 13.

8. The lipid nanoparticle for use of any one of claims 2 to 7, wherein the permanently cationic lipid comprises a quaternary ammonium group.

9. The lipid nanoparticle for use of any one of claims 2 to 8, wherein the permanently cationic lipid is a compound of formula (I): 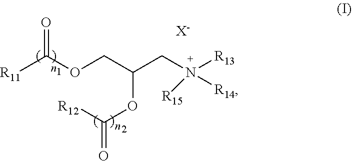
or a stereoisomer, a mixture of stereoisomers, or a pharmaceutically acceptable salt thereof, wherein R₁₁ and R₁₂ are each independently C₆₋₃₀ alkyl, C₆₋₃₀ alkenyl, or C₆₋₃₀ alkynyl, and wherein the alkyl, alkenyl and alkynyl are independently optionally substituted with one or more groups selected from hydroxyl, halogen, cyano, C₁₋₃₀ alkyl, C₁₋₃₀ haloalkyl, C₁₋₃₀ alkoxy, —S—C₁₋₃₀ alkyl, amino, —NH—C₁₋₃₀ alkyl, and —N(C₁₋₃₀ alkyl)₂;
R₁₃, R₁₄, and R₁₅ are each independently C₁₋₆ alkyl, C₁₋₆ haloalkyl, C₂₋₆ alkenyl, C₂₋₆ alkynyl, or any two of R₁₃, R₁₄, and R₁₅ together with the nitrogen atom they are attached to form a 4 to 8-membered ring, and
wherein the alkyl, haloalkyl, alkenyl, alkynyl, and ring are optionally substituted;
X⁻ is an anion; and
n₁ and n₂ are each independently 0 or 1.

10. The lipid nanoparticle for use of claim 9, wherein R₁₁ and R₁₂ are each independently C₁₅₋₂₀ alkyl, C₁₅₋₂₀ alkenyl, or C₁₅₋₂₀ alkynyl, and wherein the alkyl, alkenyl and alkynyl are independently optionally substituted with one or more groups selected from hydroxyl, halogen, cyano, C₁₋₂₀ alkyl, C₁₋₂₀ haloalkyl, C₁₋₂₀ alkoxy, —S—C₁₋₂₀ alkyl, amino, —NH—C₁₋₂₀ alkyl, and —N(C₁₋₂₀ alkyl)₂.

11. The lipid nanoparticle for use of claim 9 or 10, wherein R₁₃, R₁₄, and R₁₅ are each independently C₁₋₆ alkyl optionally substituted with hydroxyl, halogen, cyano, C₁₋₆ alkoxy, —S—C₁₋₆ alkyl, amino, —NH—C₁₋₆ alkyl, or —N(C₁₋₆ alkyl)₂.

12. The lipid nanoparticle for use of any one of claims 2 to 8, wherein the permanently cationic lipid is a compound of formula (II): 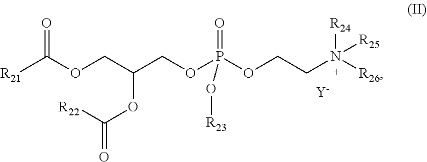
or a stereoisomer, a mixture of stereoisomers, or a pharmaceutically acceptable salt thereof, wherein R₂₁ and R₂₂ are each independently C₆₋₃₀ alkyl, C₆₋₃₀ alkenyl, or C₆₋₃₀ alkynyl, and wherein the alkyl, alkenyl and alkynyl are independently optionally substituted with one or more groups selected from hydroxyl, halogen, cyano, C₁₋₃₀ alkyl, C₁₋₃₀ haloalkyl, C₁₋₃₀ alkoxy, —S—C₁₋₃₀ alkyl, amino, —NH—C₁₋₃₀ alkyl, and —N(C₁₋₃₀ alkyl)₂;
R₂₃ is C₁₋₆ alkyl, C₁₋₆ haloalkyl, C₂₋₆ alkenyl, or C₂₋₆ alkynyl, and wherein R₂₃ is optionally substituted with one or more groups selected from halogen, hydroxyl, C₁₋₆ alkyl, C₁₋₆ haloalkyl, C₁₋₆ alkoxy, —OC(═O)R₂ₐ, —C(═O)OR₂ₐ, —C(═O)NHR₂ₐ, and —NHC(═O)R₂ₐ;
R₂ₐ is hydrogen, C₁₋₆ alkyl, or C₁₋₆ haloalkyl;
R₂₄, R₂₅, and R₂₆ are each independently C₁₋₆ alkyl, C₁₋₆ haloalkyl, C₂₋₆ alkenyl, C₂₋₆ alkynyl, or any two of R₂₄, R₃₅, and R₂₆ together with the nitrogen atom they are attached to form a 4 to 8-membered ring, and wherein the alkyl, haloalkyl, alkenyl, alkynyl, and ring are optionally substituted; and
Y⁻ is an anion.

13. The lipid nanoparticle for use of claim 12, wherein:
(i) R₂₁ and R₂₂ are each independently C₁₀₋₂₅ alkyl, C₁₀₋₂₅ alkenyl, or C₁₀₋₂₅ alkynyl, and wherein the alkyl, alkenyl and alkynyl are independently optionally substituted with one or more groups selected from hydroxyl, halogen, cyano, C₁₋₂₅ alkyl, C₁₋₂₅ haloalkyl, C₁₋₂₅ alkoxy, —S—C₁₋₂₅ alkyl, amino, —NH—C₁₋₂₅ alkyl, and —N(C₁₋₂₅ alkyl)₂;
(ii) R₂₃ is C₁₋₆ alkyl or C₁₋₆ haloalkyl; or
(iii) R₂₄, R₂₅, and R₂₆ are each independently C₁₋₆ alkyl optionally substituted with hydroxyl, halogen, cyano, C₁₋₆ alkoxy, —S—C₁₋₆ alkyl, amino, —NH—C₁₋₆ alkyl, or —N(C₁₋₆ alkyl)₂, or any two of R₂₄, R₂₅, and R₂₆ together with the nitrogen atom they are attached to form a 5 to 6-membered ring.

14. The lipid nanoparticle for use of any one of claims 1 to 8, wherein the permanently cationic lipid is a pharmaceutically acceptable salt of 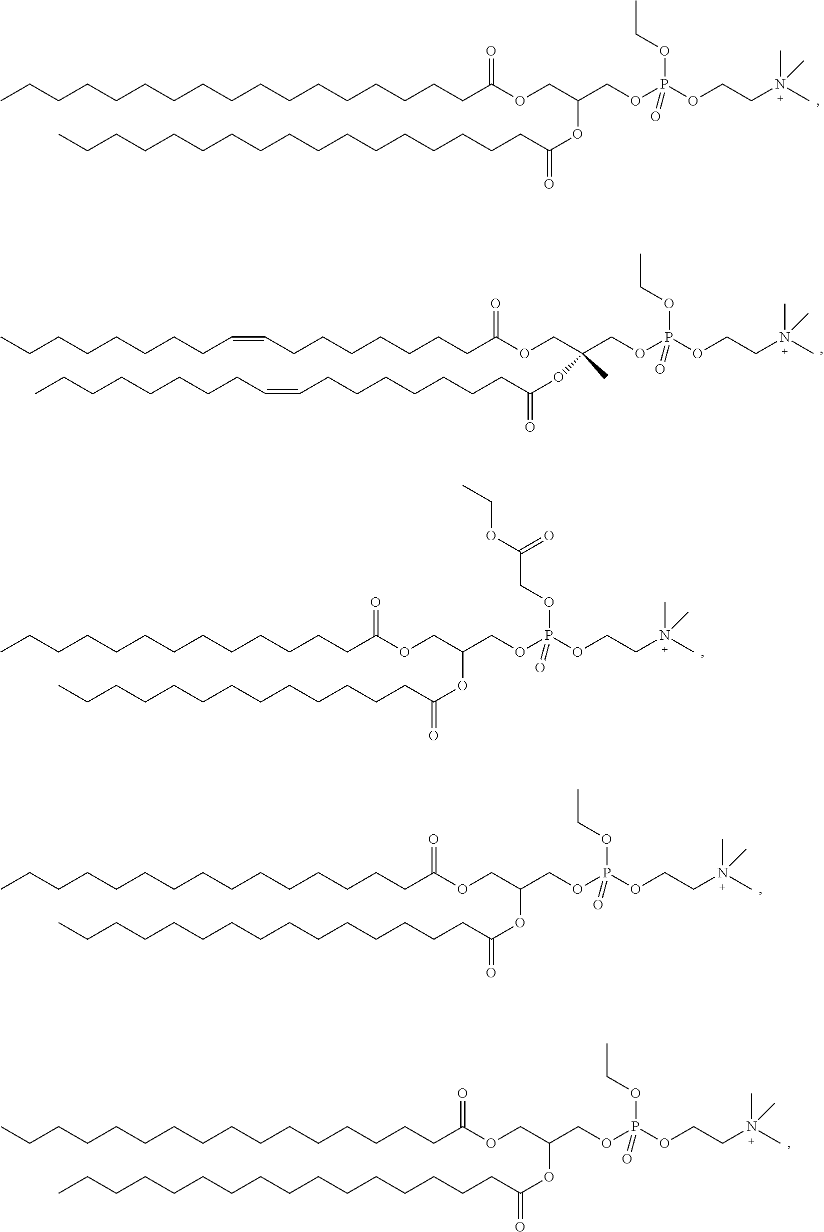 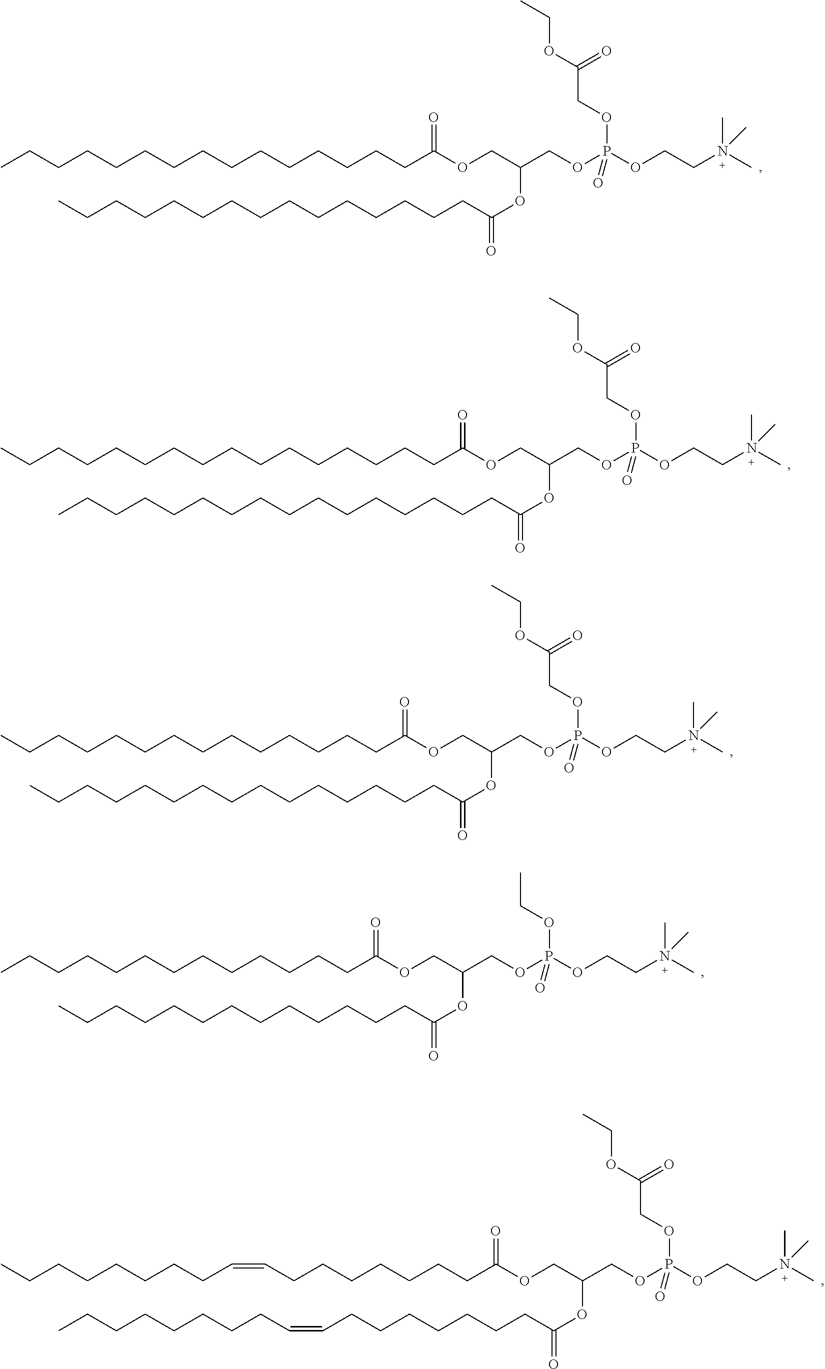 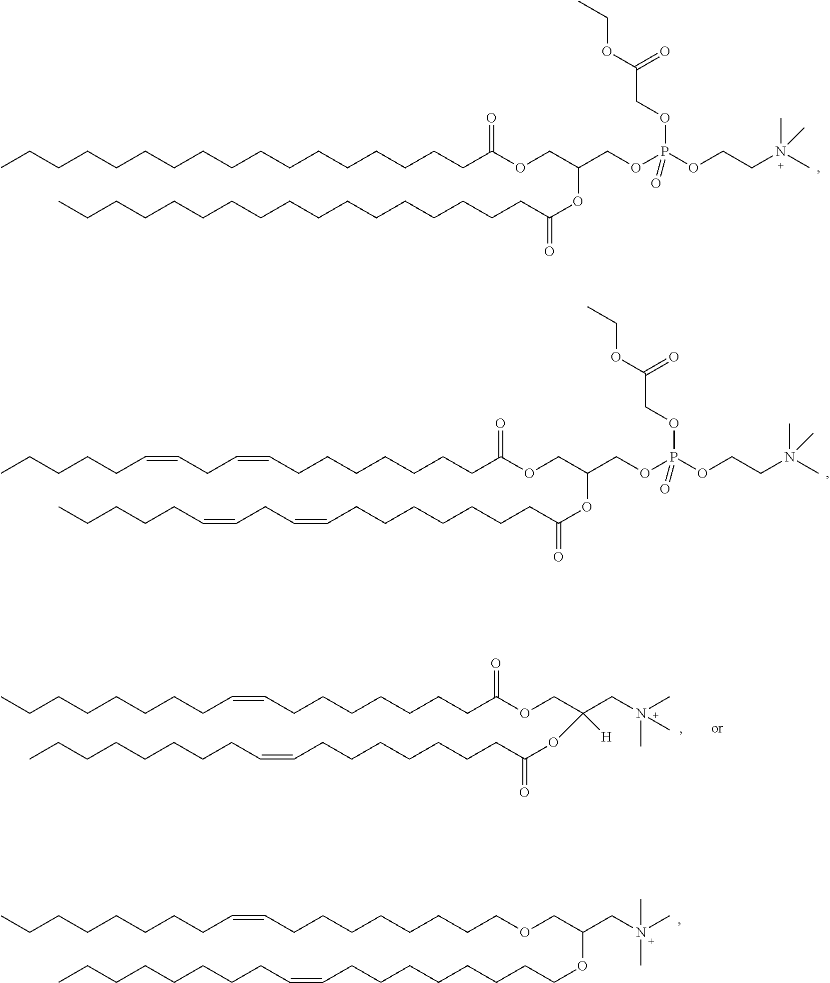
or a stereoisomer, or a mixture of stereoisomers thereof.

15. The lipid nanoparticle for use of any one of claims 1 to 8, wherein the permanently cationic lipid is DOTMA, DOTAP, MVL5, DOGS, DC-Chol, DDAB, EPC, or a mixture thereof.

16. The lipid nanoparticle for use of any one of claims 1 to 15, wherein the amount of the ionizable lipid is from about 15 mol % to about 60 mol %; optionally wherein the amount of the ionizable lipid is from about 15 mol % to about 40 mol %, or from about 20 mol % to about 30 mol % of the total lipid present in the lipid nanoparticle.

17. The lipid nanoparticle for use of any one of claims 1 to 16, wherein:
(i) the amount of the permanently cationic lipid is from about 15 mol % to about 90 mol % of the total lipid present in the lipid nanoparticle, and the amount of the ionizable lipid is from about 15 mol % to about 60 mol % of the total lipid present in the lipid nanoparticle;
(ii) the amount of the permanently cationic lipid is from about 40 mol % to about 60 mol % of the total lipid present in the lipid nanoparticle, and the amount of the ionizable lipid is from about 15 mol % to about 40 mol % of the total lipid present in the lipid nanoparticle; or
(iii) the amount of the permanently cationic lipid is from about 45 mol % to about 55 mol % of the total lipid present in the lipid nanoparticle, and the amount of the ionizable lipid is from about 20 mol % to about 30 mol % of the total lipid present in the lipid nanoparticle.

18. The lipid nanoparticle for use of any one of claims 1 to 17, wherein the ionizable lipid has a pKa of from about 7 to about 13, from about 7 to about 11, or from about 7 to about 9.

19. The lipid nanoparticle for use of any one of claims 1 to 18, wherein the lipid nanoparticle further comprises a phospholipid; optionally wherein the phospholipid is DSPC, DMPC, DOPC, DPPC, POPC, DOPE, DMPE, POPOE, or DPPE, or a mixture thereof.

20. The lipid nanoparticle for use of any one of claims 1 to 18, wherein the lipid nanoparticle does not comprise a phospholipid or comprises a phospholipid in an amount less than about 15 mol %, less than about 10 mol %, less than about 8 mol %, less than about 5 mol %, less than about 3 mol %, or less than about 1 mol % of the total lipid present in the lipid nanoparticle.

21. The lipid nanoparticle for use of any one of claims 1 to 20, wherein the lipid nanoparticle further comprises a steroid, optionally wherein the steroid is cholesterol, campesterol, stigmasterol, sitosterol, brassicasterol, ergosterol, solanine, ursolic acid, alpha-tocopherol, beta-sitosterol, avenasterol, calciferol, or canola sterol.

22. The lipid nanoparticle for use of claim 21, wherein the amount of the steroid is from about 5 mol % to about 60 mol %, from about 10 mol % to about 50 mol %, from about 10 mol % to about 40 mol %, from about 20 mol % to about 30 mol %, or about 25 mol % of the total lipid present in the lipid nanoparticle.

23. The lipid nanoparticle for use of any one of claims 1 to 22, wherein the lipid nanoparticle further comprises a pegylated lipid, optionally wherein:
(i) a pegylated moiety of the pegylated lipid has a molecule weight of from about 1000 Da to about 10,000 Da, from about 1000 Da to about 5000 Da, or from about 1000 Da to about 2000 Da;
(ii) the pegylated lipid is ALC-0159, DMG-PEG2000, DMPE-PEG1000, DPPE-PEG1000, DSPE-PEG1000, DOPE-PEG1000, Ceramide-PEG2000, DMPE-PEG2000, DPPE-PEG2000, DSPE-PEG2000, DSPE-PEG2000-Mannose, Ceramide-PEG5000, DSPE-PEG5000, or DSPE-PEG2000 amine; and/or
(iii) the amount of the pegylated lipid is from about 0.1 mol to about 5 mol %, from about 0.1 mol to about 3 mol %, from about 0.25 mol to about 2 mol %, from about 0.5 mol to about 1.5 mol %, or about 1 mol % of the total lipid present in the lipid nanoparticle.

24. The lipid nanoparticle for use of any one of claims 1 to 23, wherein:
(i) the lipid nanoparticle comprises a permanently cationic lipid in an amount from about 15 mol % to about 90 mol % of the total lipid present in the lipid nanoparticle, an ionizable lipid in an amount from about 15 mol % to about 60 mol % of the total lipid present in the lipid nanoparticle, a steroid in an amount from about 5 mol % to about 60 mol % of the total lipid present in the lipid nanoparticle, and a pegylated lipid in an amount from about 0.1 mol % to about 5 mol % of the total lipid present in the lipid nanoparticle;
(ii) the lipid nanoparticle comprises a permanently cationic lipid in an amount from about 30 mol % to about 70 mol % of the total lipid present in the lipid nanoparticle, an ionizable lipid in an amount from about 15 mol % to about 40 mol % of the total lipid present in the lipid nanoparticle, a steroid in an amount from about 15 mol % to about 40 mol % of the total lipid present in the lipid nanoparticle, and a pegylated lipid in an amount from about 0.25 mol % to about 3 mol % of the total lipid present in the lipid nanoparticle; or
(iii) the lipid nanoparticle comprises a permanently cationic lipid in an amount from about 45 mol % to about 55 mol % of the total lipid present in the lipid nanoparticle, an ionizable lipid in an amount from about 20 mol % to about 30 mol % of the total lipid present in the lipid nanoparticle, a steroid in an amount from about 20 mol % to about 30 mol % of the total lipid present in the lipid nanoparticle, and a pegylated lipid in an amount from about 0.5 mol % to about 1.5 mol % of the total lipid present in the lipid nanoparticle.

25. The lipid nanoparticle for use of any one of claims 1 to 24, wherein the therapeutic agent is nucleic acid, optionally wherein the nucleic acid is antisense oligonucleotide (ASO), DNA, or RNA, optionally wherein the RNA is RNA interference (RNAi), small interfering RNA (siRNA), short hairpin RNA (shRNA), antisense RNA (aRNA), messenger RNA (mRNA), modified messenger RNA (mmRNA), long noncoding RNA (lncRNA), microRNA (miRNA), small activating RNA (saRNA), multicoding nucleic acid (MCNA), polymer-coded nucleic acid (PCNA), guide RNA (gRNA), CRISPR RNA (crRNA), or any other RNA in the ribozyme.

26. The lipid nanoparticle for use of claim 25, wherein the ratio of total number of nitrogen atoms in the permanently cationic lipid and ionizable lipid and total number of phosphate atoms in the nucleic acid is from about 1:1 to about 20:1, about 1:1 to about 15:1, from about 3:1 to about 12:1, or from about 4:1 to about 9:1.

27. The lipid nanoparticle for use of any one of claims 1 to 26, wherein the lipid nanoparticle has an apparent pKa of greater than about 7, greater than about 8, greater than about 9, greater than about 10, from about 7 to about 10, or greater than about 10.

28. The lipid nanoparticle for use of any one of claims 1 to 27, wherein the amount of the therapeutic agent delivered or expressed in the lung of the subject is higher than the amount of the therapeutic agent delivered or expressed in the liver of the subject, optionally wherein the amount of the therapeutic agent delivered or expressed in the lung of the subject is at least 1 time, at least 2 times, at least 3 times, at least 4 times, at least 5 times, at least 10 times, at least 20 times, at least 40 times, at least 60 times, or at least 100 times higher than the amount of the therapeutic agent delivered or expressed in the liver of the subject.

29. The lipid nanoparticle for use of any one of claims 1 to 28, wherein the subject has a lung disease.

30. A lipid nanoparticle comprising:
(i) a permanently cationic lipid in an amount from about 15 mol % to about 90 mol % of the total lipid present in the lipid nanoparticle; and
(ii) an ionizable lipid in an amount from about 15 mol % to about 60 mol % of the total lipid present in the lipid nanoparticle;
wherein the lipid nanoparticle has a diameter of from about 160 nm to about 900 nm; and
wherein the permanently cationic lipid is a compound of formula (I) or (II); 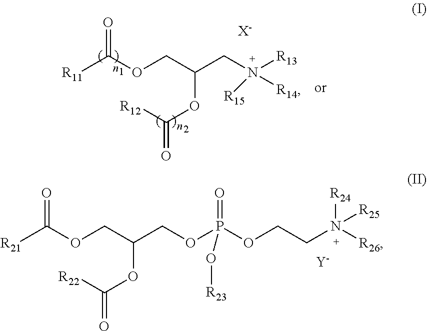
or a stereoisomer, a mixture of stereoisomers, or a pharmaceutically acceptable salt thereof, wherein:
R₁₁, R₁₂, R₂₁ and R₂₂ are each independently C₆₋₃₀ alkyl, C₆₋₃₀ alkenyl, or C₆₋₃₀ alkynyl, and wherein the alkyl, alkenyl and alkynyl are independently optionally substituted with one or more groups selected from hydroxyl, halogen, cyano, C₁₋₃₀ alkyl, C₁₋₃₀ haloalkyl, C₁₋₃₀ alkoxy, —S—C₁₋₃₀ alkyl, amino, —NH—C₁₋₃₀ alkyl, and —N(C₁₋₃₀ alkyl)₂;
R₁₃, R₁₄, and R₁₅ are each independently C₁₋₆ alkyl, C₁₋₆ haloalkyl, C₂₋₆ alkenyl, C₂₋₆ alkynyl, or any two of R₁₃, R₁₄, and R₁₅ together with the nitrogen atom they are attached to form a 4 to 8-membered ring, and wherein the alkyl, haloalkyl, alkenyl, alkynyl, and ring are optionally substituted;
R₂₄, R₂₅, and R₂₆ are each independently C₁₋₆ alkyl, C₁₋₆ haloalkyl, C₂₋₆ alkenyl, C₂₋₆ alkynyl, or any two of R₂₄, R₃₅, and R₂₆ together with the nitrogen atom they are attached to form a 4 to 8-membered ring, and wherein the alkyl, haloalkyl, alkenyl, alkynyl, and ring are optionally substituted;
R₂₃ is C₁₋₆ alkyl, C₁₋₆ haloalkyl, C₂₋₆ alkenyl, or C₂₋₆ alkynyl, and wherein R₂₃ is optionally substituted with one or more groups selected from halogen, hydroxyl, C₁₋₆ alkyl, C₁₋₆ haloalkyl, C₁₋₆ alkoxy, —OC(═O)R₂ₐ, —C(═O)OR₂ₐ, —C(═O)NHR₂ₐ, and —NHC(═O)R₂ₐ;
R₂ₐ is hydrogen, C₁₋₆ alkyl, or C₁₋₆ haloalkyl;
X⁻ and Y⁻ are each independently an anion; and
n₁ and n₂ are each independently 0 or 1.

31. A population of lipid nanoparticles comprising the lipid nanoparticle of claim 30, wherein the population of lipid nanoparticles have an average diameter of from about 160 nm to about 900 nm, optionally wherein the average diameter is determined by dynamic light scattering (DLS).

32. A pharmaceutical composition comprising the lipid nanoparticle of claim 30 or the population of lipid nanoparticles of claim 31 and a pharmaceutically acceptable carrier.

33. A method of delivering or expressing a therapeutic agent in the lung of a subject or treating or preventing a lung disease in a subject, wherein the method comprises using a lipid nanoparticle comprising the therapeutic agent, wherein the lipid nanoparticle is administered intravenously, intraarterially, or intraperitoneally to the subject, wherein the lipid nanoparticle has a positive surface charge, and wherein the lipid nanoparticle has a diameter of from about 160 nm to about 900 nm.

34. A method of delivering or expressing a therapeutic agent in the lung of a subject or treating or preventing a lung disease in a subject, wherein the method comprises using a lipid nanoparticle comprising the therapeutic agent, wherein the lipid nanoparticle comprises a permanently cationic lipid and an ionizable lipid, and wherein the lipid nanoparticle has a diameter of from about 160 nm to about 900 nm.

35. A method of treating or preventing a lung disease in a subject, comprising administering to the subject a therapeutically effective amount of the lipid nanoparticle of claim 30, the population of lipid nanoparticles of claim 31, or the pharmaceutical composition of claim 32.

36. The method of claim 35, wherein the administration is intravenous administration, intraarterial administration, or intraperitoneal administration.

37. A method of producing the lipid nanoparticle of claim 30 or the population of lipid nanoparticles of claim 31 comprising the steps of:
(i) dissolving in a first solution a mixture comprising a permanently cationic lipid and an ionizable lipid to form a lipid solution, wherein the lipid solution is formed in an organic solvent, optionally wherein the organic solvent is ethanol;
(ii) dissolving in a second solution a therapeutic agent to form a therapeutic agent solution, optionally wherein the second solution is sodium acetate buffer having a pH of about 4.5; and
(iii) mixing the lipid solution and the therapeutic agent solution at a mixing speed of about 1 mL/min to about 18 mL/min, about 1 mL/min to about 10 mL/min, or about 2 mL/min to about 6 mL/min, optionally wherein the lipid solution and the therapeutic agent solution are mixed at a volumetric ratio of from about 1:1 to about 1:10, from about 1:1 to about 1:6, or from about 1:1 to about 1:4.
